(19) United States
(12) Patent Application Publication
(10) Pub. No.: US 2025/0213527 A1
(43) Pub. Date: Jul. 3, 2025

(54) FORMULATIONS OF PSILOCYBIN ANALOGS AND METHODS OF USE

(71) Applicant: Cybin IRL Limited, Dublin (IE)

(72) Inventors: Alex Nivorozhkin, West Roxbury, MA (US); Michael Palfreyman, St. Petersburg, FL (US); Pradip M. Pathare, Lexington, MA (US); Kenneth L. Avery, Merrimack, NH (US); Mohammed I. Shukoor, Lexington, MA (US); James He Huang, Parkland, FL (US); Michael E. Morgan, San Diego, CA (US); Joan M. Krakowsky, North Palm Beach, FL (US)

(21) Appl. No.: 18/707,825

(22) PCT Filed: Sep. 20, 2022
(86) PCT No.: PCT/EP2022/076073

§ 371 Date: May 6, 2024

Related U.S. Application Data
(60) Provisional application No. 63/276,117, filed on Nov. 5, 2021.

(30) Foreign Application Priority Data
Mar. 17, 2022 (WO) PCT/EP2022/056991

(51) Int. Cl. A61K 31/4045; A61K 9/20; A61K 47/12

(52) U.S. Cl. CPC A61K 31/4045; A61K 9/2063; A61K 47/12

(57) ABSTRACT

The present disclosure relates to pharmaceutical compositions formulated with psilocin and/or deuterated psilocin compounds or pharmaceutically acceptable salts, polymorphs, stereoisomers, or solvates thereof, and an organic acid agent as a pharmaceutically acceptable vehicle. Uses in the treatment of diseases, such as those associated, with a 5-HT₂ receptor, are also disclosed.

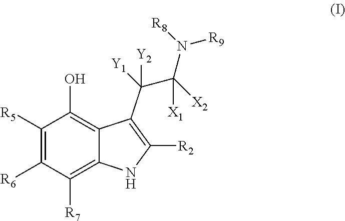

### CROSS REFERENCE TO RELATED APPLICATIONS

This application claims the benefit of U.S. Provisional Application No. 63/276,117 filed Nov. 5, 2021, and Patent Cooperation Treaty Application No. PCT/EP/2022/056991, filed Mar. 17, 2022, each incorporated herein by reference in its entirety.

### FIELD

The present disclosure relates generally to psilocin compounds and pharmaceutically acceptable salts, polymorphs, stereoisomers, or solvates thereof, compositions, and, in some embodiments, to serotonin 5-HT₂ receptor agonists and uses in the treatment of diseases associated with a 5-HT₂ receptor.

### BACKGROUND

Psilocybin (PY) and psilocin (PI) are tryptamine alkaloids and structural analogs of the neurotransmitter serotonin. Psilocybin is a prodrug of psilocin. That is, when consumed, psilocybin is rapidly metabolized into the active form, psilocin (4-hydroxy-N,N-dimethyltryptamine). Specifically, a chemical process called dephosphorylation removes the phosphate group on psilocybin, creating psilocin.

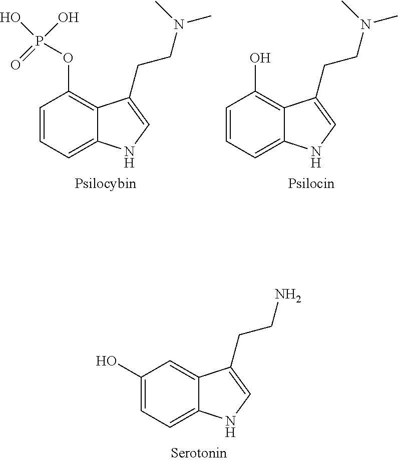 [SMILES: CN(C)CCc1c[nH]c2cccc(OP(=O)(O)O)c12 ; CN(C)CCc1c[nH]c2cccc(O)c12 ; NCCc1c[nH]c2ccc(O)cc12]

Outside the body, psilocin is reported to be a short-lived and unstable molecule. For this reason, psilocin has been rarely studied and not generally recognized as a viable therapeutic option. Vaupel et al. studied the effects of psilocin ascorbate on food intake on dogs (D. B. Vaupel, M. Nozaki, W. R. Martin, L. D. Bright, E. C. Morton, The inhibition of food intake in the dog by LSD, mescaline, psilocin, d-amphetamine and phenylisopropylamine derivatives, Life Sciences, Volume 24, Issue 26, 1979, 2427-2431).

Migliaccio et al. studied the solution confirmation of psilocin monooxalate in water (Gerald P. Migliaccio, Tiee-Leou N. Shieh, Stephen R. Byrn, Bruce A. Hathaway, and David E. Nichols, Comparison of solution conformational preferences for the hallucinogens bufotenin and psilocin using 360-MHz proton NMR spectroscopy, Journal of Medicinal Chemistry, 1981 24, 2, 206-209).

Aghajanian et al. studied the effects of psilocin tartrate on serotonergic neurons in rats using microiontophoretic techniques (Aghajanian G K, Hailgler H J. Hallucinogenic indoleamines: Preferential action upon presynaptic serotonin receptors. Psychopharmacol Commun. 1975, 1, 6, 619-29).

Kuhnert-Brandstatter et al. describe the preparation of three polymorphs of psilocin (Kuhnert, M. et al., Polymorphe Modifikationen und Solvate von Psilocin und Psilocybin [Polymorphic Modifications and Solvates of Psilocin and Psilocybin], 1976, Archiv der Pharmazie, 309:625-631).

U.S. Pat. No. 11,312,684 B1 describes psilocin salts with improved physical properties and handling characteristics.

Therefore, therapeutic applications involving the use of psilocin are generally accomplished by administration of the precursor, psilocybin, or other prodrug approaches. However, psilocybin has slow onset and a long duration of drug action, often requiring 7-8 hours of supervised clinical observation of a patient before discharge. Psilocybin is also associated with high levels of variability in delivery as it requires metabolism to release the active. Therefore, there is a need for a stabilized psilocin, that does not rely on breakdown of a prodrug to provide pharmacologically active drug, that offers less variability in drug exposure, a faster/quicker therapeutic onset, and a shorter duration of drug action (i.e., shorter duration of therapeutic effect) than psilocybin.

### SUMMARY

The present disclosure is based at least in part on the identification of novel stabilized forms of psilocin and deuterated psilocin, including novel polymorphs of psilocin/deuterated psilocin, novel salt forms of psilocin/deuterated psilocin and their polymorphs, as well as compositions thereof, such as those which provide a fast therapeutic onset, a shortened duration of drug action, and less variability in drug exposure (e.g., compared to psilocybin or other prodrug approaches), and methods of using the same to treat diseases associated with a serotonin 5-HT₂ receptor. More specifically, the present disclosure provides stabilized forms of psilocin and deuterated psilocin and compositions thereof, that can be used to treat neuropsychiatric disorders, central nervous system (CNS) disorders, and other disorders, such as those associated with inflammation, for example, through various dosing regimens (e.g., once, once-daily, once-weekly, sub-psychedelic dosing, etc.) to selectively engage 5-HT_{2A}Rs without producing psychedelic side effects.

The disclosed stabilized forms of psilocin and deuterated psilocin do not rely on prodrug metabolism for release of active agent, as is the case with psilocybin administration or related prodrug approaches, and thus can provide a faster/quicker therapeutic onset, a shorter duration of drug action (i.e., short duration of therapeutic effect), and less inter-subject variability. Instead, the inventors have identified dosage forms which provide rapid release of psilocin and deuterated psilocin, in stabilized form, and with fast and reliable onset characteristics, including intraoral dosage forms which allow for pre-gastric absorption of the compounds herein, e.g., when administered through the mucosal linings of the oral cavity.

Thus, the present disclosure provides:

(1) A pharmaceutical composition, comprising:
a compound of Formula (I), or a pharmaceutically acceptable salt, polymorph, stereoisomer, or solvate thereof; and
a pharmaceutically acceptable vehicle comprising an organic acid agent,

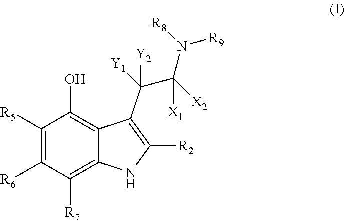

wherein:
R₂, R₅, R₆, and R₇ are independently selected from the group consisting of hydrogen and deuterium,
R₈ and R₉ are independently selected from the group consisting of —CH₃, —CH₂D, —CHD₂, and —CD₃, and
X₁, X₂, Y₁, and Y₂ are independently selected from the group consisting of hydrogen and deuterium.

(2) The pharmaceutical composition of (1), wherein R₂, R₅, R₆, and R₇ are hydrogen.

(3) The pharmaceutical composition of (1), wherein at least one of R₂, R₅, R₆, and R₇ is deuterium.

(4) The pharmaceutical composition of any one of (1) to (3), wherein R₈ and R₉ are —CH₃.

(5) The pharmaceutical composition of any one of (1) to (3), wherein R₈ and R₉ are —CD₃.

(6) The pharmaceutical composition of any one of (1) to (5), wherein X₁, X₂, Y₁, and Y₂ are deuterium.

(7) The pharmaceutical composition of any one of (1) to (6), wherein X₁ and X₂ are deuterium.

(8) The pharmaceutical composition of any one of (1) to (7), wherein Y₁ and Y₂ are deuterium.

(9) The pharmaceutical composition of any one of (1) to (5) or (7), wherein Y₁ and Y₂ are hydrogen.

(10) The pharmaceutical composition of any one of (1) to (9), wherein the compound of Formula (I) is at least one selected from the group consisting of:

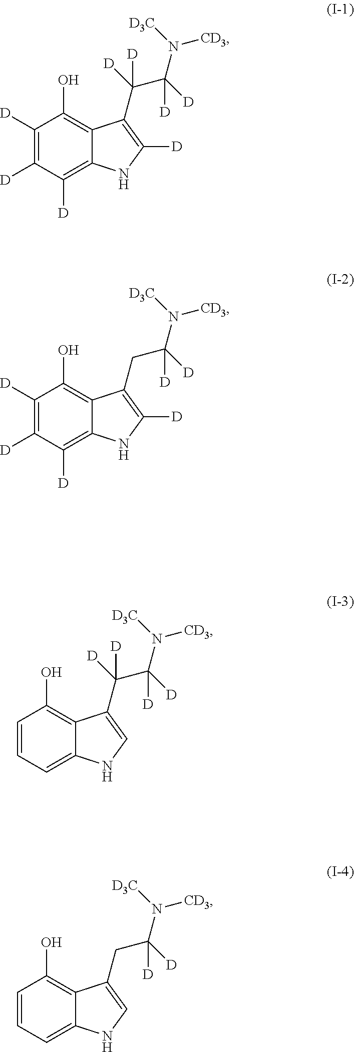 [SMILES: [2H]c1[nH]c2c([2H])c([2H])c([2H])c(O)c2c1C([2H])([2H])C([2H])([2H])N(C([2H])([2H])[2H])C([2H])([2H])[2H] ; [2H]c1[nH]c2c([2H])c([2H])c([2H])c(O)c2c1CC([2H])([2H])N(C([2H])([2H])[2H])C([2H])([2H])[2H] ; [2H]C([2H])([2H])N(C([2H])([2H])[2H])C([2H])([2H])C([2H])([2H])c1c[nH]c2cccc(O)c12 ; [2H]C([2H])([2H])N(C([2H])([2H])[2H])C([2H])([2H])Cc1c[nH]c2cccc(O)c12]

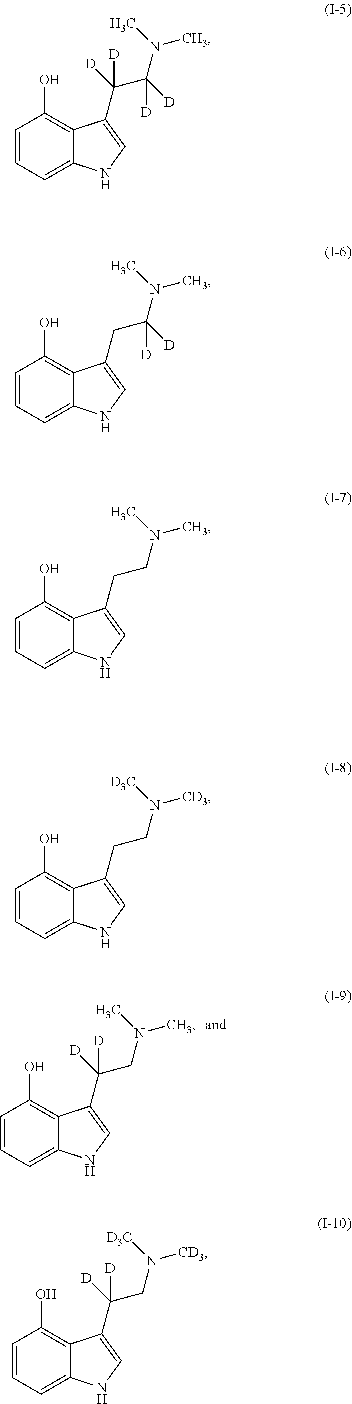 [SMILES: [2H]C([2H])(c1c[nH]c2cccc(O)c12)C([2H])([2H])N(C)C ; [2H]C([2H])(Cc1c[nH]c2cccc(O)c12)N(C)C ; CN(C)CCc1c[nH]c2cccc(O)c12 ; [2H]C([2H])([2H])N(CCc1c[nH]c2cccc(O)c12)C([2H])([2H])[2H] ; [2H]C([2H])(CN(C)C)c1c[nH]c2cccc(O)c12 ; [2H]C([2H])(CN(C([2H])([2H])[2H])C([2H])([2H])[2H])c1c[nH]c2cccc(O)c12]

or a pharmaceutically acceptable salt, polymorph, stereoisomer, or solvate thereof.

(11) The pharmaceutical composition of (1), wherein the compound of Formula (I) is

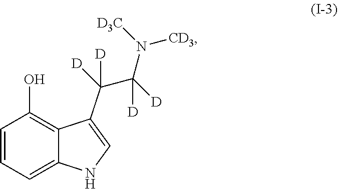 [SMILES: [2H]C([2H])([2H])N(C([2H])([2H])[2H])C([2H])([2H])C([2H])([2H])c1c[nH]c2cccc(O)c12]

or a pharmaceutically acceptable salt, polymorph, stereoisomer, or solvate thereof.

(12) The pharmaceutical composition of (1), wherein the compound of Formula (I) is

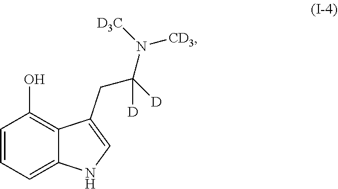 [SMILES: [2H]C([2H])([2H])N(C([2H])([2H])[2H])C([2H])([2H])Cc1c[nH]c2cccc(O)c12]

or a pharmaceutically acceptable salt, polymorph, stereoisomer, or solvate thereof.

(13) The pharmaceutical composition of (1), wherein the compound of Formula (I) is

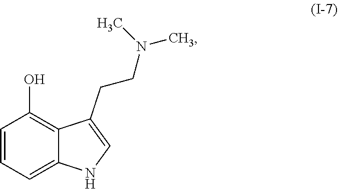

or a pharmaceutically acceptable salt, polymorph, stereoisomer, or solvate thereof.

(14) The pharmaceutical composition of (11), wherein the compound of Formula (I) is a crystalline form of 3-(2-(bis(methyl-d₃)amino)ethyl-1,1,2,2-d₄)-1H-indol-4-ol (I-3), as determined by X-ray powder diffraction.

(15) The pharmaceutical composition of (14), wherein the crystalline form of 3-(2-(bis(methyl-d₃)amino)ethyl-1,1,2,2-d₄)-1H-indol-4-ol (I-3) is characterized by an X-ray powder diffraction pattern containing at least three characteristic peaks at diffraction angles (2θ±0.2°) selected from 7.582°, 8.395°, 9.647°, 10.444°, 11.319°, 12.614°, 13.372°, 14.222°, 15.157°, 16.524°, 16.787°, 17.693°, 19.468°, 19.699°, 20.901°, 21.132°, 21.859°, 22.547°, 23.699°, 24.630°, 25.034°, 25.264°, 26.867°, 27.399°, 27.929°, 28.219°, 28.871°, 29.430°, 30.120°, 30.675°, 31.373°, 32.365°, 33.880°, 34.418°, 34.792°, 35.884°, 36.254°, 37.156°, 38.200°, and 38.417°.

(16) The pharmaceutical composition of (14), wherein the crystalline form of 3-(2-(bis(methyl-d₃)amino)ethyl-1,1,2,2-d₄)-1H-indol-4-ol (I-3) is characterized by an X-ray powder diffraction pattern containing at least three characteristic peaks at diffraction angles (2θ±0.2°) selected from 8.124°, 8.357°, 10.059°, 12.630°, 13.420°, 13.743°, 14.053°, 15.220°, 16.272°, 16.763°, 16.954°, 17.328°, 17.662°, 18.062°, 18.742°, 19.413°, 19.658°, 20.172°, 20.836°, 21.2670, 21.833°, 22.213°, 22.504°, 23.334°, 23.701°, 24.385°, 25.431°, 25.721°, 26.049°, 27.291°, 28.368°, 30.349°, 30.656°, 31.337°, 31.538°, 32.091°, 35.870°, 38.514°, and 41.361°.

(17) The pharmaceutical composition of (13), wherein the compound of Formula (I) is a crystalline form of 3-(2-(dimethylamino)ethyl)-1H-indol-4-ol (I-7), as determined by X-ray powder diffraction.

(18) The pharmaceutical composition of (17), wherein the crystalline form of 3-(2-(dimethylamino)ethyl)-1H-indol-4-ol (I-7) is characterized by an X-ray powder diffraction pattern containing at least three characteristic peaks at diffraction angles (2θ±0.2°) selected from 7.563°, 8.375°, 12.626°, 13.383°, 15.211°, 16.753°, 17.671°, 19.668°, 21.112°, 21.863°, 22.201°, 22.560°, 23.711°, 24.592°, 25.415°, 26.820°, 27.357°, 27.921°, 28.228°, 29.253°, 30.653°, 31.364°, 32.401°, 33.797°, 34.445°, and 39.867°.

(19) The pharmaceutical composition of any one of (1) to (13), wherein the compound of Formula (I) is amorphous as determined by X-ray powder diffraction.

(20) The pharmaceutical composition of (19), wherein the compound of Formula (I) is amorphous as determined by X-ray powder diffraction, and has a glass transition temperature of about 26° C. to about 30° C. as determined by differential scanning calorimetry (DSC).

(21) The pharmaceutical composition of (19) or (20), wherein the compound of Formula (I) in amorphous form is prepared by melting a crystalline form of the compound of Formula (I) to beyond a melting point of the crystalline form, and then rapidly cooling to a glass transition temperature.

(22) The pharmaceutical composition of any one of (19) to (21), wherein the compound of Formula (I) is an amorphous form of 3-(2-(bis(methyl-d₃)amino)ethyl-1,1,2,2-d₄)-1H-indol-4-ol (I-3), as determined by X-ray powder diffraction.

(23) The pharmaceutical composition of any one of (19) to (21), wherein the compound of Formula (I) is an amorphous form of 3-(2-(dimethylamino)ethyl)-1H-indol-4-ol (I-7), as determined by X-ray powder diffraction.

(24) The pharmaceutical composition of any one of (1) to (13), wherein the compound of Formula (I) is present as a pharmaceutically acceptable salt of the compound of Formula (I).

(25) The pharmaceutical composition of (24), wherein the pharmaceutically acceptable salt of the compound of Formula (I) is a benzenesulfonate salt, a tartrate salt, a hemi-fumarate salt, an acetate salt, a citrate salt, a hemi-malonate salt, a fumarate salt, a hemi-succinate salt, an oxalate salt, a benzoate salt, a salicylate salt, an ascorbate salt, a hydrochloride salt, a maleate salt, a malate salt, a methanesulfonate salt, a toluenesulfonate salt, a glucuronate salt, or a glutarate salt of the compound of Formula (I).

(26) The pharmaceutical composition of (24) or (25), wherein the pharmaceutically acceptable salt of the compound of Formula (I) is a benzenesulfonate salt, a tartrate salt, a hemi-fumarate salt, an acetate salt, a citrate salt, a hemi-malonate salt, a fumarate salt, a hemi-succinate salt, an oxalate salt, a benzoate salt, or a salicylate salt of the compound of Formula (I).

(27) The pharmaceutical composition of any one of (24) to (26), wherein the pharmaceutically acceptable salt of the compound of Formula (I) is a benzenesulfonate salt of the compound of Formula (I).

(28) The pharmaceutical composition of (27), wherein the benzenesulfonate salt of the compound of Formula (I) is a benzenesulfonate salt of 3-(2-(bis(methyl-d₃)amino)ethyl-1,1,2,2-d₄)-1H-indol-4-ol (I-3a).

(29) The pharmaceutical composition of (28), wherein the benzenesulfonate salt of 3-(2-(bis(methyl-d₃)amino)ethyl-1,1,2,2-d₄)-1H-indol-4-ol (I-3a) is crystalline and characterized by an X-ray powder diffraction pattern containing at least three characteristic peaks at diffraction angles (2θ±0.2°) selected from 7.023°, 7.767°, 11.822°, 12.550°, 12.860°, 13.994°, 15.521°, 18.436°, 19.503°, 20.760°, 21.070°, 22.007°, 22.745°, 23.340°, 24.187°, 25.532°, 26.880°, 27.856°, 28.163°, 31.267°, 33.024°, 35.030°, 36.835°, 39.312°, 40.545°, and 40.988°.

(30) The pharmaceutical composition of (27), wherein the benzenesulfonate salt of the compound of Formula (I) is a benzenesulfonate salt of 3-(2-(dimethylamino)ethyl)-1H-indol-4-ol (I-7a).

(31) The pharmaceutical composition of (30), wherein the benzenesulfonate salt of 3-(2-(dimethylamino)ethyl)-1H-indol-4-ol (I-7a) is crystalline and characterized by an X-ray powder diffraction pattern containing at least three characteristic peaks at diffraction angles (2θ±0.2°) selected from 7.002°, 7.733°, 11.768°, 12.516°, 12.882°, 13.546°, 13.968°, 14.788°, 15.225°, 15.474°, 18.370°, 19.737°, 20.703°, 21.050°, 21.873°, 21.982°, 22.315°, 22.639°, 23.282°, 23.775°, 24.125°, 25.193°, 25.475°, 25.931°, 26.813°, 27.778°, 28.127°, 30.866°, 31.207°, 32.941°, 33.222°, 33.698°, 36.803°, 38.668°, and 39.289°.

(32) The pharmaceutical composition of any one of (24) to (26), wherein the pharmaceutically acceptable salt of the compound of Formula (I) is a tartrate salt of the compound of Formula (I).

(33) The pharmaceutical composition of (32), wherein the tartrate salt of the compound of Formula (I) is a tartrate salt of 3-(2-(bis(methyl-d₃)amino)ethyl-1,1,2,2-d₄)-1H-indol-4-ol (I-3b).

(34) The pharmaceutical composition of (33), wherein the tartrate salt of 3-(2-(bis(methyl-d₃)amino)ethyl-1,1,2,2-d₄)-1H-indol-4-ol (I-3b) is crystalline and characterized by an X-ray powder diffraction pattern containing at least three characteristic peaks at diffraction angles (2θ±0.2°) selected from 6.732°, 12.708°, 13.470°, 14.774°, 15.921°, 16.268°, 17.295°, 18.869°, 20.079°, 20.208°, 20.877°, 21.894°, 22.657°, 23.4910, 23.702°, 24.636°, 24.882°, 25.569°, 26.685°, 27.060°, 27.502°, 28.179°, 28.597°, 29.035°, 29.257°, 29.527°, 31.017°, 31.527°, 32.059°, 32.307°, 33.012°, 34.024°, 34.388°, 34.905°, 35.361°, 36.183°, 37.372°, 37.764°, 38.657°, and 41.049°.

(35) The pharmaceutical composition of (32), wherein the tartrate salt of the compound of Formula (I) is a tartrate salt of 3-(2-(dimethylamino)ethyl)-1H-indol-4-ol (I-7b).

(36) The pharmaceutical composition of (35), wherein the tartrate salt of 3-(2-(dimethylamino)ethyl)-1H-indol-4-ol (I-7b) is crystalline and characterized by an X-ray powder diffraction pattern containing at least three characteristic peaks at diffraction angles (2θ±0.2°) selected from 6.798°, 11.360°, 12.764°, 13.535°, 14.837°, 15.973°, 16.351°, 17.367°, 18.937°, 20.168°, 20.929°, 21.946°, 22.719°, 23.604°, 23.814°, 24.874°, 25.609°, 26.745°, 27.111°, 27.558°, 28.653°, 29.630°, 31.129°, 31.567°, 32.180°, 33.073°, 34.096°, 34.460°, 36.226°, 37.497°, 38.727°, and 41.126°.

(37) The pharmaceutical composition of (35), wherein the tartrate salt of 3-(2-(dimethylamino)ethyl)-1H-indol-4-ol (I-7b) is crystalline and characterized by an X-ray powder diffraction pattern containing at least three characteristic peaks at diffraction angles (2θ±0.2°) selected from 6.479°, 10.486°, 10.862°, 11.913°, 12.222°, 12.972°, 13.161°, 13.467°, 14.230°, 15.372°, 15.736°, 16.053°, 16.457°, 16.613°, 17.009°, 17.695°, 17.913°, 18.486°, 18.795°, 19.479°, 20.101°, 20.416°, 20.818°, 21.352°, 22.106°, 22.320°, 22.629°, 22.964°, 23.698°, 23.950°, 24.1750, 24.439°, 24.818°, 25.079°, 25.880°, 26.528°, 27.297°, 27.752°, 28.124°, 28.349°, 28.6310, 29.075°, 29.819°, 30.202°, 30.562°, 31.025°, 31.207°, 31.650°, 31.953°, 33.721°, 34.362°, 34.651°, 34.994°, 35.512°, 35.982°, 36.450°, 37.476°, 38.287°, 39.699°, 39.980°, 40.951°, and 41.870°.

(38) The pharmaceutical composition of any one of (24) to (26), wherein the pharmaceutically acceptable salt of the compound of Formula (I) is a hemi-fumarate salt of the compound of Formula (I).

(39) The pharmaceutical composition of (38), wherein the hemi-fumarate salt of the compound of Formula (I) is a hemi-fumarate salt of 3-(2-(bis(methyl-d₃)amino)ethyl-1,1,2,2-d₄)-1H-indol-4-ol (I-3c).

(40) The pharmaceutical composition of (39), wherein the hemi-fumarate salt of 3-(2-(bis(methyl-d₃)amino)ethyl-1,1,2,2-d₄)-1H-indol-4-ol (I-3c) is crystalline and characterized by an X-ray powder diffraction pattern containing at least three characteristic peaks at diffraction angles (2θ±0.2°) selected from 9.713°, 11.209°, 11.605°, 12.338°, 12.852°, 13.718°, 15.117°, 16.066°, 16.627°, 19.026°, 19.427°, 20.108°, 21.068°, 21.335°, 21.837°, 22.429°, 23.262°, 23.478°, 23.900°, 24.720°, 25.318°, 27.912°, 28.532°, 29.565°, 30.457°, 32.698°, 34.155°, 37.910°, 39.566°, and 40.999°.

(41) The pharmaceutical composition of (38), wherein the hemi-fumarate salt of the compound of Formula (I) is a hemi-fumarate salt of 3-(2-(dimethylamino)ethyl)-1H-indol-4-ol (I-7c).

(42) The pharmaceutical composition of (41), wherein the hemi-fumarate salt of 3-(2-(dimethylamino)ethyl)-1H-indol-4-ol (I-7c) is crystalline and characterized by an X-ray powder diffraction pattern containing at least three characteristic peaks at diffraction angles (2θ±0.2°) selected from 8.483°, 8.733°, 11.080°, 11.351°, 11.622°, 12.615°, 13.258, 14.977°, 15.557°, 16.089°, 16.319°, 16.606°, 17.013°, 18.928°, 18.884°, 19.429°, 19.734°, 20.643°, 21.484°, 22.067°, 23.433°, 24.466°, 24.885°, 26.740°, 27.900°, 28.557°, 29.523°, 32.888°, 34.183°, and 36.808°.

(43) The pharmaceutical composition of (41), wherein the hemi-fumarate salt of 3-(2-(dimethylamino)ethyl)-1H-indol-4-ol (I-7c) is crystalline and characterized by an X-ray powder diffraction pattern containing at least three characteristic peaks at diffraction angles (2θ±0.2°) selected from 9.746°, 11.354°, 12.338°, 13.762°, 16.111°, 16.644°, 19.929°, 20.180°, 21.576°, 22.758°, 23.348°, 23.938°, 24.724°, 25.226°, 26.203°, 27.910°, 29.056°, 29.499°, 32.753°, 35.567°, 37.279°, 37.347°, and 39.481°.

(44) The pharmaceutical composition of any one of (24) to (26), wherein the pharmaceutically acceptable salt of the compound of Formula (I) is a citrate salt of the compound of Formula (I).

(45) The pharmaceutical composition of (44), wherein the citrate salt of the compound of Formula (I) is a citrate salt of 3-(2-(bis(methyl-d₃)amino)ethyl-1,1,2,2-d₄)-1H-indol-4-ol (I-3e).

(46) The pharmaceutical composition of (45), wherein the citrate salt of 3-(2-(bis(methyl-d₃)amino)ethyl-1,1,2,2-d₄)-1H-indol-4-ol (I-3e) is amorphous by X-ray powder diffraction.

(47) The pharmaceutical composition of (44), wherein the citrate salt of the compound of Formula (I) is a citrate salt of 3-(2-(dimethylamino)ethyl)-1H-indol-4-ol (I-7e).

(48) The pharmaceutical composition of (47), wherein citrate salt of 3-(2-(dimethylamino)ethyl)-1H-indol-4-ol (I-7e) is amorphous by X-ray powder diffraction.

(49) The pharmaceutical composition of any one of (24) to (26), wherein the pharmaceutically acceptable salt of the compound of Formula (I) is a hemi-succinate salt of the compound of Formula (I).

(50) The pharmaceutical composition of any one of (24) to (26), wherein the pharmaceutically acceptable salt of the compound of Formula (I) is a benzoate salt of the compound of Formula (I).

(51) The pharmaceutical composition of (50), wherein the benzoate salt of the compound of Formula (I) is a benzoate salt of 3-(2-(bis(methyl-d₃)amino)ethyl-1,1,2,2-d₄)-1H-indol-4-ol (I-3j).

(52) The pharmaceutical composition of (51), wherein the benzoate salt of 3-(2-(bis(methyl-d₃)amino)ethyl-1,1,2,2-d₄)-1H-indol-4-ol (I-3j) is crystalline and characterized by an X-ray powder diffraction pattern containing at least three characteristic peaks at diffraction angles (2θ±0.2°) selected from 9.486°, 11.006°, 12.379°, 13.428°, 14.608°, 15.446°, 16.389°, 18.247°, 18.977°, 19.346°, 19.831°, 20.868°, 21.447°, 22.860°, 23.878°, 24.944°, 25.737°, 26.144°, 26.341°, 26.990°, 27.708°, 28.595°, 30.048°, 30.763°, 31.127°, 31.839°, 32.800°, 34.460°, 35.444°, 37.725°, and 38.597°.

(53) The pharmaceutical composition of (50), wherein the benzoate salt of the compound of Formula (I) is a benzoate salt of 3-(2-(dimethylamino)ethyl)-1H-indol-4-ol (I-7j).

(54) The pharmaceutical composition of (53), wherein the benzoate salt of 3-(2-(dimethylamino)ethyl)-1H-indol-4-ol (I-7j) is crystalline and characterized by an X-ray powder diffraction pattern containing at least three characteristic peaks at diffraction angles (2θ±0.2°) selected from 9.492°, 11.011°, 12.391°, 13.440°, 14.609°, 15.432°, 16.394°, 18.259°, 18.967°, 19.356°, 19.827°, 20.843°, 21.476°, 22.062°, 22.805°, 23.862°, 24.963°, 25.734°, 26.170°, 26.992°, 27.738°, 28.593°, 30.073°, 30.746°, 31.041°, 31.799°, 32.794°, 33.551°, 34.480°, 35.430°, 37.685°, and 38.643°.

(55) The pharmaceutical composition of (24), wherein the pharmaceutically acceptable salt of the compound of Formula (I) is a fatty acid salt of the compound of Formula (I).

(56) The pharmaceutical composition of (55), wherein the fatty acid salt of the compound of Formula (I) is an adipate salt, a laurate salt, a linoleate salt, a myristate salt, a caprate salt, a stearate salt, an oleate salt, a caprylate salt, a palmitate salt, a sebacate salt, an undecylenate salt, or a caproate salt of the compound of Formula (I).

(57) The pharmaceutical composition of any one of (1) to (56), wherein the organic acid agent is a hydroxy acid and/or an enedioic acid.

(58) The pharmaceutical composition of any one of (1) to (57), wherein the organic acid agent is at least one selected from the group consisting of glycolic acid, lactic acid, citric acid, tartaric acid, malic acid, fumaric acid, and maleic acid.

(59) The pharmaceutical composition of any one of (1) to (58), wherein the organic acid agent is citric acid and/or tartaric acid.

(60) The pharmaceutical composition of any one of (1) to (59), wherein the organic acid agent is citric acid.

(61) The pharmaceutical composition of any one of (1) to (60), wherein the organic acid agent is uncoated.

(62) The pharmaceutical composition of any one of (1) to (60), wherein the organic acid agent is coated.

(63) The pharmaceutical composition of (62), wherein the organic acid agent is coated with a water-soluble polymer.

(64) The pharmaceutical composition of (62), wherein the organic acid agent is coated with an anti-caking agent.

(65) The pharmaceutical composition of (62), wherein the organic acid agent is coated with a pH modifier.

(66) The pharmaceutical composition of (65), wherein the pH modifier is an alkali metal salt of an organic acid agent.

(67) The pharmaceutical composition of (66), wherein the organic acid agent is citric acid, and the alkali metal salt of an organic acid agent is sodium citrate.

(68) The pharmaceutical composition of any one of (62) to (67), wherein the organic acid agent is coated and is present in the pharmaceutical composition in the form of agglomerated granules together with a source of carbon dioxide.

(69) The pharmaceutical composition of any one of (1) to (68), wherein the organic acid agent is present in the pharmaceutical composition in an amount of at least 5% by weight and up to 40% by weight, based on a total weight of the pharmaceutical composition (on a dry basis).

(70) The pharmaceutical composition of any one of (1) to (69), which is in solid dosage form.

(71) The pharmaceutical composition of any one of (1) to (70), which is in solid dosage form adapted for oral administration.

(72) The pharmaceutical composition of (71), which is an intraoral dosage form.

(73) The pharmaceutical composition of (71) or (72), which is an orodispersible dosage form.

(74) The pharmaceutical composition of any one of (71) to (73), which is in a form of an orally disintegrating tablet (ODT).

(75) The pharmaceutical composition of any one of (1) to (74), which is an effervescent dosage form.

(76) The pharmaceutical composition of (75), wherein the pharmaceutically acceptable vehicle further comprises a source of carbon dioxide.

(77) The pharmaceutical composition of (76), wherein the source of carbon dioxide is at least one selected from the group consisting of sodium bicarbonate, sodium carbonate, potassium carbonate, potassium bicarbonate, magnesium carbonate, calcium carbonate, and sesquicarbonate.

(78) An oral liquid dosage form, prepared by reconstituting the pharmaceutical 20 composition of any one of (1) to (77) in solid dosage form, in a pharmaceutically acceptable aqueous medium.

(79) The oral liquid dosage form of (78), wherein the pharmaceutically acceptable aqueous medium is water or a juice.

(80) A method of treating a subject with a disease or disorder associated with a serotonin 5-HT₂ receptor, comprising:
administering to the subject a therapeutically effective amount of the pharmaceutical composition of any one of (1) to (77).

(81) The method of (80), wherein the disease or disorder is a central nervous system (CNS) disorder.

(82) The method of (81), wherein the central nervous system (CNS) disorder is at least one selected from the group consisting of major depressive disorder (MDD), treatment-resistant depression (TRD), post-traumatic stress disorder (PTSD), bipolar and related disorders, obsessive-compulsive disorder (OCD), generalized anxiety disorder (GAD), social anxiety disorder, a substance use disorder, an eating disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, suicidal ideation, suicidal behavior, major depressive disorder with suicidal ideation or suicidal behavior, melancholic depression, atypical depression, dysthymia, non-suicidal self-injury disorder (NSSID), chronic fatigue syndrome, Lyme's disease, gambling disorder, a paraphilic disorder, sexual dysfunction, peripheral neuropathy, and obesity.

(83) The method of (81), wherein the central nervous system (CNS) disorder is major depressive disorder (MDD).

(84) The method of (81), wherein the central nervous system (CNS) disorder is treatment-resistant depression (TRD).

(85) The method of (81), wherein the central nervous system (CNS) disorder is generalized anxiety disorder (GAD).

(86) The method of (81), wherein the central nervous system (CNS) disorder is social anxiety disorder.

(87) The method of (81), wherein the central nervous system (CNS) disorder is obsessive-compulsive disorder (OCD).

(88) The method of (81), wherein the central nervous system (CNS) disorder is cluster headaches or migraine.

(89) The method of (81), wherein the central nervous system (CNS) disorder is a substance use disorder.

(90) The method of (89), wherein the substance use disorder is alcohol use disorder and/or nicotine use disorder.

(91) The method of (80), wherein the disease or disorder is an autonomic nervous system (ANS) condition.

(92) The method of any one of (80) to (91), wherein the pharmaceutical composition is administered orally to the subject.

(93) The method of any one of (80) to (92), wherein the pharmaceutical composition is administered intraorally to the subject.

(94) The method of any one of (80) to (92), wherein the pharmaceutical composition is administered by reconstituting the pharmaceutical composition in solid dosage form in a pharmaceutically acceptable aqueous medium to form an oral liquid dosage form, followed by administering orally to the subject the oral liquid dosage form.

(95) The method of any one of (80) to (94), wherein the pharmaceutical composition is administered to the subject in an amount which provides the compound of Formula (I) at a psychedelic dose of about 0.083 mg/kg to about 5 mg/kg.

(96) The method of (95), wherein the pharmaceutical composition is administered to provide the psychedelic dose once per week or less over a treatment course.

(97) The method of any one of (80) to (94), wherein the pharmaceutical composition is administered to the subject in an amount which provides the compound of Formula (I) at a sub-psychedelic dose of about 0.00001 mg/kg to less than about 0.083 mg/kg.

(98) The method of (97), wherein the pharmaceutical composition is administered to provide the sub-psychedelic dose once per day or more over a treatment course.

(99) Use of the pharmaceutical composition of any one of (1) to (77) for treating a subject with a disease or disorder associated with a serotonin 5-HT₂ receptor.

(100) Use of the oral liquid dosage form of (78) or (79) for treating a subject with a disease or disorder associated with a serotonin 5-HT₂ receptor.

### BRIEF DESCRIPTION OF THE DRAWINGS

The forgoing paragraphs have been provided by way of general introduction and are not intended to limit the scope of the following claims. The described embodiments, together with further advantages, will be best understood by reference to the following detailed description when considered in conjunction with the accompanying drawings, wherein:

FIGS. 1A-1D show a synthetic route (FIG. 1A), a ¹H NMR spectrum (FIGS. 1B-1C), and a high resolution mass spectrometry (HRMS) spectrum (FIG. 1D) for compound I-3 (PI-d₁₀);

FIGS. 2A-2C show the X-ray powder diffraction (XRPD) pattern (pattern 1) of compound I-3, with FIGS. 2B and 2C being zoomed in and annotated;

FIGS. 3A-3D show the X-ray powder diffraction (XRPD) pattern of I-7a (pattern 1) (FIG. 3A), with FIG. 3B being zoomed in and annotated, the XRPD pattern of I-7 (PI-d₀, free base) (pattern 1) (FIG. 3C), and a comparison between the XRPD patterns of I-7a (benzenesulfonate salt) and I-7 (PI-d₀, free base) (pattern 1)(FIG. 3D);

FIG. 4 shows a differential scanning calorimetry (DSC) curve of I-7a;

FIG. 5 shows a thermogravimetric analysis (TGA) curve of I-7a;

FIGS. 6A and 6B show a ¹H NMR spectrum of I-7a;

FIG. 7 shows the ultra performance liquid chromatogram (UPLC) of I-7a;

FIG. 8 shows a DVS isotherm plot of I-7a;

FIG. 9 shows the XRPD patterns of I-7a (pattern 1) pre- and post-DVS analysis;

FIG. 10 shows the XRPD patterns of I-7a after storing solid samples for 22 days under the following conditions: i) 25° C., closed vial, ii) 25° C./96% RH, and iii) 40° C./75% RH, and comparing to fresh sample;

FIG. 11 shows the XRPD patterns of I-7a after maturation in 12 different solvents;

FIG. 12 shows the XRPD pattern of two different crystalline polymorphs of I-7b, pattern 1 (made from acetonitrile or THF), and pattern 2 (made from 1,4-dioxane);

FIG. 13 shows the DSC curve of I-7b (pattern 1);

FIG. 14 shows the TGA curve of I-7b (pattern 1);

FIGS. 15A-15B show the ¹H NMR spectrum of I-7b (pattern 1);

FIG. 16 shows a DVS isotherm plot of I-7b (pattern 1);

FIG. 17 shows a DVS change in mass plot of I-7b (pattern 1);

FIG. 18 shows the XRPD patterns of I-7b (pattern 1) after storing solid samples for 22 days under the following conditions: i) 25° C., closed vial, ii) 25° C./96% RH, and iii) 40° C./75% RH, and comparing to fresh sample, with samples ii), iii) and post DVS indicating a change in form to polymorph of pattern 3;

FIGS. 19A-19B show the DSC plots of I-7b (pattern 1) pre-DVS (FIG. 19A) and post-DVS (FIG. 19B);

FIGS. 20A-20B show the TGA plots of I-7b (pattern 1) pre-DVS (FIG. 20A) and post-DVS (FIG. 20B);

FIG. 21 shows the XRPD patterns of I-7b (pattern 1) after maturation in 12 different solvents;

FIG. 22 shows the XRPD patterns of I-7b (amorphous) obtained from salt formation with 0.5 eq of L-tartaric acid from either 1,4-dioxane or THF;

FIG. 23 shows the XRPD pattern of three different crystalline polymorphs of I-7c: a polymorph having pattern 1 (made from THF), a polymorph having pattern 2 (made from acetonitrile), a polymorph having pattern 3 (made from 1,4-dioxane);

FIG. 24 shows the DSC curve of I-7c (pattern 1);

FIG. 25 shows the TGA plot of I-7c (pattern 1);

FIGS. 26A-26B show a DSC (FIG. 26A) and TGA (FIG. 26B) plot of I-7c (pattern 2);

FIG. 27 shows the DSC curve of I-7c (pattern 3);

FIG. 28 shows the TGA plot of I-7c (pattern 3);

FIG. 29 shows the XRPD pattern of four different crystalline polymorphs of I-7c: a polymorph having pattern 1 (made from either 0.5 eq or 1 eq fumaric acid and THF), a polymorph having pattern 2 (made from 0.5 eq fumaric acid and acetonitrile), a polymorph having pattern 3 (made from either 0.5 eq or 1 eq fumaric acid in 1,4-dioxane), and a polymorph having pattern 4 (made from 1 eq fumaric acid in acetonitrile);

FIGS. 30A-30B show a DSC (FIG. 30A) and TGA (FIG. 30B) of I-7c (pattern 4);

FIGS. 31A-31B show a DVS (FIG. 31A) and a DVS change in mass plot (FIG. 31B) of I-7c (pattern 4);

FIG. 32 shows the XRPD pattern of two different crystalline polymorphs of I-7d: a polymorph having pattern 1 (made from 1,4-dioxane), and a polymorph having pattern 2 (made from THF/heptane);

FIG. 33 shows the DSC curve of I-7d (pattern 1);

FIG. 34 shows the TGA plot of I-7d (pattern 1);

FIG. 35 shows the DSC curve of I-7d (pattern 2);

FIG. 36 shows the TGA curve of I-7d (pattern 2);

FIGS. 37A-37B show the XRPD pattern of I-7e (amorphous) after freeze drying (FIG. 37A) and slurrying in THF (FIG. 37B);

FIGS. 38A-38B shows the ¹H NMR spectrum of I-7e;

FIG. 39 shows the XRPD pattern of I-7f (pattern 1) compared to free base;

FIG. 40 shows the DSC curve of I-7f;

FIG. 41 shows the TGA plot of I-7f;

FIG. 42 shows the XRPD pattern of I-7c pre-DVS (pattern 5, obtained from scale-up using 1 eq fumaric acid in acetonitrile) and post-DVS (pattern 6);

FIG. 43 shows the DSC plot of I-7c pre-DVS (polymorph 5, obtained from scale-up using 1 eq fumaric acid in acetonitrile);

FIG. 44 shows the DSC plot of I-7c polymorph 5 obtained post-DVS (pattern 6);

FIGS. 45A-45B show the TGA plot of I-7c pre-DVS (FIG. 45A, polymorph 5, obtained from scale-up using 1 eq fumaric acid in acetonitrile) and post-DVS (FIG. 45B, pattern 6);

FIG. 46 shows the XRPD patterns of I-7c (pattern 5) after maturation in 12 different solvents, forming polymorphs of patterns (P) 1, 6, 7, 8, 9, 10, and 11;

FIG. 47 shows the XRPD pattern of I-7h (pattern 1) formed from either 1,4-dioxane or THF;

FIG. 48 shows the DSC curve of I-7h (pattern 1);

FIG. 49 shows TGA plot of I-7h (pattern 1);

FIG. 50 shows the XRPD pattern of six different crystalline polymorphs of I-7i: a polymorph having pattern 1 (made from 0.5 eq oxalic acid and THF), a polymorph having pattern 2 (made from 1 eq oxalic acid and THF), a polymorph having pattern 3 (made from 0.5 eq oxalic acid and acetonitrile), a polymorph having pattern 4 (made from 1 eq oxalic acid and acetonitrile), a polymorph having pattern 5 (made from 0.5 eq oxalic acid and 1,4-dioxane), and a polymorph having pattern 6 (made from 1 eq oxalic acid and 1,4-dioxane);

FIG. 51 shows the DSC curve of I-7i (polymorphs of patterns 1-6);

FIG. 52 shows the TGA plot of I-7i (polymorphs of patterns 2-6);

FIGS. 53A-53B show the XRPD pattern of I-7j (pattern 1), with FIG. 53B being zoomed in and annotated.

FIG. 54 shows the TGA plot of I-7j (pattern 1);

FIG. 55 shows the DSC curve of I-7j (pattern 1);

FIG. 56 shows the XRPD patterns of I-7j (pattern 1) after storing solid samples for 22 days under the following conditions: i) 25° C., closed vial, ii 25°) C/96% RH, and iii) 40° C./75% RH, and comparing to fresh sample;

FIG. 57 shows the XRPD patterns of I-7j (pattern 1) after maturation in 12 different solvents;

FIG. 58 shows the DVS isotherm of I-7j (pattern 1);

FIGS. 59A-59C show that no changes to I-7j (pattern 1) took place after being subjected to DVS conditions (post-DVS) by XRPD (FIG. 59A, compared to pattern before DVS from material obtained from THF and acetonitrile) and by ¹H NMR (FIGS. 59B and 59C);

FIG. 60 shows the XRPD pattern of three different crystalline polymorphs of 1-7k: a polymorph having pattern 1 (made from acetonitrile/TBME), a polymorph having pattern 2 (made from THF/heptane), and a polymorph having pattern 3 (made from 1,4-dioxane/heptane);

FIG. 61 shows the DSC curve of three different crystalline polymorphs of 1-7k;

FIG. 62 shows the TGA plot of three different crystalline polymorphs of I-7k;

FIGS. 63A-63F show the XRPD pattern of I-3a (pattern 1) (FIG. 63A), zoomed in and annotated versions of the XRPD plot (FIGS. 63B-63C), a comparative XRPD plot of I-3a (pattern 1) to I-7a seeds (FIG. 63D); and a single crystal X-ray structure of I-3a (pattern 1)(FIGS. 63E-63F);

FIGS. 64A-64B show a comparison of I-3a (pattern 1) to I-7a seeds by DSC (FIG. 64A) and TGA (FIG. 64B);

FIGS. 65A-65B shows the ¹H NMR spectrum of I-3a (pattern 1);

FIG. 66 shows the XRPD pattern of I-3b (pattern 1, obtained from non-seeded experiments) compared to crystalline polymorphs of I-7b of pattern 1 (from THF) and pattern 2 (from 1,4-dioxane);

FIG. 67 shows DSC curve of I-3b (pattern 1, obtained from non-seeded experiments) compared to crystalline polymorphs of I-7b of pattern 1 (from THF) and pattern 2 (from 1,4-dioxane);

FIG. 68 shows the TGA plot of I-3b (pattern 1, obtained from non-seeded experiments) compared to crystalline polymorphs of I-7b of pattern 1 (from THF) and pattern 2 (from 1,4-dioxane);

FIGS. 69A-69D show the XRPD pattern of I-3b (pattern 2, obtained from seeded experiments), compared to the seeds of crystalline polymorph of I-7b of pattern 1, and the crystalline polymorph of I-3b of pattern 1 obtained from the non-seeded experiments (FIG. 69A), the zoomed in and annotated XRPD of I-3b (pattern 2, obtained from seeded experiments) (FIG. 69B); and the single crystal X-ray structure of I-3b (pattern 2)(FIGS. 69C-69D);

FIG. 70 shows the DSC curve of I-3b (pattern 2);

FIG. 71 shows the TGA plot of I-3b (pattern 2);

FIG. 72 shows the XRPD pattern of I-3c (pattern 1, obtained from non-seeded experiments) to the crystalline polymorphs of I-7c of patterns 1 through 4;

FIG. 73 shows the DSC curve of I-3c (pattern 1) compared to that of the polymorph patterns 1 through 4 of I-7c;

FIG. 74 shows the TGA plot of I-3c (pattern 1) compared to that of the polymorph patterns 1 through 4 of I-7c;

FIGS. 75A-75B show the XRPD. pattern of I-3c (pattern 2, obtained from seeded experiments) compared to crystalline polymorph of I-3c of pattern 1 obtained from the non-seeded experiments and the seeds of I-7c crystalline polymorph pattern 4 (FIG. 75A), and the XRPD pattern of I-3c (pattern 2, obtained from seeded experiments) alone (FIG. 75B);

FIG. 76 shows the DSC curve of I-3c (pattern 2, obtained from seeded experiments) compared to crystalline polymorph of I-3c of pattern 1 obtained from the non-seeded experiments and the seeds of I-7c crystalline polymorph pattern 4;

FIG. 77 shows the TGA plot of I-3c (pattern 2, obtained from seeded experiments) compared to crystalline polymorph of I-3c of pattern 1 obtained from the non-seeded experiments and the seeds of I-7c crystalline polymorph pattern 4;

FIGS. 78A-78E shows the XRPD pattern of I-3j (pattern 1) (FIG. 78A), a zoomed in and annotated version (FIG. 78B), a comparison of the XRPD pattern of I-3j (pattern 1) to that of the I-7j seed (FIG. 78C), a single crystal X-ray structure of I-3j (pattern 1) (FIGS. 78D-78E);

FIGS. 79A-79B show the ¹H NMR spectrum of I-3j (pattern 1);

FIG. 80 shows the DSC plot of I-3j (pattern 1) compared to I-7j (pattern 1);

FIG. 81 shows the DVS isotherm plot of I-3j (pattern 1);

FIG. 82 shows the DVS change in mass plot of I-3j (pattern 1);

FIG. 83 shows the XRPD patterns of I-3j (pattern 1) after storing solid samples for 22 days under the following conditions: i) 25° C., closed vial, ii) 25° C./96% RH, and iii) 40° C./75% RH, and comparing to fresh sample and post DVS sample;

FIG. 84 shows the XRPD patterns of I-3j (pattern 1) after maturation in 12 different solvents;

FIG. 85 shows XRPD diffraction peaks of compound I-3 (pattern 1) obtained from crash cooling and freeze-drying solutions of I-3 (PI-d₁₀, free base) in 1,4-dioxane, t-BuOH, 1,4-dioxane/water, MeCN/water,

FIG. 86 shows a DSC plot of compound I-3 (PI-d₁₀, free base) (pattern 1);

FIG. 87 shows an XRPD of the amorphous form of compound I-3 (PI-d₁₀, free base) obtained from melt/crash cooling experiment (>185° C./30° C.) in DSC compared to the XRPD pattern of compound I-3 (pattern 2) which resulted from the amorphous form crystallizing overnight upon standing;

FIG. 88 shows the XRPD pattern of I-3 (pattern 2) obtained from DSC scale-up experiments;

FIG. 89 shows the annotated XRPD pattern of I-3 (pattern 2) obtained from DSC scale-up experiments;

FIG. 90 shows the XRPD pattern of I-3m (pattern 1) compared to diffraction patterns 1 and 2 of the free base I-3;

FIG. 91 shows the XRPD pattern of I-3n (pattern 1) compared to diffraction patterns 1 and 2 of the free base I-3;

FIG. 92 shows the XRPD pattern of I-3o (pattern 1) compared to diffraction patterns 1 and 2 of the free base I-3;

FIG. 93 shows the XRPD pattern of I-3p (pattern 1) compared to diffraction patterns 1 and 2 of the free base I-3;

FIG. 94 shows the XRPD pattern of two different polymorphs of I-3q (pattern 1 obtained from commercially available stearic acid, and pattern 2 obtained from desalting sodium stearate) compared to the diffraction patterns I and 2 of the free base I-3;

FIG. 95 shows the XRPD pattern of two different polymorphs of I-3r (pattern 1 obtained from desalting sodium oleate, and pattern 2 obtained from commercially available oleic acid) compared to the diffraction patterns 1 and 2 of the free base I-3;

FIG. 96 shows the XRPD pattern of I-3s (pattern 1) compared to diffraction patterns 1 and 2 of the free base I-3;

FIG. 97 shows the stability of I-7 (PI-d₀) over 24 hours in 0.1 M solutions of acetic acid, with or without metal ions, compared to those solutions without acetic acid, at 40° C.;

FIG. 98 shows the stability of I-7 (PI-d₀) over 24 hours in 0.1 M solutions of ascorbic acid, with or without metal ions, compared to those solutions without ascorbic acid, at 40° C.;

FIG. 99 shows the stability of I-7 (PI-d₀) over 24 hours in 0.1 M solutions of benzenesulfonic acid, with or without metal ions, compared to those solutions without benzenesulfonic acid, at 40° C.;

FIG. 100 shows the stability of I-7 (PI-d₀) over 24 hours in 0.1 M solutions of fumaric acid, with or without metal ions, compared to those solutions without fumaric acid, at 40° C.;

FIG. 101 shows the stability of I-7 (PI-d₀) over 24 hours in 0.1 M solutions of malonic acid, with or without metal ions, compared to those solutions without malonic acid, at 40° C.;

FIG. 102 shows the stability of I-7 (PI-d₀) over 24 hours in 0.1 M solutions of succinic acid, with or without metal ions, compared to those solutions without succinic acid, at 40° C.;

FIG. 103 shows the stability of I-7 (PI-d₀) over 24 hours in 0.1 M solutions of tartaric acid, with or without metal ions, compared to those solutions without tartaric acid, at 40° C.;

FIG. 104 shows the stability of I-7 (PI-d₀) over 24 hours in 0.1 M solutions of citric acid, with or without metal ions, compared to those solutions without citric acid, at 40° C.;

FIG. 105 shows the stability of I-7 (PI-d₀) over 24 hours in dilute solutions of citric acid, with or without metal ions, compared to those solutions without citric acid, at 4° C.;

FIG. 106 shows the stability of I-7 (PI-d₀) over 24 hours in dilute solutions of citric acid, with or without metal ions, compared to those solutions without citric acid, at 23° C.;

FIG. 107 shows the stability of I-7 (PI-d₀) over 24 hours in dilute solutions of citric acid, with or without metal ions, compared to those solutions without citric acid, at 40° C.;

FIGS. 108A-108C shows the stability of I-7 (PI-d₀) over 24 hours in 0.1M solutions of sodium citrate buffer, with or without metal ions, compared to those solutions without sodium citrate buffer, at 4° C. (FIG. 108A), 23° C. (FIG. 108B), 40° C. (FIG. 108C);

FIG. 109 shows the stability of I-7 (PI-d₀) over 24 hours in 0.1M solutions of phosphate buffer (pH 6.0), phosphate buffer (pH 7.5), and sodium citrate buffer (6.0) at 40° C.;

FIG. 110 shows the long-term stability (up to 25 days) of I-7 (PI-d₀) in a sodium citrate buffer (0.1 M, pH 6.01) at 4° C. and 23° C.;

FIG. 111 shows the long-term stability (up to 25 days) of I-7 (PI-d₀) in a citric acid solution (0.1 M, pH 1.60) at 4° C. and 23° C.;

FIG. 112 shows the stability of I-7 (PI-d₀) over 24 hours in 20 μM solutions of ethylenediaminetetraacetic acid (EDTA), with or without metal ions, compared to those solutions without ethylenediaminetetraacetic acid (EDTA), at 40° C.;

FIG. 113 shows the stability of I-7 (PI-d₀) over 24 hours in 20 μM solutions of ascorbic acid, with or without metal ions, compared to those solutions without ascorbic acid, at 40° C.;

FIG. 114 shows the stability of I-7 (PI-d₀) over 24 hours in 20 μM solutions of sodium metabisulfite, with or without metal ions, compared to those solutions without sodium metabisulfite, at 40° C.;

FIG. 115 shows the stability of I-7 (PI-d₀) over 24 hours in 20 μM solutions of L-cysteine, with or without metal ions, compared to those solutions without L-cysteine, at 40° C.;

FIG. 116 shows the stability of I-7 (PI-d₀) over 24 hours in 20 μM solutions of propyl gallate, with or without metal ions, compared to those solutions without propyl gallate, at 40° C.;

FIG. 117 shows the stability of I-7 (PI-d₀) over 24 hours in 1% w/w solutions of CAVASOL® W7 HP, with or without metal ions, compared to those solutions without CAVASOL® W7 HP, at 40° C.;

FIG. 118 shows the stability of I-7 (PI-d₀) over 24 hours in 1% w/w solutions of CAVASOL® W7 M, with or without metal ions, compared to those solutions without CAVASOL® W7 M, at 40° C.;

FIG. 119 shows the stability of I-7 (PI-d₀) over 24 hours in 1% w/w solutions of CAVITRON® W7 HP7, with or without metal ions, compared to those solutions without CAVITRON® W7 HP7, at 40° C.;

FIG. 120 shows the solubility of I-3 (PI-d₁₀) (pattern 1), I-7 (PI-d₀) (pattern 1), I-3j (pattern 1), I-7a (pattern 1), I-7b (pattern 1), I-7c (pattern 5), and I-7j (pattern 1) in FaSSGF (Fasted State Simulated Gastric Fluid)(pH 1.6), at 37° C. for 2 and 6 hours;

FIG. 121 shows the solubility of I-3 (PI-d₁₀) (pattern 1), I-7 (PI-d₀) (pattern 1), I-3j (pattern 1), I-7a (pattern 1), I-7b (pattern 1), I-7c (pattern 5), and I-7j (pattern 1) in water at room temperature for 2 and 6 hours;

FIG. 122 shows the TGA plot of I-7 (API) used in the ODT formulations;

FIG. 123 show the DSC curve of 1-7 (API) used in the ODT formulations;

FIG. 124 shows the XRPD pattern of I-7 (pattern 1)(API) used in the ODT formulations;

FIG. 125 shows the TGA plot of the ODT dosage form formed from batch 1a (SH24) formulated with the citrate salt of psilocin at pH 3.55;

FIG. 126 shows the DSC curve of the ODT dosage form formed from batch 1a (SH24) formulated with the citrate salt of psilocin at pH 3.55;

FIG. 127 shows the XRPD pattern of the ODT dosage form formed from batch 1a (SH24) formulated with the citrate salt of psilocin at pH 3.55;

FIG. 128 shows the appearance of the ODT dosage form formed from batch 1a (SH24) formulated with the citrate salt of psilocin at pH 3.55;

FIG. 129 shows the DSC plot of the ODT dosage form formed from batch 1b (SH24) formulated with the citrate salt of psilocin at pH 4.50;

FIG. 130 shows the XRPD pattern of the ODT dosage form formed from batch 1b (SH24) formulated with the citrate salt of psilocin at pH 4.50;

FIG. 131 shows the appearance of the ODT dosage form formed from batch 1b (SH24) formulated with the citrate salt of psilocin at pH 4.50;

FIG. 132 shows the DSC plot of the ODT dosage form formed from batch 1c (SH24) formulated with the citrate salt of psilocin at pH 7.56;

FIG. 133 shows the XRPD pattern of the ODT dosage form formed from batch 1c (SH24) formulated with the citrate salt of psilocin at pH 7.56;

FIG. 134 shows the appearance of the ODT dosage form formed from batch 1c (SH24) formulated with the citrate salt of psilocin at pH 7.56;

FIG. 135 shows the DSC curve of the ODT dosage form formed from batch 2a (SH24) formulated with the tartrate salt of psilocin at pH 3.13;

FIG. 136 shows the XRPD pattern of the ODT dosage form formed from batch 2a (SH24) formulated with the tartrate salt of psilocin at pH 3.13;

FIG. 137 shows the appearance of the ODT dosage form formed from batch 2a (SH24) formulated with the tartrate salt of psilocin at pH 3.13;

FIG. 138 shows the DSC plot of the ODT dosage form formed from batch 2b (SH24) formulated with the tartrate salt of psilocin at pH 4.33;

FIG. 139 shows the XRPD pattern of the ODT dosage form formed from batch 2b (SH24) formulated with the tartrate salt of psilocin at pH 4.33;

FIG. 140 shows the appearance of the ODT dosage form formed from batch 2b (SH24) formulated with the tartrate salt of psilocin at pH 4.33;

FIG. 141 shows the DSC curve of the ODT dosage form formed from batch 2c (SH24) formulated with the tartrate salt of psilocin at pH 7.94;

FIG. 142 shows the XRPD pattern of the ODT dosage form formed from batch 2c (SH24) formulated with the tartrate salt of psilocin at pH 7.94;

FIG. 143 shows the TGA plot of the placebo ODT dosage form;

FIG. 144 shows the DSC curve of the placebo ODT dosage form;

FIG. 145 shows the XRPD pattern of the placebo ODT dosage form;

FIG. 146 shows a plasma concentration-time curve of psilocybin dosed orally and intravenously in rats;

FIG. 147 is a plasma concentration-time curve of PI-d₀+PI-d₁₀ (PI-tot) from co-dosing PI-d₀ and PI-d₁₀ orally and intravenously in rats;

FIG. 148 is a plasma concentration-time curve comparing PI-tot plasma levels after oral PI-d₀+PI-d₁₀ and oral psilocybin in rats;

FIG. 149 is a tissue concentration-time curve comparing brain and plasma psilocybin levels after intravenous dosing of psilocybin in rats;

FIG. 150 is a tissue concentration-time curve comparing brain and plasma PI-tot levels after intravenous co-dosing of PI-d₀ and PI-d₁₀ in rats;

FIG. 151 is a brain concentration-time curve comparing brain PI levels after intravenous dosing of psilocybin and PI-tot levels after intravenous co-dosing of PI-d₀ and PI-d₁₀ in rats;

FIGS. 152A-152B show a plasma concentration-time curve following intravenous and oral administration of psilocin-d₁₀ to dogs (FIG. 152A), and a bioavailability profile of psilocin-d₁₀ to dogs of 91.3% (FIG. 152B);

FIGS. 153A-153B show the plasma concentration-time profiles for PI-d₀ after psilocybin dosing (FIG. 153A) and for PI-d₁₀ after PI-d₁₀ (FIG. 153B) with orally disintegrating tablets (ODT) and powder in capsule (PIC) dosage forms;

FIG. 154 shows the exposure comparison between PI-d₀ after psilocybin dosing and PI-d₁₀ after PI-d₁₀ dosing for both ODT and PIC dosage forms as assessed by Cmax; and

FIG. 155 shows the exposure comparison between PI-d₀ after psilocybin dosing and PI-d₁₀ after PI-d₁₀ dosing for both ODT and PIC dosage forms as assessed by AUCinf.

### DETAILED DESCRIPTION

In the following detailed description of the embodiments of the instant disclosure, numerous specific details are set forth in order to provide a thorough understanding of the disclosed embodiments. However, it will be obvious to one skilled in the art that the embodiments of this disclosure may be practiced without these specific details. In other instances, well known methods, procedures, components, and circuits have not been described in detail so as not to unnecessarily obscure aspects of the embodiments of the instant disclosure.

### Definitions

Unless defined otherwise, all technical and scientific terms used herein have the same meaning as is commonly understood by one of skill in the art to which this disclosure belongs.

When it is stated that a substituent or group “comprise(s) deuterium” or is “comprising deuterium,” it is to be understood that the substituent or group may itself be deuterium, or the substituent or group may contain at least one deuterium substitution in its chemical structure. For example, when substituent “-R” is defined to comprise deuterium, it is to be understood that -R may be -D (-deuterium), or a group such as -CD₃ that is consistent with the other requirements set forth of -R.

As used herein, the term “fatty” describes a compound with a long-chain (linear) hydrophobic portion made up of hydrogen and anywhere from 4 to 26 carbon atoms, which may be fully saturated or partially unsaturated.

The phrases “pharmaceutically acceptable,” “physiologically acceptable,” and the like, are employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio. When referencing salts, the phrases “pharmaceutically acceptable salt,” “physiologically acceptable salt,” and the like, means a salt which is acceptable for administration to a patient, such as a mammal (salts with counterions having acceptable mammalian safety for a given dosage regime). As is well known in the art, such salts can be derived from pharmaceutically acceptable inorganic or organic bases, by way of example, sodium, potassium, calcium, magnesium, ammonium, and tetraalkylammonium salts, and the like, and when the molecule contains a basic functionality, addition salts with inorganic acids, such as hydrochloride, hydrobromide, sulfate, sulfamate, phosphate, nitrate, perchlorate salts, and the like, and addition salts with organic acids, such as formate, tartrate, besylate, mesylate, acetate, maleate, malonate, oxalate, fumarate, benzoate, salicylate, succinate, oxalate, glycolate, hemi-oxalate, hemi-fumarate, propionate, stearate, tartrate, lactate, citrate, ascorbate, pamoate, hydroxymaleate, phenylacetate, glutamate, 2-acetoxybenzoate, tosylate, ethanedisulfonate, isethionate salts, and the like. The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient.

By way of example, salts of the present compounds include those wherein the compound is protonated by an inorganic or organic acid to form a cation, with the conjugate base of the inorganic or organic acid as the anionic component of the salt.

“Solvate” refers to a physical association of a compound or salt of the present disclosure with one or more solvent molecules, whether organic, inorganic, or a mixture of both. This physical association includes hydrogen bonding. In certain instances, the solvate will be capable of isolation, for example when one or more solvent molecules are incorporated in the crystal lattice of a crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Some examples of solvents include, but are not limited to, methanol, ethanol, isopropanol, N,N-dimethylformamide, tetrahydrofuran, dimethylsulfoxide, and water. When the solvent is water, the solvate formed is a hydrate (e.g., monohydrate, dihydrate, etc.). Exemplary solvates thus include, but are not limited to, hydrates, methanolates, ethanolates, isopropanolates, etc. Methods of solvation are generally known in the art.

“Stereoisomer” and “stereoisomers” refer to compounds that have same atomic connectivity but different atomic arrangement in space. Stereoisomers include cis-trans isomers, E and Z isomers, enantiomers, and diastereomers. All forms such as racemates and optically pure stereoisomers of the compounds are contemplated herein. Chemical formulas and compounds which possess at least one stereogenic center, but are drawn without reference to stereochemistry, are intended to encompass both the racemic compound, as well as the separate stereoisomers, e.g., R- and/or S-stereoisomers, each permutation of diastereomers so long as those diastereomers are geometrically feasible, etc.

“Tautomer” refers to alternate forms of a molecule that differ only in electronic bonding of atoms and/or in the position of a proton, such as enol-keto, imine-enamine, and neutral/zwitterionic tautomers, or the tautomeric forms of heteroaryl groups containing a —N═C(H)—NH— ring atom arrangement, such as pyrazoles, imidazoles, benzimidazoles, triazoles, and tetrazoles. Other tautomeric ring atom arrangements are also possible.

A “crystalline” solid is a type of solid whose fundamental three-dimensional structure contains a highly regular pattern of atoms or molecules—with long range order—forming a crystal lattice, and thus displays sharp characteristic crystalline peak(s) in its X-ray power diffraction (XRPD) pattern. In some instances, crystalline solids can exist in different crystalline forms known as “polymorphs,” which have the same chemical composition, but differ in packing, geometric arrangement, and other descriptive properties of the crystalline solid state. As such, polymorphs may have different solid-state physical properties to affect, for example, the solubility, dissolution rate, bioavailability, chemical and physical stability, flowability, and compressibility, etc. of the compound as well as the safety and efficacy of drug products based on the compound. In the process of preparing a polymorph, further purification, in terms of gross physical purity or optical purity, may be accomplished as well. A material's crystalline form, including polymorphic forms, may be designated by “pattern” number throughout the present disclosure (e.g., pattern 1, pattern 2, etc.) based on its characterized X-ray power diffraction (XRPD) pattern. As used herein, the term “amorphous” refers to a solid material having substantially no long range order in the position of its molecules—the molecules are arranged in a random manner so that there is effectively no well-defined arrangement, e.g., molecular packing, and no long range order. Amorphous solids are generally isotropic, i.e., exhibit similar properties in all directions and do not have definite melting points. For example, an amorphous material is a solid material having substantially no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. Thus, an “amorphous” subject compound/material is one characterized as having substantially no crystallinity-less than 10% crystallinity, less than 8% crystallinity, less than 6% crystallinity, less than 4% crystallinity, less than 2% crystallinity, less than 1% crystallinity, or 0% crystallinity—i.e., is at least 90%, at least 92%, at least 94%, at least 96%, at least 98%, or 100% amorphous, as determined for example by XRPD. For example, the % crystallinity can in some embodiments be determined by measuring the intensity of one or more peaks in the XRPD diffractogram compared to a reference peak, which may be that of a known standard or an internal standard. Other characterization techniques, such as modulated differential scanning calorimetry (mDSC) analysis, Fourier transform infrared spectroscopy (FTIR), and other quantitative methods, may also be employed to determine the percent a subject compound/material is amorphous or crystalline, including quantitative methods which provide the above percentages in terms of weight percent.

References to X-ray powder diffraction (XRPD) patterns of materials, compounds, salts, etc. of the present disclosure being characterized by an X-ray powder diffraction pattern containing “at least three characteristic peaks” should be understood to include those materials/compounds/salts characterized as having 3, 4, 5, 6, 7, 8, 9, 10, 11, 12, 13, 14, 15 or more (including all) of the recited characteristic XRPD diffraction peaks. Further, materials/compounds/salts containing “at least three characteristic peaks at diffraction angles (2θ±0.2°) selected from . . . ” are open to inclusion of other XRPD diffraction peaks not recited.

It will be appreciated that the compounds herein can exist in different salt, solvate, stereoisomer, tautomer, crystalline/amorphous (or polymorphic) forms, and the present disclosure is intended to include all permutations thereof, such as a solvate of a pharmaceutically acceptable salt of a stereoisomer of the subject compound.

As used herein, the term “steady” describes the stable or steady-state level of a molecule concentration, e.g., concentration of any compound described herein.

The term “stable,” “stability,” and the like, as used herein includes chemical stability and solid state (physical) stability. The term “chemical stability” means that the compound can be stored in an isolated form, or in the form of a formulation in which it is provided in admixture with for example, pharmaceutically acceptable carriers, diluents or adjuvants as described herein, under normal storage conditions, with little or no chemical degradation or decomposition. “Solid-state stability” means the compound can be stored in an isolated solid form, or the form of a solid formulation in which it is provided in admixture with, for example, pharmaceutically acceptable carriers, diluents or adjuvants as described herein, under normal storage conditions, with little or no solid-state transformation (e.g., hydration, dehydration, solvatization, desolvatization, crystallization, recrystallization or solid-state phase transition).

A “psilocybin-based” drug is any prodrug of a psilocin-type compound, such as an alkyl/aryl ester, an α-amino ester (e.g., an amino acid ester), a hemi-ester, a bis-ester, a phosphate ester, a sulfate ester, etc., that when administered releases psilocin or a deuterated analog thereof (e.g., a compound of Formula (I)) as the active component. A psilocybin-based drug includes psilocybin itself (dihydrogen phosphate ester of psilocin, in either neutral or zwitterionic form).

As used herein, the term “composition” is equivalent to the term “formulation.”

As used herein, the term “active ingredient” is equivalent to the term “active pharmaceutical ingredient” (API).

The language “tamper resistant” is art-recognized to describe aspects of a drug formulation that make it more difficult to use the formulation to abuse the drug moiety of the formulation through extraction for intravenous use, intradermal use, etc. use, or crushing for freebase use; and therefore reduce the risk for abuse of the drug.

The term “treating” or “treatment” as used herein means the treating or treatment of a disease or medical condition in a patient, such as a mammal (particularly a human) that includes: ameliorating the disease or medical condition, such as, eliminating or causing regression of the disease or medical condition in a patient; suppressing the disease or medical condition, for example by, slowing or arresting the development of the disease or medical condition in a patient; or alleviating one or more symptoms of the disease or medical condition in a patient. In an embodiment, prophylactic treatment can result in preventing the disease or medical condition from occurring, in a subject.

A “patient” or “subject,” used interchangeably herein, can be any mammal including, for example, a human and non-human subjects. A patient or subject can have a condition to be treated or can be susceptible to a condition to be treated.

As used herein, and unless otherwise specified, the terms “prevent,” “preventing” and “prevention” refer to the prevention of the onset, recurrence or spread of a disease, disorder, or condition, or of one or more symptoms thereof. The terms encompass the inhibition or reduction of a symptom of the particular disease, disorder, or condition. Subjects with familial history of a disease, disorder, or condition, in particular, are candidates for preventive regimens in certain embodiments. In addition, subjects who have a history of recurring symptoms are also potential candidates for the prevention. In this regard, the term “prevention” may be interchangeably used with the term “prophylactic treatment.” As used herein, and unless otherwise specified, the terms “manage,” “managing” and “management” refer to preventing or slowing the progression, spread or worsening of a disease, disorder, or condition, or of one or more symptoms thereof. Often, the beneficial effects that a subject derives from a prophylactic and/or therapeutic agent do not result in a cure of the disease, disorder, or condition. In this regard, the term “managing” encompasses treating a subject who had suffered from the particular disease, disorder, or condition in an attempt to prevent or minimize the recurrence of the disease, disorder, or condition, or of one or more symptoms thereof. “Therapeutically effective amount” refers to an amount of a compound(s) or its salt form sufficient to treat a specified disorder or disease or one or more of its symptoms and/or to prevent the occurrence of the disease or disorder (prophylactically effective amount).

As used herein, and unless otherwise specified, a “prophylactically effective amount” of an active ingredient, is an amount sufficient to prevent a disease, disorder, or condition, or prevent its recurrence. The term “prophylactically effective amount” can encompass an amount that improves overall prophylaxis or enhances the prophylactic efficacy of another prophylactic agent.

The term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”. A “continuous” administration schedule means administration every day without interruption during the treatment course. If the administration schedule follows an “intermittent” administration schedule, then days of administration may be followed by “rest days” or days of non-administration of drug within the course. A “drug holiday” indicates that the drug is not administered in a predetermined administration schedule. For example, after undergoing one or several courses of treatment, a subject may be prescribed a regulated drug holiday as part of the administration schedule, e.g., prior to re-recommencing active treatment.

The language “toxic spikes” is used herein to describe neurological spikes in concentration of any compound described herein that would produce side-effects of sedation or psychotomimetic effects (e.g., hallucination, dizziness, and nausea), or any unwanted and/or unintended secondary effects caused by the administration of a medicament to an individual resulting in subjective experiences being qualitatively different from those of ordinary consciousness. These experiences can include derealization, depersonalization, hallucinations and/or sensory distortions in the visual, auditory, olfactory, tactile, proprioceptive and/or interoceptive spheres and/or any other perceptual modifications, and/or any other. substantial subjective changes in cognition, memory, emotion and consciousness. Such side effects, when unwanted and/or unintended, can not only have immediate repercussions, but also effect treatment compliance. In particular, side effects may become more pronounced at blood concentration levels of about 250, 300, 400, 500 ng/L or more.

As used herein, and unless otherwise specified, a “neuropsychiatric disease or disorder” is a behavioral or psychological problem associated with a known neurological condition, and typically defined as a cluster of symptoms that co-exist. Examples of neuropsychiatric disorders include, but are not limited to, attention deficit disorder, attention deficit hyperactivity disorder, bipolar and manic disorders, depression, or any combinations thereof. “Inflammatory conditions” or “inflammatory disease,” as used herein, refers broadly to chronic or acute inflammatory diseases, including, but not limited to, rheumatic diseases (e.g., rheumatoid arthritis, osteoarthritis, psoriatic arthritis) spondyloarthropathies (e.g., ankylosing spondylitis, reactive arthritis, Reiter's syndrome), crystal arthropathies (e.g., gout, pseudogout, calcium pyrophosphate deposition disease), multiple sclerosis, Lyme disease, polymyalgia rheumatica; connective tissue diseases (e.g., systemic lupus erythematosus, systemic sclerosis, polymyositis, dermatomyositis, Sjogren's syndrome); vasculitides (e.g., polyarteritis nodosa, Wegener's granulomatosis, Churg-Strauss syndrome); inflammatory conditions including consequences of trauma or ischaemia, sarcoidosis; vascular diseases including atherosclerotic vascular disease, atherosclerosis, and vascular occlusive disease (e.g., atherosclerosis, ischaemic heart disease, myocardial infarction, stroke, peripheral vascular disease), and vascular stent restenosis; ocular diseases including uveitis, corneal disease, iritis, iridocyclitis, glaucoma, and cataracts.

All diseases and disorders listed herein may be defined as described in the Diagnostic and Statistical Manual of Mental Disorders (DSM-5), published by the American Psychiatric Association, or in International Classification of Diseases (ICD), published by the World Health Organization.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

### Compounds

Disclosed herein is a compound of Formula (I), or a pharmaceutically acceptable salt, polymorph, stereoisomer, or solvate thereof,

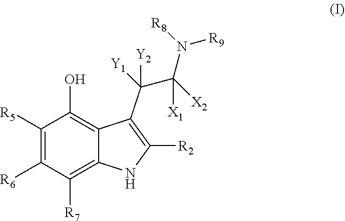

wherein:
R₂, R₅, R₆, and R₇ are independently selected from the group consisting of hydrogen and deuterium,
R₈ and R₉ are independently selected from the group consisting of —CH₃, —CH₂D, —CHD₂, and —CD₃, and
X₁, X₂, Y₁, and Y₂ are independently selected from the group consisting of hydrogen and deuterium.

In some embodiments, R₂, R₅, R₆, and R₇ are independently selected from the group consisting of hydrogen and deuterium. In some embodiments, R₂ is deuterium. In some embodiments, R₂ is hydrogen. In some embodiments, Rs is deuterium. In some embodiments, Rs is hydrogen. In some embodiments, R₆ is deuterium. In some embodiments, R₆ is hydrogen. In some embodiments, R₇ is deuterium. In some embodiments, R₇ is hydrogen.

R₂, R₅, R₆, and R₇ may be the same, for example, R₂, R₅, R₆, and R₇ may each be hydrogen, or alternatively, R₂, R₅, R₆, and R₇ may each be deuterium. In some embodiments, at least one of R₂, R₅, R₆, and R₇ is deuterium. In some embodiments, at least two of R₂, R₅, R₆, and R₇ are deuterium. In some embodiments, at least three of R₂, R, Ru, and R₇ are deuterium.

In some embodiments, R₈ and R₉ are independently selected from the group consisting of —CH₃, —CH₂D, —CHD₂, and —CD₃. R₈ and R₉ may be the same, or different. In some embodiments, R₈ and R₉ are the same. In some embodiments, R₈ and R₉ are independently selected from the group consisting of —CH₃ and —CD₃. In some embodiments, R₈ and R₉ are methyl (—CH₃). In some embodiments, R₈ and R₉ are a partially deuterated methyl group, i.e., —CDH₂ or —CD₂H. In some embodiments, R₈ and R₉ are a fully deuterated methyl group (—CD₃). In some embodiments, at least one of R₈ and R₉ is —CD₃.

In some embodiments, X₁, X₂, Y₁, and Y₂ are independently selected from the group consisting of hydrogen and deuterium. X₁ and X₂ may be the same, or different. In some embodiments, X₁ and X₂ are the same. In some embodiments, X₁ and X₂ are hydrogen. In some embodiments, X₁ and X₂ are deuterium.

Y₁ and Y₂ may be the same, or different. In some embodiments, Y₁ and Y₂ are the same.

In some embodiments, Y₁ and Y₂ are hydrogen. In some embodiments, Y₁ and Y₂ are deuterium.

In some embodiments, X₁, X₂, Y₁, and Y₂ are hydrogen. In some embodiments, X₁, X₂, Y₁, and Y₂ are deuterium.

In some embodiments, X₁, X₂, Y₁, Y₂, R₂, R₅, R₆, R₇, R₈, and R₉ are each hydrogen. In some embodiments, at least one of X₁, X₂, Y₁, Y₂, R₂, R₅, R₆, R₇, R₈, and R₉ comprises deuterium. In some embodiments, at least X₁, X₂, R₈, and R₉ comprise deuterium. In some embodiments, at least X₁, X₂, Y₁, Y₂, R₈, and R₉ comprise deuterium. In some embodiments, X₁, X₂, Y₁, and Y₂ are deuterium, and R₈ and R₉ are a fully deuterated methyl group (—CD₃).

The compounds of Formula (I) may contain a stereogenic center. In such cases, the compounds may exist as different stereoisomeric forms, even though Formula (I) is drawn without reference to stereochemistry. Accordingly, the present disclosure includes all possible stereoisomers and includes not only racemic compounds but the individual enantiomers (enantiomerically pure compounds), individual diastereomers (diastereomerically pure compounds), and their non-racemic mixtures as well. When a compound is desired as a single enantiomer, such may be obtained by, e.g., stereospecific synthesis, as is known in the art.

In some embodiments, the compounds described herein, e.g., compounds of Formula (I), are non-stereogenic. In some embodiments, the compounds described herein, e.g., compounds of Formula (I), are racemic. In some embodiments, the compounds described herein, e.g., compounds of Formula (I), are enantiomerically enriched (one enantiomer is present in a higher percentage), including enantiomerically pure. In some embodiments, the compounds described herein, e.g., compounds of Formula (I), are provided as a single diastereomer. In some embodiments, the compounds described herein, e.g., compounds of Formula (I), are provided as a mixture of diastereomers. When provided as a mixture of diastereomers, the mixtures may include equal mixtures, or mixtures which are enriched with a particular diastereomer (one diastereomer is present in a higher percentage than another).

In some embodiments, the compound of Formula (I) is an agonist of a serotonin 5-HT₂ receptor.

In some embodiments, the compound of Formula (I) is an agonist of a serotonin 5-HT_{2A}receptor.

In some embodiments, the compound of Formula (I) is selected from the group consisting of:

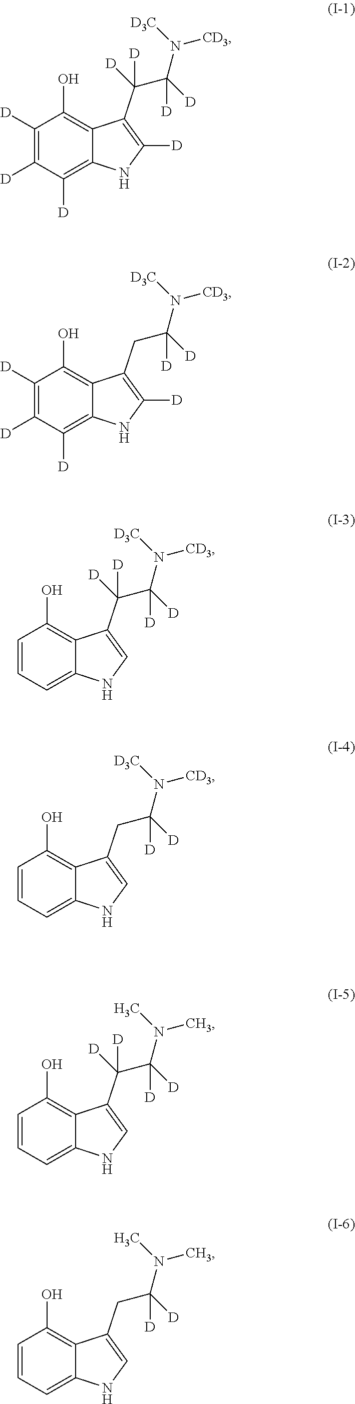 [SMILES: [2H]c1[nH]c2c([2H])c([2H])c([2H])c(O)c2c1C([2H])([2H])C([2H])([2H])N(C([2H])([2H])[2H])C([2H])([2H])[2H] ; [2H]c1[nH]c2c([2H])c([2H])c([2H])c(O)c2c1CC([2H])([2H])N(C([2H])([2H])[2H])C([2H])([2H])[2H] ; [2H]C([2H])([2H])N(C([2H])([2H])[2H])C([2H])([2H])C([2H])([2H])c1c[nH]c2cccc(O)c12 ; [2H]C([2H])([2H])N(C([2H])([2H])[2H])C([2H])([2H])Cc1c[nH]c2cccc(O)c12 ; [2H]C([2H])(c1c[nH]c2cccc(O)c12)C([2H])([2H])N(C)C ; [2H]C([2H])(Cc1c[nH]c2cccc(O)c12)N(C)C]

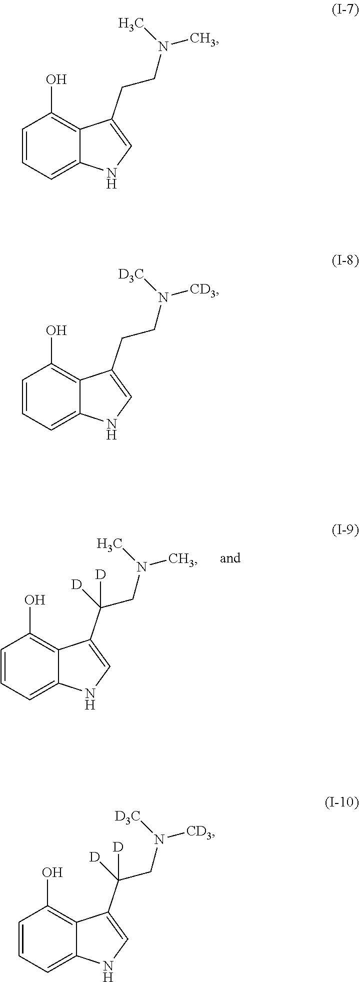 [SMILES: CN(C)CCc1c[nH]c2cccc(O)c12 ; [2H]C([2H])([2H])N(CCc1c[nH]c2cccc(O)c12)C([2H])([2H])[2H] ; [2H]C([2H])(CN(C)C)c1c[nH]c2cccc(O)c12 ; [2H]C([2H])(CN(C([2H])([2H])[2H])C([2H])([2H])[2H])c1c[nH]c2cccc(O)c12]

or a pharmaceutically acceptable salt, a polymorph, stereoisomer, or solvate thereof.

The compound number, IUPAC name, and substituent listing for the above-identified compounds are provided in Table 1.

TABLE 1
Exemplary compounds of Formula (I)

| | | Formula (I) | | | | | | |
|---|---|---|---|---|---|---|---|---|
| Compound identifier and name | | X₁, X₂ | Y₁, Y₂ | R₂ | R₅ | R₆ | R₇ | R₈, R₉ |
| I-1 | 3-(2-(bis(methyl-d₃)amino)ethyl- | D, D | D, D | D | D | D | D | —CD₃, —CD₃ |
| | 1,1,2,2-d₄)-1H-indol-2,5,6,7-d₄-4-ol | | | | | | | |
| I-2 | 3-(2-(bis(methyl-d₃)amino)ethyl-2,2- | D, D | H, H | D | D | D | D | —CD₃, —CD₃ |
| | d₂)-1H-indol-2,5,6,7-d₄-4-ol | | | | | | | |
| I-3 | 3-(2-(bis(methyl-d₃)amino)ethyl- | D, D | D, D | H | H | H | H | —CD₃, —CD₃ |
| | 1,1,2,2-d₄)-1H-indol-4-ol | | | | | | | |
| I-4 | 3-(2-(bis(methyl-d₃)amino)ethyl-2,2- | D, D | H, H | H | H | H | H | —CD₃, —CD₃ |
| | d₂)-1H-indol-4-ol | | | | | | | |
| I-5 | 3-(2-(dimethylamino)ethyl-1,1,2,2-d₄)- | D, D | D, D | H | H | H | H | —CH₃, —CH₃ |
| | 1H-indol-4-ol | | | | | | | |
| I-6 | 3-(2-(dimethylamino)ethyl-2,2-d₂)-1H- | D, D | H, H | H | H | H | H | —CH₃, —CH₃ |
| | indol-4-ol | | | | | | | |
| I-7 | 3-(2-(dimethylamino)ethyl)-1H-indol- | H, H | H, H | H | H | H | H | —CH₃, —CH₃ |
| | 4-ol | | | | | | | |
| I-8 | 3-(2-(bis(methyl-d₃)amino)ethyl)-1H- | H, H | H, H | H | H | H | H | —CD₃, —CD₃ |
| | indol-4-ol | | | | | | | |
| I-9 | 3-(2-(dimethylamino)ethyl-1,1-d₂)-1H- | H, H | D, D | H | H | H | H | —CH₃, —CH₃ |
| | indol-4-ol | | | | | | | |
| I-10 | 3-(2-(bis(methyl-d₃)amino)ethyl-1,1- | H, H | D, D | H | H | H | H | —CD₃, —CD₃ |
| | d₂)-1H-indol-4-ol | | | | | | | |

In some embodiments, the compounds of the present disclosure are provided as a free base in crystalline form, e.g., as determined by XRPD and/or mDSC. Accordingly, pharmaceutical compositions may be prepared from compounds of Formula (I) as a free base, in one or more crystalline (e.g., polymorphic) forms, and may be used for treatment as set forth herein. In some embodiments, a crystalline form of a compound of Formula (I) as a free base is provided. For example, the pharmaceutical composition may comprise a free base of a compound of Formula (I), wherein at least 50%, at least 60%, at least 70%, at least 80%, at least 90%, at least 95%, at least 99%, or at least 99.5% by weight of the free base of the compound of Formula (I) present in the pharmaceutical composition is in crystalline form, e.g., as determined by X-ray powder diffraction and/or mDSC. In some embodiments, a highly pure crystalline form of a compound of Formula (I) as a free base is provided. For example, the pharmaceutical composition may comprise a free base of a compound of Formula (I), wherein at least 90%, at least 95%, at least 99%, or at least 99.5% by weight of the free base of the compound of Formula (I) present in the pharmaceutical composition is in crystalline form, e.g., as determined by X-ray powder diffraction and/or mDSC.

In some embodiments, the compound of Formula (I) is a crystalline form of 3-(2-(bis(methyl-d₃)amino)ethyl-1,1,2,2-d₄)-1H-indol-2,5,6,7-d₄-4-ol (I-1), as determined by X-ray powder diffraction.

In some embodiments, the compound of Formula (I) is a crystalline form of 3-(2-(bis(methyl-d₃)amino)ethyl-2,2-d₂)-1H-indol-2,5,6,7-d₄-4-ol (I-2), as determined by X-ray powder diffraction.

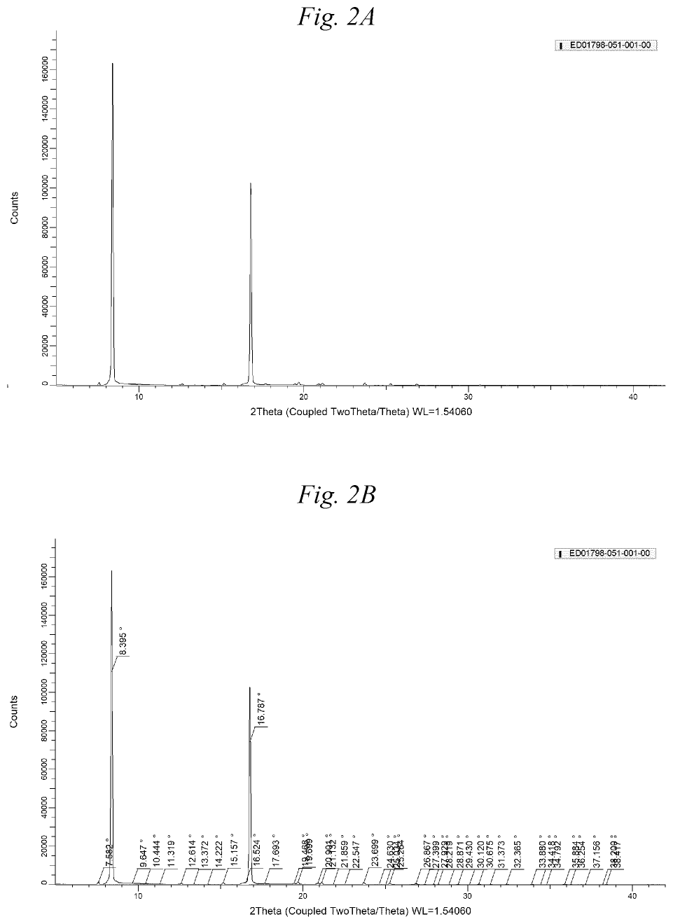

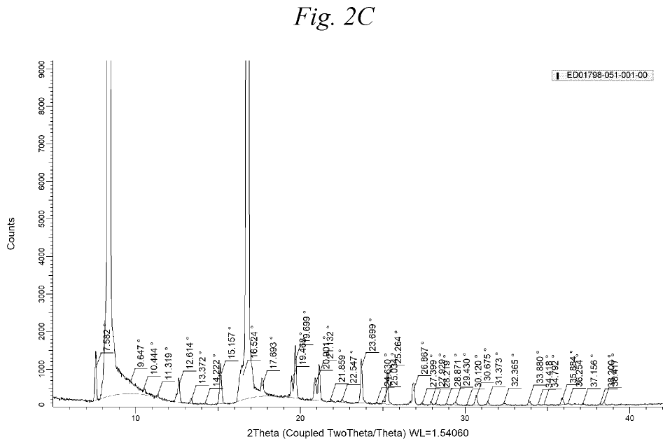

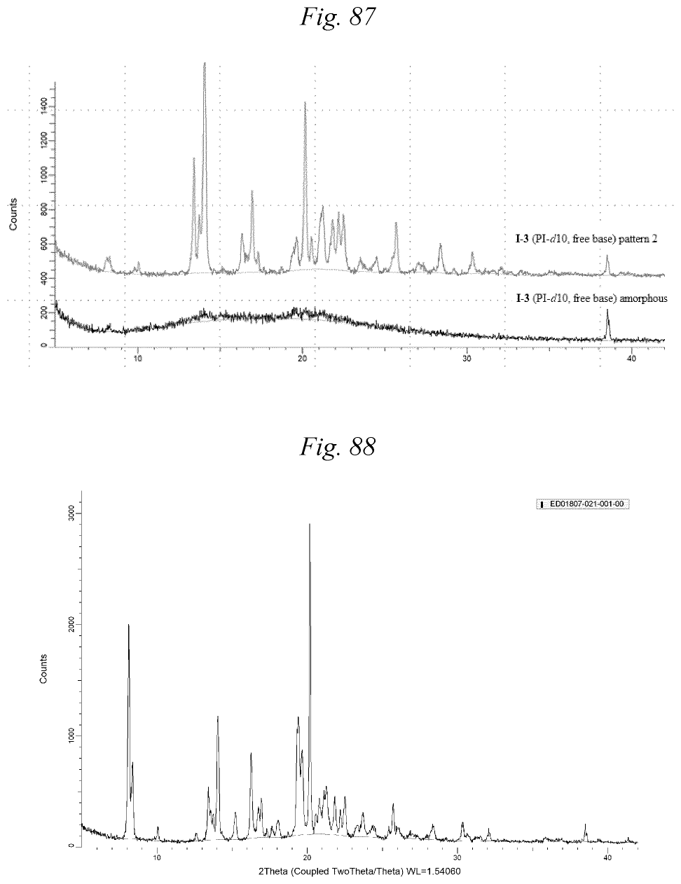

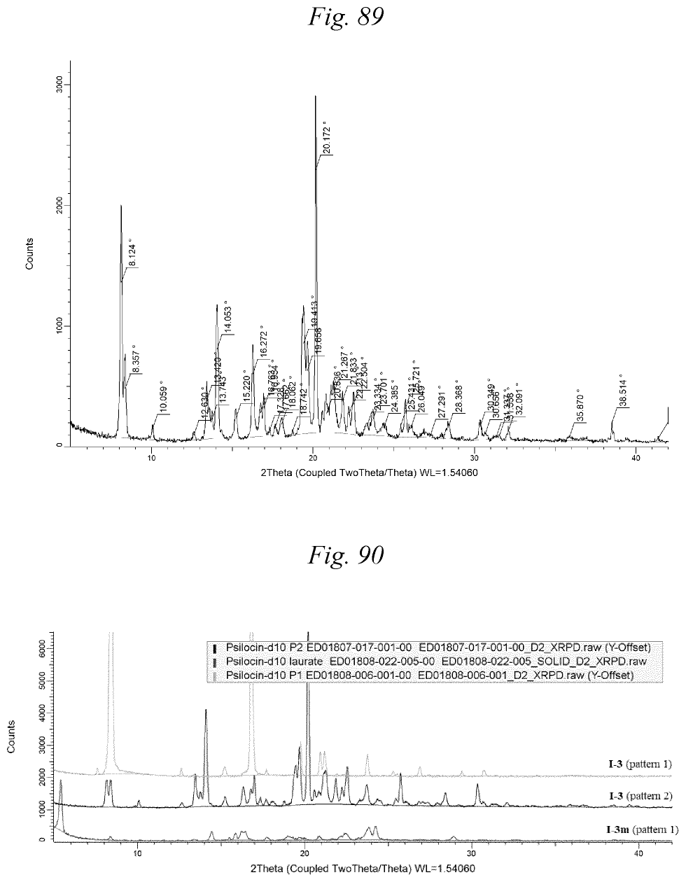

In some embodiments, the compound of Formula (I) is a crystalline form of 3-(2-(bis(methyl-d₃)amino)ethyl-1,1,2,2-d₄)-1H-indol-4-ol (I-3), as determined by X-ray powder diffraction. In some embodiments, I-3 is a crystalline solid form (pattern 1) characterized by an X-ray powder diffraction pattern containing at least three characteristic peaks at diffraction angles (2θ±0.2°) selected from 7.582°, 8.395°, 9.647°, 10.444°, 11.319°, 12.614°, 13.372°, 14.222°, 15.157°, 16.524°, 16.787°, 17.693°, 19.468°, 19.699°, 20.901°, 21.132°, 21.859°, 22.547°, 23.699°, 24.630°, 25.034°, 25.264°, 26.867°, 27.399°, 27.929°, 28.219°, 28.871°, 29.430°, 30.120°, 30.675°, 31.373°, 32.365°, 33.880°, 34.418°, 34.792°, 35.884°, 36.254°, 37.156°, 38.200°, and 38.417°, as determined by XRPD using a CuKα radiation source, for example, as shown in FIGS. 2A-2C. In some embodiments, I-3 is a crystalline solid form (pattern 2) characterized by an X-ray powder diffraction pattern containing at least three characteristic peaks at diffraction angles (2θ±0.2°) selected from 8.124°, 8.357°, 10.059°, 12.630°, 13.420°, 13.743°, 14.053°, 15.220°, 16.272°, 16.763°, 16.954°, 17.328°, 17.662°, 18.062°, 18.742°, 19.413°, 19.658°, 20.172°, 20.836°, 21.267°, 21.833°, 22.213°, 22.504°, 23.334°, 23.701°, 24.385°, 25.431°, 25.721°, 26.049°, 27.291°, 28.368°, 30.349°, 30.656°, 31.337°, 31.538°, 32.091°, 35.870°, 38.514°, and 41.361°, as determined by XRPD using a CuKα radiation source, for example, as shown in FIGS. 88-89.

In some embodiments, the compound of Formula (I) is a crystalline form of 3-(2-(bis(methyl-d₃)amino)ethyl-2,2-d₂)-1H-indol-4-ol (I-4), as determined by X-ray powder diffraction.

In some embodiments, the compound of Formula (I) is a crystalline form of 3-(2-(dimethylamino)ethyl-1,1,2,2-d₄)-1H-indol-4-ol (I-5), as determined by X-ray powder diffraction.

In some embodiments, the compound of Formula (I) is a crystalline form of 3-(2-(dimethylamino)ethyl-2,2-d₂)-1H-indol-4-ol (I-6), as determined by X-ray powder diffraction.

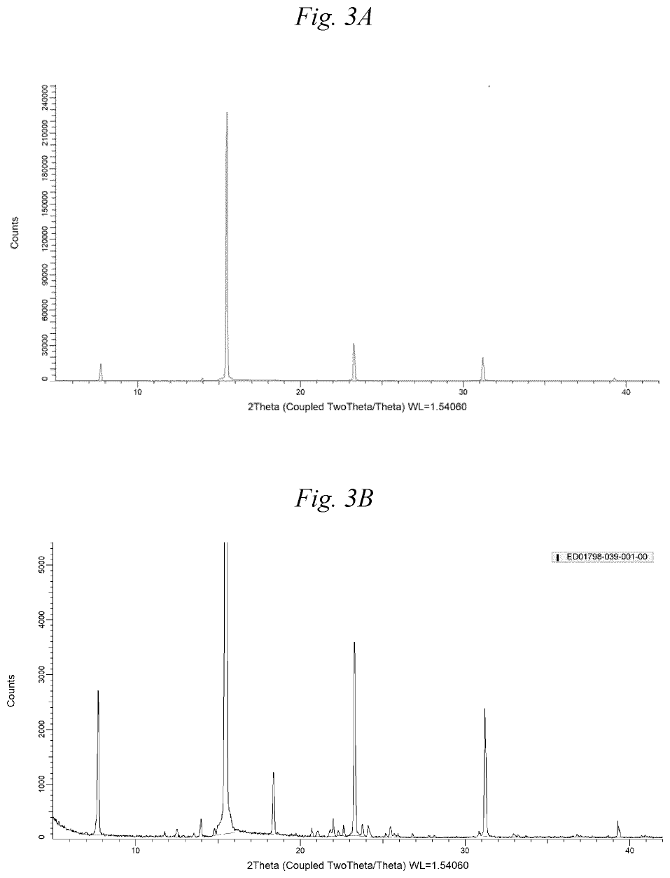

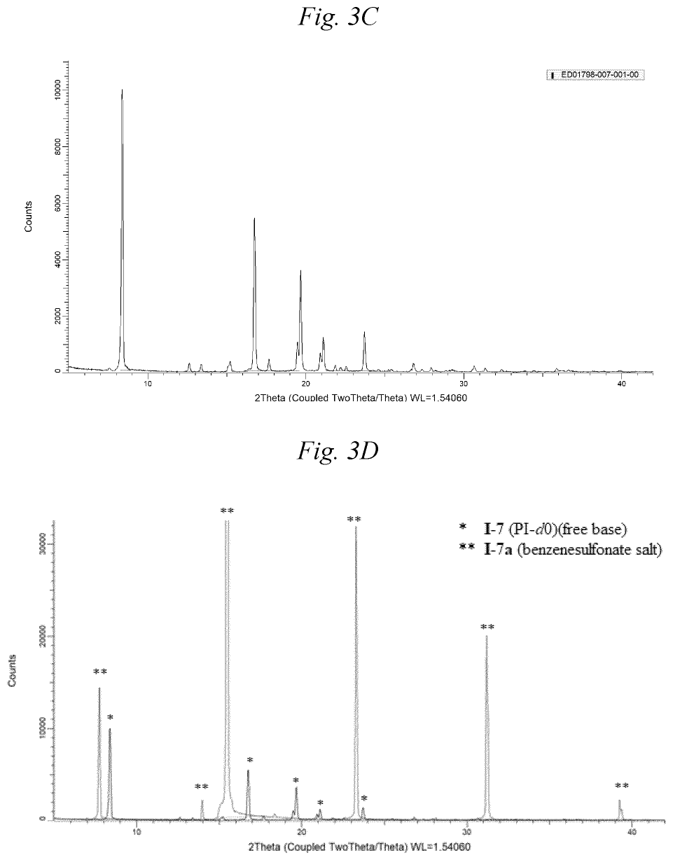

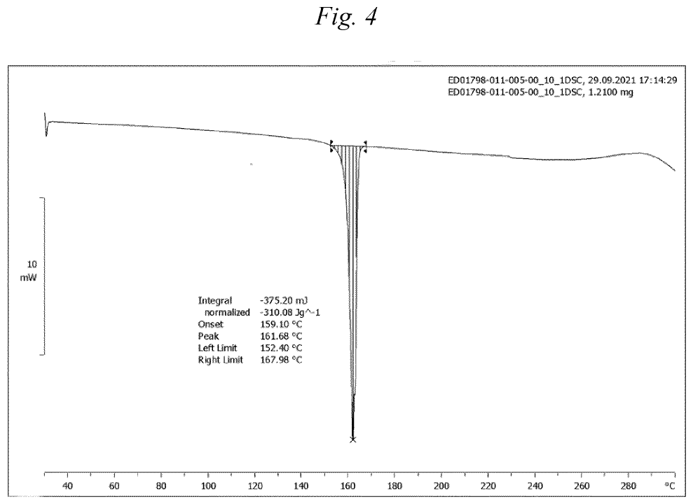

In some embodiments, the compound of Formula (I) is a crystalline form of 3-(2-(dimethylamino)ethyl)-1H-indol-4-ol (I-7), as determined by X-ray powder diffraction. In some embodiments, I-7 is a crystalline solid form (pattern 1) characterized by an X-ray powder diffraction pattern containing at least three characteristic peaks at diffraction angles (2θ±0.2°) selected from 7.563°, 8.375°, 12.626°, 13.383°, 15.211°, 16.753°, 17.671°, 19.668°, 21.112°, 21.863°, 22.2010, 22.560°, 23.711°, 24.592°, 25.415°, 26.820°, 27.357°, 27.921°, 28.228°, 29.253°, 30.653°, 31.364°, 32.401°, 33.797°, 34.445°, and 39.867°, as determined by XRPD using a CuKα radiation source, for example, as shown in FIG. 3C.

In some embodiments, the compound of Formula (I) is a crystalline form of 3-(2-(bis(methyl-d₃)amino)ethyl)-1H-indol-4-ol (I-8), as determined by X-ray powder diffraction.

In some embodiments, the compound of Formula (I) is a crystalline form of 3-(2-(dimethylamino)ethyl-1,1-d₂)-1H-indol-4-ol (I-9), as determined by X-ray powder diffraction.

In some embodiments, the compound of Formula (I) is a crystalline form of 3-(2-(bis(methyl-d₃)amino)ethyl-1,1-d₂)-1H-indol-4-ol (I-10), as determined by X-ray powder diffraction.

In some embodiments, the compounds of the present disclosure are provided as a free base in amorphous form, e.g., as determined by XRPD and/or mDSC. Accordingly, pharmaceutical compositions may be prepared from compounds of Formula (I) as a free base, in one or more amorphic forms, and may be used for treatment as set forth herein. In some embodiments, a highly pure amorphous form of a compound of Formula (I) as a free base is provided. For example, the pharmaceutical composition may comprise a free base of a compound of Formula (I), wherein at least 92%, at least 94%, at least 96%, at least 98%, at least 99%, or at least 99.5% by weight of the free base of the compound of Formula (I) present in the pharmaceutical composition is in amorphous form, e.g., as determined by X-ray powder diffraction and/or mDSC.

Numerous attempts to make an amorphous form of the compounds of the present disclosure proved unsuccessful, including crash cooling/freeze drying, fast evaporation from numerous organic solvents, and anti-solvent precipitation. Crash cooling/freeze drying and fast evaporation techniques each gave only crystalline material, while anti-solvent precipitation failed to produce solid material. After significant experimentation, it has been discovered that amorphous forms of the compounds of Formula (I), e.g., compound I-3 (psilocin-d₁₀) can be prepared through a melt/crash cooling procedure. Briefly, crystalline free base material may be heated beyond its melting point, e.g., to at least 180° C., at least 181° C., at least 182° C., at least 183° C., at least 184° C., at least 185° C. using DSC or similar technique, followed by rapid cooling to near (e.g., ±5° C.) the glass transition of the material, e.g., to about 26° C., about 27° C., about 28° C., about 29° C., about 30° C., as determined by differential scanning calorimetry (DSC). For example, it has been found that amorphous I-3 (PI-d₁₀, free base) can be prepared by a melt/crash cooling procedure in DSC in which crystalline I-3 is heated to beyond the melting point (to 185° C.) and then rapidly cooled to 30° C. (glass transition temperature of 27° C.). The amorphous nature of the compound of Formula (I) can be determined e.g., by XRPD.

In some embodiments, the compound of Formula (I) is an amorphous form of 3-(2-(bis(methyl-d₃)amino)ethyl-1,1,2,2-d₄)-1H-indol-2,5,6,7-d₄-4-ol (I-1), as determined by X-ray powder diffraction. In some embodiments, the compound of Formula (I) is an amorphous form of 3-(2-(bis(methyl-d₃)amino)ethyl-2,2-d₂)-1H-indol-2,5,6,7-d₄-4-ol (I-2), as determined by X-ray powder diffraction. In some embodiments, the compound of Formula (I) is an amorphous form of 3-(2-(bis(methyl-d₃)amino)ethyl-1,1,2,2-d₄)-1H-indol-4-ol (I-3), as determined by X-ray powder diffraction. In some embodiments, the compound of Formula (I) is an amorphous form of 3-(2-(bis(methyl-d₃)amino)ethyl-2,2-d₂)-1H-indol-4-ol (I-4), as determined by X-ray powder diffraction. In some embodiments, the compound of Formula (I) is an amorphous form of 3-(2-(dimethylamino)ethyl-1,1,2,2-d₄)-1H-indol-4-ol (I-5), as determined by X-ray powder diffraction.

In some embodiments, the compound of Formula (I) is an amorphous form of 3-(2-(dimethylamino)ethyl-2,2-d₂)-1H-indol-4-ol (I-6), as determined by X-ray powder diffraction. In some embodiments, the compound of Formula (I) is an amorphous form of 3-(2-(dimethylamino)ethyl)-1H-indol-4-ol (I-7), as determined by X-ray powder diffraction. In some embodiments, the compound of Formula (I) is an amorphous form of 3-(2-(bis(methyl-d₃)amino)ethyl)-1H-indol-4-ol (I-8), as determined by X-ray powder diffraction. In some embodiments, the compound of Formula (I) is an amorphous form of 3-(2-(dimethylamino)ethyl-1,1-d₂)-1H-indol-4-ol (I-9), as determined by X-ray powder diffraction. In some embodiments, the compound of Formula (I) is an amorphous form of 3-(2-(bis(methyl-d₃)amino)ethyl-1,1-d₂)-1H-indol-4-ol (I-10), as determined by X-ray powder diffraction.

Such amorphous forms of the compounds of Formula (I) (free base) may be advantageous in terms of dissolution rates in water, compared to crystalline forms, thereby enabling rapid systemic absorption for quick therapeutic onset and a short duration of drug action. Further, in some embodiments, pharmaceutical compositions may be prepared which comprise the amorphous forms of the compounds of Formula (I) (free base). The pharmaceutical compositions of the present disclosure, such as those set forth herein, may act to stabilize the amorphous forms of the compounds of Formula (I), which tend to be unstable and have a tendency to crystallize.

Accordingly, the pharmaceutical compositions can be used to stabilize and deliver these amorphous forms to subjects in need of treatment, e.g., for the treatment of a condition or disease associate with a serotonin 5-HT₂ receptor.

### Salt Forms

Also disclosed herein is a pharmaceutically acceptable salt of the compound of Formula (I), or a pharmaceutically acceptable polymorph, stereoisomer, or solvate thereof. The acid used to form the pharmaceutically acceptable salt of the compound of Formula (I) may be a monoacid, a diacid, a triacid, a tetraacid, or may contain a higher number of acid groups. The acid groups may be, e.g., a carboxylic acid, a sulfonio acid, a phosphonic acid, or other acidic moieties containing at least one replaceable hydrogen atom. Examples of acids, which may be organic or inorganic acids, for use in the preparation of the pharmaceutically acceptable (acid addition) salts disclosed herein include, but are not limited to, acetic acid, 2,2-dichloroacetic acid, phenylacetic acid, acylated amino acids, alginic acid, ascorbic acid, L-aspartic acid, sulfonic acids (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.), benzoic acids (e.g., benzoic acid, 4-acetamidobenzoic acid, 2-acetoxybenzoic acid, salicylic acid, 4-amino-salicylic acid, gentisic acid, etc.), boric acid, (+)-camphoric acid, cinnamic acid, citric acid, cyclamic acid, cyclohexanesulfamic acid, dodecylsulfuric acid, formic acid, fumaric acid, galactaric acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (−)-D-lactic acid, (t)-DL-lactic acid, lactobionic acid, maleic acid, malic acid, (−)-L-malic acid, (+)-D-malic acid, hydroxymaleic acid, malonic acid, (f)-DL-mandelic acid, isethionic acid, 1-hydroxy-2-naphthoic acid, nicotinic acid, nitric acid, orotic acid, oxalic acid, pamoic acid, perchloric acid, phosphoric acid, L-pyroglutamic acid, saccharic acid, succinic acid, sulfuric acid, sulfamic acid, tannic acid, tartaric acids (e.g., DL-tartaric acid, (+)-L-tartaric acid, (−)-D-tartaric acid), thiocyanic acid, propionic acid, valeric acid, and fatty acids (including fatty mono- and di-acids, e.g., adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc.).

Certain salts are preferred among the list above because they possess physical and pharmaceutical characteristics/properties which make them more suitable for pharmaceutical preparation and administration. For example, preferred salt forms of the compounds disclosed herein (e.g., compounds of Formula (I)) are those that possess one or more of the following characteristics: are easy to prepare in high yield with a propensity towards salt formation; are stable and have well-defined physical properties such as crystallinity, defined and reproducible polymorphism insofar as polymorphism exists, and high melting/enthalpy of fusion; have slight or no hygroscopicity; are free flowing, do not cohere/adhere to surfaces, and possess a regular morphology; have acceptable aqueous solubility and rate of dissolution for the intended dosage form; and/or are physiologically acceptable, e.g., do not cause excessive irritation.

### Crystallinity

The pharmaceutically acceptable salt of the compound of Formula (I) may be crystalline or amorphous, as determined e.g., by X-ray powder diffraction (XRPD) and/or mDSC. In some embodiments, the salt of the compound of Formula (I) is amorphous. Amorphous forms typically possess higher aqueous solubility and rates of dissolution compared to their crystalline counterparts, and thus may be well suited for quick acting dosage forms adapted to rapidly release the active ingredient, such as orodispersible dosage forms (ODxs), immediate release (IR) dosage forms, and the like. The salts of the compound of Formula (I) can be in a stable amorphous form.

In some embodiments, the pharmaceutically acceptable salt of the compound of Formula (I) is provided in amorphous form, e.g., as determined by XRPD and/or mDSC. Accordingly, pharmaceutical compositions may be prepared from pharmaceutically acceptable salt forms of compounds of Formula (I), in one or more amorphic forms, and may be used for treatment as set forth herein. In some embodiments, a highly pure amorphous form of a pharmaceutically acceptable salt of a compound of Formula (I) is provided. For example, the pharmaceutical composition may comprise a pharmaceutically acceptable salt of a compound of Formula (I), wherein at least 92%, at least 94%, at least 96%, at least 98%, at least 99%, or at least 99.5% by weight of the pharmaceutically acceptable salt of the compound of Formula (I) present in the pharmaceutical composition is in amorphous form, e.g., as determined by X-ray powder diffraction and/or mDSC.

In some embodiments, the salt of the compound of Formula (I) is crystalline. Crystalline forms are advantageous in terms of stability and providing well-defined physical properties, which is desirable for pharmaceutical preparation and administration. The salts of the compound of Formula (I) can be in a stable crystalline form. In some embodiments, the pharmaceutically acceptable salt of the compound of Formula (I) has a percent crystallinity of at least 50%, at least 60%, at least 70%, at least 80%, at least 90%, at least 95%, at least 99%, or at least 99.5%, and up to 100%, as determined by XRPD and/or mDSC analysis. For example, a pharmaceutical composition may be provided which comprises a pharmaceutically acceptable salt of a compound of Formula (I), wherein at least 50%, at least 60%, at least 70%, at least 80%, at least 90%, at least 95%, at least 99%, or at least 99.5% by weight of the pharmaceutically acceptable salt of the compound of Formula (I) present in the pharmaceutical composition is in crystalline form, e.g., as determined by X-ray powder diffraction and/or mDSC. In some embodiments, a highly pure crystalline form of a pharmaceutically acceptable salt of a compound of Formula (I) is provided. For example, the pharmaceutical composition may comprise a pharmaceutically acceptable salt of a compound of Formula (I), wherein at least 90%, at least 95%, at least 99%, or at least 99.5% by weight of the pharmaceutically acceptable salt of the compound of Formula (I) present in the pharmaceutical composition is in crystalline form, e.g., as determined by X-ray powder diffraction and/or mDSC. Preference is given to salt forms with high crystallinity, as determined e.g., by discrete and sharp Bragg diffractions in the X-ray diffractograms.

XRPD analyses can be carried out, e.g., on a Bruker AXS D2 diffractometer using CuKα radiation (wavelength=1.54060 Å). The instrument may be equipped with a fine focus X-ray tube. The tube voltage and amperage can be set to 30 kV and 10 mA, respectively, and a 0-0 geometry can be used, using a LynxEye detector from 5-42° 2θ, with a step size of 0.024° 2θ and a collection time of 0.1 seconds per step.

In terms of pharmaceutical production processes, advantageous salt forms of the compounds of Formula (I) are those that readily afford a solid material, either a crystalline solid or an amorphous solid, in acceptable yield without proceeding via an oil, and with favorable volume factors, making them suitable for mass production.

Salts forms of the compound of Formula (I) can exist in different polymorphs (i.e., forms having a different crystal structure), however, preferred salt forms of the present disclosure are those which can be generated as a single crystalline form or single polymorph (including a single amorphous form), as determined by XRPD and/or mDSC and/or differential scanning calorimetry (DSC). It is also generally desirable for the salts to be free flowing, not cohere/adhere to surfaces, and possess a regular morphology.

### Chemical/Solid-State Stability

In some embodiments, the pharmaceutically acceptable salt of the compound of Formula (I) has a melt onset of from about 90° C., from about 100° C., from about 110° C., from about 120° C., from about 130° C., from about 140° C., from about 150° C., from about 160° C., from about 170° C., from about 180° C., from about 190° C., and up to about 250° C., up to about 240° C., up to about 230° C., up to about 225° C., up to about 210° C., up to about 200° C., as determined by DSC.

Pharmaceutically acceptable salts of the compound of Formula (I) may also be characterized as non-hygroscopic or slightly hygroscopic, preferably non-hygroscopic. The hygroscopicity may be measured herein by performing a moisture adsorption-desorption isotherm using a dynamic vapor sorption (DVS) analyzer with a starting exposure of 40% relative humidity (RH), increasing humidity up to 90% RH, decreasing humidity to 0% RH, increasing humidity to 90% RH, decreasing humidity to 0% RH, and finally increasing the humidity back to the starting 40% RH, and classified according to the following:
non-hygroscopic: <0.2%; slightly hygroscopic: ≥0.2% and <2%; hygroscopic: ≥2% and <15%; very hygroscopic: ≥15%; deliquescent: sufficient water is absorbed to form a liquid; all values measured as weight increase (w/w due to acquisition of water) at >90% RH and 25° C.

In some embodiments, the pharmaceutically acceptable salt of the compound of Formula (I) has a weight increase at >90% RH of less than 1% w/w, less than 0.8% w/w, less than 0.6% w/w, less than 0.5% w/w, less than 0.4% w/w, less than 0.3% w/w, less than 0.2% w/w, less than 0.1% w/w, less than 0.08% w/w, less than 0.06% w/w, less than 0.05% w/w, less than 0.02% w/w, as determined by DVS.

Dry powder samples of free base and salts can be maintained/stored in open or closed environments, such as in open or closed flasks/vials, under ambient or stress conditions e.g., 25° C./90+% RH, 40° C./75% RH, etc. without appreciable degradation or physical changes (e.g., changed forms, deliquesced, etc.). For example, dry powder samples of free base and salts forms disclosed herein may have a purity or form change of less than 10%, less than 5%, less than 1%, when stored under ambient conditions or stress conditions (e.g., increased temperature, e.g., 40° C., and/or humidity).

Solution-phase compositions of the free base and salts can be maintained/stored in open or closed environments, such as in open or closed flasks/vials, under ambient or stress conditions e.g., 25° C./90+% RH, 40° C./75% RH, etc. without appreciable degradation. Thus, in some embodiments, the present disclosure provides stable solution-phase compositions of free base and salt forms of the compounds of Formula (I) (e.g., stable solvates of free base or salt forms of compounds of Formula (I) which are in solvated form, preferably fully solvated form), which can be stored as a solution, such as in the form of an aqueous solution, an organic solvent solution, or a mixed aqueous-organic solvent solution, for prolonged periods of time without appreciable degradation or physical changes, such as oiling out of solution. Solvents which can be used to form the solution-phase compositions can be any one or more solvents set forth herein, e.g., water, ethanol, fruit juice, etc. In some embodiments, the solution-phase composition is an aqueous solution-phase composition comprising the free base or a pharmaceutically acceptable salt of the compound of Formula (I) solvated with water (and optionally comprising other components such as those found in fruit juice). The identification of stable solution-phase compositions of compounds of Formula (I) and their salts is advantageous at least because such compositions do not require use immediately after being prepared, such as within 5 minutes, within 4 minutes, within 3 minutes, within 2 minutes, within 1 minute, within 45 seconds, within 30 seconds, within 15 seconds, within 10 seconds of being prepared. Instead, the stable solution-phase compositions of the compounds of Formula (I) and salts thereof described herein can be prepared in advance, when desired, optionally stored, and can be administered hours, days, or even weeks after being prepared, without materially effecting efficacy, e.g., without appreciable degradation of the psilocin or psilocin-type active.

In some embodiments, aqueous solutions formed from the pharmaceutically acceptable salt of the compound of Formula (I) are characterized by increased stability compared to aqueous solutions that are prepared from the compound of Formula (I) (free base) but are otherwise substantially the same. For example, the pharmaceutically acceptable salt of the compound of Formula (I) may be at least 5%, at least 10%, at least 15%, at least 20%, at least 25%, at least 30%, at least 35%, at least 40%, at least 45%, at least 50%, at least 55%, at least 60%, at least 65%, at least 70% more stable in aqueous solution subjected to 40° C. for 24 hours, with or without the presence of metal ions, in terms of % (active) remaining, compared to aqueous solutions prepared with the compound of Formula (I) (free base) but are otherwise substantially the same. Such improved stability behavior can also be found in pharmaceutical compositions of the present disclosure.

Samples can be pulled at pre-determined time-points and analyzed for stability, changes in form, etc. for example, by ¹H NMR, XRPD, HPLC with UV-visible multiple wavelength detector, UPLC, etc.

### Physiologically Acceptability

Suitable salt forms of the compounds of Formula (I) are physiologically acceptable. Accordingly, preferred addition salts of the compound of Formula (I) are those formed with an organic acid, preferably an organic acid with a medium or mild acidity, for example an organic acid with a pKₐ in water of no less than −3.0, no less than −2.0, no less than −1.0, no less than 0, no less than 1.0, no less than 1.5, no less than 2.0, no less than 2.5, no less than 3.0, no less than 3.5, no less than 4.0, no less than 4.5, for example, from 3.0 to 6.5. Further, it may also be desirable to use acid addition salts that impart a pleasant taste profile (e.g., sweet, citrus flavored, etc.), although poor tasting salt forms (e.g., bitter, harsh, etc.) may still be acceptable depending on, for example, the mute of administration and the optional use of taste masking agents such as sweetening agents, flavoring agents, etc.

### Solubility

The aqueous solubility of the salt forms of the compounds of Formula (I) can be determined by equilibrating excess solid with 1 mL of water for 24 hours at 22° C. A 200 μL aliquot can be centrifuged at 15,000 rpm for 15 minutes. The supernatant can be analyzed by HPLC and the solubility can be expressed as its free base equivalent (mg FB/mL). For example, pharmaceutically acceptable salts of compound of Formula (I) can be prepared and the solubility and solution pH can be measured.

In some embodiments, the pharmaceutically acceptable salt of the compound of Formula (I) has a water solubility at 22° C. of from about 1 mg/mL to about 400 mg/mL. In some embodiments, the pharmaceutically acceptable salt of the compound of Formula (I) has a water solubility of from about 1 mg/mL, from about 2 mg/mL, from about 3 mg/mL, from about 5 mg/mL, from about 10 mg/mL, from about 20 mg/mL, from about 30 mg/mL, from about 40 mg/mL, from about 50 mg/mL, from about 60 mg/mL, from about 70 mg/mL, from about 80 mg/mL, from about 90 mg/mL, from about 100 mg/mL, from about 110 mg/mL, from about 120 mg/mL, from about 130 mg/mL, from about 140 mg/mL, from about 150 mg/mL, and up to about 400 mg/mL, up to about 380 mg/mL, up to about 360 mg/mL, up to about 340 mg/mL, up to about 320 mg/mL, up to about 300 mg/mL, up to about 280 mg/mL, up to about 260 mg/mL, up to about 250 mg/mL. Several salt forms of the compounds described herein can exhibit the above solubilities, yielding a final water pH approximately between pH 3 to 6 without gelling.

In some embodiments, the salt of the compound of Formula (I) has a water solubility from about 200 mg/mL to about 400 mg/mL. In some embodiments, the salt of the compound of Formula (I) has a water solubility from about 150 mg/mL to about 250 mg/mL. In some embodiments, the salt of the compound of Formula (I) has a water solubility of greater than about 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL.

In some embodiments, salt forms of the compounds of Formula (I) possess dissolution rates which enable rapid systemic absorption for quick therapeutic onset and a short duration of drug action. In some embodiments, the salt of the compound of Formula (I) is capable of dissolution in an aqueous medium below about pH 7.5, such as from pH 1-7, from pH 3-7, or from pH 4-7.

In some embodiments, the pharmaceutically acceptable salt of the compound of Formula (I) is a benzenesulfonate salt, a tartrate salt, a hemi-fumarate salt, an acetate salt, a citrate salt, a hemi-malonate salt, a malonate salt, a fumarate salt, a succinate salt, a hemi-succinate salt, an oxalate salt, a benzoate salt, a salicylate salt, an ascorbate salt, a hydrochloride salt, a maleate salt, a malate salt, a methanesulfonate salt, a toluenesulfonate salt, a glucuronate salt, or a glutarate salt of the compound of Formula (I). In some embodiments, the pharmaceutically acceptable salt of the compound of Formula (I) is a salt formed from a sulfonic acid (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.). In some embodiments, the pharmaceutically acceptable salt of the compound of Formula (I) is a salt formed from a benzoic acid (e.g., benzoic acid, 4-acetamidobenzoic acid, 2-acetoxybenzoic acid, salicylic acid, 4-amino-salicylic acid, etc.). The pharmaceutically acceptable salt of the compound of Formula (I) may be a hemi-acid salt of any of the salts listed above when the acid used to form the salt contains more than one acidic group (e.g., more than one carboxylic acid moiety).

In some embodiments, the pharmaceutically acceptable salt of the compound of Formula (I) is a benzenesulfonate salt. In some embodiments, the pharmaceutically acceptable salt of the compound of Formula (I) is a tartrate salt. In some embodiments, the pharmaceutically acceptable salt of the compound of Formula (I) is a hemi-fumarate salt. In some embodiments, the pharmaceutically acceptable salt of the compound of Formula (I) is an acetate salt. In some embodiments, the pharmaceutically acceptable salt of the compound of Formula (I) is a citrate salt. In some embodiments, the pharmaceutically acceptable salt of the compound of Formula (I) is a hemi-malonate salt. In some embodiments, the pharmaceutically acceptable salt of the compound of Formula (I) is a fumarate salt. In some embodiments, the pharmaceutically acceptable salt of the compound of Formula (I) is a hemi-succinate salt. In some embodiments, the pharmaceutically acceptable salt of the compound of Formula (I) is an oxalate salt. In some embodiments, the pharmaceutically acceptable salt of the compound of Formula (I) is a benzoate salt. In some embodiments, the pharmaceutically acceptable salt of the compound of Formula (I) is a salicylate salt. In some embodiments, the pharmaceutically acceptable salt of the compound of Formula (I) is an ascorbate salt. In some embodiments, the pharmaceutically acceptable salt of the compound of Formula (I) is a hydrochloride salt. In some embodiments, the pharmaceutically acceptable salt of the compound of Formula (I) is a maleate salt. In some embodiments, the pharmaceutically acceptable salt of the compound of Formula (I) is a malate salt. In some embodiments, the pharmaceutically acceptable salt of the compound of Formula (I) is a methanesulfonate salt. In some embodiments, the pharmaceutically acceptable salt of the compound of Formula (I) is a toluenesulfonate salt. In some embodiments, the pharmaceutically acceptable salt of the compound of Formula (I) is a glucuronate salt. In some embodiments, the pharmaceutically acceptable salt of the compound of Formula (I) is a glutarate salt.

In some embodiments, the pharmaceutically acceptable salt of the compound of Formula (I) is a benzenesulfonate salt, a tartrate salt, a hemi-fumarate salt, an acetate salt, a citrate salt, a hemi-malonate salt, a fumarate salt, a hemi-succinate salt, an oxalate salt, a benzoate salt, or a salicylate salt of the compound of Formula (I), with a benzenesulfonate salt, a hemi-succinate salt, or a benzoate salt of the compound of Formula (I) being preferred, and with a benzenesulfonate salt or a benzoate salt of the compound of Formula (I) being particularly preferred.

Exemplary pharmaceutically acceptable salt forms (i.e., addition salt forms) of the above-identified compounds are provided in Table 2.

TABLE 2
Exemplary pharmaceutically acceptable
salts of compounds of Formula (I)

| Salt form identifier | Salt type of compound |
|---|---|
| I-1a | Benzenesulfonate of I-1 |
| I-1b | Tartrate of I-1 |
| I-1c | Hemi-fumarate of I-1 |
| I-1d | Acetate of I-1 |
| I-1e | Citrate of I-1 |
| I-1f | Hemi-malonate of I-1 |
| I-1g | Fumarate of I-1 |
| I-1h | Hemi-succinate of I-1 |
| I-1i | Oxalate of I-1 |
| I-1j | Benzoate of I-1 |
| I-1k | Salicylate of I-1 |
| I-2a | Benzenesulfonate of I-2 |
| I-2b | Tartrate of I-2 |
| I-2c | Hemi-fumarate of I-2 |
| I-2d | Acetate of I-2 |
| I-2e | Citrate of I-2 |
| I-2f | Hemi-malonate of I-2 |
| I-2g | Fumarate of I-2 |
| I-2h | Hemi-succinate of I-2 |
| I-2i | Oxalate of I-2 |
| I-2j | Benzoate of I-2 |
| I-2k | Salicylate of I-2 |
| I-3a | Benzenesulfonate of I-3 |
| I-3b | Tartrate of I-3 |
| I-3c | Hemi-fumarate of I-3 |
| I-3d | Acetate of I-3 |
| I-3e | Citrate of I-3 |
| I-3f | Hemi-malonate of I-3 |
| I-3g | Fumarate of I-3 |
| I-3h | Hemi-succinate of I-3 |
| I-3i | Oxalate of I-3 |
| I-3j | Benzoate of I-3 |
| I-3k | Salicylate of I-3 |
| I-4a | Benzenesulfonate of I-4 |
| I-4b | Tartrate of I-4 |
| I-4c | Hemi-fumarate of I-4 |
| I-4d | Acetate of I-4 |
| I-4e | Citrate of I-4 |
| I-4f | Hemi-malonate of I-4 |
| I-4g | Fumarate of I-4 |
| I-4h | Hemi-succinate of I-4 |
| I-4i | Oxalate of I-4 |
| I-4j | Benzoate of I-4 |
| I-4k | Salicylate of I-4 |
| I-5a | Benzenesulfonate of I-5 |
| I-5b | Tartrate of I-5 |
| I-5c | Hemi-fumarate of I-5 |
| I-5d | Acetate of I-5 |
| I-5e | Citrate of I-5 |
| I-5f | Hemi-malonate of I-5 |
| I-5g | Fumarate of I-5 |
| I-5h | Hemi-succinate of I-5 |
| I-5i | Oxalate of I-5 |
| I-5j | Benzoate of I-5 |
| I-5k | Salicylate of I-5 |
| I-6a | Benzenesulfonate of I-6 |
| I-6b | Tartrate of I-6 |
| I-6c | Hemi-fumarate of 1-6 |
| I-6d | Acetate of I-6 |
| I-6e | Citrate of I-6 |
| I-6f | Hemi-malonate of I-6 |
| I-6g | Fumarate of I-6 |
| I-6h | Hemi-succinate of I-6 |
| I-6i | Oxalate of I-6 |
| I-6j | Benzoate of I-6 |
| I-6k | Salicylate of I-6 |
| I-7a | Benzenesulfonate of I-7 |
| I-7b | Tartrate of I-7 |
| I-7c | Hemi-fumarate of I-7 |
| I-7d | Acetate of I-7 |
| I-7e | Citrate of I-7 |
| I-7f | Hemi-malonate of I-7 |
| I-7g | Fumarate of I-7 |
| I-7h | Hemi-succinate of 1-7 |
| I-7i | Oxalate of I-7 |
| I-7j | Benzoate of I-7 |
| I-7k | Salicylate of I-7 |
| I-8a | Benzenesulfonate of I-8 |
| I-8b | Tartrate of I-8 |
| I-8c | Hemi-fumarate of I-8 |
| I-8d | Acetate of I-8 |
| I-8e | Citrate of I-8 |
| I-8f | Hemi-malonate of I-8 |
| I-8g | Fumarate of I-8 |
| I-8h | Hemi-succinate of I-8 |
| I-8i | Oxalate of I-8 |
| I-8j | Benzoate of I-8 |
| I-8k | Salicylate of I-8 |
| I-9a | Benzenesulfonate of I-9 |
| I-9b | Tartrate of I-9 |
| I-9c | Hemi-fumarate of. I-9 |
| I-9d | Acetate of I-9 |
| I-9e | Citrate of I-9 |
| I-9f | Hemi-malonate of I-9 |
| I-9g | Fumarate of I-9 |
| I-9h | Hemi-succinate of I-9 |
| I-9i | Oxalate of I-9 |
| I-9j | Benzoate of I-9 |
| I-9k | Salicylate of I-9 |
| I-10a | Benzenesulfonate of I-10 |
| I-10b | Tartrate of I-10 |
| I-10c | Hemi-fumarate of I-10 |
| I-10d | Acetate of I-10 |
| I-10e | Citrate of I-10 |
| I-10f | Hemi-malonate of I-10 |
| I-10g | Fumarate of I-10 |
| I-10h | Hemi-succinate of I-10 |
| I-10i | Oxalate of I-10 |
| I-10j | Benzoate of I-10 |
| I-10k | Salicylate of I-10 |

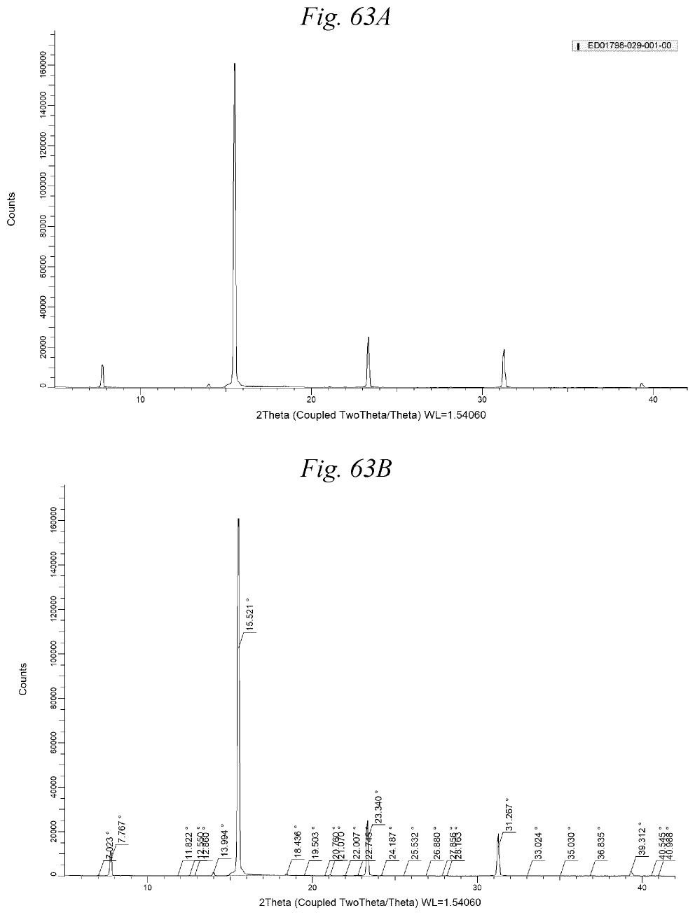

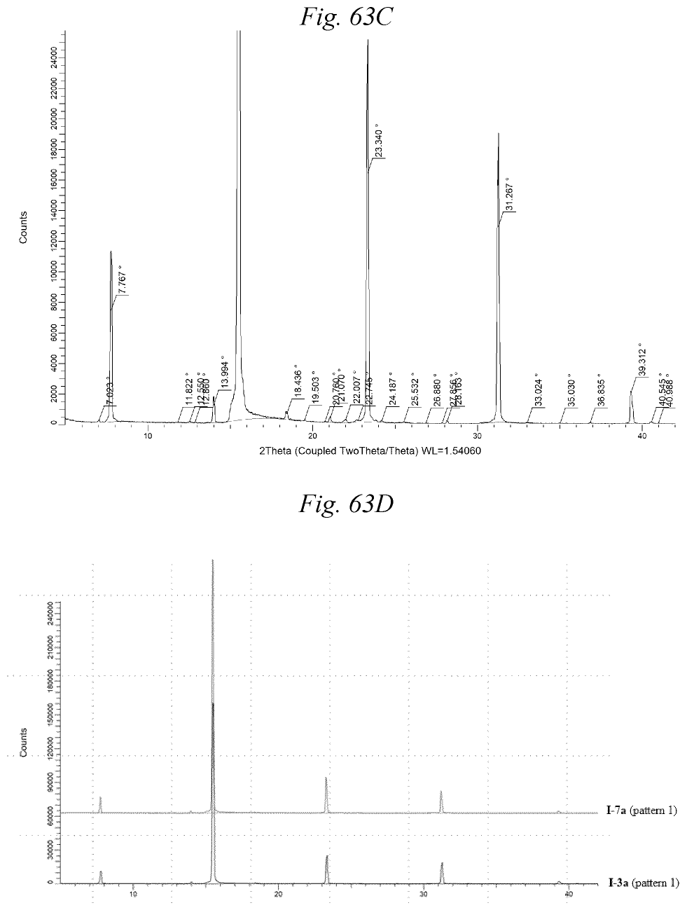

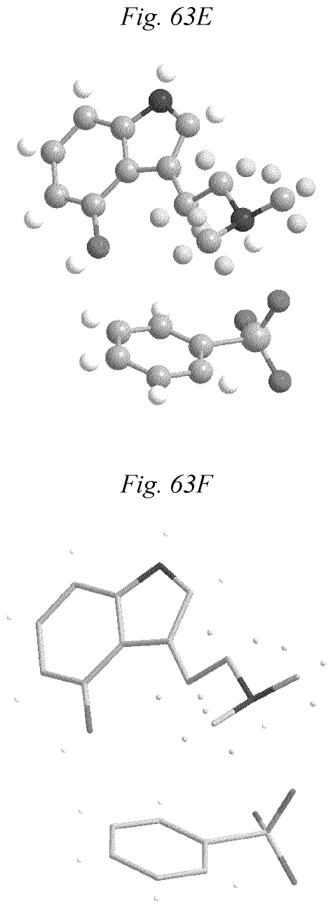

In some embodiments, the pharmaceutically acceptable salt is a benzenesulfonate salt of 3-(2-(bis(methyl-d₃)amino)ethyl-1,1,2,2-d₄)-1H-indol-4-ol (I-3a). In some embodiments, salt I-3a is in a crystalline solid form (pattern 1) characterized by an X-ray powder diffraction pattern containing at least three characteristic peaks at diffraction angles (2θ±0.2°) selected from 7.023°, 7.767°, 11.822°, 12.550°, 12.860°, 13.994°, 15.521°, 18.436°, 19.503°, 20.760°, 21.070°, 22.007°, 22.745°, 23.340°, 24.187°, 25.532°, 26.880°, 27.856°, 28.163°, 31.267°, 33.024°, 35.030°, 36.835°, 39.312°, 40.545°, and 40.988°, as determined by XRPD using a CuKα radiation source, for example, as shown in FIGS. 63A-63D.

In some embodiments, the pharmaceutically acceptable salt is a benzenesulfonate salt of 3-(2-(dimethylamino)ethyl)-1H-indol-4-ol (I-7a). In some embodiments, salt I-7a is in a crystalline solid form (pattern 1) characterized by an X-ray powder diffraction pattern containing at least three characteristic peaks at diffraction angles (2θ±0.20) selected from 7.002°, 7.733°, 11.768°, 12.516°, 12.882°, 13.546°, 13.968°, 14.788°, 15.225°, 15.474°, 18.370°, 19.737°, 20.703°, 21.050°, 21.873°, 21.982°, 22.315°, 22.639°, 23.282°, 23.775°, 24.125°, 25.193°, 25.475°, 25.931°, 26.813°, 27.778°, 28.127°, 30.866°, 31.207°, 32.941°, 33.222°, 33.698°, 36.803°, 38.668°, and 39.289°, as determined by XRPD using a CuKα radiation source, for example, as shown in FIGS. 3A-3B.

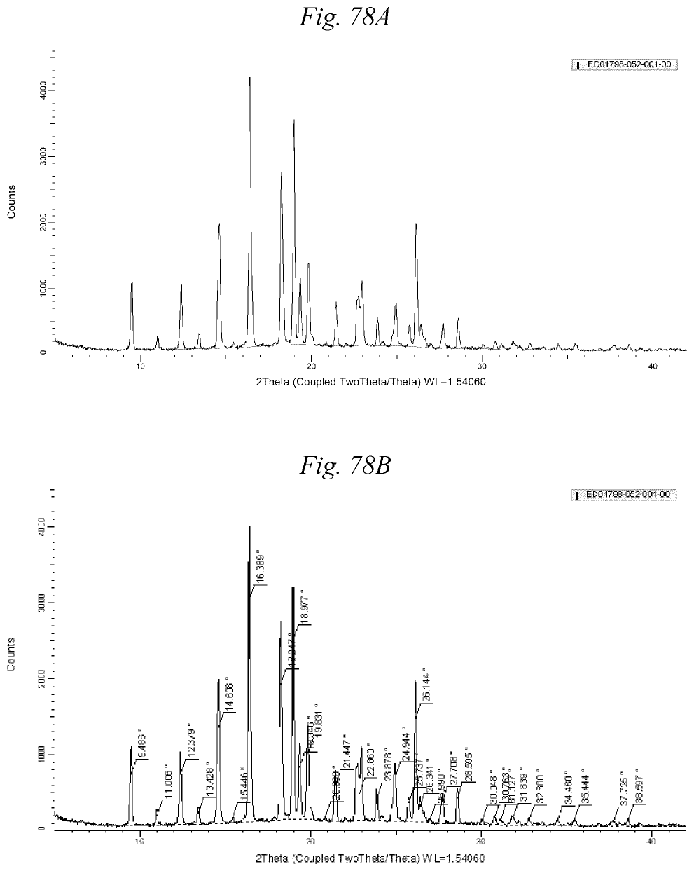

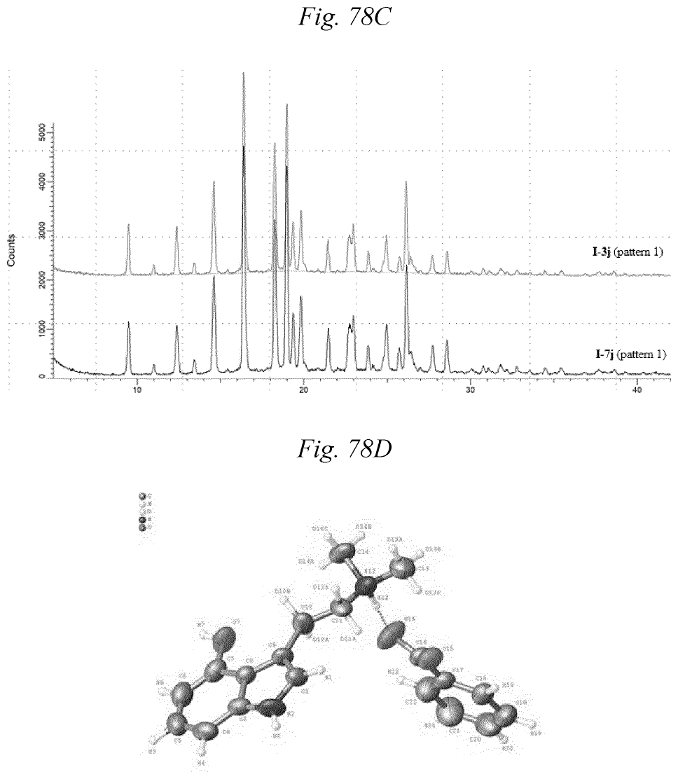

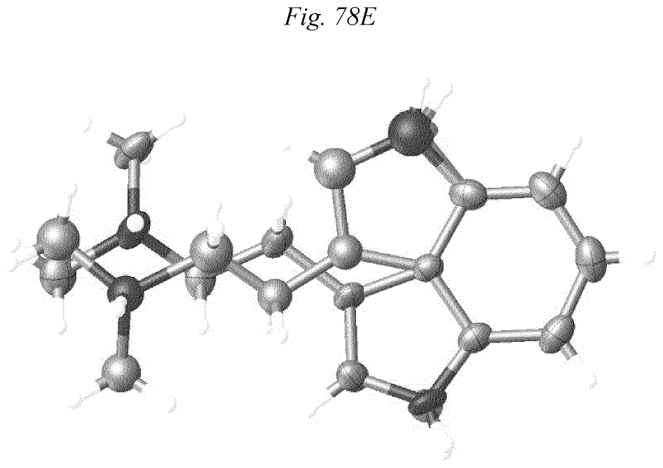

In some embodiments, the pharmaceutically acceptable salt is a benzoate salt of 3-(2-(bis(methyl-d₃)amino)ethyl-1,1,2,2-d₄)-1H-indol-4-ol (I-3j). In some embodiments, salt I-3j is in a crystalline solid form (pattern 1) characterized by an X-ray powder diffraction pattern containing at least three characteristic peaks at diffraction angles (2θ±0.2°) selected from 9.486°, 11.006°, 12.379°, 13.428°, 14.608°, 15.446°, 16.389°, 18.247°, 18.977°, 19.346°, 19.831°, 20.868°, 21.447°, 22.860°, 23.878°, 24.944°, 25.737°, 26.144°, 26.341°, 26.990°, 27.708°, 28.595°, 30.048°, 30.763°, 31.127°, 31.839°, 32.800°, 34.460°, 35.444°, 37.725°, and 38.597°, as determined by XRPD using a CuKα radiation source, for example, as shown in FIGS. 78A-78C.

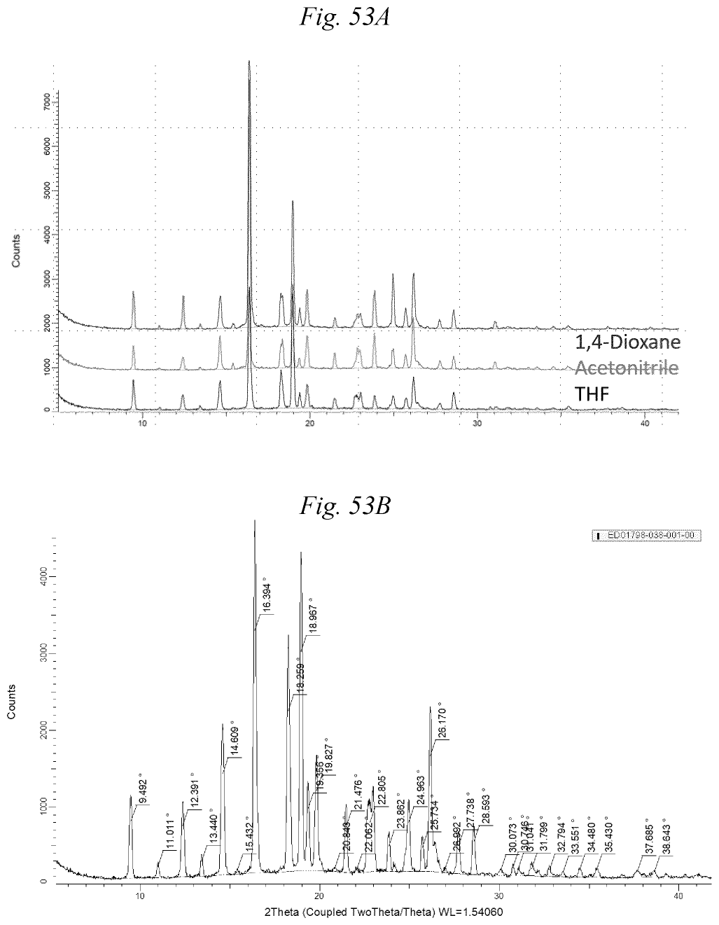

In some embodiments, the pharmaceutically acceptable salt is a benzoate salt of 3-(2-(dimethylamino)ethyl)-1H-indol-4-ol (I-7j). In some embodiments, salt I-7j is in a crystalline solid form (pattern 1) characterized by an X-ray powder diffraction pattern containing at least three characteristic peaks at diffraction angles (2θ±0.2°) selected from 9.492°, 11.011°, 12.391°, 13.440°, 14.609°, 15.432°, 16.394°, 18.259°, 18.967°, 19.356°, 19.827°, 20.843°, 21.476°, 22.062°, 22.805°, 23.862°, 24.963°, 25.734°, 26.170°, 26.992°, 27.738°, 28.593°, 30.073°, 30.746°, 31.041°, 31.799°, 32.794°, 33.551°, 34.480°, 35.430°, 37.685°, and 38.643°, as determined by XRPD using a CuKα radiation source, for example, as shown in FIGS. 53A-53B.

In some embodiments, the pharmaceutically acceptable salt is a citrate salt of 3-(2-(bis(methyl-d₃)amino)ethyl-1,1,2,2-d₄)-1H-indol-4-ol (I-3e). In some embodiments, salt I-3e is in the form of an amorphous solid as characterized by an X-ray powder diffraction (XRPD).

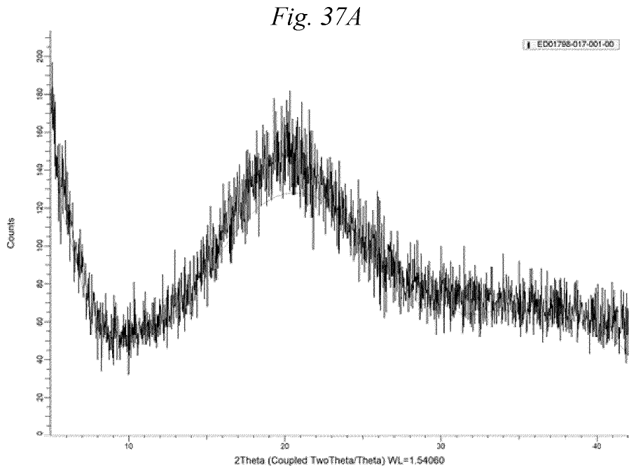

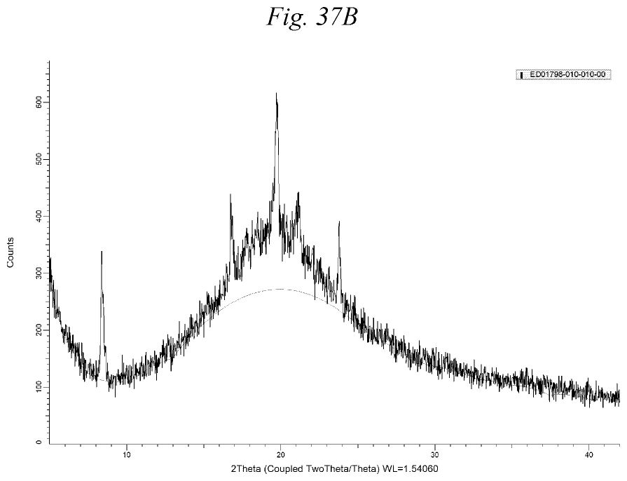

In some embodiments, the pharmaceutically acceptable salt is a citrate salt of 3-(2-(dimethylamino)ethyl)-1H-indol-4-ol (I-7e). In some embodiments, salt I-7e is in the form of an amorphous solid as characterized by an X-ray powder diffraction (XRPD), for example, as shown in FIGS. 37A-37B.

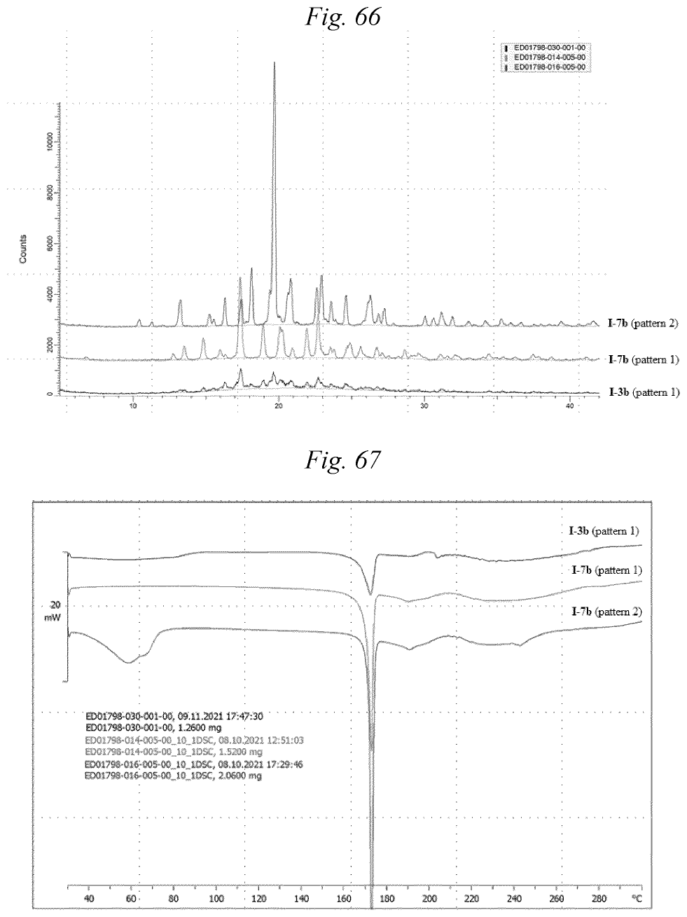

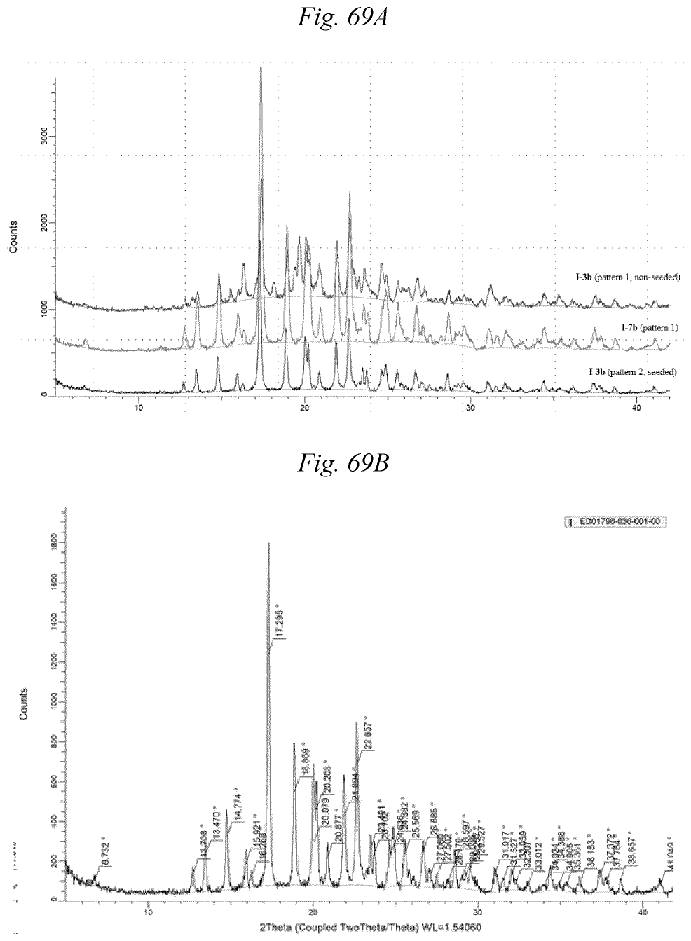

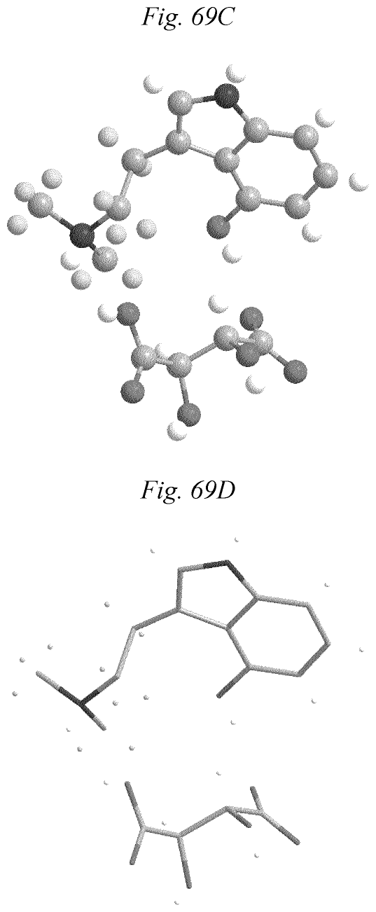

In some embodiments, the pharmaceutically acceptable salt is a tartrate salt of 3-(2-(bis(methyl-d₃)amino)ethyl-1,1,2,2-d₄)-1H-indol-4-ol (I-3b). In some embodiments, salt I-3b is in a crystalline solid form of pattern 1 characterized by, e.g., an X-ray powder diffraction pattern shown in FIG. 66. In some embodiments, salt I-3b is in a crystalline solid form (pattern 2) characterized by an X-ray powder diffraction pattern containing at least three characteristic peaks at diffraction angles (2θ±0.2°) selected from 6.732°, 12.708°, 13.470°, 14.774°, 15.921°, 16.268°, 17.295°, 18.869°, 20.079°, 20.208°, 20.877°, 21.894°, 22.657°, 23.491°, 23.702°, 24.636°, 24.882°, 25.569°, 26.685°, 27.060°, 27.502°, 28.179°, 28.597°, 29.035°, 29.257°, 29.527°, 31.017°, 31.527°, 32.059°, 32.307°, 33.012°, 34.024°, 34.388°, 34.905°, 35.361°, 36.183°, 37.372°, 37.764°, 38.657°, and 41.049°, as determined by XRPD using a CuKα radiation source, for example, as shown in FIGS. 69A-69B.

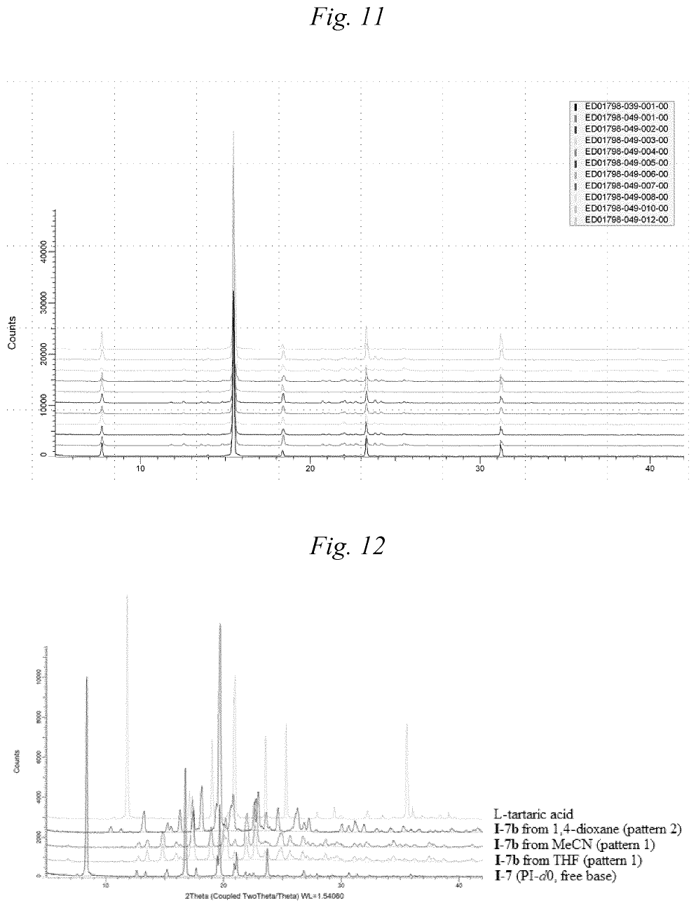

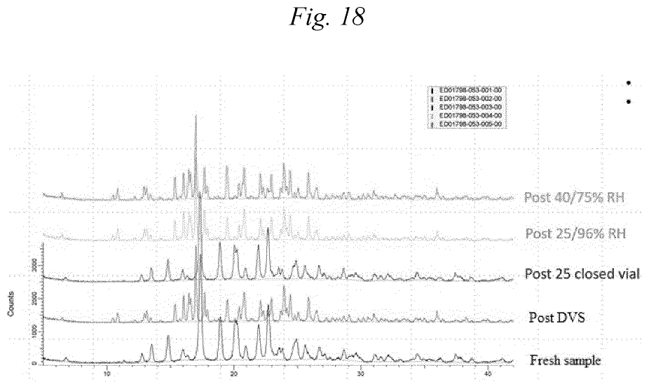

In some embodiments, the pharmaceutically acceptable salt is a tartrate salt of 3-(2-(dimethylamino)ethyl)-1H-indol-4-ol (I-7b). In some embodiments, salt I-7b is in a crystalline solid form (pattern 1) characterized by an X-ray powder diffraction pattern containing at least three characteristic peaks at diffraction angles (2θ±0.2°) selected from 6.798°, 11.360°, 12.764°, 13.535°, 14.837°, 15.973°, 16.351°, 17.367°, 18.937°, 20.168°, 20.929°, 21.946°, 22.719°, 23.604°, 23.814°, 24.874°, 25.609°, 26.745°, 27.111°, 27.558°, 28.653°, 29.630°, 31.129°, 31.567°, 32.180°, 33.073°, 34.096°, 34.460°, 36.226°, 37.497°, 38.727°, and 41.126°, as determined by XRPD using a CuKα radiation source, for example, as shown in FIG. 12. In some embodiments, salt I-7b is in a crystalline solid form of pattern 2 characterized by, e.g., an X-ray powder diffraction pattern as shown in FIG. 12. In some embodiments, salt I-7b is in a crystalline solid form (pattern 3) characterized by an X-ray powder diffraction pattern containing at least three characteristic peaks at diffraction angles (2θ±0.2°) selected from 6.479°, 10.486°, 10.862°, 11.913°, 12.222°, 12.972°, 13.161°, 13.467°, 14.230°, 15.372°, 15.736°, 16.053°, 16.457°, 16.613°, 17.009°, 17.695°, 17.913°, 18.486°, 18.795°, 19.479°, 20.101°, 20.416°, 20.818°, 21.352°, 22.106°, 22.320°, 22.629°, 22.964°, 23.698°, 23.950°, 24.175°, 24.439°, 24.818°, 25.079°, 25.880°, 26.528°, 27.297°, 27.752°, 28.124°, 28.349°, 28.631°, 29.075°, 29.819°, 30.202°, 30.562°, 31.025°, 31.207°, 31.650°, 31.953°, 33.721°, 34.362°, 34.651°, 34.994°, 35.512°, 35.982°, 36.450°, 37.476°, 38.287°, 39.699°, 39.980°, 40.951°, and 41.870°, as determined by XRPD using a CuKα radiation source, for example, as shown in FIG. 18.

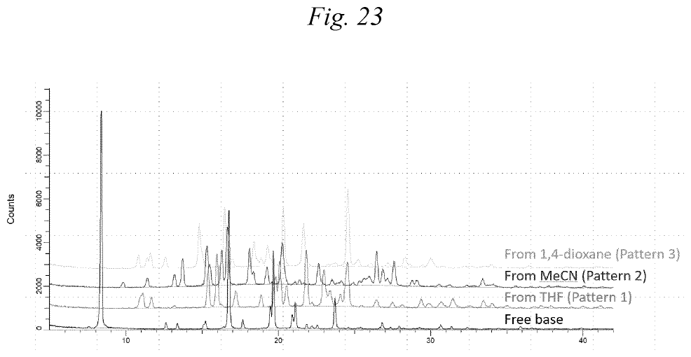

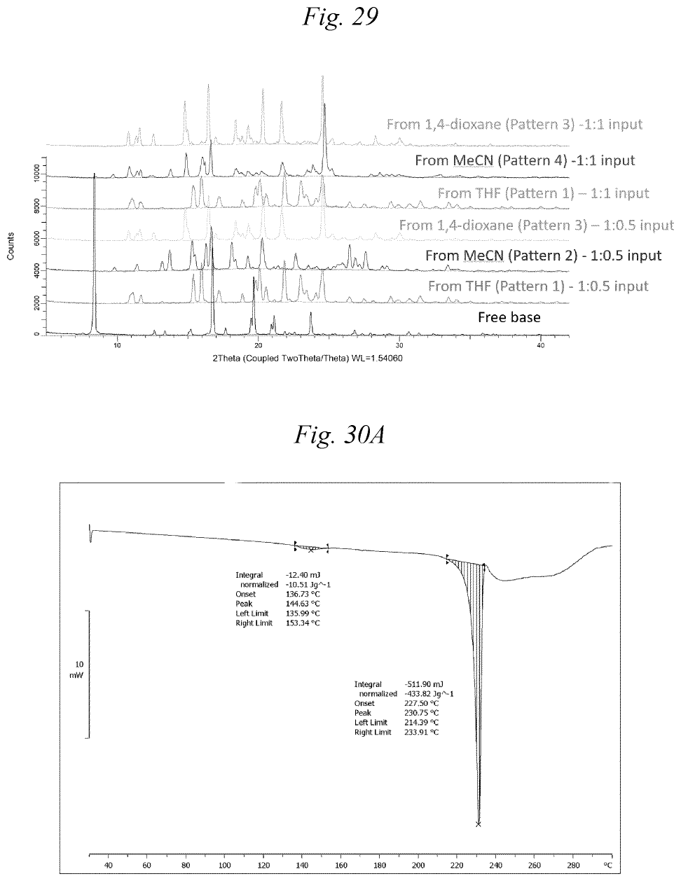

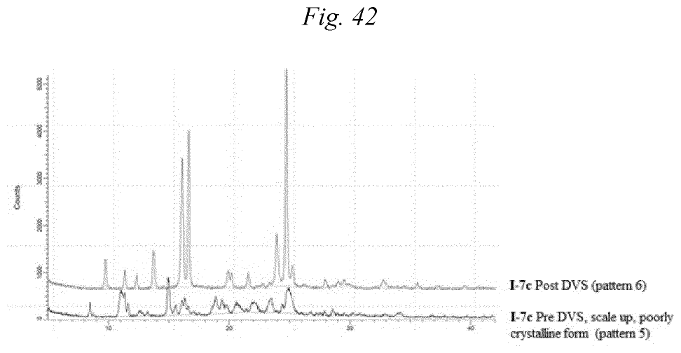

In some embodiments, the pharmaceutically acceptable salt is a hemi-fumarate salt of 3-(2-(dimethylamino)ethyl)-1H-indol-4-ol (I-7c). In some embodiments, salt I-7c is in a crystalline solid form of pattern 1, 2, 3, or 4, characterized by, e.g., an X-ray powder diffraction pattern as shown in FIGS. 23 and 29. In some embodiments, salt I-7c is in a crystalline solid form (pattern 5) characterized by an X-ray powder diffraction pattern containing at least three characteristic peaks at diffraction angles (2θ±0.2°) selected from 8.483°, 8.733°, 11.080°, 11.351°, 11.622°, 12.615°, 13.258, 14.977°, 15.557°, 16.089°, 16.319°, 16.606°, 17.013°, 18.928°, 18.884°, 19.429°, 19.734°, 20.643°, 21.484°, 22.067°, 23.433°, 24.466°, 24.885°, 26.740°, 27.900°, 28.557°, 29.523°, 32.888°, 34.183°, and 36.808°, as determined by XRPD using a CuKα radiation source, for example, as shown in FIG. 42. In some embodiments, salt I-7c is in a crystalline solid form (pattern 6) characterized by an X-ray powder diffraction pattern containing at least three characteristic peaks at diffraction angles (2θ±0.2°) selected from 9.746°, 11.354°, 12.338°, 13.762°, 16.111°, 16.644°, 19.929°, 20.180°, 21.576°, 22.758°, 23.348°, 23.938°, 24.724°, 25.226°, 26.203°, 27.910°, 29.056°, 29.499°, 32.753°, 35.567°, 37.279°, 37.347°, and 39.481°, as determined by XRPD using a CuKα radiation source, for example, as shown in FIG. 42.

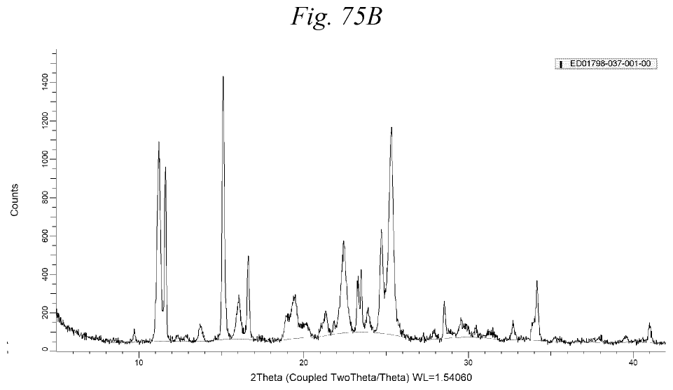

In some embodiments, the pharmaceutically acceptable salt is a hemi-fumarate salt of 3-(2-(bis(methyl-d₃)amino)ethyl-1,1,2,2-d₄)-1H-indol-4-ol (I-3c). In some embodiments, salt I-3c is in a crystalline solid form of pattern 1 characterized by, e.g., an X-ray powder diffraction pattern as shown in FIGS. 72 and 75A. In some embodiments, salt I-3c is in a crystalline solid form (pattern 2) characterized by an X-ray powder diffraction pattern containing at least three characteristic peaks at diffraction angles (2θ±0.2°) selected from 9.713°, 11.209°, 11.605°, 12.338°, 12.852°, 13.718°, 15.117°, 16.066°, 16.627°, 19.026°, 19.427°, 20.108°, 21.068°, 21.335°, 21.837°, 22.429°, 23.262°, 23.478°, 23.900°, 24.720°, 25.318°, 27.912°, 28.532°, 29.565°, 30.457°, 32.698°, 34.155°, 37.910°, 39.566°, and 40.999°, as determined by XRPD using a CuKα radiation source, for example, as shown in FIG. 75B.

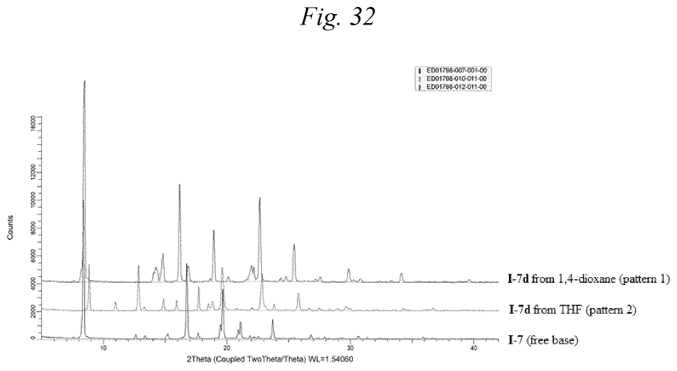

In some embodiments, the pharmaceutically acceptable salt is an acetate salt of 3-(2-(dimethylamino)ethyl)-1H-indol-4-ol (I-7d). In some embodiments, salt I-7d is in a crystalline solid form of pattern 1 or 2 characterized by, e.g., an X-ray powder diffraction pattern as shown in FIG. 32.

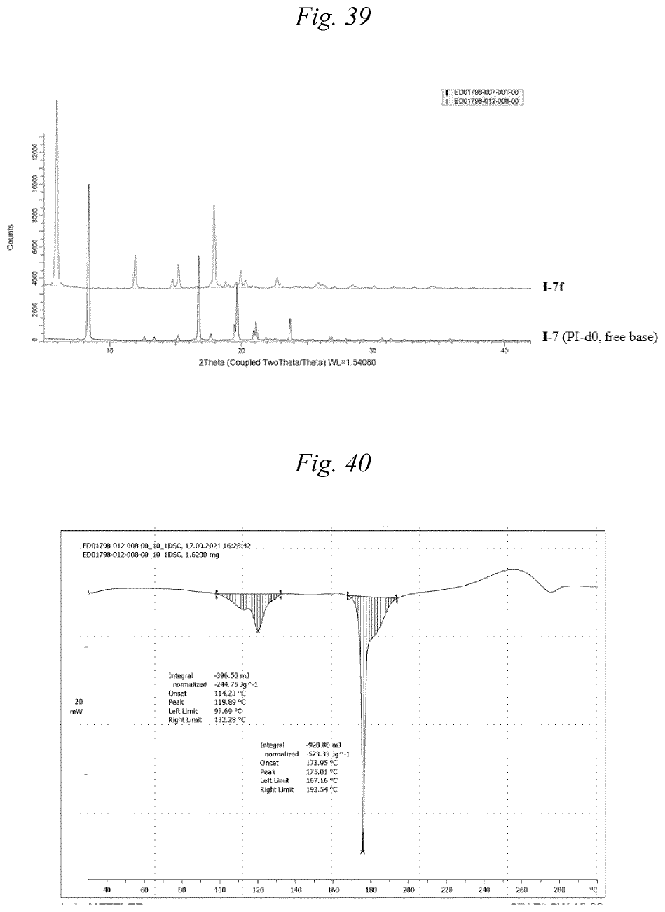

In some embodiments, the pharmaceutically acceptable salt is a hemi-malonate salt of 3-(2-(dimethylamino)ethyl)-1H-indol-4-01 (I-7f). In some embodiments, salt I-7f is in a crystalline solid form of pattern 1 characterized by, e.g., an X-ray powder diffraction pattern as shown in FIG. 39.

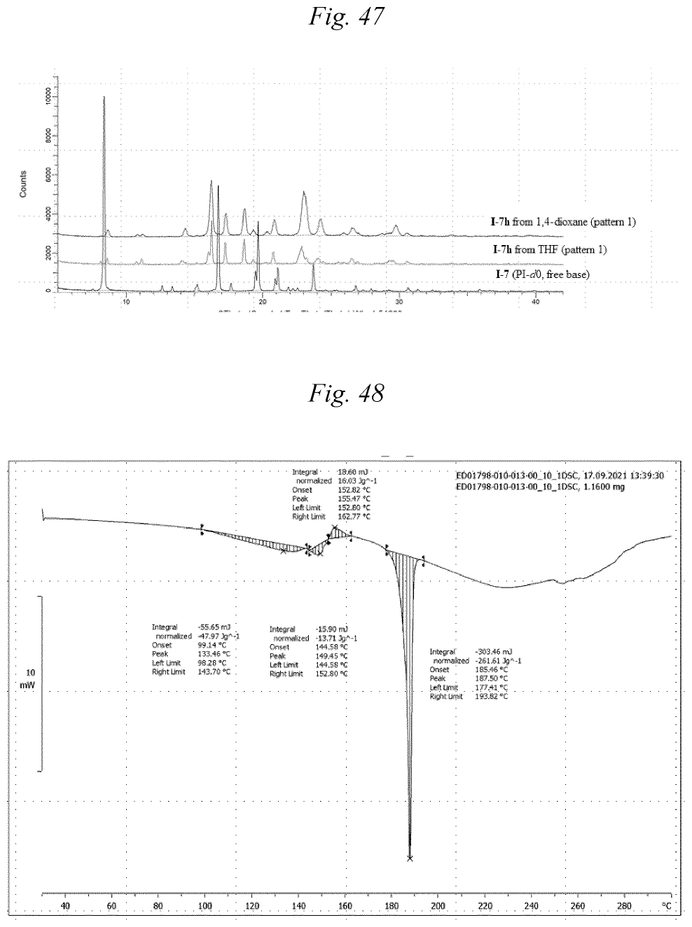

In some embodiments, the pharmaceutically acceptable salt is a hemi-succinate salt of 3-(2-(dimethylamino)ethyl)-1H-indol-4-ol (I-7h). In some embodiments, salt I-7h is in a crystalline solid form of pattern 1 characterized by, e.g., an X-ray powder diffraction as shown in FIG. 47. In some embodiments, the pharmaceutically acceptable salt is an oxalate salt of 3-(2-(dimethylamino)ethyl)-1H-indol-4-ol (I-7i). In some embodiments, salt I-7iis in a crystalline solid form of pattern 1, 2, 3, 4, 5, or 6 characterized by, e.g., an X-ray powder diffraction pattern as shown in FIG. 50.

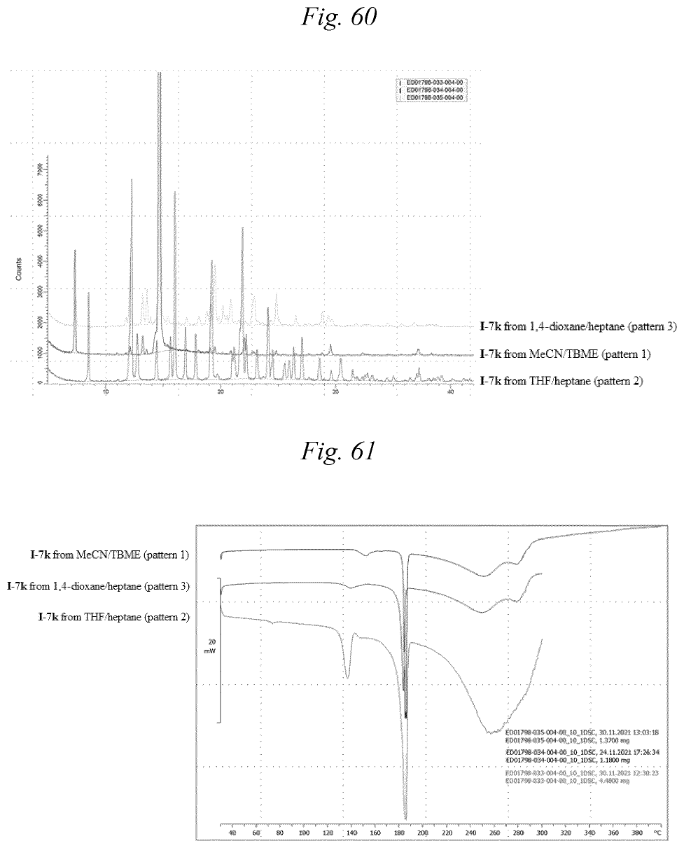

In some embodiments, the pharmaceutically acceptable salt is a salicylate salt of 3-(2-(dimethylamino)ethyl)-1H-indol-4-ol (I-7k). In some embodiments, salt 1-7k is in a crystalline solid form of pattern 1, 2, or 3 characterized by, e.g., an X-ray powder diffraction pattern as shown in FIG. 60.

Without being bound to any particular theory, it is believed that the novel salts of the compounds of Formula (I) are stable and have a faster/quicker therapeutic onset, a shorter duration of drug action (i.e., short duration of therapeutic effect), and less variability in exposures than psilocybin-based drugs (e.g., psilocybin).

In some embodiments, the pharmaceutically acceptable salt of the compound of Formula (I) is a fatty acid salt. The fatty acid used to make the fatty acid salt of the compound of Formula (I) may be a fatty monoacid or a fatty diacid, and may contain a fatty hydrocarbon portion made up of hydrogen and anywhere from 4, from 6, from 8, from 10, from 12, from 14, from 16, and up to 26, up to 24, up to 22, up to 20, up to 18 carbon atoms, which may be fully saturated or partially unsaturated. In some embodiments, the pharmaceutically acceptable salt of the compound of Formula (I) is an adipate salt, a laurate salt, a linoleate salt, a myristate salt, a caprate salt, a stearate salt, an oleate salt, a caprylate salt, a palmitate salt, a sebacate salt, an undecylenate salt, or a caproate salt of the compound of Formula (I). In some embodiments, the pharmaceutically acceptable salt of the compound of Formula (I) is an adipate salt, a laurate salt, a linoleate salt, a myristate salt, a caprate salt, a stearate salt, an oleate salt, or a caprylate salt of the compound of Formula (I), with a laurate salt, a linoleate salt, a caprate salt, or a caprylate salt of the compound of Formula (I) being preferred.

Exemplary pharmaceutically acceptable fatty acid salt forms (i.e., addition salt forms) of the above-identified compounds are provided in Table 3.

TABLE 3
Exemplary pharmaceutically acceptable fatty
acid salts of compounds of Formula (I)

| Salt form identifier | Salt type of compound |
|---|---|
| I-1l | Adipate of I-1 |
| I-1m | Laurate of I-1 |
| I-1n | Linoleate of I-1 |
| I-1o | Myristate of I-1 |
| I-1p | Caprate of I-1 |
| I-1q | Stearate of I-1 |
| I-1r | Oleate of I-1 |
| I-1s | Caprylate of I-1 |
| I-2l | Adipate of I-2 |
| I-2m | Laurate of I-2 |
| I-2n | Linoleate of I-2 |
| I-2o | Myristate of I-2 |
| I-2p | Caprate of I-2 |
| I-2q | Stearate of I-2 |
| I-2r | Oleate of I-2 |
| I-2s | Caprylate of I-2 |
| I-3l | Adipate of I-3 |
| I-3m | Laurate of I-3 |
| I-3n | Linoleate of I-3 |
| I-3o | Myristate of I-3 |
| I-3p | Caprate of I-3 |
| I-3q | Stearate of I-3 |
| I-3r | Oleate of I-3 |
| I-3s | Caprylate of I-3 |
| I-4l | Adipate of I-4 |
| I-4m | Laurate of I-4 |
| I-4n | Linoleate of I-4 |
| I-4o | Myristate of I-4 |
| I-4p | Caprate of I-4 |
| I-4q | Stearate of I-4 |
| I-4r | Oleate of I-4 |
| I-4s | Caprylate of I-4 |
| I-5l | Adipate of I-5 |
| I-5m | Laurate of I-5 |
| I-5n | Linoleate of I-5 |
| I-5o | Myristate of I-5 |
| I-5p | Caprate of I-5 |
| I-5q | Stearate of I-5 |
| I-5r | Oleate of I-5 |
| I-5s | Caprylate of I-5 |
| I-6l | Adipate of I-6 |
| I-6m | Laurate of I-6 |
| I-6n | Linoleate of I-6 |
| I-6o | Myristate of I-6 |
| I-6p | Caprate of I-6 |
| I-6q | Stearate of I-6 |
| I-6r | Oleate of I-6 |
| I-6s | Caprylate of I-6 |
| I-7l | Adipate of I-7 |
| I-7m | Laurate of I-7 |
| I-7n | Linoleate of I-7 |
| I-7o | Myristate of I-7 |
| I-7p | Caprate of 1-7 |
| I-7q | Stearate of I-7 |
| I-7r | Oleate of I-7 |
| I-7s | Caprylate of I-7 |
| I-8l | Adipate of I-8 |
| I-8m | Laurate of I-8 |
| I-8n | Linoleate of I-8 |
| I-8o | Myristate of I-8 |
| I-8p | Caprate of I-8 |
| I-8q | Stearate of I-8 |
| I-8r | Oleate of I-8 |
| I-8s | Caprylate of I-8 |
| I-9l | Adipate of I-9 |
| I-9m | Laurate of I-9 |
| I-9n | Linoleate of I-9 |
| I-9o | Myristate of I-9 |
| I-9p | Caprate of I-9 |
| I-9q | Stearate of I-9 |
| I-9r | Oleate of I-9 |
| I-9s | Caprylate of I-9 |
| I-10l | Adipate of I-10 |
| I-10m | Laurate of I-10 |
| I-10n | Linoleate of I-10 |
| I-10o | Myristate of I-10 |
| I-10p | Caprate of I-10 |
| I-10q | Stearate of I-10 |
| I-10r | Oleate of I-10 |
| I-10s | Caprylate of I-10 |

In some embodiments, the pharmaceutically acceptable salt is a laurate salt of 3-(2-(bis(methyl-d₃)amino)ethyl-1,1,2,2-d₄)-1H-indol-4-ol (I-3m). In some embodiments, salt I-3m is in a crystalline solid form of pattern 1 characterized by, e.g., an X-ray powder diffraction pattern as shown in FIG. 90.

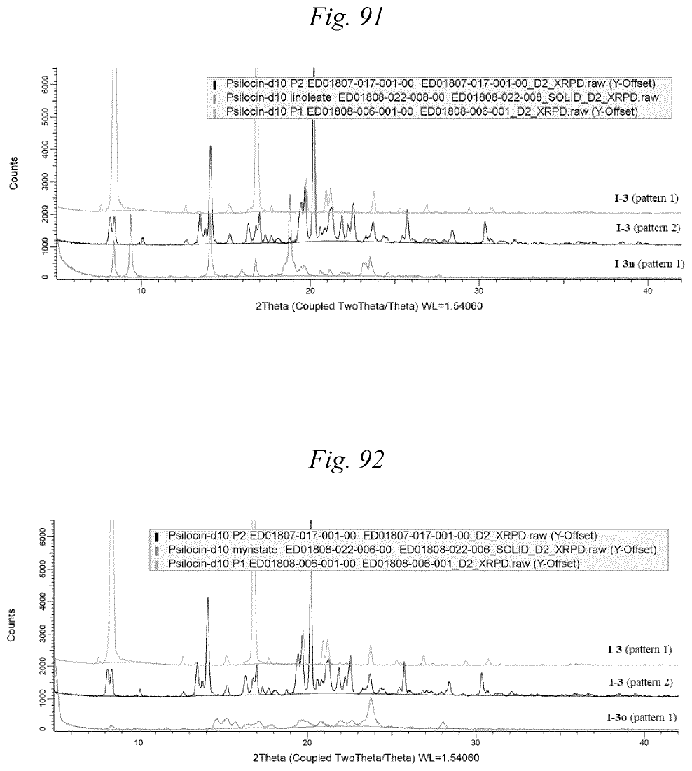

In some embodiments, the pharmaceutically acceptable salt is a linoleate salt of 3-(2-(bis(methyl-d₃)amino)ethyl-1,1,2,2-d₄)-1H-indol-4-ol (I-3n). In some embodiments, salt I-3n is in a crystalline solid form of pattern 1 characterized by, e.g., an X-ray powder diffraction pattern as shown in FIG. 91.

In some embodiments, the pharmaceutically acceptable salt is a myristate salt of 3-(2-(bis(methyl-d₃)amino)ethyl-1,1,2,2-d₄)-1H-indol-4-ol (I-3o). In some embodiments, salt I-3o is in a crystalline solid form of pattern 1 characterized by, e.g., an X-ray powder diffraction pattern as shown in FIG. 92.

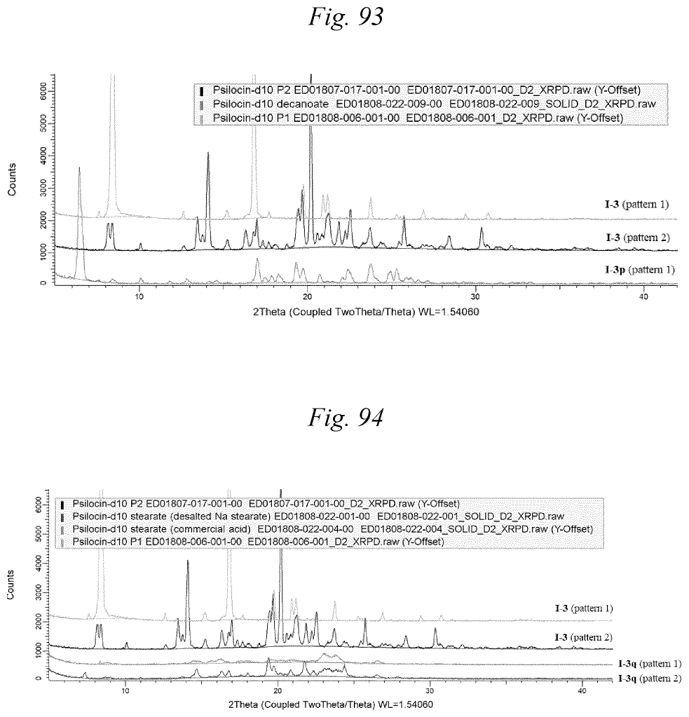

In some embodiments, the pharmaceutically acceptable salt is a caprate salt of 3-(2-(bis(methyl-d₃)amino)ethyl-1,1,2,2-d₄)-1H-indol-4-ol (I-3p). In some embodiments, salt I-3p is in a crystalline solid form of pattern 1 characterized by, e.g., an X-ray powder diffraction pattern as shown in FIG. 93.

In some embodiments, the pharmaceutically acceptable salt is a stearate salt of 3-(2-(bis(methyl-d₃)amino)ethyl-1,1,2,2-d₄)-1H-indol-4-ol (I-3q). In some embodiments, salt I-3q is in a crystalline solid form of pattern 1 or 2 characterized by, e.g., an X-ray powder diffraction pattern as shown in FIG. 94.

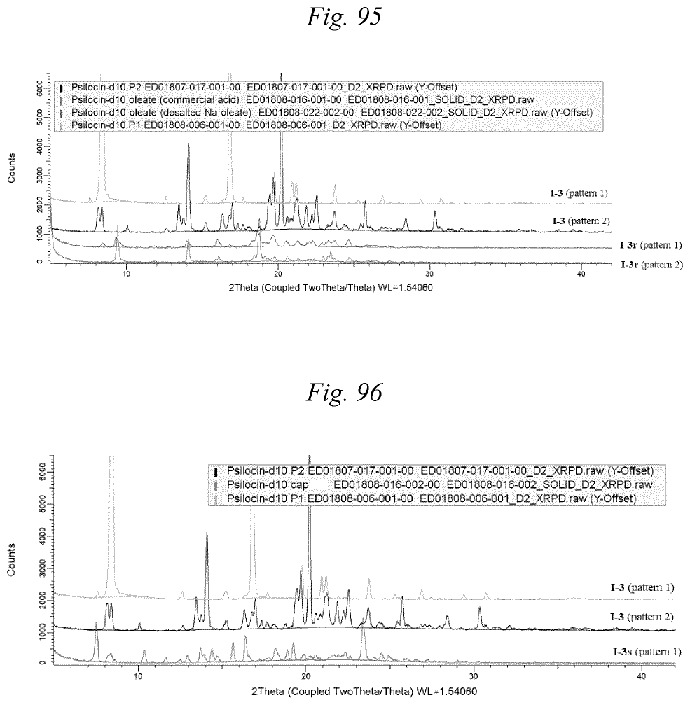

In some embodiments, the pharmaceutically acceptable salt is a oleate salt of 3-(2-(bis(methyl-d₃)amino)ethyl-1,1,2,2-d₄)-1H-indol-4-ol (I-3r). In some embodiments, salt I-3r is in a crystalline solid form of pattern 1 or 2 characterized by, e.g., an X-ray powder diffraction pattern as shown in FIG. 95.

In some embodiments, the pharmaceutically acceptable salt is a caprylate salt of 3-(2-(bis(methyl-d₃)amino)ethyl-1,1,2,2-d₄)-1H-indol-4-ol (I-3s). In some embodiments, salt I-3s is in a crystalline solid form of pattern 1 characterized by, e.g., an X-ray powder diffraction pattern as shown in FIG. 96.

In some embodiments, the pharmaceutically acceptable salt of the compound of Formula (I) has a solubility in corn oil at 22° C. of froth about 0.4 mg/mL, from about 0.5 mg/mL, from about 0.6 mg/mL, from about 0.7 mg/mL, from about 0.8 mg/mL, from about 0.9 mg/mL, from about 1 mg/mL, and up to about 2 mg/mL, up to about 1.9 mg/mL, up to about 1.8 mg/mL, up to about 1.7 mg/mL, up to about 1.6 mg/mL, up to about 1.5 mg/mL, up to about 1.4 mg/mL, up to about 1.3 mg/mL, up to about 1.2 mg/mL.

In some embodiments, the pharmaceutically acceptable salt of the compound of Formula (I) has a solubility in Crodamol® GTCC (medium chain glyceride, from Croda) at 22° C. of from about 0.4 mg/mL, from about 0.6 mg/mL, from about 0.8 mg/mL, from about 1 mg/mL, from about 1.2 mg/mL, from about 1.4 mg/mL, from about 1.6 mg/mL, and up to about 4 mg/mL, up to about 3.8 mg/mL, up to about 3.6 mg/mL, up to about 3.4 mg/mL, up to about 3.2 mg/mL, up to about 3 mg/mL, up to about 2.8 mg/mL, up to about 2.6 mg/mL, up to about 2.4 mg/mL, up to about 2.2 mg/mL.

In some embodiments, the pharmaceutically acceptable salt of the compound of Formula (I) has a solubility in Maisine® CC (mixture of unsaturated mono-, di-, and triglycerides, from Gattefosse) at 22° C. of from about 0.8 mg/mL, from about 1 mg/mL, from about 1.2 mg/mL, from about 1.4 mg/mL, from about 1.6 mg/mL, from about 1.8 mg/mL, from about 2 mg/mL, and up to about 5 mg/mL, up to about 4.8 mg/mL, up to about 4.6 mg/mL, up to about 4.4 mg/mL, up to about 4.2 mg/mL, up to about 4 mg/mL, up to about 3.8 mg/mL, up to about 3.6 mg/mL, up to about 3.4 mg/mL, up to about 3.2 mg/mL, up to about 3 mg/mL, up to about 2.8 mg/mL, up to about 2.6 mg/mL, up to about 2.4 mg/mL, up to about 2.2 mg/mL.

Owing to their relatively hydrophobic nature, fatty acid salts of the compounds of Formula (I) may be advantageous when used in medications adapted for a modified, controlled, slow, or extended release profile. As a result, the fatty acid salts of the compounds of Formula (I) may be well suited for routes of administration (e.g., subcutaneous, transdermal, etc.) and/or dosage forms adapted for providing low doses of active pharmaceutical ingredient (API) over extended periods of time, as may be the case for sub-psychedelic dosing regimens. Non-limiting examples of such dosage forms include, but are not limited to, depots, patches including microneedle patches, liposomes, micelles, microspheres, nanosystems, or other controlled release devices, such as those set forth herein.

Also disclosed herein is a method for stabilizing a compound of Formula (I). The method includes preparing a pharmaceutically acceptable salt of the compound of Formula (I).

Also disclosed herein is a method for preparing a pharmaceutically acceptable salt of the compound of Formula (I). In some embodiments, the method includes:
(a) suspending the free base of the compound of Formula (I) in a solvent or mixture of solvents;
(b) contacting an acid with the compound of Formula (I) to provide a mixture;
(c) optionally heating the mixture;
(d) optionally cooling the mixture; and
(e) isolating the salt.

Various solvents may be used in the disclosed methods, including one or more protic solvents, one or more aprotic solvents, or mixtures thereof. In some embodiments, the solvent(s) used in the method of preparing the salt is/are a protic solvent(s). In some embodiments, the solvent used in the method of preparing the salt is selected from the group consisting of methanol, ethanol, propanol, isopropanol, butanol, 2-butanol, acetone, butanone, dioxanes (1,4-dioxane), water, tetrahydrofuran (THF), acetonitrile (MeCN), ether solvents (e.g., t-butylmethyl ether (TBME)), hexane, heptane, and octane, and combinations thereof. In some embodiments, the solvent is ethanol. In some embodiments, the solvent is 1,4-dioxane. In some embodiments, the solvent is acetonitrile. In some embodiments; the solvent is tetrahydrofuran.

Suitable acids for use in the preparation of pharmaceutically acceptable acid addition salts may include those described heretofore. The acid may be an inorganic acid such as hydrochloric acid, or an organic acid, with organic acids being preferred. In some embodiments, the acid is an organic acid selected from the group consisting of ascorbic acid, citric acid, fumaric acid, maleic acid, malonic acid, (−)-L-malic acid, (+)-L-tartaric acid, methanesulfonic acid, benzenesulfonic acid, toluenesulfonic acid, benzoic acid, salicylic acid, succinic acid, oxalic acid, D-glucuronic acid, glutaric acid salt, and acetic acid. In some embodiments, the acid is an organic acid selected from the group consisting of benzenesulfonic acid, (+)-L-tartaric acid, fumaric acid, acetic acid, citric acid, malonic acid, succinic acid, oxalic acid, benzoic acid, and salicylic acid, with benzenesulfonic acid, succinic acid, and benzoic acid being preferred. In some embodiments, the acid is a fatty acid, such as adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc., with particular mention being made to adipic (hexandioic) acid, lauric (dodecanoic) acid, linoleic acid, myristic (tetradecanoic) acid, capric (decanoic) acid, stearic (octadecanoic) acid, oleic acid, and caprylic (octanoic) acid.

In some embodiments, a stoichiometric (or superstoichiometric) quantity of the acid is contacted with the compound of Formula (I). In some embodiments, a sub-stoichiometric (e.g., 0.5 molar equivalents) quantity of the acid is contacted with the compound of Formula (I). The use of sub-stoichiometric quantities of the acid may be desirable when, for example, the acid contains at least two acidic protons (e.g., two or more carboxylic acid groups) and the target salt is a hemi-acid salt.

In some embodiments, the mixture is heated, e.g., refluxed, prior to cooling.

In some embodiments, the mixture is cooled and the salt is precipitated out of the solution. In some embodiments, the salt is precipitated out of solution in crystalline form. In some embodiments, the salt is precipitated out of solution in amorphous form.

Isolation of the salt may be performed by various well-known isolation techniques, such as filtration, decantation, and the like. In some embodiments, the isolating step includes filtering the mixture.

After isolation, additional crystallization and/or recrystallization steps may also optionally be performed, if desired, for example to increase purity, crystallinity, etc.

In some embodiments, compounds of the present disclosure, e.g., a compound of Formula (I), or a pharmaceutically acceptable salt, a polymorph, or stereoisomer thereof, is in the form of a solvate. Examples of solvate forms include, but are not limited to, hydrates, methanolates, ethanolates, isopropanolates, etc., with hydrates and ethanolates being preferred. The solvate may be formed from stoichiometric or nonstoichiometric quantities of solvent molecules. Solvates of the compounds herein may be in the form of isolable solvates. In one non-limiting example, as a hydrate, the compound may be a monohydrate, a dihydrate, etc. Solvates of the compounds herein also include solution-phase forms. Thus, in some embodiments, the present disclosure provides solution-phase compositions of the compounds of the present disclosure, or any pharmaceutically acceptable salts thereof, which are in solvated form, preferably fully solvated form.

### Pharmaceutical Compositions

Also disclosed herein is a pharmaceutical composition comprising a compound of Formula (I), or a pharmaceutically acceptable salt, polymorph, stereoisomer, or solvate thereof, and a pharmaceutically acceptable vehicle. The pharmaceutical compositions may contain one, or more than one, compound, salt form, polymorph, stereoisomer, and/or solvate of the present disclosure.

The pharmaceutical composition may comprise a single compound of Formula (I), or a pharmaceutically acceptable salt, polymorph, stereoisomer, or solvate thereof, or a mixture of compounds of Formula (I), in either free base or salt form, including one or more polymorphs of such materials. The pharmaceutical composition may be formed from an isotopologue mixture of the disclosed compounds. In some embodiments, a subject compound of Formula (I) may be present in the pharmaceutical composition at a purity of at least 50% by weight, at least 60% by weight, at least 70% by weight, at least 80% by weight, at least 90% by weight, at least 95% by weight, at least 99% by weight, based on a total weight of isotopologues of the compound of Formula (I) present in the pharmaceutical composition. For example, a pharmaceutical composition formulated with psilocin d-10 (compound I-3; 3-(2-(bis(methyl-d₃)amino)ethyl-1,1,2,2-d₄-1H-indol-4-ol), in either free base or salt form, stereoisomers, solvates, or mixtures thereof as the subject compound, may additionally contain isotopologues of the subject compound, e.g., psilocin d-9, psilocin d-8 (compound I-4; 3-(2-(bis(methyl-d₃)amino)ethyl-2,2-d₂)-1H-indol-4-ol), etc., as free-base or salt forms, polymorphs, stereoisomers, solvates, or mixtures thereof. In some embodiments, the composition is substantially free of other isotopologues of the compound, in either free base or salt form, e.g., the composition has less than 20, 15, 10, 9, 8, 7, 6, 5, 4, 3, 2 or 1 or 0.5 mole percent of other isotopologues of the compound.

In some embodiments, any position in the compound having deuterium has a minimum deuterium incorporation that is greater than that found naturally occurring in hydrogen (about 0.016 atom %). In some embodiments, any position in the compound having deuterium has a minimum deuterium incorporation of at least 10 atom %, at least 20 atom %, at least 25 atom %, at least 30 atom %, at least 40 atom %, at least 45 atom %, at least 50 atom %, at least 60 atom %, at least 70 atom %, at least 80 atom %, at least 90 atom %, at least 95 atom %, at least 99 atom % at the site of deuteration.

The pharmaceutical composition may be formulated with an enantiomerically pure compound of the present disclosure, e.g., a compound of Formula (I), or a racemic mixture of the compounds. As described herein, a racemic compound of Formula (I) may contain about 50% of the R- and S-stereoisomers based on a molar ratio (about 48 to about 52 mol %, or about a 1:1 ratio)) of one of the isomers. In some embodiments, a composition, medicament, or method of treatment may involve combining separately produced compounds of the R- and S-stereoisomers in an approximately equal molar ratio (e.g., about 48 to 52%). In some embodiments, a medicament or pharmaceutical composition may contain a mixture of separate compounds of the R- and S-stereoisomers in different ratios. In some embodiments, the pharmaceutical composition contains an excess (greater than 50%) of the R-enantiomer. Suitable molar ratios of R/S may be from about 1.5:1, 2:1, 3:1, 4:1, 5:1, 10:1, or higher. In some embodiments, a pharmaceutical composition may contain an excess of the S-enantiomer, with the ratios provided for R/S reversed. Other suitable amounts of R/S may be selected. For example, the R-enantiomer may be enriched, e.g., may be present in amounts of at least about 55% to 100%, or at least 65%, at least 75%, at least 80%, at least 85%, at least 90%, about 95%, about 98%, or 100%. In other embodiments, the S-enantiomer may be enriched, e.g., in amounts of at least about 55% to 100%, or at least 65%, at least 75%, at least 80%, at least 85%, at least 90%, about 95%, about 98%, or 100%. Ratios between all these exemplary embodiments as well as greater than and less than them while still within the disclosure, all are included. Compositions may contain a mixture of the racemate and a separate compound of Formula (I), or a pharmaceutically acceptable salt, polymorph, stereoisomer, or solvate thereof.

The pharmaceutical composition may be formulated with one or more polymorphs of the compounds of Formula (I) and/or their salt forms, including crystalline and/or amorphous polymorphs of the compounds or salts thereof. In some embodiments, the pharmaceutical composition includes a mixture of crystalline polymorphs. In some embodiments, the pharmaceutical composition includes a single crystalline polymorph. In some embodiments, the pharmaceutical composition includes a mixture of amorphous polymorphs. In some embodiments, the pharmaceutical composition includes a single amorphous polymorph. In some embodiments, the pharmaceutical composition includes a mixture of crystalline and amorphous polymorphs.

In some embodiments, the pharmaceutical composition comprises a compound of Formula (I) (or a pharmaceutically acceptable salt, stereoisomer, or solvate thereof) in crystalline form. In some embodiments, the pharmaceutical composition comprises a highly pure crystalline form of a compound of Formula (I) as a free base. For example, the pharmaceutical composition may comprise a free base of a compound of Formula (I), wherein at least 90%, at least 95%, at least 99%, or at least 99.5% by weight of the free base of the compound of Formula (I) present in the pharmaceutical composition is in crystalline form, e.g., as determined by X-ray powder diffraction and/or mDSC. In some embodiments, the pharmaceutical composition comprises a highly pure crystalline form of a pharmaceutically acceptable salt of a compound of Formula (I). For example, the pharmaceutical composition may comprise a pharmaceutically acceptable salt of a compound of Formula (I), wherein at least 90%, at least 95%, at least 99%, or at least 99.5% by weight of the pharmaceutically acceptable salt of the compound of Formula (I) present in the pharmaceutical composition is in crystalline form, e.g., as determined by X-ray powder diffraction and/or mDSC.

In some embodiments, the pharmaceutical composition comprises a compound of Formula (I) (or a pharmaceutically acceptable salt, stereoisomer, or solvate thereof) in amorphous form. In some embodiments, only the amorphous form of the compound of Formula (I) (or a pharmaceutically acceptable salt, stereoisomer, or solvate thereof) is present in the pharmaceutical composition, e.g., no crystalline forms of the compound of Formula (I) are detectable, for example by XRPD. In some embodiments, the pharmaceutical composition comprises a highly pure amorphous form of a compound of Formula (I) as a free base. For example, the pharmaceutical composition may comprise a free base of a compound of Formula (I), wherein at least 92%, at least 94%, at least 96%, at least 98%, at least 99%, or at least 99.5% by weight of the free base of the compound of Formula (I) present in the pharmaceutical composition is in amorphous form, e.g., as determined by X-ray powder diffraction and/or mDSC. In some embodiments, the pharmaceutical composition comprises a highly pure amorphous form of a pharmaceutically acceptable salt of a compound of Formula (I). For example, the pharmaceutical composition may comprise a pharmaceutically acceptable salt of a compound of Formula (I), wherein at least 92%, at least 94%, at least 96%, at least 98%, at least 99%, or at least 99.5% by weight of the pharmaceutically acceptable salt of the compound of Formula (I) present in the pharmaceutical composition is in amorphous form, e.g., as determined by X-ray powder diffraction and/or mDSC.

In some embodiments, the compound of Formula (I)(or a pharmaceutically acceptable salt, polymorph, stereoisomer, or solvate thereof) is chemically pure, for example has a chemical purity of greater than 90%, 92%, 94%, 96%, 97%, 98%, or 99% by HPLC. In some embodiments, the compound of Formula (I) (or a pharmaceutically acceptable salt, polymorph, stereoisomer, or solvate thereof) has no single impurity of greater than 1%, greater than 0.5%, greater than 0.4%, greater than 0.3%, or greater than 0.2%, measured by HPLC. In some embodiments, the compound of Formula (I) (or a pharmaceutically acceptable salt, polymorph, stereoisomer, or solvate thereof) has a chemical purity of greater than 97 area %, greater than 98 area %, or greater than 99 area % by HPLC. In some embodiments, the compound of Formula (I) (or a pharmaceutically acceptable salt, polymorph, stereoisomer, or solvate thereof) has no single impurity greater than 1 area %, greater than 0.5 area %, greater than 0.4 area %, greater than 0.3 area %, or greater than 0.2 area % as measured by HPLC.

Pharmaceutical compositions may be generally provided herein which comprise about 0.1 to about 1000 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg of one or more compounds as disclosed herein, in either free base or salt form, as active pharmaceutical ingredient (API). The quantity of compound of Formula (I) (on active basis) in a unit dose preparation may be varied or adjusted within the above ranges as deemed appropriate using sound medical judgment, according to the particular application, administration route, potency of the active component, etc. The composition can, if desired, also contain other compatible therapeutic agents.

In some embodiments, the pharmaceutical composition comprises at least 0.1% by weight, at least 0.5% by weight, at least 1% by weight, at least 5% by weight, at least 10% by weight, at least 15% by weight, at least 20% by weight, at least 25% by weight, at least 30% by weight, at least 35% by weight, at least 40% by weight, at least 45% by weight, at least 50% by weight, and up to 99.9% by weight, up to 99.5% by weight, up to 99% by weight, up to 98% by weight, up to 97% by weight, up to 95% by weight, up to 90% by weight, up to 85% by weight, up to 80% by weight, up to 75% by weight, up to 70% by weight, up to 65% by weight, up to 60% by weight, up to 55% by weight of the compound of Formula (I) (active basis), based on a total weight of the pharmaceutical composition (on a dry basis), or any range therebetween. Dry basis may refer to pharmaceutical compositions which are in solid dosage form, or liquid dosage forms after subtracting the weight contribution from water or other pharmaceutically acceptable aqueous medium (e.g., fruit juice).

In addition to a compound of Formula (I), or a pharmaceutically acceptable salt, polymorph, stereoisomer, or solvate thereof; pharmaceutical compositions of the present disclosure also comprise a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the present disclosure is formulated for administration to a mammal. Such pharmaceutically acceptable vehicles can be solids or liquids. The pharmaceutically acceptable vehicles can include water, saline, juice including fruit juice (e.g., orange juice such as Tang, grape juice, apple juice, cranberry juice, pineapple juice, etc.), oils, including those of petroleum, animal, vegetable or synthetic origin, such as peanut oil, soybean oil, mineral oil, sesame oil and the like, gum acacia, gelatin, starch paste, talc, keratin, colloidal silica, urea, and the like. Pharmaceutically acceptable vehicles can include, but are not limited to, auxiliary agents, stabilizing agents, solubilizing agents, thickening agents, lubricants, binders, granulators, fillers, diluents, disintegrants, wetting agents, glidants, anti-caking agents, coloring agents, sweetening agents, dye-migration inhibitors, preservatives, antioxidants, lyoprotectants, complexing agents, flavoring agents, matrix-forming agents, dispersing agents, performance modifiers, controlled-release polymers, solvents, pH modifiers, sources of carbon dioxide, or other pharmaceutical additives set forth herein.

Of these pharmaceutically acceptable vehicles, some organic acids have been identified as providing both a stabilizing function and a solubilizing function to the psilocin and deuterated psilocin compounds of the present disclosure (e.g., compounds of Formula (I) or a pharmaceutically acceptable salt, polymorph, stereoisomer, or solvate thereof), thereby improving the delivery and therapeutic characteristics of the disclosed dosage forms. These organic acid vehicles which provide the unique, stabilizing and solubilizing effect (act as a stabilizing/solubilizing agent) may be referred to herein as an “organic acid agent.” In preferred embodiments, the pharmaceutical composition comprises a compound of Formula (I), or a pharmaceutically acceptable salt, polymorph, stereoisomer, or solvate thereof, and an organic acid agent. The pharmaceutical composition can optionally be formulated with other pharmaceutically acceptable vehicles as needed or desired.

In some embodiments, solid dosage forms are formulated with an organic acid agent, wherein the organic acid agent is considered separate and distinct from the compound of Formula (I) or a pharmaceutically acceptable salt, polymorph, stereoisomer, or solvate thereof, i.e., when formulated in solid dosage form, the organic acid agent is not considered to form a salt with the compound of Formula (I). For example, in these embodiments where the pharmaceutical composition is a solid dosage form formulated with a free base of a compound of Formula (I), the organic acid agent is not considered to form an addition salt with the compound of Formula (I), and instead the compound of Formula (I) remains as a free base, at least until the point of dissolution/disintegration in an appropriate medium (e.g., water, juice, saline, saliva, etc.). In another example, where the pharmaceutical composition is formulated with a salt form of a compound of Formula (I), the organic acid agent remains separate from the salt form and provides a stabilizing/solubilizing effect above that provided by the salt form of the compound of Formula (I) alone.

Organic acid agents may be any organic acid described herein, and may be a monoacid, a diacid, a triacid, a tetraacid, or may contain a higher number of acid groups. One organic acid agent or mixtures of organic acid agents may be used. In addition to an acid group(s) (e.g., one or more carboxylic acid moieties), the organic acid agent may also contain one or more hydroxyl functionalities as part of its structure (i.e., the organic acid agent may be a hydroxy acid). In some embodiments, the organic acid agent is an α-hydroxy acid. In some embodiments, the organic acid agent is a β-hydroxy acid. In some embodiments, the organic acid agent is a γ-hydroxy acid.

Examples of hydroxy acids include, but are not limited to, glycolic acid, lactic acid, citric acid, tartaric acid, and malic acid. In some embodiments, the organic acid agent is citric acid and/or tartaric acid. In some embodiments, the organic acid agent is citric acid. In some embodiments, the organic acid agent is tartaric acid. In some embodiments, the organic acid agent is an enedioic acid, examples of which may include, but are not limited to, fumaric acid and maleic acid. In some embodiments, the organic acid agent is fumaric acid. In some embodiments, the organic acid agent is maleic acid. Mixtures and/or hydrates of the disclosed organic acid agent may also be used in the disclosed pharmaceutical compositions. In some embodiments, the organic acid agent is not a sulfonic acid (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.). In some embodiments, the organic acid agent is not a benzoic acid (e.g., benzoic acid, 4-acetamidobenzoic acid, 2-acetoxybenzoic acid, salicylic acid, 4-amino-salicylic acid, gentisic acid, etc.).

In some embodiments, the pharmaceutical composition comprises at least 0.5% by weight, at least 1% by weight, at least 2% by weight, at least 3% by weight, at least 4% by weight, at least 5% by weight, at least 6% by weight, at least 7% by weight, at least 8% by weight, at least 9% by weight, at least 10% by weight, at least 11% by weight, at least 12% by weight, at least 13% by weight, at least 14% by weight, at least 15% by weight, and up to 60% by weight, up to 55% by weight, up to 50% by weight, up to 45% by weight, up to 40% by weight, up to 35% by weight, up to 30% by weight, up to 27% by weight, up to 25% by weight, up to 23% by weight, up to 20% by weight, up to 18% by weight, up to 16% by weight of the organic acid agent, based on a total weight of the pharmaceutical composition (on a dry basis), or any range therebetween. For example, the pharmaceutical composition may contain from 5% to 40% by weight of the organic acid agent, or from 10% to 30% by weight of organic agent, or from 15 to 20% of organic acid agent, based on a total weight of the pharmaceutical composition (on a dry basis). Dry basis may refer to pharmaceutical compositions which are in solid dosage form, or liquid dosage forms after subtracting the weight contribution from water or other pharmaceutically acceptable aqueous medium (e.g., fruit juice).

In some embodiments, a weight ratio of the organic acid agent to the compound of Formula (I)(active basis) is from 1:1, from 1.5:1, from 2:1, from 2.5:1, from 3:1, from 3.5:1, from 4:1, from 4.5:1, from 5:1, and up to 20:1, up to 15:1, up to 10:1, up to 9:1, up to 8:1, up to 7:1, up to 6:1, or any range therebetween.

When the pharmaceutical composition is formulated with a pharmaceutically acceptable salt of a compound of Formula (I), the acid used in forming the pharmaceutically acceptable salt of a compound of Formula (I) and the organic acid agent (vehicle) can be the same. For example, the pharmaceutical composition may comprise a tartrate salt of a compound of Formula (I) (e.g., I-1b, I-2b, I-3b, I-4b, I-5b, I-6b, I-7b, I-8b, I-9b, and/or I-10b), and tartaric acid as organic acid agent (vehicle). In another example, the pharmaceutical composition may comprise a citrate salt of a compound of Formula (I) (e.g., I-le, I-2e, I-3e, I-4e, I-5e, I-6e, I-7e, I-8e, I-9e, and/or I-10e), and citric acid as organic acid agent (vehicle).

When the pharmaceutical composition is formulated with a pharmaceutically acceptable salt of a compound of Formula (I), the acid used in forming the pharmaceutically acceptable salt of a compound of Formula (I) and the organic acid agent (vehicle) can be different. For example, the pharmaceutical composition may comprise a benzenesulfonate salt of a compound of Formula (I) (e.g., I-1a, I-2a, I-3a, I-4a, I-5a, I-6a, I-7h, I-8a, I-9a, and/or I-10a), and citric acid and/or tartaric acid, etc., as organic acid agent (vehicle). In another example, the pharmaceutical composition may comprise a benzoate salt of a compound of Formula (I) (e.g., I-1j, I-2j, I-3j, I-4j, I-5j, I-6j, I-7j, I-8j, I-9j, and/or I-10j), and citric acid and/or tartaric acid, etc., as organic acid agent (vehicle).

Any of the pharmaceutical compositions disclosed herein formulated with an organic acid agent may contain an organic acid agent which is uncoated, or alternatively, may contain an organic acid agent which is coated (a “coated organic acid agent”) with a pharmaceutically acceptable vehicle. Examples of coated organic acid agents are set forth hereinafter.

The pharmaceutical compositions disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. Administration of the subject compounds may be systemic or local. In some instances, the pharmaceutical compositions are formulated for administration in accordance with routine procedures as a pharmaceutical composition adapted for oral, intravenous, or intradermal administration, or other routes of administration as set forth herein, to humans. Examples of suitable pharmaceutically acceptable vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference. The choice of vehicle will be determined in part by the particular compound, salt form, as well as by the particular method used to administer the composition. Accordingly, there is a wide variety of suitable formulations of the subject pharmaceutical compositions. Liquid form preparations include solutions and emulsions, for example, water, water/propylene glycol solutions, or organic solvents. When administered to a mammal, the compounds and compositions of the present disclosure and pharmaceutically acceptable vehicles may be sterile. In some instances, an aqueous medium is employed as a vehicle e.g., when the subject compound is administered orally, intravenously, or intradermally, such as water, saline solutions, fruit juices, and aqueous dextrose and glycerol solutions.

Any of the pharmaceutical compositions described herein can comprise (as the active component) at least one compound of Formula (I), or a pharmaceutically acceptable salt, polymorph, stereoisomer, or solvate thereof. As described below, pharmaceutical compositions comprising a compound disclosed herein may be formulated in various dosage forms, and specially formulated for administration in solid, semi-solid, or liquid form, including those adapted for the following:
A. Oral administration, for example, drenches (aqueous or non-aqueous solutions or suspensions), tablets, films, or capsules, e.g., those targeted for buccal, sublingual, and systemic absorption, boluses, powders, granules, syrups, pastes for application to the tongue;
B. Parenteral administration, for example, by subcutaneous, intradermal, intramuscular, intravenous or epidural injection as, for example, a sterile solution or suspension, or sustained release formulation;
C. Topical application/transdermal administration, for example, as a cream, ointment, or a controlled release patch or spray applied to the skin, or orifices and/or mucosal surfaces such as intravaginally or intrarectally, for example, as a pessary, cream or foam;
D. Modified release dosage forms, including delayed-, extended-, prolonged-, sustained-, pulsatile-, controlled-, accelerated-, fast-, targeted-, programmed-release, and gastric retention dosage forms, such modified release dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126).

Tamper resistant dosage forms/packaging of any of the disclosed pharmaceutical compositions are contemplated.

### A. Oral Administration

The pharmaceutical compositions disclosed herein may be provided in solid, semisolid, or liquid dosage forms for oral administration. As used herein, oral administration includes gastric (enteral) delivery, for example whereby the medication is taken by mouth and swallowed, as well as intraoral administration such as through the mucosal linings of the oral cavity, e.g., buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent dosage forms (e.g., effervescent or non-effervescent tablets, films, powders or granules), solutions, emulsions, suspensions, solutions, wafers, films, sprinkles, elixirs, and syrups. In addition to the active ingredient(s), the pharmaceutical compositions may contain one or more pharmaceutically acceptable vehicles (e.g., carriers or excipients), including, but not limited to, auxiliary agents, stabilizing agents, solubilizing agents, thickening agents, lubricants, binders, granulators, fillers, diluents, disintegrants, wetting agents, glidants, anti-caking agents, coloring agents, sweetening agents, dye-migration inhibitors, preservatives, antioxidants, lyoprotectants, complexing agents, flavoring agents, matrix-forming agents, dispersing agents, performance modifiers, controlled-release polymers, solvents, pH modifiers, and sources of carbon dioxide. In some embodiments, the pharmaceutically acceptable vehicle comprises an organic acid agent, which as discussed herein, has been found to provide unique benefits as both a stabilizing agent and a solubilizing agent to aid release from the disclosed dosage forms and to provide stabilization of the compounds herein.

In some embodiments, pharmaceutical compositions of the present disclosure may be in orodispersible dosage forms (ODxs), including sublingual dosage forms, buccal dosage forms, e.g., orally disintegrating tablets (ODTs) (also sometimes referred to as fast disintegrating tablets, orodispersible tablets, or fast dispersible tablets) or orodispersible films (ODFs) (or wafers). Such dosage forms may be particularly advantageous in the present disclosure as they allow for pre-gastric absorption of the compounds/salts herein, e.g., when administered intraorally through the mucosal linings of the oral cavity, e.g., buccal, lingual, and sublingual administration, for increased bioavailability and faster onset compared to oral administration through the gastrointestinal tract. Additionally, orodispersible dosage forms may be advantageous for the treatment of pediatric/adolescent patients or patients that have general difficulty swallowing traditional dosage forms such as general tablets or capsules.

In some embodiments, the orodispersible dosage form (ODx) is a sublingual dosage form to be disintegrated/dissolved under the tongue, whereby the contents (e.g., the compounds of the present disclosure) are absorbed through the mucous membrane beneath the tongue where they enter venous circulation. In some embodiments, the sublingual dosage form is disintegrated/dissolved under the tongue, whereby the contents are converted into a liquid or semi-solid dosage form, such as a solution, syrup, or paste upon mixing with the saliva, and subsequently swallowed. In some embodiments, the orodispersible dosage form (ODx) is a buccal dosage form to be disintegrated/dissolved in the buccal cavity, whereby the contents (e.g., the compounds of the present disclosure) are absorbed through the oral mucosa lining the mouth where they enter venous circulation. In some embodiments, the buccal dosage form is disintegrated/dissolved in the buccal cavity, whereby the contents are converted into a liquid or semi-solid dosage form, such as a solution, syrup, or paste upon mixing with the saliva, and subsequently swallowed. In addition to the active ingredient(s), the pharmaceutical compositions in orodispersible dosage form (ODxs) may contain one or more pharmaceutically acceptable vehicles (e.g., one or more of a binder, a filler, a diluent, a disintegrant, a lyoprotectaut, a preservative, an antioxidant, a stabilizing agent, a solubilizing agent, a flavoring agent, a source of carbon dioxide, a bioadhesive agent, etc., and/or any other pharmaceutically acceptable vehicle set forth herein, with specific mention being made to an organic acid agent).

Orodispersible dosage forms can be prepared by different techniques, such as freeze drying (lyophilization), molding, spray drying, mass extrusion or compressing. In some embodiments, the orodispersible dosage forms are prepared by lyophilization. In some embodiments, the orodispersible dosage forms disintegrate in less than about 90 seconds, in less than about 60 seconds, in less than about 30 seconds, in less than about 20, in less than about 10 seconds, in less than about 5 seconds, or in less than about 2 seconds after being received in the oral cavity. In some embodiments, the orodispersible dosage forms dissolve in less than about 90 seconds, in less than about 60 seconds, or in less than about 30 seconds after being received in the oral cavity. In some embodiments, the orodispersible dosage forms disperse in less than about 90 seconds, in less than about 60 seconds, in less than about 30 seconds, in less than about 20, in less than about 10 seconds, in less than about 5 seconds, or in less than about 2 seconds after being received in the oral cavity. In some embodiments, the pharmaceutical compositions are in the form of orodispersible dosage forms, such as oral disintegrating tablets (ODTs), having a disintegration time according to the United States Pharmacopeia (USP) disintegration test <701> of not more than about 30 seconds, not more than about 20, not more than about 10 seconds, not more than about 5 seconds, not more than about 2 seconds. Orodispersible dosage forms having longer disintegration times according to the United States Pharmacopeia (USP) disintegration test <701>, such as when adapted for extended release, for example 2 minutes, 3 minutes, 4 minutes, 5 minutes, 10 minutes, 15 minutes, 20 minutes, 25 minutes, 30 minutes, 45 minutes, 60 minutes, or any range therebetween, or longer, are also contemplated.

In some embodiments, the pharmaceutical compositions are in the form of sublingual tablets, prepared by direct compression, compression molding, or lyophilization. In some embodiments, the sublingual tablets are created by direct compression, whereby directly compressible pharmaceutical vehicles such as organic acid agent (optionally coated), binder, filler, lubricant, etc. are mixed with the compound of Formula (I) (or a pharmaceutically acceptable salt, polymorph, stereoisomer, or solvate thereof) and compressed into tablets by direct compression. In some embodiments, the sublingual tablet contains one or more binders/fillers/diluents such as lactose, mannitol, microcrystalline cellulose, polyvinylpyrrolidone (PVP). In some embodiments, the sublingual tablet contains a lubricant e.g., magnesium stearate. Other pharmaceutically acceptable vehicles such as soluble excipients, dry binders, pH modifiers/buffers, surface-active agents, sweetening agents, flavoring agents, etc. may also be used. A non-limiting example of sublingual tablet formulation is one that includes a compound of Formula (I) (or a pharmaceutically acceptable salt, polymorph, stereoisomer, or solvate thereof), an organic acid agent such as citric acid (which may be optionally coated), lactose, mannitol, PVP, and magnesium stearate, and optionally one or more additional pharmaceutically acceptable vehicles set forth herein.

In some embodiments, the sublingual tablet can comprise a monolayer, bilayer, or trilayer. In some embodiments, the monolayer sublingual tablet contains an active ingredient (e.g., a compound of Formula (I), or a pharmaceutically acceptable salt, polymorph, stereoisomer, or solvate thereof) and one or more pharmaceutically acceptable vehicles (e.g., an organic acid agent such as citric acid). In some embodiments, the monolayer sublingual tablet is effervescent and is formulated with an “effervescent couple,” i.e., a combination of an organic acid agent and a source of carbon dioxide. In some embodiments, the bilayer sublingual tablet contains one or more pharmaceutically acceptable vehicles (e.g., an organic acid agent such as citric acid) in a first layer, and an active ingredient (e.g., a compound of Formula (I), or a pharmaceutically acceptable salt, polymorph, stereoisomer, or solvate thereof) in the second layer. The second layer may optionally contain one or more pharmaceutically acceptable vehicles. This configuration allows the active ingredient to be stored separately from all, or certain, pharmaceutically acceptable vehicles so that contact between the active ingredient and those vehicles is minimized or altogether prevented, which can in some instances increase the stability of the active ingredient and optionally increase the shelf life of the composition compared to the case where the vehicles and the active ingredient were contained in a single layer. In some embodiments, the bilayer sublingual tablet is an effervescent sublingual tablet whereby the first layer is effervescent comprising an effervescent couple and optionally other pharmaceutically acceptable vehicles, and the second layer comprises the active ingredient (e.g., a compound of Formula (I), or a pharmaceutically acceptable salt, polymorph, stereoisomer, or solvate thereof, and optionally one or more pharmaceutically acceptable vehicles, the second layer being either non-effervescent or effervescent. For trilayer sublingual tablets, each of the layers may be different or two of the layers, such as the upper and lower layers, may have substantially the same composition. In some embodiments, the lower and upper layers surround a core layer containing the active ingredient (e.g., a compound of Formula (I), or a pharmaceutically acceptable salt, polymorph, stereoisomer, or solvate thereof). In some embodiments, the lower and upper layers may contain one or more vehicle components such as a solubilizing agent, stabilizing agent, etc. (e.g., an organic acid agent such as citric acid). In some embodiments, the lower and upper layers have the same composition. Alternatively, the lower and upper layers may contain different vehicles or different amounts of the same vehicle. The core layer typically contains the active ingredient, optionally with one or more pharmaceutically acceptable vehicles. As described above, such a trilayer sublingual tablet configuration allows the active ingredient to be stored separately from all, or certain, pharmaceutically acceptable vehicles so that contact between the active ingredient and those vehicles is minimized or altogether prevented. In some embodiments, the trilayer sublingual tablet is an effervescent sublingual tablet whereby at least one of, at least two of, or all three of the layers are effervescent (formulated with an effervescent couple). In some embodiments, the lower and upper layers are effervescent, comprising an organic acid agent (e.g., citric acid), a source of carbon dioxide, and optionally other pharmaceutically acceptable vehicles, and the core layer comprises the active ingredient (e.g., a compound of Formula (I), or a pharmaceutically acceptable salt, polymorph, stereoisomer, or solvate thereof, and optionally one or more pharmaceutically acceptable vehicles, the core layer being either non-effervescent or effervescent.

In some embodiments, the pharmaceutical compositions are in the form of lyophilized orodispersible dosage forms, such as lyophilized ODTs. In some embodiments, the lyophilized orodispersible dosage forms (e.g., lyophilized ODTs) are created by creating a porous matrix by subliming the water from pre-frozen aqueous formulation of the drug containing matrix-forming agents and other vehicles such as those set forth herein, e.g., one or more lyoprotectants, preservatives, antioxidants, stabilizing agents, solubilizing agents, flavoring agents, etc. In some embodiments, the orodispersible dosage forms comprise two component frameworks of a lyophilized matrix system that work together to ensure the development of a successful formulation. In some embodiments, the first component is a water-soluble polymer such as gelatin, dextran, alginate, and maltodextrin. This component maintains the shape and provides mechanical strength to the dosage form (binder). In some embodiments, the second constituent is a matrix-supporting/disintegration-enhancing agent such as sucrose, lactose, mannitol, xylitol, microcrystalline cellulose, calcium diphosphate, and/or starch, which acts by cementing the porous framework, provided by the water-soluble polymer and accelerates the disintegration of the orodispersible dosage forms. In some embodiments, the lyophilized orodispersible dosage form (e.g., lyophilized ODT) includes gelatin and mannitol. In some embodiments, the lyophilized orodispersible dosage form (e.g., lyophilized ODT) includes gelatin, mannitol, and one or more of a lyoprotectant, a preservative, an antioxidant, a stabilizing agent, a solubilizing agent, a flavoring agent, and/or another pharmaceutically acceptable vehicle set forth herein, with particular mention being made to an organic acid agent (e.g., citric acid). A non-limiting example of an ODT formulation is Zydis® orally dispersible tablets (available from Catalent). In some embodiments, the ODT formulation (e.g., Zydis® orally dispersible tablets) includes one or more water-soluble polymers, such as gelatin, one or more matrix materials, fillers, or diluents, such as mannitol, a compound of Formula (I), or a pharmaceutically acceptable salt, polymorph, stereoisomer, or solvate thereof, and optionally a lyoprotectant, a preservative, an antioxidant, a stabilizing agent, a solubilizing agent, a flavoring agent, and/or another pharmaceutically acceptable vehicle set forth herein. In some embodiments, the ODT formulation (e.g., Zydis® orally dispersible tablets) includes gelatin, mannitol, a compound of Formula (I), or a pharmaceutically acceptable salt, polymorph, stereoisomer, or solvate thereof, and citric acid and/or tartaric acid.

In some embodiments, the ODT can comprise a monolayer, bilayer, or trilayer. In some embodiments, the monolayer ODT contains an active ingredient (e.g., a compound of Formula (I), or a pharmaceutically acceptable salt, polymorph, stereoisomer, or solvate thereof) and one or more pharmaceutically acceptable vehicles (e.g., an organic acid agent such as citric acid). In some embodiments, the monolayer ODT is effervescent and is formulated with an “effervescent couple,” i.e., a combination of an organic acid agent and a source of carbon dioxide. In some embodiments, the bilayer ODT contains one or more pharmaceutically acceptable vehicles (e.g., an organic acid agent such as citric acid) in a first layer, and an active ingredient (e.g., a compound of Formula (I), or a pharmaceutically acceptable salt, polymorph, stereoisomer, or solvate thereof) in the second layer. The second layer may optionally contain one or more pharmaceutically acceptable vehicles. This configuration allows the active ingredient to be stored separately from all, or certain, pharmaceutically acceptable vehicles so that contact between the active-ingredient and those vehicles is minimized or altogether prevented, which can in some instances increase the stability of the active ingredient and optionally increase the shelf life of the composition compared to the case where the vehicles and the active ingredient were contained in a single layer. In some embodiments, the bilayer ODT is an effervescent ODT whereby the first layer is effervescent comprising an effervescent couple and optionally other pharmaceutically acceptable vehicles, and the second layer comprises the active ingredient (e.g., a compound of Formula (I), or a pharmaceutically acceptable salt, polymorph, stereoisomer, or solvate thereof, and optionally one or more pharmaceutically acceptable vehicles, the second layer being either non-effervescent or effervescent. For trilayer ODTs, each of the layers may be different or two of the layers, such as the upper and lower layers, may have substantially the same composition. In some embodiments, the lower and upper layers surround a core layer containing the active ingredient (e.g., a compound of Formula (I), or a pharmaceutically acceptable salt, polymorph, stereoisomer, or solvate thereof). In some embodiments, the lower and upper layers may contain one or more vehicle components such as a solubilizing agent, stabilizing agent, etc. (e.g., an organic acid agent such as citric acid). In some embodiments, the lower and upper layers have the same composition. Alternatively, the lower and upper layers may contain different vehicles or different amounts of the same vehicle. The core layer typically contains the active ingredient, optionally with one or more pharmaceutically acceptable vehicles. As described above, such a trilayer ODT configuration allows the active ingredient to be stored separately from all, or certain, pharmaceutically acceptable vehicles so that contact between the active ingredient and those vehicles is minimized or altogether prevented. In some embodiments, the trilayer ODT is an effervescent ODT whereby at least one of, at least two of, or all three of the layers are effervescent (formulated with an effervescent couple). In some embodiments, the lower and upper layers are effervescent, comprising an organic acid agent (e.g., citric acid), a source of carbon dioxide, and optionally other pharmaceutically acceptable vehicles, and the core layer comprises the active ingredient (e.g., a compound of Formula (I), or a pharmaceutically acceptable salt, polymorph, stereoisomer, or solvate thereof, and optionally one or more pharmaceutically acceptable vehicles, the core layer being either non-effervescent or effervescent.

In some embodiments, the pharmaceutical compositions are in the form of lyophilized orodispersible films (ODFs) (or wafers). In some embodiments, the pharmaceutical compositions are in the form of lyophilized ODFs protected for the long-term storage by a specialty packaging excluding moisture, oxygen, and light. In some embodiments, the lyophilized ODFs are created by creating a porous matrix by subliming the water from pre-frozen aqueous formulation of the drug containing matrix-forming agents and other vehicles such as those set forth herein, e.g., one or more of a lyoprotectant, a preservative, an antioxidant, a stabilizing agent, a solubilizing agent, a flavoring agent, and/or another pharmaceutically acceptable vehicle set forth herein. In some embodiments, the lyophilized ODF includes a thin water-soluble film matrix. In some embodiments, the ODFs comprise two component frameworks of a lyophilized matrix system that work together to ensure the development of a successful formulation. In some embodiments, the first component is a water-soluble polymer such as gelatin, dextran, alginate, and maltodextrin. This component maintains the shape and provides mechanical strength to the film/wafer (binder). In some embodiments, the second constituent is a matrix-supporting/disintegration-enhancing agent such as sucrose, lactose, mannitol, xylitol, microcrystalline cellulose, calcium diphosphate, and/or starch, which acts by cementing the porous framework, provided by the water-soluble polymer and accelerates the disintegration of the wafer. In some embodiments, the lyophilized ODFs include gelatin and mannitol. In some embodiments, the lyophilized ODFs include gelatin, mannitol, and one or more of a lyoprotectant, a preservative, an antioxidant, a stabilizing agent, a solubilizing agent, a flavoring agent, and/or another pharmaceutically acceptable vehicle set forth herein, with particular mention being made to an organic acid agent (e.g., citric acid).

In some embodiments, the ODF (or wafer) can comprise a monolayer, bilayer, or trilayer. In some embodiments, the monolayer ODF (or wafer) contains an active ingredient (e.g., a compound of Formula (I), or a pharmaceutically acceptable salt, polymorph, stereoisomer, or solvate thereof) and one or more pharmaceutically acceptable vehicles (e.g., an organic acid agent such as citric acid). In some embodiments, the monolayer ODF (or wafer) is effervescent and is formulated with an effervescent couple. In some embodiments, the bilayer ODF (or wafer) contains one or more pharmaceutically acceptable vehicles (e.g., an organic acid agent such as citric acid) in a first layer, and an active ingredient (e.g., a compound of Formula (I), or a pharmaceutically acceptable salt, polymorph, stereoisomer, or solvate thereof) in the second layer. The second layer may optionally contain one or more pharmaceutically acceptable vehicles. This configuration allows the active ingredient to be stored separately from all, or certain, pharmaceutically acceptable vehicles so that contact between the active ingredient and those vehicles is minimized or altogether prevented, which can in some instances increase the stability of the active ingredient and optionally increase the shelf life of the composition compared to the case where the vehicles and the active ingredient were contained in a single layer. In some embodiments, the bilayer ODF (or wafer) is an effervescent ODF (or wafer) whereby the first layer is effervescent comprising an effervescent couple and optionally other pharmaceutically acceptable vehicles, and the second layer comprises the active ingredient (e.g., a compound of Formula (I), or a pharmaceutically acceptable salt, polymorph, stereoisomer, or solvate thereof, and optionally one or more pharmaceutically acceptable vehicles, the second layer being either non-effervescent or effervescent. For trilayer ODFs (or wafer), each of the layers may be different or two of the layers, such as the upper and lower layers, may have substantially the same composition. In some embodiments, the lower and upper layers surround a core layer containing the active ingredient (e.g., a compound of Formula (I), or a pharmaceutically acceptable salt, polymorph, stereoisomer, or solvate thereof). In some embodiments, the lower and upper layers may contain one or more vehicle components such as a solubilizing agent, stabilizing agent, etc. (e.g., an organic acid agent such as citric acid). In some embodiments, the lower and upper layers have the same composition. Alternatively, the lower and upper layers may contain different vehicles or different amounts of the same vehicle. The core layer typically contains the active ingredient, optionally with one or more pharmaceutically acceptable vehicles. As described above, such a trilayer ODF (or wafer) configuration allows the active ingredient to be stored separately from all, or certain, pharmaceutically acceptable vehicles so that contact between the active ingredient and those vehicles is minimized or altogether prevented. In some embodiments, the trilayer ODF (or wafer) is an effervescent ODF (or wafer) whereby at least one of, at least two of, or all three of the layers are effervescent (formulated with an effervescent couple). In some embodiments, the lower and upper layers are effervescent, comprising an organic acid agent (e.g., citric acid), a source of carbon dioxide, and optionally other pharmaceutically acceptable vehicles, and the core layer comprises the active ingredient (e.g., a compound of Formula (I), or a pharmaceutically acceptable salt, polymorph, stereoisomer, or solvate thereof, and optionally one or more pharmaceutically acceptable vehicles, the core layer being either non-effervescent or effervescent.

Examples of pharmaceutically acceptable lyoprotectants include, but are not limited to, disaccharides such as sucrose and trehalose, anionic polymers such as sulfobutylether-β-cyclodextrin (SBECD) and hyaluronic acid, and hydroxylated cyclodextrins.

Examples of pharmaceutically acceptable preservatives include, but are not limited to, glycerin, methyl and propylparaben, benzoic acid, sodium benzoate and alcohol.

Examples of pharmaceutically acceptable antioxidants, which may act to further enhance stability of the composition, include, but are not limited to: (1) water-soluble antioxidants, such as ascorbic acid, cysteine or salts thereof (cysteine hydrochloride), sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of pharmaceutically acceptable stabilizing agents include, but are not limited to, organic acid agents (e.g., citric acid), fatty acids, fatty alcohols, alcohols, long chain fatty acid esters, long chain ethers, hydrophilic derivatives of fatty acids, polyvinylpyrrolidones, polyvinyl ethers, polyvinyl alcohols, hydrocarbons, hydrophobic polymers, moisture-absorbing polymers, glycerol, methionine, monothioglycerol, ascorbic acid, polysorbate, arginine, cyclodextrins, microcrystalline cellulose, modified celluloses (e.g., carboxymethylcellulose, sodium salt), sorbitol, and cellulose gel.

Examples of pharmaceutically acceptable solubilizing agents (or dissolution aids) include, but are not limited to, organic acid agents (e.g., citric acid, fumaric acid, DL-malic acid, tartaric acid, lactic acid, maleic acid, etc.), hydroxypropylcellulose, hydroxypropylmethylcellulose, sodium stearyl fumarate, methacrylic acid copolymer LD, methylcellulose, sodium lauryl sulfate, polyoxyl 40 stearate, purified shellac, sodium dehydroacetate, L-ascorbyl stearate, L-asparagine acid, adipic acid, aminoalkyl methacrylate copolymer E, propylene glycol alginate, casein, casein sodium, a carboxyvinyl polymer, carboxymethylethylcellulose, powdered agar, guar gum, succinic acid, copolyvidone, cellulose acetate phthalate, dioctylsodium sulfosuccinate, zein, powdered skim milk, sorbitan trioleate, aluminum lactate, ascorbyl palmitate, hydroxyethylmethylcellulose, hydroxypropylmethylcelluloseacetate succinate, polyoxyethylene (105) polyoxypropylene (5) glycol, polyoxyethylene hydrogenated castor oil 60, polyoxyl 35 castor oil, poly(sodium 4-styrenesulfonate), polyvinylacetaldiethylamino acetate, polyvinyl alcohol, methacrylic acid copolymer S, lauromacrogol, sulfuric acid, aluminum sulfate, phosphoric acid, calcium dihydrogen phosphate, sodium dodecylbenzenesulfonate, a vinyl pyrrolidone-vinyl acetate copolymer, sodium lauroyl sarcosinate, acetyl tryptophan, sodium methyl sulfate, sodium ethyl sulfate, sodium butyl sulfate, sodium octyl sulfate, sodium decyl sulfate, sodium tetradecyl sulfate, sodium hexadecyl sulfate, and sodium octadecyl sulfate. Of these, in some embodiments, citric acid is preferred.

Flavoring agents include natural flavors extracted from plants, such as fruits, and synthetic blends of compounds which produce a pleasant taste sensation or taste masking effect. Examples of flavoring agents include, but are not limited to, aspartame, saccharin (as sodium, potassium or calcium saccharin), cyclamate (as a sodium, potassium or calcium salt), sucralose, acesulfame-K, thaumatin, neohesperidin, dihydrochalcone, ammoniated glycyrrhizin, dextrose, maltodextrin, fructose, levulose, sucrose, glucose, wild orange peel, citric acid, tartaric acid, oil of wintergreen, oil of peppermint, methyl salicylate, oil of spearmint, oil of sassafras, oil of clove, cinnamon, anethole, menthol, thymol, eugenol, eucalyptol, orange flavor, lemon, lime, and lemon-lime.

Cyclodextrins such as α-cyclodextrin, β-cyclodextrin, γ-cyclodextrin, methyl-β-cyclodextrin, hydroxyethyl β-cyclodextrin, hydroxypropyl-β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

Pharmaceutical compositions adapted for oral administration, e.g., tablets, including compressed tablets, may be formulated with various vehicles such as those set forth herein. Examples of suitable vehicles may include, but are not limited to, binders, fillers, diluents, disintegrants, wetting agents, lubricants, glidants, anti-caking agents, coloring agents, dye-migration inhibitors, sweetening agents, preservatives, antioxidants, stabilizing agents, solubilizing agents, flavoring agents, auxiliary agents, thickening agents, lubricants, granulators, lyoprotectants, complexing agents, matrix-forming agents, dispersing agents, performance modifiers, controlled-release polymers, solvents, pH modifiers, and sources of carbon dioxide.

Binders or granulators impart cohesiveness to a tablet to ensure the tablet remains intact after compression. Suitable binders or granulators include, but are not limited to, starches, such as corn starch, potato starch, and pre-gelatinized starch (e.g., STARCH 1500); gelatin; sugars, such as sucrose, glucose, dextrose, dextrins, molasses, and lactose; natural and synthetic gums, such as acacia (gum arabic), alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, partially hydrolyzed starch (e.g., maltodextrin) and mixtures thereof. In some embodiments, the binder, granulator, or filler is present from about 1%, about 5%, about 10%, about 20%, about 30%, about 40%, about 50% to about 99%, about 90%, about 80%, about 70%, about 60% by weight, based on a total weight of the pharmaceutical compositions disclosed herein, or any range therebetween.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar, bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, pre-gelatinized starch, and partially hydrolyzed starch; clays; aligns; and mixtures thereof. The amount of disintegrant in the pharmaceutical compositions disclosed herein varies upon the type of formulation, and is readily discernible to those of ordinary skill in the art. In some embodiments, the pharmaceutical compositions disclosed herein contain e.g., from about 0.5%, about 1%, about 3%, about 5%, about 10%, about 15%, to about 50%, about 40%, about 30%, about 20% by weight of a disintegrant, based on a total weight of the pharmaceutical composition, e.g., from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG) (e.g., PEG 4,000, PEG 6,000, PEG 8,000, etc., where the number refers to the approximate average molecular weight of the PEG); stearic acid; sodium lauryl sulfate; sodium stearyl fumarate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W. R. Grace Co., Baltimore, Md.) and CAB-O-SIL® (Cabot Co. of Boston, Mass.); and mixtures thereof. In some embodiments, the pharmaceutical compositions disclosed herein contain e.g., from about 0.5%, about 1%, about 2%, about 3%, about 4%, about 5%, to about 20%, about 15%, about 10%, about 7% by weight of a lubricant, based on a total weight of the pharmaceutical composition, e.g., from about 0.1% to about 5% by weight of a lubricant.

Suitable glidants include, but are not limited to, colloidal silicon dioxide, CAB-O-SIL® (Cabot Co. of Boston, Mass.), and asbestos-free talc.

Suitable anti-caking agents include, but are not limited to, silicon dioxide.

Coloring agents include any of the approved, certified, water-soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

Sweetening agents include, but are not limited to, sucrose, lactose, mannitol, syrups, glycerin, sucralose, and artificial sweeteners, such as saccharin and aspartame.

Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate.

Suspending and dispersing agents include, but are not limited to, sodium carboxymethylcellulose, pectin, tragacanth, Veegum, acacia, sodium carbomethylcellulose, hydroxypropyl methylcellulose, and polyvinylpyrolidone.

Preservatives include, but are not limited to, glycerin, methyl and propylparaben, benzoic add, sodium benzoate and alcohol.

Wetting agents include, but are not limited to, propylene glycol monostearate, sorbitan monooleate, diethylene glycol monolaurate, and polyoxyethylene lauryl ether.

Solvents include, but are not limited to, glycerin, sorbitol, ethyl alcohol, and syrup. Examples of non-aqueous liquids utilized in emulsions include, but are not limited to, mineral oil and cottonseed oil.

Examples of pH modifiers include acids (including organic acid agents), such as citric acid, acetic acid, ascorbic acid, lactic acid, aspartic acid, succinic acid, phosphoric acid, and the like; bases including salts of organic acid agents, such as sodium acetate, potassium acetate, sodium citrate (e.g., monosodium citrate, disodium citrate, and/or trisodium citrate), potassium citrate (e.g., monopotassium citrate, dipotassium citrate, and/or tripotassium citrate), sodium tartrate (e.g., monosodium tartrate and/or disodium tartrate), potassium tartrate (e.g., monopotassium tartrate and/or dipotassium tartrate), potassium sodium tartrate, ammonium citrate (e.g., monoammonium citrate, diammonium citrate, and/or triammonium citrate), ammonium tartrate (e.g., monoammonium tartrate and/or diammonium tartrate), sodium fumarate (e.g., monosodium fumarate and/or disodium fumarate), potassium fumarate (e.g., monopotassium fumarate and/or dipotassium fumarate), sodium maleate (e.g., monosodium maleate and/or disodium maleate), potassium maleate (e.g., monopotassium maleate and/or dipotassium maleate), sodium lactate, potassium lactate, calcium oxide, magnesium oxide, trisodium phosphate, sodium hydroxide, calcium hydroxide, aluminum hydroxide, and the like, and buffers generally comprising mixtures of acids and the salts of said acids.

The source of carbon dioxide may include, but is not limited to, sodium bicarbonate, sodium carbonate, potassium carbonate, potassium bicarbonate, magnesium carbonate, calcium carbonate, and sesquicarbonate. The source of carbon dioxide can be used singly, or in combination.

As described above, preferred dosage forms are those formulated with an organic acid agent, which may act as a stabilizing agent and/or solubilizing agent in the disclosed pharmaceutical compositions. The organic acid agent may be any set forth herein, with specific mention being made to citric and/or tartaric acid.

In some embodiments, the tablet (e.g., general tablets including compressed tablets) can comprise a monolayer, bilayer, or trilayer. In some embodiments, the monolayer tablet contains an active ingredient (e.g., a compound of Formula (I), or a pharmaceutically acceptable salt, polymorph, stereoisomer, or solvate thereof) and one or more pharmaceutically acceptable vehicles (e.g., an organic acid agent such as citric acid). In some embodiments, the monolayer tablet is effervescent and is formulated with an effervescent couple. In some embodiments, the bilayer tablet contains one or more pharmaceutically acceptable vehicles (e.g., an organic acid agent such as citric acid) in a first layer, and an active ingredient (e.g., a compound of Formula (I), or a pharmaceutically acceptable salt, polymorph, stereoisomer, or solvate thereof) in the second layer. The second layer may optionally contain one or more pharmaceutically acceptable vehicles. This configuration allows the active ingredient to be stored separately from all, or certain, pharmaceutically acceptable vehicles so that contact between the active ingredient and those vehicles is minimized or altogether prevented, which can in some instances increase the stability of the active ingredient and optionally increase the shelf life of the composition compared to the case where the vehicles and the active ingredient were contained in a single layer. In some embodiments, the bilayer tablet is an effervescent tablet whereby the first layer is effervescent comprising an effervescent couple and optionally other pharmaceutically acceptable vehicles, and the second layer comprises the active ingredient (e.g., a compound of Formula (I), or a pharmaceutically acceptable salt, polymorph, stereoisomer, or solvate thereof, and optionally one or more pharmaceutically acceptable vehicles, the second layer being either non-effervescent or effervescent. For trilayer tablets, each of the layers may be different or two of the layers, such as the upper and lower layers, may have substantially the same composition. In some embodiments, the lower and upper layers surround a core layer containing the active ingredient (e.g., a compound of Formula (I), or a pharmaceutically acceptable salt, polymorph, stereoisomer, or solvate thereof). In some embodiments, the lower and upper layers may contain one or more vehicle components such as a solubilizing agent, stabilizing agent, etc. (e.g., an organic acid agent such as citric acid). In some embodiments, the lower and upper layers have the same composition. Alternatively, the lower and upper layers may contain different vehicles or different amounts of the same vehicle. The core layer typically contains the active ingredient, optionally with one or more pharmaceutically acceptable vehicles. As described above, such a trilayer tablet configuration allows the active ingredient to be stored separately from all, or certain, pharmaceutically acceptable vehicles so that contact between the active ingredient and those vehicles is minimized or altogether prevented. In some embodiments, the trilayer tablet is an effervescent tablet whereby at least one of, at least two of, or all three of the layers are effervescent (formulated with an effervescent couple). In some embodiments, the lower and upper layers are effervescent, comprising an organic acid agent (e.g., citric acid), a source of carbon dioxide, and optionally other pharmaceutically acceptable vehicles, and the core layer comprises the active ingredient (e.g., a compound of Formula (I), or a pharmaceutically acceptable salt, polymorph, stereoisomer, or solvate thereof, and optionally one or more pharmaceutically acceptable vehicles, the core layer being either non-effervescent or effervescent.

It should be understood that many vehicles (carriers, excipients, etc.) may serve several functions, even within the same formulation. Particular mention is made to pharmaceutical compositions herein containing an organic acid agent such as citric acid, which may play multiple roles as a stabilizing agent, e.g., to stabilize the psilocin compound of the present disclosure in free base or salt form, as a solubilizing agent to provide fast dissolution of the active for rapid onset, etc., particularly for dosage forms adapted for rapid onset and a shorter duration of drug action, such as orodispersible dosage forms (e.g., ODTs and ODFs), as a flavoring agent, a pH modifier, and/or as an antioxidant.

The tablet dosage forms may be prepared from the active ingredient in powdered, crystalline, or granular forms, alone or in combination with one or more vehicles (e.g., carriers or excipients) described herein, including binders, disintegrants, controlled-release polymers, pH modifiers, lubricants, diluents, and/or coloring agents. Flavoring and sweetening agents are especially useful in the formation of chewable tablets and lozenges.

The pharmaceutical compositions herein may be in the form of compressed tablets, tablet triturates, chewable lozenges, rapidly dissolving tablets, multiple compressed tablets, or any of the above which are coated, such as enteric-coating tablets, sugar-coated, or film-coated tablets. Coated tablets are tablets covered with one or more layers of pharmaceutically acceptable vehicle or mixtures of vehicles such as natural or synthetic resins, polymers, gums, fillers, sugars, plasticizers, polyols, waxes, organic bases, coloring matters authorized by the appropriate national or regional authority, and flavoring substances. Such coating materials generally do not contain any active ingredient, e.g., any of the compounds described herein (e.g., compound of Formula (I), or a pharmaceutically acceptable salt, polymorph, stereoisomer, or solvate thereof). The tablets may be coated for a variety of reasons such as protection of the active ingredients from burst release from the matrix, air, moisture or light, masking of unpleasant tastes and odors or improvement of appearance. The substance used for coating may be applied as a solution or suspension. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

In some embodiments, the pharmaceutical composition (e.g., a tablet composition formulated for oral administration such as a monolayer tablet composition), comprises any of the compounds described herein (e.g., compound of Formula (I), or a pharmaceutically acceptable salt, polymorph, stereoisomer, or solvate thereof), and a polymer.

In some embodiments, the tablet composition is a modified-release tablet adapted for sustained release and preferably maximum sustained release. In some embodiments, the release period of any of the compounds described herein (e.g., compound of Formula (I), or a pharmaceutically acceptable salt, polymorph, stereoisomer, or solvate thereof), in the formulations of the disclosure is greater than 4 hours, greater than 6 hours, greater than 8 hours, greater than 10 hours, greater than 12 hours, greater than 16 hours, greater than 20 hours, greater than 24 hours, greater than 28 hours, greater than 32 hours, greater than 36 hours, greater than 48 hours.

In some embodiments, the tablet composition is adapted for tamper resistance. In some embodiments, the tablet composition comprises polyethylene oxide (PEO), e.g., MW about 2,000 to about 7,000 KDa, in combination with HPMC. In some embodiments, the tablet composition may further comprise polyethylene glycol (PEG), e.g., PEG 8,000. In some embodiments, the tablet composition may further comprise a polymer carrying one or more negatively charged groups, e.g., polyacrylic acid. In some embodiments, the tablet composition comprising PEO is further subjected to heating/annealing, e.g., extrusion conditions.

In some embodiments, the pharmaceutical composition comprises a combination of (i) a water-insoluble neutrally charged non-ionic matrix; (ii) a polymer carrying one or more negatively charged groups; and (iii) any of the compounds described herein (e.g., compound of Formula (I), or a pharmaceutically acceptable salt, polymorph, stereoisomer, or solvate thereof).

In some embodiments, the polymer carrying one or more negatively charged groups is selected from the group consisting of polyacrylic acid, polylactic acid, polyglycolic acid, polymethacrylate carboxylates, cation-exchange resins, clays, zeolites, hyaluronic acid, anionic gums, salts thereof, and mixtures thereof. In some embodiments, the anionic gum is selected from the group consisting of naturally occurring materials and semi-synthetic materials. In some embodiments, the naturally occurring material is selected from the group consisting of alginic acid, pectin, xanthan gum, carrageenan, locust bean gum, gum arabic, gum karaya, guar gum, and gum tragacanth. In some embodiments, the semi-synthetic material is selected from the group consisting of carboxymethyl-chitin and cellulose gum.

Moreover, without wishing to be bound by theory, in some embodiments, the role of the polymer carrying one or more negatively charged groups, e.g., moieties of acidic nature as in those of the acidic polymers described herein, surprisingly offers significant retention of any of the compounds described herein (e.g., compound of Formula (I), or a pharmaceutically acceptable salt, polymorph, stereoisomer, or solvate thereof), in the matrix. In some embodiments, this negative charge may be created in situ, for example, based on release of a proton due to pKa and under certain pH conditions or through electrostatic interaction/creation of negative charge. Further noting that acidic polymers may be the salts of the corresponding weak acids that will be the related protonated acids in the stomach; which, and without wishing to be bound by theory, will neutralize the charge and may reduce the interactions of any of the compounds described herein (e.g., a compound of compound of Formula (I), or a pharmaceutically acceptable salt, polymorph, stereoisomer, or solvate thereof), with the matrix. In addition, the release matrix may be further complemented by other inactive pharmaceutical ingredients to aid in preparation of the appropriate solid dose form such as fillers, disintegrants, flow improving agents, lubricants, colorants, taste maskers.

In some embodiments, the water-insoluble neutrally charged non-ionic matrix is selected from cellulose-based polymers such as HPMC, alone or enhanced by mixing with components selected from the group consisting of starches; waxes; neutral gums; polymethacrylates; PVA; PVA/PVP blends; and mixtures thereof. In some embodiments, the cellulose-based polymer is hydroxypropyl methylcellulose (HPMC).

In some embodiments, the cellulose-based polymer is hydroxypropyl methylcellulose (HPMC). In some embodiments, the tablet composition comprises about 1%, about 5%, about 10%, about 20%, about 30%, about 40%, about 50%, about 60%, about 70%, about 80% hydroxypropyl methylcellulose by weight, based on a total weight of the pharmaceutical composition, or any range therebetween. In some embodiments, the pharmaceutical composition comprises starch, e.g., about 1%, about 5%, about 10%, about 15%, about 20%, about 25%, about 30%, about 35%, about 40%, about 45%, about 50% starch by weight, based on a total weight of the pharmaceutical composition, or any range therebetween. In some embodiments, the pharmaceutical comprises a combination of HPMC and starch.

Disclosed herein are pharmaceutical compositions in modified release dosage forms, which comprise a compound as disclosed herein (e.g., a compound of Formula (I), or a pharmaceutically acceptable salt, polymorph, stereoisomer, or solvate thereof) and one or more release controlling vehicles as described herein. Suitable modified release controlling vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling vehicles.

In some embodiments, the oral pharmaceutical composition is for low dose maintenance therapy that can be constructed using the compounds described herein, capitalizing on their ability to bind with anionic polymers.

Further disclosed herein are pharmaceutical compositions in enteric coated dosage forms, which comprise a compound as disclosed herein (e.g., a compound of Formula (I), or a pharmaceutically acceptable salt, polymorph, stereoisomer, or solvate thereof) and one or more release controlling vehicles for use in an enteric coated dosage form. The pharmaceutical compositions may also comprise non-release controlling vehicles.

Further disclosed herein are pharmaceutical compositions in effervescent dosage form, which comprise a compound as disclosed herein (e.g., a compound of Formula (I), or a pharmaceutically acceptable salt, polymorph, stereoisomer, or solvate thereof) and one or more pharmaceutically acceptable vehicles, which may be release controlling vehicles and/or non-release controlling vehicles. Effervescent means that the dosage form, when mixed with liquid, including water, juice, saliva, etc., evolves a gas. In general, the effervescent dosage forms of the present disclosure comprise an organic acid agent and a source of carbon dioxide, referred to herein as an “effervescent couple.” Such effervescent dosage forms effervesce (evolve gas) through chemical reaction between the organic acid agent and the source of carbon dioxide, which takes place upon exposure to an aqueous environment, such as upon placement in water, juice, or other drinkable fluid, or from the aqueous environment in the oral cavity, such as saliva in the mouth. Specifically, the reaction between the organic acid agent and the source of carbon dioxide produces carbon dioxide gas upon contact with an aqueous medium such as water, juice, or saliva. While use of disintegrants are optional, effervescent dosage forms do not require a disintegrant as the evolution of the gas in situ facilitates the disintegration process.

For clarity, an “effervescent couple” refers to at least one organic acid agent and at least one source of carbon dioxide being contained in a dosage form, regardless of assembly—for example, the organic acid agent and the source of carbon dioxide can be admixed (as powders), layered on top of one another, agglomerated or otherwise “glued” together in granular form, or held separately from one another such as in separate layers within the dosage form. Further, the term “couple” in this context is not meant to be limited to only an organic acid agent and a source of carbon dioxide and is open to the inclusion of other materials unless specified otherwise; for example, effervescent agglomerates/granules made from bringing together (or “gluing”) an organic acid agent and a source of carbon dioxide may include other vehicles including binders (the “glue”) and the effervescent agglomerates/granules may nonetheless be referred to as an effervescent couple.

In some embodiments, the source of carbon dioxide is sodium bicarbonate. In some embodiments, the source of carbon dioxide is sodium carbonate. In some embodiments, the source of carbon dioxide is potassium carbonate. In some embodiments, the source of carbon dioxide is potassium bicarbonate. However, reactants which evolve oxygen or other gases besides carbon dioxide, and which are safe for human consumption, are also contemplated for use in the disclosed effervescent dosage forms, in addition to or in lieu of the source of carbon dioxide. While not wishing to be bound by theory, it is believed that the effervescence can help quickly break up the dosage form, and in some routes of administration such as intraoral routes, can help reduce the perception of grittiness by providing a distracting sensory experience of effervescence.

In some embodiments, the effervescent dosage form is to be reconstituted in a drinkable fluid such as water or juice, thereby forming an oral liquid dosage form (e.g., solution), prior to consumption. In some embodiments, the effervescent dosage form is to be placed in the oral cavity, where contact with the aqueous environment (saliva) causes disintegration/dissolution of the dosage form along with effervescence. Here, the contents of the effervescent dosage form may be converted into a liquid or semi-solid dosage form, such as a solution, syrup, or paste upon mixing with the saliva, and subsequently swallowed. Alternatively, the effervescent dosage form may be an intraoral dosage form, e.g., a buccal, lingual, or sublingual dosage form, whereby placement in the aqueous environment (saliva) of the oral cavity causes disintegration/dissolution of the dosage form along with effervescence, and pre-gastric absorption of the contents through the oral mucosa. Such pre-gastric absorption may provide for increased bioavailability and faster onset compared to oral administration through the gastrointestinal tract. In some embodiments, the effervescent dosage form is a sublingual dosage form to be disintegrated/dissolved under the tongue, whereby the contents (e.g., the compounds of the present disclosure) are absorbed through the mucous membrane beneath the tongue where they enter venous circulation. In some embodiments, the effervescent dosage form is a buccal dosage form to be disintegrated/dissolved in the buccal cavity, whereby the contents (e.g., the compounds of the present disclosure) are absorbed through the oral mucosa lining the mouth where they enter venous circulation. Effervescent dosage forms may be advantageous for the treatment of pediatric/adolescent patients or patients that have general difficulty swallowing traditional dosage forms such as general tablets or capsules, since effervescent dosage forms can be reconstituted into easy to swallow liquid or semi-solid dosage forms or taken intraorally.

When adapted for intraoral administration, it may be beneficial to formulate the effervescent dosage form with a bioadhesive agent, in addition to the effervescent couple. “Bioadhesive agents” are substances which promote adhesion or adherence to a biological surface, such as mucous membranes. For example, bioadhesive agents are themselves capable of adhering to a biological surface when placed in contact with that surface (e.g., mucous membrane) in order to enable compositions of the disclosure to adhere to that surface, which promotes more efficient transfer of the contents from the dosage form to the biological surface. A variety of polymers known in the art can be used as bioadhesive agents, for example polymeric substances, preferably with an average (weight average) molecular weight above 5,000 g/mol. It is preferred that such polymeric materials are capable of rapid swelling when placed in contact with an aqueous medium such a water or saliva, and/or are substantially insoluble in water at room temperature and atmospheric pressure. Examples of suitable bioadhesive agents include, but are not limited to, cyclodextrin, cellulose derivatives such as hydroxypropylmethyl cellulose (HPMC), hydroxyethyl cellulose (HEC), hydroxypropyl cellulose (HPC), methyl cellulose, ethyl hydroxyethyl cellulose, carboxymethyl cellulose, modified cellulose gum and sodium carboxymethyl cellulose (NaCMC); starch derivatives such as moderately cross-linked starch, modified starch and sodium starch glycolate; acrylic polymers such as carbomer and its derivatives (polycarbophyl, Carbopol®, etc.); polyvinylpyrrolidone (PVP); polyethylene oxide (PEO); chitosan (poly-(D-glucosamine)); natural polymers such as gelatin, sodium alginate, pectin; scleroglucan; xanthan gum; guar gum; poly co-(methylvinyl ether/maleic anhydride); and crosscarmellose (e.g. crosscarmellose sodium). Such polymers may be crosslinked. Combinations of two or more bioadhesive agents can also be used.

An effervescent couple can be coated with a pharmaceutically acceptable vehicle, e.g., with a binder, a protective coating such as a solvent protective coating, an enteric coating, an anti-caking agent, and/or a pH modifier to prevent premature reaction, e.g., with air, moisture, and/or other ingredients contained in the pharmaceutical composition. Each component of the effervescent couple, e.g., the organic acid agent and/or the source of carbon dioxide, can also individually be coated with a pharmaceutically acceptable vehicle, e.g., with a binder, a protective coating such as a solvent protective coating, an enteric coating, an anti-caking agent, and/or a pH. modifier to prevent premature reaction, e.g., with air, moisture, and/or other ingredients contained in the pharmaceutical composition. The effervescent couple can also be mixed with previously lyophilized particles, such as one or more pharmaceutically active ingredients coated with a solvent protective or enteric coating.

The effervescent dosage form may be prepared by methods known to those skilled in the art, including, but not limited to, slugging, direct compression, roller compaction, dry or wet granulation, fusion granulation, melt-granulation, vacuum granulation, and fluid bed spray granulation, any of which may be optionally followed by compression/tableting.

The pharmaceutical compositions disclosed herein may be formulated as non-effervescent or effervescent granules and powders. The non-effervescent or effervescent granules and powders may be reconstituted into a liquid dosage form, or alternatively, compressed to form tablet dosage forms which are either non-effervescent or effervescent, respectively. Pharmaceutically acceptable vehicles used in the non-effervescent or effervescent granules or powders may include, but are not limited to, binders, granulators, fillers, diluents, sweetening agent, wetting agents, stabilizing agents, solubilizing agents, anti-caking agents, pH modifiers, or any other pharmaceutical vehicle described herein. In some embodiments, the pharmaceutically acceptable vehicle comprises an organic acid agent, such as glycolic acid, lactic acid, citric acid, tartaric acid, malic acid, fumaric acid, and/or maleic acid.

Pharmaceutically acceptable vehicles used in the effervescent granules or powders include an effervescent couple, i.e., an organic acid agent and a source of carbon dioxide. Effervescent powders may be produced by blending or admixing the organic acid agent and the source of carbon dioxide (the effervescent couple) and optionally any other desired pharmaceutically acceptable vehicle. Effervescent granules may be produced by physically adhering or “gluing” the effervescent couple (the organic acid agent and the source of carbon dioxide) together using an edible or pharmaceutically acceptable binder such as polyvinylpyrrolidone, polyvinyl alcohol, L-leucine, polyethylene glycol, gum arabic, or the like, including combinations thereof. These types of granules are made by processes generically known as “wet granulation.” Granulating solvents such as ethanol and/or isopropyl alcohol are often used to aid this type of granulation process. Since the effervescent couple is physically bound together in the granule, the gas generating reaction is usually quite vigorous, leading to rapid dissolution times. Another type of “wet granulation” product that is specific to effervescent products is known as “fusion” type granules. These granules are formed by reacting the organic acid agent and source of carbon dioxide with a small amount of water (or sometimes a hydrous alcohol granulating solvent, such as various commercial grades of ethanol or isopropyl alcohol) in a highly controlled way. Since the effervescent reaction generates carbon dioxide, fusion granules tend to be quite porous, which decreases their density and also their dissolution time. Accordingly, effervescent granules prepared by wet granulation or fusion type processes may be desirable for making orodispersible dosage forms (ODxs) or other dosage forms where quick dissolving/disintegrating properties are sought. Effervescent tablet dosage forms prepared through tableting, e.g., compression, of effervescent granules or powders are also included in the present disclosure.

Additionally disclosed are pharmaceutical compositions in a dosage form that has an instant releasing component and at least one delayed releasing component, and is capable of giving a discontinuous release of the compound in the form of at least two consecutive pulses separated in time from about 0.1 up to about 24 hours (e.g., about 0.1, 0.5, 1, 2, 4, 6, 8, 10, 12, 14, 16, 18, 10, 22, or 24 hours). The pharmaceutical compositions comprise a compound as disclosed herein (e.g., a compound of Formula (I), or a pharmaceutically acceptable salt, polymorph, stereoisomer, or solvate thereof) and one or more release controlling and/or non-release controlling vehicles, such as those excipients or carriers suitable for a disruptable semipermeable membrane and as swellable substances.

Disclosed herein also are pharmaceutical compositions in a dosage form for oral administration to a subject, which comprise a compound disclosed herein (e.g., compound of Formula (I), or a pharmaceutically acceptable salt, polymorph, stereoisomer, or solvate thereof) and one or more pharmaceutically acceptable vehicles (e.g., excipients or carriers), enclosed in an intermediate reactive layer comprising a gastric juice-resistant polymeric layered material partially neutralized with alkali and having cation exchange capacity and a gastric juice-resistant outer layer.

The dosage form may be an immediate release (IR) dosage form, examples of which include, but are not limited to, an immediate release (IR) tablets or an immediate release (IR) capsule. In addition to the API, dosage forms adapted for immediate release may include one or more pharmaceutically acceptable vehicles which readily disperse, dissolve, or otherwise breakdown in the gastric environment so as not to delay or prolong dissolution/absorption of the API. Examples of pharmaceutically acceptable vehicles for immediate release dosage forms include, but are not limited to, one or more auxiliary agents, stabilizing agents, solubilizing agents, thickening agents, lubricants, binders, granulators, fillers, diluents, disintegrants, wetting agents, glidants, anti-caking agents, coloring agents, sweetening agents, dye-migration inhibitors, preservatives, antioxidants, lyoprotectants, complexing agents, flavoring agents, matrix-forming agents, dispersing agents, and performance modifiers. In some embodiments, the immediate release (IR) dosage form is an immediate release (IR) tablet comprising one or more of microcrystalline cellulose, sodium carboxymethylcellulose, magnesium stearate, mannitol, crospovidone, and sodium stearyl fumarate. In some embodiments, the immediate release (IR) dosage form comprises microcrystalline cellulose, sodium carboxymethylcellulose, and magnesium stearate. In some embodiments, the immediate release (IR) dosage form comprises mannitol, crospovidone, and sodium stearyl fumarate. In some embodiments, the immediate release (IR) dosage form comprises an organic acid agent.

The pharmaceutical compositions disclosed herein may be disclosed as soft or hard capsules, which can be made from gelatin, methylcellulose, starch, or calcium alginate. The hard gelatin capsule, also known as dry-filled capsule (DFC) or powder in capsule (PIC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

In some embodiments, the pharmaceutical compositions are in the form of immediate-release capsules for oral administration, and may further comprise cellulose, iron oxides, lactose, magnesium stearate, and sodium starch glycolate.

In some embodiments, the pharmaceutical compositions are in the form of delayed-release capsules for oral administration, and may further comprise cellulose, ethylcellulose, gelatin, hypromellose, iron oxide, and titanium dioxide.

In some embodiments, the pharmaceutical compositions are in the form of enteric coated delayed-release tablets for oral administration, and may further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In some embodiments, the pharmaceutical compositions are in the form of enteric coated delayed-release tablets for oral administration, and may further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

Any of the pharmaceutical compositions disclosed herein formulated with an organic acid agent may contain an organic acid agent which is uncoated, or alternatively, may contain an organic acid agent which is coated (a “coated organic acid agent”) with a pharmaceutically acceptable vehicle. Various pharmaceutical acceptable vehicles can be used as coating materials to modify the properties of the organic acid agent and/or to prevent undesired or premature reactions, e.g., with air, moisture, and/or other ingredients contained in the pharmaceutical composition, without losing the desired function of the organic acid agent. The coated organic acid agent may comprise a core of organic acid agent, and a thin film coating such as a thin film powder coating or a thin film polymeric coating. The coated organic acid agent may be in the form of a core-shell material, comprising a core of organic acid agent, and a protective coating surrounding the core, i.e., a shell. Any of the organic acid agents disclosed herein may be coated, including, but not limited to, glycolic acid, lactic acid, citric acid, tartaric acid, malic acid, fumaric acid, and maleic acid.

In some embodiments, the coated organic acid agent contains at least 0.01% by weight, at least 0.05% by weight, at least 0.1% by weight, at least 0.5% by weight, at least 1% by weight, at least 1.5% by weight, at least 2% by weight, at least 2.5% by weight, at least 3% by weight, at least 3.5% by weight, and up to 15% by weight, up to 10% by weight, up to 9% by weight, up to 8% by weight, up to 7% by weight, up to 6/o by weight, up to 5% by weight, up to 4% by weight, by weight of the coating, based on a total weight of the coated organic acid agent, or any range therebetween; the balance being the organic acid agent when the coated organic acid agent is formulated substantially with only the organic acid agent and the coating.

In some embodiments, the organic acid agent is coated with a water-soluble polymer, binder, granulator, filler, and the like. A non-limiting example of this type of coated organic acid agent is Citric acid DC (available from Jungbunzlauer), which is a direct compressible granular powder type of citric acid coated with a thin layer of maltodextrin.

In some embodiments, the organic acid agent is coated with an anti-caking agent. Such coated organic acid agents display a high ability to absorb spurs of humidity. A non-limiting example of this type of coated organic acid agent is Citric acid S40 (available from Jungbunzlauer), which is very fine (pulverized) granular powder of citric acid coated with silicon dioxide.

In some embodiments, the organic acid agent is coated with a pH modifier. In some embodiments, the organic acid agent is coated with a salt of an organic acid agent (i.e., a conjugate base salt of an organic acid agent). The salt of an organic acid agent may be an alkali metal salt of an organic acid agent, an alkaline earth salt of an organic acid agent, an ammonium salt of an organic acid agent, or mixtures thereof including mixed salts (e.g., sodium and potassium mixed salt) of an organic acid agent. The salt of an organic acid agent may be monobasic, dibasic, tribasic, etc. Where the salt of the organic acid agent is polybasic (dibasic, tribasic, etc.), the salt may be formed from one type of cation (e.g., sodium cation), or two or more different cations (e.g., a mixed salt with both sodium and potassium cations). Examples of salts of an organic acid agent which may be used as coating materials, include, but are not limited to, sodium citrate (e.g., monosodium citrate, disodium citrate, and/or trisodium citrate), potassium citrate (e.g., monopotassium citrate, dipotassium citrate, and/or tripotassium citrate), sodium tartrate (e.g., monosodium tartrate and/or disodium tartrate), potassium tartrate (e.g., monopotassium tartrate and/or dipotassium tartrate), potassium sodium tartrate, ammonium citrate (e.g., monoammonium citrate, diammonium citrate, and/or triammonium citrate), ammonium tartrate (e.g., monoammonium tartrate and/or diammonium tartrate), sodium fumarate (e.g., monosodium fumarate and/or disodium fumarate), potassium fumarate (e.g., monopotassium fumarate and/or dipotassium fumarate), sodium maleate (e.g., monosodium maleate and/or disodium maleate), potassium maleate (e.g., monopotassium maleate and/or dipotassium maleate), sodium lactate, and potassium lactate, including mixtures and/or hydrates thereof. Organic acid agents coated with a salt of an organic acid agent may be in the form of core-shell materials. The organic acid agent (core) and the salt of an organic acid agent (shell) may belong to the same conjugate acid-base pair. For example, the organic acid agent (core) may be citric acid and the salt of the organic acid agent (shell) may be an alkali metal salt, an alkaline earth salt, and/or an ammonium salt of citric acid. In another example, the organic acid agent (core) may be tartaric acid and the salt of the organic acid agent (shell) may be an alkali metal salt, an alkaline earth salt, and/or an ammonium salt of tartaric acid. In yet another example, the organic acid agent (core) may be fumaric acid and the salt of the organic acid agent (shell) may be an alkali metal salt, an alkaline earth salt, and/or an ammonium salt of fumaric acid. Alternatively, the organic acid agent (core) and the salt of an organic acid agent (shell) may belong to the different conjugate acid-base pairs. For example, the organic acid agent (core) may be citric acid and the salt of the organic acid agent (shell) may be an alkali metal salt, an alkaline earth salt, and/or an ammonium salt of tartaric acid. In another example, the organic acid agent (core) may be citric acid and the salt of the organic acid agent (shell) may be an alkali metal salt, an alkaline earth salt, and/or an ammonium salt of fumaric acid.

In yet another example, the organic acid agent (core) may be tartaric acid and the salt of the organic acid agent (shell) may be an alkali metal salt, an alkaline earth salt, and/or an ammonium salt of citric acid. A non-limiting example of an organic acid agent coated with a salt of an organic acid agent is Citrocoat® N (available from Jungbunzlauer), which is a granular powder made from citric acid as core material with a layer of monosodium citrate (1.5-3.5%) as a shell.

Coated organic acid agents may also be utilized in the disclosed effervescent dosage forms. Here, effervescent couples may be formed from any of the coated organic acid agents disclosed herein and a source of carbon dioxide. The use of a coated organic acid agent in the effervescent couple, as opposed to an uncoated organic acid agent, may advantageously provide improved storage stability to the effervescent dosage form without significantly sacrificing reactivity when placed into an aqueous environment, such as upon placement in water, juice, or other drinkable fluid, or from the aqueous environment in the oral cavity, such as saliva in the mouth. A non-limiting example of an effervescent couple formulated with a coated organic acid agent is Citrocoat® EP (available from Jungbunzlauer), which is an agglomerated granule made by bringing together Citrocoat® N (citric acid core coated with a layer of monosodium citrate, 1.5-3.5%, as a shell) and sodium bicarbonate using gum arabic as binder).

In some embodiments, the pharmaceutical composition comprises a compound of Formula (I) as a free base (e.g., I-1, I-2, I-3, I-4, I-5, I-6, I-7, I-8, I-9, and/or I-10), in crystalline form, and a coated organic acid agent such as coated citric acid, coated tartaric acid, coated fumaric acid, etc.

For effervescent dosage forms, a source of carbon dioxide (e.g., sodium bicarbonate) is also included with the coated organic acid agent. In some embodiments, the compound of Formula (I) is a crystalline form of 3-(2-(bis(methyl-d)amino)ethyl-1,1,2,2-d₄)-1H-indol-2,5,6,7-d₄-4-ol (I-1), as determined by X-ray powder diffraction. In some embodiments, the compound of Formula (I) is a crystalline form of 3-(2-(bis(methyl-d₃)amino)ethyl-2,2-d₂)-1H-indol-2,5,6,7-d₄-4-ol (I-2), as determined by X-ray powder diffraction. In some embodiments, the compound of Formula (I) is a crystalline form of 3-(2-(bis(methyl-d₃)amino)ethyl-1,1,2,2-d₄)-1H-indol-4-ol (I-3), as determined by X-ray powder diffraction. In some embodiments, I-3 is a crystalline solid form (pattern 1) characterized by an X-ray powder diffraction pattern containing at least three characteristic peaks at diffraction angles (2θ±0.2°) selected from 7.582°, 8.395°, 9.647°, 10.444°, 11.319°, 12.614°, 13.372°, 14.222°, 15.157°, 16.524°, 16.787°, 17.693°, 19.468°, 19.699°, 20.901°, 21.132°, 21.859°, 22.547°, 23.699°, 24.630°, 25.034°, 25.264°, 26.867°, 27.399°, 27.929°, 28.219°, 28.871°, 29.430°, 30.120°, 30.675°, 31.373°, 32.365°, 33.880°, 34.418°, 34.792°, 35.884°, 36.254°, 37.156°, 38.200°, and 38.417°, as determined by XRPD using a CuKα radiation source, for example, as shown in FIGS. 2A-2C. In some embodiments, I-3 is a crystalline solid form (pattern 2) characterized by an X-ray powder diffraction pattern containing at least three characteristic peaks at diffraction angles (2θ±0.2°) selected from 8.124°, 8.357°, 10.059°, 12.630°, 13.420°, 13.743°, 14.053°, 15.220°, 16.272°, 16.763°, 16.954°, 17.328°, 17.662°, 18.062°, 18.742°, 19.413°, 19.658°, 20.172°, 20.836°, 21.267°, 21.833°, 22.213°, 22.504°, 23.334°, 23.701°, 24.385°, 25.431°, 25.721°, 26.049°, 27.291°, 28.368°, 30.349°, 30.656°, 31.337°, 31.538°, 32.091°, 35.870°, 38.514°, and 41.361°, as determined by XRPD using a CuKα radiation source, for example, as shown in FIGS. 88-89. In some embodiments, the compound of Formula (I) is a crystalline form of 3-(2-(bis(methyl-d₃)amino)ethyl-2,2-d₂)-1H-indol-4-ol (I-4), as determined by X-ray powder diffraction. In some embodiments, the compound of Formula (I) is a crystalline form of 3-(2-(dimethylamino)ethyl-1,1,2,2-d₄)-1H-indol-4-ol (I-5), as determined by X-ray powder diffraction. In some embodiments, the compound of Formula (I) is a crystalline form of 3-(2-(dimethylamino)ethyl-2,2-d₂)-1H-indol-4-ol (I-6), as determined by X-ray powder diffraction. In some embodiments, the compound of Formula (I) is a crystalline form of 3-(2-(dimethylamino)ethyl)-1H-indol-4-ol (I-7), as determined by X-ray powder diffraction. In some embodiments, I-7 is a crystalline solid form (pattern 1) characterized by an X-ray powder diffraction pattern containing at least three characteristic peaks at diffraction angles (2θ±0.2°) selected from 7.563°, 8.375°, 12.626°, 13.383°, 15.211°, 16.753°, 17.671°, 19.668°, 21.112°, 21.863°, 22.201°, 22.560°, 23.711°, 24.592°, 25.415°, 26.820°, 27.357°, 27.921°, 28.228°, 29.253°, 30.653°, 31.364°, 32.401°, 33.797°, 34.445°, and 39.867°, as determined by XRPD using a CuKα radiation source, for example, as shown in FIG. 3C. In some embodiments, the compound of Formula (I) is a crystalline form of 3-(2-(bis(methyl-d₃)amino)ethyl)-1H-indol-4-ol (I-8), as determined by X-ray powder diffraction. In some embodiments, the compound of Formula (I) is a crystalline form of 3-(2-(dimethylamino)ethyl-1,1-d₂)-1H-indol-4-ol (I-9), as determined by X-ray powder diffraction. In some embodiments, the compound of Formula (I) is a crystalline form of 3-(2-(bis(methyl-d₃)amino)ethyl-1,1-d₂)-1H-indol-4-ol (I-10), as determined by X-ray powder diffraction.

In some embodiments, the pharmaceutical composition comprises a compound of Formula (I) as a free base (e.g., I-1, I-2, I-3, I-4, I-5, I-6, I-7, I-8, I-9, and/or 1-10), in amorphous form, and a coated organic acid agent such as coated citric acid, coated tartaric acid, coated fumaric acid, etc. For effervescent dosage forms, a source of carbon dioxide (e.g., sodium bicarbonate) is also included with the coated organic acid agent. In some embodiments, the compound of Formula (I) is an amorphous form of 3-(2-(bis(methyl-d₃)amino)ethyl-1,1,2,2-d₄)-1H-indol-2,5,6,7-d₄-4-ol (I-1), as determined by X-ray powder diffraction. In some embodiments, the compound of Formula (I) is an amorphous form of 3-(2-(bis(methyl-d₃)amino)ethyl-2,2-d₂)-1H-indol-2,5,6,7-d₄-4-ol (I-2), as determined by X-ray powder diffraction. In some embodiments, the compound of Formula (I) is an amorphous form of 3-(2-(bis(methyl-d₃)amino)ethyl-1,1,2,2-d₄)-1H-indol-4-ol (I-3), as determined by X-ray powder diffraction. In some embodiments, the compound of Formula (I) is an amorphous form of 3-(2-(bis(methyl-d₃)amino)ethyl-2,2-d₂)-1H-indol-4-ol (I-4), as determined by X-ray powder diffraction. In some embodiments, the compound of Formula (I) is an amorphous form of 3-(2-(dimethylamino)ethyl-1,1,2,2-d₄)-1H-indol-4-ol (I-5), as determined by X-ray powder diffraction. In some embodiments, the compound of Formula (I) is an amorphous form of 3-(2-(dimethylamino)ethyl-2,2-d₂)-1H-indol-4-ol (I-6), as determined by X-ray powder diffraction. In some embodiments, the compound of Formula (I) is an amorphous form of 3-(2-(dimethylamino)ethyl)-1I-indol-4-ol (I-7), as determined by X-ray powder diffraction. In some embodiments, the compound of Formula (I) is an amorphous form of 3-(2-(bis(methyl-d₃)amino)ethyl)-1H-indol-4-ol (I-8), as determined by X-ray powder diffraction. In some embodiments, the compound of Formula (I) is an amorphous form of 3-(2-(dimethylamino)ethyl-1,1-d₂)-1H-indol-4-ol (I-9), as determined by X-ray powder diffraction. In some embodiments, the compound of Formula (I) is an amorphous form of 3-(2-(bis(methyl-d₃)amino)ethyl-1,1-d₂)-1H-indol-4-ol (I-10), as determined by X-ray powder diffraction.

In some embodiments, the pharmaceutical composition comprises a pharmaceutically acceptable salt of a compound of Formula (I), in crystalline form, and a coated organic acid agent such as coated citric acid, coated tartaric acid, coated fumaric acid, etc. For effervescent dosage forms, a source of carbon dioxide (e.g., sodium bicarbonate) is also included with the coated organic acid agent. In some embodiments, the pharmaceutically acceptable salt is a benzenesulfonate salt of 3-(2-(bis(methyl-d₃)amino)ethyl-1,1,2,2-d₄)-1H-indol-4-ol (I-3a). In some embodiments, salt I-3a is in a crystalline solid form characterized by an X-ray powder diffraction pattern containing at least three characteristic peaks at diffraction angles (2θ±0.2°) selected from 7.023°, 7.767°, 11.822°, 12.550°, 12.860°, 13.994°, 15.521°, 18.436°, 19.503°, 20.760°, 21.070°, 22.007°, 22.745°, 23.340°, 24.187°, 25.532°, 26.880°, 27.856°, 28.163°, 31.267°, 33.024°, 35.030°, 36.835°, 39.312°, 40.545°, and 40.988°, as determined by XRPD using a CuKα radiation source, for example, as shown in FIGS. 63A-63D (pattern 1). In some embodiments, the pharmaceutically acceptable salt is a benzenesulfonate salt of 3-(2-(dimethylamino)ethyl)-1H-indol-4-ol (I-7a). In some embodiments, salt I-7a is in a crystalline solid form characterized by an X-ray powder diffraction pattern containing at least three characteristic peaks at diffraction angles (2θ±0.2°) selected from 7.002°, 7.733°, 11.768°, 12.516°, 12.882°, 13.546°, 13.968°, 14.788°, 15.225°, 15.474°, 18.370°, 19.737°, 20.703°, 21.050°, 21.873°, 21.982°, 22.315°, 22.639°, 23.282°, 23.775°, 24.125°, 25.193°, 25.475°, 25.931°, 26.813°, 27.778°, 28.127°, 30.866°, 31.207°, 32.941°, 33.222°, 33.698°, 36.803°, 38.668°, and 39.289°, as determined by XRPD using a CuKα radiation source, for example, as shown in FIGS. 3A-3B (pattern 1). In some embodiments, the pharmaceutically acceptable salt is a benzoate salt of 3-(2-(bis(methyl-d₃)amino)ethyl-1,1,2,2-d₄)-1H-indol-4-ol (I-3j). In some embodiments, salt I-3j is in a crystalline solid form characterized by an X-ray powder diffraction pattern containing at least three characteristic peaks at diffraction angles (2θ±0.2°) selected from 9.486°, 11.006°, 12.379°, 13.428°, 14.608°, 15.446°, 16.389°, 18.247°, 18.977°, 19.346°, 19.831°, 20.868°, 21.447°, 22.860°, 23.878°, 24.944°, 25.737°, 26.144°, 26.341°, 26.990°, 27.708°, 28.595°, 30.048°, 30.763°, 31.127°, 31.839°, 32.800°, 34.460°, 35.444°, 37.725°, and 38.597°, as determined by XRPD using a CuKα radiation source, for example, as shown in FIGS. 78A-78C (pattern 1). In some embodiments, the pharmaceutically acceptable salt is a benzoate salt of 3-(2-(dimethylamino)ethyl)-1H-indol-4-ol (I-7j). In some embodiments, salt I-7j is in a crystalline solid form characterized by an X-ray powder diffraction pattern containing at least three characteristic peaks at diffraction angles (2θ±0.2°) selected from 9.492°, 11.011°, 12.391°, 13.440°, 14.609°, 15.432°, 16.394°, 18.259°, 18.967°, 19.356°, 19.827°, 20.843°, 21.476°, 22.062°, 22.805°, 23.862°, 24.963°, 25.734°, 26.170°, 26.992°, 27.738°, 28.593°, 30.073°, 30.746°, 31.041°, 31.799°, 32.794°, 33.551°, 34.480°, 35.430°, 37.685°, and 38.643°, as determined by XRPD using a CuKα radiation source, for example, as shown in FIGS. 53A-53B (pattern 1). In some embodiments, the pharmaceutically acceptable salt is a tartrate salt of 3-(2-(bis(methyl-d₃)amino)ethyl-1,1,2,2-d₄)-1H-indol-4-ol (I-3b). In some embodiments, salt I-3b is in a crystalline solid form of pattern 1 characterized by, e.g., an X-ray powder diffraction pattern shown in FIG. 66. In some embodiments, salt I-3b is in a crystalline solid form characterized by an X-ray powder diffraction pattern containing at least three characteristic peaks at diffraction angles (2θ±0.2°) selected from 6.732°, 12.708°, 13.470°, 14.774°, 15.921°, 16.268°, 17.295°, 18.869°, 20.079°, 20.208°, 20.877°, 21.894°, 22.657°, 23.491°, 23.702°, 24.636°, 24.882°, 25.569°, 26.685°, 27.060°, 27.502°, 28.179°, 28.597°, 29.035°, 29.257°, 29.527°, 31.017°, 31.527°, 32.059°, 32.307°, 33.012°, 34.024°, 34.388°, 34.905°, 35.361°, 36.183°, 37.372°, 37.764°, 38.657°, and 41.049°, as determined by XRPD using a CuKα radiation source, for example, as shown in FIGS. 69A-69B (pattern 2). In some embodiments, the pharmaceutically acceptable salt is a tartrate salt of 3-(2-(dimethylamino)ethyl)-1H-indol-4-ol (I-7b). In some embodiments, salt I-7b is in a crystalline solid form characterized by an X-ray powder diffraction pattern containing at least three characteristic peaks at diffraction angles (2θ±0.2°) selected from 6.798°, 11.360°, 12.764°, 13.535°, 14.837°, 15.973°, 16.351°, 17.367°, 18.937°, 20.168°, 20.929°, 21.946°, 22.719°, 23.604°, 23.814°, 24.874°, 25.609°, 26.745°, 27.111°, 27.558°, 28.653°, 29.630°, 31.129°, 31.567°, 32.180°, 33.073°, 34.096°, 34.460°, 36.226°, 37.497°, 38.727°, and 41.126°, as determined by XRPD using a CuKα radiation source, for example, as shown in FIG. 12 (pattern 1).

In some embodiments, salt I-7b is in a crystalline solid form of pattern 2 characterized by, e.g., an X-ray powder diffraction pattern as shown in FIG. 12. In some embodiments, salt I-7b is in a crystalline solid form characterized by an X-ray powder diffraction pattern containing at least three characteristic peaks at diffraction angles (2θ±0.2°) selected from 6.479°, 10.486°, 10.862°, 11.913°, 12.222°, 12.972°, 13.161°, 13.467°, 14.230°, 15.372°, 15.736°, 16.053°, 16.457°, 16.613°, 17.009°, 17.695°, 17.913°, 18.486°, 18.795°, 19.479°, 20.101°, 20.416°, 20.818°, 21.352°, 22.106°, 22.320°, 22.629°, 22.964°, 23.698°, 23.950°, 24.175°, 24.439°, 24.818°, 25.079°, 25.880°, 26.528°, 27.297°, 27.752°, 28.124°, 28.349°, 28.631°, 29.075°, 29.819°, 30.202°, 30.562°, 31.025°, 31.207°, 31.650°, 31.953°, 33.721°, 34.362°, 34.651°, 34.994°, 35.512°, 35.982°, 36.450°, 37.476°, 38.287°, 39.699°, 39.980°, 40.951°, and 41.870°, as determined by XRPD using a CuKα radiation source, for example, as shown in FIG. 18 (pattern 3).

In some embodiments, the pharmaceutically acceptable salt is a hemi-fumarate salt of 3-(2-(dimethylamino)ethyl)-1H-indol-4-ol (I-7c). In some embodiments, salt I-7c is in a crystalline solid form of pattern 1, 2, 3, or 4, characterized by, e.g., an X-ray powder diffraction pattern as shown in FIGS. 23 and 29. In some embodiments, salt I-7c is in a crystalline solid form characterized by an X-ray powder diffraction pattern containing at least three characteristic peaks at diffraction angles (2θ±0.2°) selected from 8.483°, 8.733°, 11.080°, 11.351°, 11.622°, 12.615°, 13.258, 14.977°, 15.557°, 16.089°, 16.319°, 16.606°, 17.013°, 18.928°, 18.884°, 19.429°, 19.734°, 20.643°, 21.484°, 22.067°, 23.433°, 24.466°, 24.885°, 26.740°, 27.900°, 28.557°, 29.523°, 32.888°, 34.183°, and 36.808°, as determined by XRPD using a CuKα radiation source, for example, as shown in FIG. 42 (pattern 5). In some embodiments, salt I-7c is in a crystalline solid form characterized by an X-ray powder diffraction pattern containing at least three characteristic peaks at diffraction angles (2θ±0.2°) selected from 9.746°, 11.354°, 12.338°, 13.762°, 16.111°, 16.644°, 19.929°, 20.180°, 21.576°, 22.758°, 23.348°, 23.938°, 24.724°, 25.226°, 26.203°, 27.910°, 29.056°, 29.499°, 32.753°, 35.567°, 37.279°, 37.347°, and 39.481°, as determined by XRPD using a CuKα radiation source, for example, as shown in FIG. 42 (pattern 6). In some embodiments, the pharmaceutically acceptable salt is a hemi-fumarate salt of 3-(2-(bis(methyl-d₃)amino)ethyl-1,1,2,2-d₄)-1H-indol-4-ol (I-3c). In some embodiments, salt I-3c is in a crystalline solid form of pattern 1 characterized by, e.g., an X-ray powder diffraction pattern as shown in FIGS. 72 and 75A. In some embodiments, salt I-3c is in a crystalline solid form characterized by an X-ray powder diffraction pattern containing at least three characteristic peaks at diffraction angles (2θ±0.2°) selected from 9.713°, 11.209°, 11.605°, 12.338°, 12.852°, 13.718°, 15.117°, 16.066°, 16.627°, 19.026°, 19.427°, 20.108°, 21.068°, 21.335°, 21.837°, 22.429°, 23.262°, 23.478°, 23.900°, 24.720°, 25.318°, 27.912°, 28.532°, 29.565°, 30.457°, 32.698°, 34.155°, 37.910°, 39.566°, and 40.999°, as determined by XRPD using a CuKα radiation source, for example, as shown in FIG. 75B (pattern 2). In some embodiments, the pharmaceutically acceptable salt is an acetate salt of 3-(2-(dimethylamino)ethyl)-1H-indol-4-ol (I-7d). In some embodiments, salt I-7d is in a crystalline solid form of pattern 1 or 2 characterized by, e.g., an X-ray powder diffraction pattern as shown in FIG. 32. In some embodiments, the pharmaceutically acceptable salt is a hemi-malonate salt of 3-(2-(dimethylamino)ethyl)-1H-indol-4-ol (I-7f). In some embodiments, salt I-7f is in a crystalline solid form of pattern 1 characterized by, e.g., an X-ray powder diffraction pattern as shown in FIG. 39. In some embodiments, the pharmaceutically acceptable salt is a hemi-succinate salt of 3-(2-(dimethylamino)ethyl)-1H-indol-4-ol (I-7h). In some embodiments, salt I-7h is in a crystalline solid form of pattern 1 characterized by, e.g., an X-ray powder diffraction as shown in FIG. 47. In some embodiments, the pharmaceutically acceptable salt is an oxalate salt of 3-(2-(dimethylamino)ethyl)-1H-indol-4-ol (I-7i). In some embodiments, salt I-7i is in a crystalline solid form of pattern 1, 2, 3, 4, 5, or 6 characterized by, e.g., an X-ray powder diffraction pattern as shown in FIG. 50. In some embodiments, the pharmaceutically acceptable salt is a salicylate salt of 3-(2-(dimethylamino)ethyl)-1H-indol-4-ol (I-7k). In some embodiments, salt I-7k is in a crystalline solid form of pattern 1, 2, or 3 characterized by, e.g., an X-ray powder diffraction pattern as shown in FIG. 60.

In some embodiments, the pharmaceutical composition comprises a pharmaceutically acceptable salt of a compound of Formula (I), in amorphous form, and a coated organic acid agent such as coated citric acid, coated tartaric acid, coated fumaric acid, etc. For effervescent dosage forms, a source of carbon dioxide (e.g., sodium bicarbonate) is also included with the coated organic acid agent. In some embodiments, the pharmaceutically acceptable salt is a citrate salt of 3-(2-(bis(methyl-d₃)amino)ethyl-1,1,2,2-d₄)-1H-indol-4-ol (I-3e). In some embodiments, salt I-3e is in the form of an amorphous solid as characterized by an X-ray powder diffraction (XRPD). In some embodiments, the pharmaceutically acceptable salt is a citrate salt of 3-(2-(dimethylamino)ethyl)-1H-indol-4-ol (I-7e). In some embodiments, salt I-7e is in the form of an amorphous solid as characterized by an X-ray powder diffraction (XRPD), for example, as shown in FIGS. 37A-37B.

When the pharmaceutical composition is formulated with a pharmaceutically acceptable salt of a compound of Formula (I), the acid used in forming the pharmaceutically acceptable salt of a compound of Formula (I) and the organic acid agent (vehicle) can be the same. For example, the pharmaceutical composition may comprise a tartrate salt of a compound of Formula (I) (e.g., I-1b, I-2b, I-3b, I-4b, I-5b, I-6b, and/or I-7b), and tartaric acid as organic acid agent (vehicle). In another example, the pharmaceutical composition may comprise a citrate salt of a compound of Formula (I) (e.g., I-1e, 1-2e, I-3e, I-4e, I-5e, I-6, and/or I-7e), and citric acid as organic acid agent (vehicle).

When the pharmaceutical composition is formulated with a pharmaceutically acceptable salt of a compound of Formula (I), the acid used in forming the pharmaceutically acceptable salt of a compound of Formula (I) and the organic acid agent (vehicle) can be different. For example, the pharmaceutical composition may comprise a benzenesulfonate salt of a compound of Formula (I) (e.g., I-1a, I-2a, I-3a, I-4a, I-5a, I-6a, and/or I-7a), and citric acid and/or tartaric acid, etc., as organic acid agent (vehicle). In another example, the pharmaceutical composition may comprise a benzoate salt of a compound of Formula (I) (e.g., I-1j, I-2j, I-3j, I-4j, I-5j, I-6j, and/or 1-7j), and citric acid and/or tartaric acid, etc., as organic acid agent (vehicle).

The pharmaceutical compositions disclosed herein may be disclosed in liquid and semisolid dosage forms, including emulsions, solutions, suspensions, elixirs, and syrups.

In some embodiments, oral liquid dosage forms are prepared by reconstituting a solid dosage form disclosed herein (e.g., an effervescent dosage form) into a pharmaceutically acceptable aqueous medium such as water, juice, or other drinkable fluid prior to use.

In some embodiments, the oral liquid dosage form is prepared by reconstituting into a pharmaceutically acceptable aqueous medium a solid dosage form comprising a compound of Formula (I) as a free base (e.g., I-1, I-2, I-3, I-4, I-5, I-6, I-7, I-8, I-9, and/or I-10), in crystalline form. The solid dosage form may additionally be formulated with an organic acid agent, including a coated organic acid agent. Effervescent solid dosage forms may additionally be formulated with an organic acid agent, including a coated organic acid agent, and a source of carbon dioxide.

In some embodiments, the oral liquid dosage form is prepared by reconstituting into a pharmaceutically acceptable aqueous medium a solid dosage form comprising a compound of Formula (I) as a free base (e.g., I-1, I-2, I-3, I-4, I-5, I-6, I-7, I-8, I-9, and/or I-10), in amorphous form. The solid dosage form may additionally be formulated with an organic acid agent, including a coated organic acid agent. Effervescent solid dosage forms may additionally be formulated with an organic acid agent, including a coated organic acid agent, and a source of carbon dioxide.

In some embodiments, the oral liquid dosage form is prepared by reconstituting into a pharmaceutically acceptable aqueous medium a solid dosage form comprising a pharmaceutically acceptable salt of a compound of Formula (I), in crystalline form. The solid dosage form may additionally be formulated with an organic acid agent, including a coated organic acid agent. Effervescent solid dosage forms may additionally be formulated with an organic acid agent, including a coated organic acid agent, and a source of carbon dioxide.

In some embodiments, the oral liquid dosage form is prepared by reconstituting into a pharmaceutically acceptable aqueous medium a solid dosage form comprising a pharmaceutically acceptable salt of a compound of Formula (I), in amorphous form. The solid dosage form may additionally be formulated with an organic acid agent, including a coated organic acid agent. Effervescent solid dosage forms may additionally be formulated with an organic acid agent, including a coated organic acid agent, and a source of carbon dioxide.

An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates. In some embodiments, examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Cyclodextrins such as α-cyclodextrin, β-cyclodextrin, γ-cyclodextrin, methyl-β-cyclodextrin, hydroxyethyl β-cyclodextrin, hydroxypropyl-β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

The pharmaceutical compositions disclosed herein for oral administration may be also disclosed in the forms of liposomes, micelles, microspheres, or nanosystems.

Coloring and flavoring agents can be used in all of the above dosage forms.

The pharmaceutical compositions disclosed herein may be co-formulated with other active ingredients which do not impair the desired therapeutic action, or with substances that supplement the desired action.

### B. Parenteral Administration

The pharmaceutical compositions disclosed herein may be administered parenterally by injection, infusion, or implantation, for local or systemic administration. Parenteral administration, as used herein, includes, but is not limited to, intravenous, intradermal, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable vehicles (e.g., carriers and excipients), including, but not limited to, aqueous vehicles, water-miscible vehicles, non-aqueous vehicles, antimicrobial agents or preservatives against the growth of microorganisms, stabilizing agents, solubilizing agents, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, as well as organic acid agents (e.g., citric acid, lactic acid, etc.). Suitable complexing agents include, but are not limited to, cyclodextrins, including α-cyclodextrin, β-cyclodextrin, methyl-β-cyclodextrin, hydroxypropyl-3-cyclodextrin/hydroxypropyl-β-cyclodextrin, sulfobutylether-β-cyclodextrin, and sulfobutylether 7-O-cyclodextrin (CAPTISOL®, CyDex, Lenexa, Kans.).

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In some embodiments, the pharmaceutical compositions are disclosed as ready-to-use sterile solutions. In some embodiments, the pharmaceutical compositions are disclosed as sterile dry soluble products, including lyophilized powders and hypodermic tablets, to be reconstituted with a vehicle prior to use. In some embodiments, the pharmaceutical compositions are disclosed as ready-to-use sterile suspensions. In some embodiments, the pharmaceutical compositions are disclosed as sterile dry insoluble products to be reconstituted with a vehicle prior to use. In some embodiments, the pharmaceutical compositions are disclosed as ready-to-use sterile emulsions.

The pharmaceutical compositions may be formulated as a suspension, solid, semi-solid, or thixotropic liquid, for administration as an, implanted depot. In some embodiments, the pharmaceutical compositions disclosed herein are dispersed in a solid inner matrix, which is surrounded by an outer polymeric membrane that is insoluble in body fluids but allows the active ingredient in the pharmaceutical compositions diffuse through. Fatty acid salts of the compounds of Formula (I) may be well-suited for such dosage forms.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions disclosed herein may be administered topically to the skin, orifices, or mucosa. Topical administration, as described herein, includes, but is not limited to, conjunctival, intracorneal, intraocular, ophthalmic, auricular, transdermal, nasal, vaginal, uretheral, respiratory, and rectal administration.

The pharmaceutical compositions disclosed herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions disclosed herein may contain the active ingredient(s) which may be mixed under sterile conditions with a pharmaceutically acceptable vehicle, and with any preservatives, buffers, absorption enhancers, propellants which may be required. Liposomes, micelles, microspheres, nanosystems, and mixtures thereof, may also be used.

Pharmaceutically acceptable vehicles (e.g., carriers and excipients) suitable for use in the topical formulations disclosed herein include, but are not limited to, aqueous vehicles, water-miscible vehicles, non-aqueous vehicles, antimicrobial agents or preservatives against the growth of microorganisms, stabilizing agents, solubilizing agents, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The ointments, pastes, creams and gels may contain, in addition to an active ingredient(s), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient(s), excipients such as lactose, talc, silicic acid, aluminum hydroxide, calcium silicates and polyamide powder, or mixtures of these substances. Sprays, such as those used for (intra)nasal administration, can additionally contain customary propellants, such as fluorohydrocarbons, chlorofluorohydrocarbons and volatile unsubstituted hydrocarbons, such as butane and propane.

Transdermal delivery devices (e.g., patches) may be used. Such dosage forms have the added advantage of providing controlled delivery of active ingredient(s) to the body. That is, the compounds of the present disclosure (e.g., a compound of Formula (I), or a pharmaceutically acceptable salt, polymorph, stereoisomer, or solvate thereof) can be administered via a transdermal patch at a steady state concentration, whereby the active ingredient(s) is gradually administered over time, thus avoiding drug spiking and adverse events/toxicity associated therewith.

Transdermal patch dosage forms herein may be formulated with various amounts of the active ingredient(s), depending on the disease/condition being treated, the active ingredient(s) employed, the permeation and size of the transdermal delivery device, the release time period, etc. For example, a unit dose preparation may be varied or adjusted e.g., from 5 mg, 10 mg, 15 mg, 20 mg, 25 mg, 30 mg, 35 mg, 40 mg, 45 mg, 50 mg, to 200 mg, 175 mg, 150 mg, 125 mg, 100 mg, 95 mg, 90 mg, 85 mg, 80 mg, 75 mg, 70 mg, 65 mg, 60 mg, 55 mg of the compound of Formula (I) (active basis) or otherwise as deemed appropriate using sound medical judgment, according to the particular application and the potency of compound.

Transdermal patches formulated with the disclosed compounds may be suitable for microdosing or sub-psychedelic (also referred to herein as sub-psychoactive) dosing, to achieve durable therapeutic benefits, with decreased toxicity. In some embodiments, the compound of Formula (I), or a pharmaceutically acceptable salt, polymorph, stereoisomer, or solvate thereof, is administered via a transdermal patch at sub-psychoactive (yet still potentially serotonergic concentrations) concentrations, for example, over an extended period such as over a 8, 24, 48, 72, 84, 96, or 168 hour time period.

In addition to the active ingredient(s), and any optional pharmaceutically acceptable vehicles(s), the transdermal patch may also include one or more of a pressure sensitive adhesive layer, a backing, and a release liner, as is known to those of ordinary skill in the art.

Transdermal patch dosage forms can be made by dissolving or dispersing the compounds herein in the proper medium. In some embodiments, the compounds of the present disclosure may be dissolved/dispersed directly into a polymer matrix forming the pressure sensitive adhesive layer. Such transdermal patches are called drug-in-adhesive (DIA) patches. Preferred DIA patch forms are those in which the active ingredient(s) is distributed uniformly throughout the pressure sensitive adhesive polymer matrix. In some embodiments, the active ingredient(s) may be provided in a layer containing the active ingredient(s) plus a polymer matrix which is separate from the pressure sensitive adhesive layer. In any case, the compounds of the present disclosure may optionally be formulated with suitable vehicles(s) such as carrier agents, permeation agents/absorption enhancers, humectants/crystallization inhibitors, etc. to increase the flux across the skin.

Examples of carrier agents may include, but are not limited to, C₈-C₂₂ fatty acids, such as oleic acid, undecanoic acid, valeric acid, heptanoic acid, pelargonic acid, capric acid, lauric acid, and eicosapentaenoic acid; C₈-C₂₂ fatty alcohols such as octanol, nonanol, oleyl alcohol, decyl alcohol and lauryl alcohol; lower alkyl esters of C₈-C₂₂ fatty acids such as ethyl oleate, isopropyl myristate, butyl stearate, and methyl laurate; di(lower)alkyl esters of C₆-C₂₂ diacids such as diisopropyl adipate; monoglycerides of C₈-C₂₂ fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; glycerol; ethyl acetate; acetoacetic ester; N-alkylpyrrolidone; cyclodextrins, such as α-cyclodextrin, β-cyclodextrin, γ-cyclodextrin, or derivatives such as 2-hydroxypropyl-β-cyclodextrin; and terpenes/terpenoids, such as limonene, linalool, myrcene, pinene such as α-pinene, caryophyllene, citral, eucolyptol, and the like; including mixtures thereof.

Examples of permeation agents/absorption enhancers include, but are not limited to, sulfoxides, such as dodecylmethylsulfoxide, octyl methyl sulfoxide, nonyl methyl sulfoxide, decyl methyl sulfoxide, undecyl methyl sulfoxide, 2-hydroxydecyl methyl sulfoxide, 2-hydroxy-undecyl methyl sulfoxide, 2-hydroxydodecyl methyl sulfoxide, and the like; surfactant-lecithin organogel (PLO), such as those formed from an aqueous phase with one or more of poloxamers, CARBOPOL and PEMULEN, a lipid phase formed from one or more of isopropyl palmitate and PPG-2 myristyl ether propionate, and lecithin; fatty acids, esters, and alcohols, such as oleyloleate and oleyl alcohol; keto acids such as levulinic acid; glycols and glycol ethers, such as diethylene glycol monoethyl ether; including mixtures thereof.

Examples of humectants/crystallization inhibitors include, but are not limited to, polyvinyl pyrrolidone-co-vinyl acetate, HPMC, polymethacrylate, and mixtures thereof.

The pressure sensitive adhesive layer may be formed from polymers including, but not limited to, acrylics (polyacrylates including alkyl acrylics), polyvinyl acetates, natural and synthetic rubbers (e.g., polyisobutylene), ethylenevinylacetate copolymers, polysiloxanes, polyurethanes, plasticized polyether block amide copolymers, plasticized styrene-butadiene rubber block copolymers, and mixtures thereof. The pressure-sensitive adhesive layer used in the transdermal patch of the present disclosure may be formed from an acrylic polymer pressure-sensitive adhesive, preferably an acrylic copolymer pressure sensitive adhesive. The acrylic copolymer pressure sensitive adhesive may be obtained by copolymerization of one or more alkyl (meth)acrylates (e.g., 2-ethylhexyl acrylate); aryl (meth)acrylates; arylalkyl (meth)acrylate; and (meth)acrylates with functional groups such as hydroxyalkyl (meth)acrylates (e.g., hydroxyethyl acrylate, 2-hydroxypropyl acrylate, 3-hydroxypropyl acrylate, 4-hydroxybutyl acrylate, 2-hydroxyethyl methacrylate, 2-hydroxypropyl methacrylate, 3-hydroxypropyl methacrylate, and 4-hydroxybutyl methacrylate), carboxylic acid containing (meth)acrylates (e.g., acrylic acid), and alkoxy (meth)acrylates (e.g., methoxyethyl acrylate); optionally with one or more copolymerizable monomers (e.g., vinylpyrrolidone, vinyl acetate, etc.). Specific examples of acrylic pressure-sensitive adhesives may include, but are not limited to, DURO-TAK products (Henkel) such as DURO-TAK 87-900A, DURO-TAK 87-9301, DURO-TAK 87-4098, DURO-TAK 87-2074, DURO-TAK 87-235A, DURO-TAK 87-2510, DURO-TAK 87-2287, DURO-TAK 87-4287, DURO-TAK 87-2516, DURO-TAK 387-2052, and DURO-TAK 87-2677.

The backing used in the transdermal patch of the present disclosure may include flexible backings such as films, nonwoven fabrics, Japanese papers, cotton fabrics, knitted fabrics, woven fabrics, and laminated composite bodies of a nonwoven fabric and a film. Such a backing is preferably composed of a soft material that can be in close contact with a skin and can follow skin movement and of a material that can suppress skin rash and other discomforts following prolonged use of the patch. Examples of the backing materials include, but are not limited to, polyethylene, polypropylene, polyethylene terephthalate, polybutylene terephthalate, polyethylene naphthalate, polystyrene, nylon, cotton, acetate rayon, rayon, a rayon/polyethylene terephthalate composite body, polyacrylonitrile, polyvinyl alcohol, acrylic polyurethane, ester polyurethane, ether polyurethane, a styrene-isoprene-styrene copolymer, a styrene-butadiene-styrene copolymer, a styrene-ethylene-propylene-styrene copolymer, styrene-butadiene rubber, an ethylene-vinyl acetate copolymer, or cellophane, for example. Preferred backings do not adsorb or release the active ingredient(s). In order to suppress the adsorption and release of the active ingredient(s), to improve transdermal absorbability of the active ingredient(s), and to suppress skin rash and other discomforts, the backing preferably includes one or more layers composed of the material above and has a water vapor permeability. Specific examples of backings may include, but are not limited to, 3M COTRAN products such as 3M COTRAN ethylene vinyl acetate membrane film 9702, 3M COTRAN ethylene vinyl acetate membrane film 9716, 3M COTRAN polyethylene membrane film 9720, 3M COTRAN ethylene vinyl acetate membrane film 9728, and the like.

The release liner used in the transdermal patch of the present disclosure may include, but is not limited to, a polyester film having one side or both sides treated with a release coating, a polyethylene laminated high-quality paper treated with a release coating, and a glassine paper treated with a release coating. The release coating may be a fluoropolymer, a silicone, a fluorosilicone, or any other release coating known to those of ordinary skill in the art. The release liner may have an uneven surface in order to easily take out the transdermal patch from a package. Examples of release liners may include, but are not limited to SCOTCHPAK products from 3M such as 3M SCOTCHPAK 9744, 3M SCOTCHPAK 9755, 3M SCOTCHPAK 9709, and 3M SCOTCHPAK 1022.

Other layers such as abuse deterrent layers formulated with one or more irritants (e.g., sodium lauryl sulfate, poloxamer, sorbitan monoesters, glyceryl monooleates, spices, etc.), may also be employed.

Methods disclosed herein using a transdermal patch dosage form provide for systemic delivery of small doses of active ingredient(s), preferably over extended periods of time such as up to 168 hour time periods, for example from 2 to 96 hours, or 4 to 72 hours, or 8 to 24 hours, or 10 to 18 hours, or 12 to 14 hours. In particular, the compound of Formula (I) can be delivered in small, steady, and consistent doses such that deleterious or undesirable side-effects can be avoided. In some embodiments, the compound of Formula (I) is administered transdermally at sub-psychoactive (yet still potentially serotonergic concentrations) concentrations.

Automatic injection devices offer a method for delivery of the compositions disclosed herein to patients. The compositions disclosed herein may be administered to a patient using automatic injection devices through a number of known devices, a non-limiting list of which includes transdermal, subcutaneous, and intramuscular delivery.

In some transdermal, subcutaneous, or intramuscular applications, a composition disclosed herein is absorbed through the skin. Passive transdermal patch devices often include an absorbent layer or membrane that is placed on the outer layer of the skin. The membrane typically contains a dose of a substance that is allowed to be absorbed through the skin to deliver the composition to the patient. Typically, only substances that are readily absorbed through the outer layer of the skin may be delivered with such transdermal patch devices.

Other automatic injection devices disclosed herein are configured to provide for increased skin permeability to improve delivery of the disclosed compositions. Non-limiting examples of structures used to increase permeability to improve transfer of a composition into the skin, across the skin, or intramuscularly include the use of one or more microneedles, which in some embodiments may be coated with a composition disclosed herein. Alternatively, hollow microneedles may be used to provide a fluid channel for delivery of the disclosed compositions below the outer layer of the skin. Other devices disclosed herein include transdermal delivery by iontophoresis, sonophoresis, reverse iontophoresis, or combinations thereof, and other technologies known in the art to increase skin permeability to facilitate drug delivery.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions disclosed herein may be disclosed in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions disclosed herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions disclosed herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions disclosed herein may be administered ophthalmically in the forms of solutions, suspensions, ointments, emulsions, gel-forming solutions, powders for solutions, gels, ocular inserts, and implants.

The pharmaceutical compositions disclosed herein may be administered intranasally or by inhalation to the respiratory tract. The pharmaceutical compositions may be disclosed in the form of an aerosol or solution for delivery using a pressurized container, pump, spray, atomizer, such as an atomizer using electrohydrodynamics to produce a fine mist, or nebulizer, alone or in combination with a suitable propellant, including, but not limited to, fluorohydrocarbons, chlorofluorohydrocarbons, and volatile unsubstituted hydrocarbons, such as butane, propane, 1,1,1,2-tetrafluoroethane, and/or 1,1,1,2,3,3,3-heptafluoropropane. The pharmaceutical compositions may also be disclosed as a dry powder for insufflation, alone or in combination with an inert carrier such as lactose or phospholipids; and nasal drops. For intranasal use, the powder may comprise a bioadhesive agent, e.g., chitosan and/or cyclodextrin.

Solutions or suspensions for use is a pressurized container, pump, spray, atomizer, or nebulizer may be formulated to contain ethanol, aqueous ethanol, or a suitable alternative agent for dispersing, solubilizing, or extending release of the active ingredient disclosed herein, a propellant as solvent; and/or a surfactant, such as sorbitan trioleate, oleic acid, or an oligolactic acid.

The pharmaceutical compositions disclosed herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions disclosed herein; a suitable powder base, such as lactose or starch; and a performance modifier, such as 1-leucine, mannitol, or magnesium stearate. The lactose may be anhydrous or in the form of the monohydrate. Other suitable excipients or carriers include dextran, glucose, maltose, sorbitol, xylitol, fructose, sucrose, and trehalose. The pharmaceutical compositions disclosed herein for inhaled/intranasal administration may further comprise a suitable flavor, such as menthol and levomenthol, or sweetening agent, such as saccharin or saccharin sodium.

The pharmaceutical compositions disclosed herein for topical administration may be formulated to be immediate release or modified release, including delayed-, sustained-, pulsed-, controlled-, targeted, and programmed release.

### D. Modified Release

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

### 1. Matrix Controlled Release Devices

The pharmaceutical compositions disclosed herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions disclosed herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinylpyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate, and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions disclosed herein in a modified release dosage form may be prepared by methods known to those skilled in the art, including direct compression, dry or wet granulation followed by compression, melt-granulation followed by compression.

### 2. Osmotic Controlled Release Devices

The pharmaceutical compositions disclosed herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients or carriers as described herein to promote performance or processing of the composition.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In some embodiments, the pharmaceutical compositions disclosed herein are formulated as AMT controlled-release dosage forms, which comprises an asymmetric osmotic membrane that coats a core comprising the active ingredient(s) and other pharmaceutically acceptable vehicles (e.g., excipients or carriers). The AMT controlled-release dosage forms can be prepared according to conventional methods and techniques known to those skilled in the art, including direct compression, dry granulation, wet granulation, and a dip-coating method.

In some embodiments, the pharmaceutical compositions disclosed herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions disclosed herein in a modified release dosage form may be fabricated a multiparticulate controlled release device, which comprises a multiplicity of particles, granules, or pellets, ranging from about 10 μm to about 3 mm, about 50 m to about 2.5 mm, or from about 100 m to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

The pharmaceutical compositions disclosed herein may also be formulated to be targeted to a particular tissue, receptor, or other area of the body of the subject to be treated, including liposome-, resealed erythrocyte-, and antibody-based delivery systems.

Any of the delivery devices above, e.g., controlled release device, implant, patch, pump, depot, etc., can be optionally manufactured with smart technology enabling remote activation of the drug delivery. The remote activation can be performed via computer or mobile app. To ensure security, the remote activation device can be password encoded. This technology enables a healthcare provider to perform telehealth sessions with a patient, during which the healthcare provider can remotely activate and administer the compound of Formula (I), or a pharmaceutically acceptable salt, polymorph, stereoisomer, or solvate thereof, via the desired delivery device while supervising the patient on the televisit.

### Pharmacokinetics

In some embodiments, the pharmacologic half-life (T½) of the compound of Formula (I), when administered orally to a subject via the pharmaceutical composition disclosed herein, is less than 180 minutes, less than 160 minutes, less than 140 minutes, less than 120 minutes.

In some embodiments, the time for the compound of Formula (I) to reach the maximum serum concentration (Tmax), after being administered orally to a subject via the pharmaceutical composition disclosed herein, is less than 180 minutes, less than 160 minutes, less than 140 minutes, less than 120 minutes, less than 100 minutes, less than 80 minutes, less than 60 minutes, less than 50 minutes, less than 40 minutes, less than 30 minutes. In some embodiments, the time for the compound of Formula (I) to reach the maximum serum concentration (Tmax), after being administered orally to a subject via an orally disintegrating tablet (ODT) dosage form is at least 20% lower, at least 25% lower, at least 30% lower, at least 35% lower, at least 40% lower, at least 45% lower, or at least 50% lower than oral administration of the same compound of Formula (I) via a powder in capsule (PIC) dosage form.

In some embodiments, oral administration of the pharmaceutical composition disclosed herein comprising the compound of Formula (I) provides a maximum serum concentration (Cmax) of the compound of Formula (I) which is at least 20% higher, at least 40% higher, at least 60% higher, at least 80% higher, at least 100% higher, at least 120% higher, at least 130% higher than oral administration of psilocybin in substantially the same dosage form.

In some embodiments, oral administration of the pharmaceutical composition disclosed herein comprising the compound of Formula (I), or a pharmaceutically acceptable salt, polymorph, stereoisomer, or solvate thereof, provides an exposure of the compound of Formula (I)-represented as area under the concentration time curve from the time of dosing to the time of last measurable concentration (AUClast) or area under the concentration time curve from the time of dosing extrapolated to infinity (AUCINF_obs)—which is at least 50% higher, at least 70% higher, at least 90% higher, at least 100% higher, at least 120% higher, at least 140% higher, at least 160% higher, at least 170% higher than oral administration of psilocybin in substantially the same dosage form.

In some embodiments, the volume of distribution of the compound of Formula (I) observed (Vz_F_obs) after being administered orally to a subject is at least 20% lower, at least 25% lower, at least 30% lower, at least 35% lower, at least 40% lower, at least 45% lower, at least 50% lower, at least 55% lower, at least 60% lower, than oral administration of psilocybin in substantially the same dosage form.

In some embodiments, the clearance of the compound of Formula (I) observed (Cl_F_obs; mL/kg/hr) after being administered orally to a subject via the pharmaceutical composition disclosed herein, is from 1,500, from 1,600, from 1,700, from 1,800, from 1,900, from 2,000, from 2,100, from 2,200, from 2,300, from 2,400, and up to 3,500, to 3,400, to 3,300, to 3,200, to 3,100, to 3,000, to 2,900, to 2,800, to 2,700, to 2,600 mL/kg/hr.

In some embodiments, the pharmaceutical composition has an onset of therapeutic action of 60 minutes or less, 50 minutes or less, 40 minutes or less, 30 minutes or less, 20 minutes or less, 10 minutes or less, or 5 minutes or less. In some embodiments, the pharmaceutical composition has an acute effects duration of 240 minutes or less, 180 minutes or less, 120 minutes or less, 60 minutes or less, 50 minutes or less, 40 minutes or less, 30 minutes or less, 20 minutes or less, 10 minutes or less, or 5 minutes or less. In some embodiments, the pharmaceutical composition has a drug dissolution time of 120 seconds or less, 90 seconds or less, 60 seconds or less, 50 seconds or less, 40 seconds or less, 30 seconds or less, 20 seconds or less, 10 seconds or less, or 5 seconds or less.

### Stabilized Compositions

In some embodiments, pharmaceutical compositions are provided which include the compound of Formula (I), or a pharmaceutically acceptable salt, polymorph, stereoisomer, or solvate thereof, in a stabilized form with a pharmaceutically acceptable vehicle. For example, an amorphous form of the compound of Formula (I) may be stabilized in the disclosed pharmaceutical compositions. In some embodiments, formulations of the compound of Formula (I) in which the compound of Formula (I) exists stably in amorphous form may be accomplished, for example, by immobilizing the compound within a matrix formed by a polymer, e.g., as a solid dispersion or solid molecular complex of the compound of Formula (I) and a polymer.

Provided are solid dispersions and solid molecular complexes that include the compound of Formula (I), or a pharmaceutically acceptable salt, polymorph, stereoisomer, or solvate thereof. For example, the compound of Formula (I), or a pharmaceutically acceptable salt, polymorph, stereoisomer, or solvate thereof, may be dispersed within a matrix formed by a polymer in its solid state such that it is immobilized in its amorphous form. In some embodiments, the polymer may prevent intramolecular hydrogen bonding or weak dispersion forces between two or more drug molecules of the compound of Formula (I). In some embodiments, the solid dispersion provides for a large surface area, thus further allowing for improved dissolution and bioavailability of the compound of Formula (I). In some embodiments, a solid dispersion or solid molecular complex includes a therapeutically effective amount of the compound of Formula (I), or a pharmaceutically acceptable salt, polymorph, stereoisomer, or solvate thereof.

In some embodiments, the compound of Formula (I), or a pharmaceutically acceptable salt, polymorph, stereoisomer, or solvate thereof, is present in the solid dispersion in an amount of from about 1%, about 5%, about 10%, about 15%, about 20%, about 25%, about 30%, about 35%, about 40%, about 45%, about 50% by weight, based on a total weight of the solid dispersion, or any range therebetween, e.g., from about 1% to about 50% by weight; or from about 10% to about 40% by weight; or from about 20% to about 35% by weight; or from about 25% to about 30% by weight. In some embodiments, a polymer is present in the solid dispersion in an amount of from about 0%, about 1%, about 5%, about 10%, about 15%, about 20%, about 25%, about 30%, about 35%, about 40%, about 45%, about 50%, about 60%, about 70%, about 80%, about 90% by weight, based on a total weight of the solid dispersion, or any range therebetween, e.g., from 0% to about 50% by weight; or from about 5% to about 60% by weight; or from 10% to about 70% by weight. In some embodiments, a polymer is present in the solid dispersion in an amount greater than about 10% by weight; or greater than about 20% by weight; or greater than about 30% by weight; or greater than about 40% by weight; or greater than about 50% by weight, based on a total weight of the solid dispersion. In some embodiments, the solid dispersion is about 30% by weight of the compound of Formula (I), or a pharmaceutically acceptable salt, polymorph, stereoisomer, or solvate thereof, and about 70% by weight polymer.

The solid dispersion may comprise the compound of Formula (I), or a pharmaceutically acceptable salt, polymorph, stereoisomer, or solvate thereof dispersed in a non-ionic polymer. This may be accomplished by, for example, melting the polymer and dissolving the compound in the polymer and then cooling the mixture. The resulting solid dispersion may comprise the compound dispersed in the polymer in amorphous form.

A solid dispersion may be formed by dispersing the compound of Formula (I), or a pharmaceutically acceptable salt, polymorph, stereoisomer, or solvate thereof in an ionic polymer. Such solid dispersion may result in increased stability of the compound of Formula (I), or a pharmaceutically acceptable salt, polymorph, stereoisomer, or solvate thereof. This may be accomplished by various means, including the methods described above for use in forming a dispersion in a non-ionic polymer. Because ionic polymers have pH dependent solubility in aqueous systems, the resulting solid dispersion of the compound of Formula (I) and the polymer may be stable at low pH in the stomach and release the compound of Formula (I) in the intestine at higher pH. In some embodiments, the compound of Formula (I), or a pharmaceutically acceptable salt, polymorph, stereoisomer, or solvate thereof in such solid dispersions with an ionic polymer may thus be less capable of separating from the polymer and may be immobilized by the polymer in its amorphous form. Examples of such ionic polymers include, but are not limited to, hydroxypropylmethyl cellulose acetate succinate (HPMC-AS), hydroxypropylmethyl cellulose phthalate (HPMCP), and methacrylic acid copolymers. In some embodiments, a polymer is used that is capable of immobilizing the compound of Formula (I), or a pharmaceutically acceptable salt, polymorph, stereoisomer, or solvate thereof so that it exists primarily in one particular polymorph, e.g., an amorphous form, for an extended period of time.

In some embodiments, the polymer may be linear, branched, or crosslinked. In some embodiments, the polymer may be a homopolymer or copolymer. In some embodiments, the polymer may be a synthetic polymer derived from vinyl, acrylate, methacrylate, urethane, ester and oxide monomers. In some embodiments, the polymer can be a derivative of naturally occurring polymers such as polysaccharides (e.g. chitin, chitosan, dextran and pullulan; gum agar, gum arabic, gum karaya, locust bean gum, gum tragacanth, carrageenans, gum ghatti, guar gum, xanthan gum and scleroglucan), starches (e.g. dextrin and maltodextrin), hydrophilic colloids (e.g. pectin), phosphatides (e.g. lecithin), alginates (e.g. ammonium alginate, sodium, potassium or calcium alginate, propylene glycol alginate), gelatin, collagen, and cellulose polymers. In some embodiments, the cellulose polymer is selected from the group consisting of ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In some embodiments, the polymer may be selected from the group consisting of gelatin, polyvinyl alcohol, polyvinylpyrrolidone, pullulan, and the cellulose polymers already disclosed herein. In some embodiments, the cellulose polymer comprises various grades of low viscosity, e.g., MW less than or equal to 50,000 daltons.

In some embodiments, the composition can include solid dispersions and solid molecular complexes that include the compound of Formula (I), or a pharmaceutically acceptable salt, polymorph, stereoisomer, or solvate thereof dispersed within a matrix formed by gelatin. In some embodiments, the composition can include solid dispersions and solid molecular complexes that include the compound of Formula (I), or a pharmaceutically acceptable salt, polymorph, stereoisomer, or solvate thereof dispersed within a matrix formed by gelatin and a non-reducing sugar, e.g., mannitol. In some embodiments, the composition can include solid dispersions and solid molecular complexes that include the compound of Formula (I), or a pharmaceutically acceptable salt, polymorph, stereoisomer, or solvate thereof dispersed within a matrix formed by a cellulose polymer described herein. In some embodiments, the composition can include solid dispersions and solid molecular complexes that include the compound of Formula (I), or a pharmaceutically acceptable salt, polymorph, stereoisomer, or solvate thereof dispersed within a matrix formed by a cellulose polymer described herein and polyvinylpyrrolidone.

In some embodiments, the ratio of the amount by weight of the compound of Formula (I), or a pharmaceutically acceptable salt, polymorph, stereoisomer, or solvate thereof within the solid complex to the amount by weight of the polymer therein is from about 1:9 to about 1:1. In some embodiments, the ratio of the amount by weight of the compound of Formula (I), or a pharmaceutically acceptable salt, polymorph, stereoisomer, or solvate thereof, within the solid complex to the amount by weight of the polymer therein is from about 2:8 to about 4:6. In some embodiments, the ratio of the amount by weight of the compound of Formula (I), or a pharmaceutically acceptable salt, polymorph, stereoisomer, or solvate thereof within the solid complex to the amount by weight of the polymer therein is about 3:7.

In some embodiments, the composition can further include one or more pharmaceutically acceptable vehicles, such as solubilizing agents for the compound of Formula (I), or a pharmaceutically acceptable salt, polymorph, stereoisomer, or solvate thereof. Solubilizing agents include those set forth herein, such as organic acid agents (e.g., citric acid), sodium phosphate, and natural amino acids. Other solubilizing agents include, but are not limited to, acacia, cholesterol, diethanolamine (adjunct), glyceryl monostearate, lanolin alcohols, mono- and di-glycerides, monoethanolamine (adjunct), lecithin, oleic acid (adjunct), oleyl alcohol (stabilizing agent), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, diacetate, monostearate, sodium lauryl sulfate, sodium stearate, sorbitan monolaurate, sorbitan monooleate, sorbitan monopalmitate, sorbitan monostearate, stearic acid, trolamine, and emulsifying wax.

Various additives can be mixed, ground or granulated with the solid dispersion as described herein to form a material suitable for the above dosage forms. Potentially beneficial additives may fall generally into the following classes: other matrix materials or diluents, surface active agents, drug complexing agents or solubilizing agents, fillers, disintegrants, binders, lubricants, and pH modifiers (e.g., acids, bases, or buffers). Examples of other matrix materials, fillers, or diluents include lactose, mannitol, xylitol, microcrystalline cellulose, calcium diphosphate, and starch.

Examples of surface active agents include sodium lauryl sulfate and polysorbate 80. Examples of drug complexing agents or solubilizing agents include the polyethylene glycols, caffeine, xanthene, gentisic acid and cylodextrins. Examples of disintegrants include sodium starch glycolate, sodium alginate, carboxymethyl cellulose sodium, methyl cellulose, and croscarmellose sodium. Examples of binders include methyl cellulose, microcrystalline cellulose, starch, and gums such as guar gum, and tragacanth. Examples of lubricants include magnesium stearate and calcium stearate. Examples of pH modifiers include acids (including organic acid agents), such as citric acid, acetic acid, ascorbic acid, lactic acid, aspartic acid, succinic acid, phosphoric acid, and the like; bases such as sodium acetate, potassium acetate, sodium citrate, potassium citrate, sodium tartrate, potassium tartrate, calcium oxide, magnesium oxide, trisodium phosphate, sodium hydroxide, calcium hydroxide, aluminum hydroxide, and the like, and buffers generally comprising mixtures of acids and the salts of said acids.

The composition may, in addition to the solid dispersion or solid molecular complex, also comprise therapeutically inert, inorganic or organic vehicles, such as those set forth herein.

### Therapeutic Applications and Methods

Also disclosed is a method of treating a subject with a disease or disorder comprising administering to the subject a therapeutically effective amount of a compound of Formula (I), or a pharmaceutically acceptable salt, polymorph, stereoisomer, or solvate thereof. In some embodiments, the disease or disorder is associated with a serotonin 5-HT₂ receptor.

The dosage and frequency (single or multiple doses) of the compounds herein administered can vary depending upon a variety of factors, including, but not limited to, the salt form/compound/polymorph to be administered; the disease/condition being treated; route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Therapeutically effective amounts for use in humans may be determined from animal models. For example, a dose for humans can be formulated to achieve a concentration that has been found to be effective in animals. The dosage in humans can be adjusted by monitoring response to the treatment and adjusting the dosage upwards (e.g., up-titration) or downwards (e.g., down-titration).

Dosages may be varied depending upon the requirements of the subject and the active ingredient(s) being employed. The dose administered to a subject, in the context of the pharmaceutical compositions presented herein, should be sufficient to affect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Routes of administration may include oral routes (e.g., enteral/gastric delivery, intraoral administration such buccal, lingual, and sublingual routes), parenteral routes (e.g., intravenous, intradermal, intraarterial, intraperitoneal, intrathecal, intraventricular, intraurethral, intrasternal, intracranial, intramuscular, intrasynovial, and subcutaneous administration), and topical routes (e.g., (intra)dermal, conjuctival, intracorneal, intraocular, ophthalmic, auricular, transdermal, nasal, vaginal, uretheral, respiratory, and rectal administration), or others sufficient to affect a beneficial therapeutic response.

Administration may follow a continuous administration schedule, or an intermittent administration schedule. The administration schedule may be varied depending on the active ingredient(s) employed, the condition being treated, the administration route, etc. For example, administration of a compound of Formula (I), or a pharmaceutically acceptable salt, polymorph, stereoisomer, or solvate thereof, may be performed once a day (QD), or in divided dosages throughout the day, such as 2-times a day (BID), 3-times a day (TID), 4-times a day (QID), or more. In some embodiments administration may be performed nightly (QHS). In some embodiments, administration is performed as needed (PRN). Administration may also be performed on a weekly basis, e.g., once a week, twice a week, three times a week, four times a week, every other week, every two weeks, etc., or less. The administration schedule may also designate a defined number of treatments per treatment course, for example, the compound of Formula (I), or a pharmaceutically acceptable salt, polymorph, stereoisomer, or solvate thereof, may be administered 1 time, 2 times, 3 times, 4 times, 5 times, 6 times, 7 times, or 8 times per treatment course. Other administration schedules may also be deemed appropriate using sound medical judgement.

The dosing can be continuous (7 days of administration in a week) or intermittent, for example, depending on the pharmacokinetics and a particular subject's clearance/accumulation of the drug. If intermittently, the schedule may be, for example, 4 days of administration and 3 days off (rest days) in a week or any other intermittent dosing schedule deemed appropriate using sound medical judgement. The dosing whether continuous or intermittent is continued for a particular treatment course, typically at least a 28-day cycle (1 month), which can be repeated with or without a drug holiday. Longer or shorter courses can also be used such as 14 days, 18 days, 21 days, 24 days, 35 days, 42 days, 48 days, or longer, or any range therebetween. The course may be repeated without a drug holiday or with a drug holiday depending upon the subject. Other schedules are possible depending upon the presence or absence of adverse events, response to the treatment, patient convenience, and the like.

In some embodiments, the use of compositions of the disclosure may be used as a standalone therapy. In some embodiments, the use of compositions of the disclosure may be used as an adjuvant/combination therapy.

Utilizing the teachings provided herein, an effective prophylactic or therapeutic treatment regimen can be planned that does not cause substantial toxicity or adverse side effects (e.g., caused by sedative or psychotomimetic toxic spikes in plasma concentration of any of the compounds of Formula (I)), and yet is entirely effective to treat the clinical symptoms demonstrated by the particular patient. This planning should involve the careful choice of active compound and salt form by considering factors such as compound potency, relative bioavailability, patient body weight, presence and severity of adverse side effects, preferred mode of administration, and the toxicity profile of the selected agent.

A therapeutically effective dose may vary depending on the variety of factors described above, but is typically that which provides the compound of Formula (I) in an amount of about 0.00001 mg to about 10 mg per kilogram body weight of the recipient, or any range in between, e.g., about 0.00001 mg/kg, about 0.00005 mg/kg, about 0.0001 mg/kg, about 0.0005 mg/kg, about 0.001 mg/kg, about 0.005 mg/kg, about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg of the compound of Formula (I) (on an active basis).

The compounds of the present disclosure (e.g., a compound of Formula (I), or a pharmaceutically acceptable salt, polymorph, stereoisomer, or solvate thereof) may be administered at a psychedelic dose. Psychedelic dosing, by mouth or otherwise, may in some embodiments range from about 0.083 mg/kg, about 0.09 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, and up to about 5 mg/kg, about 4 mg/kg, about 3 mg/kg, about 2 mg/kg, about 1 mg/kg, about 0.95 mg/kg, about 0.9 mg/kg, about 0.85 mg/kg, about 0.8 mg/kg, about 0.75 mg/kg, about 0.7 mg/kg, about 0.65 mg/kg, about 0.6 mg/kg, about 0.55 mg/kg of the compound of Formula (I) (on an active basis). Higher dosing may also be used in some embodiments, as described above. In some embodiments, psychedelic doses are administered once by mouth, with the possibility of repeat doses at least one week apart. In some instances, no more than 5 doses are given in any one course of treatment. Courses can be repeated as necessary, with or without a drug holiday. Such acute treatment regimens may be accompanied by psychotherapy, before, during, and/or after the psychedelic dose. These treatments are appropriate for a variety of mental health disorders disclosed herein, examples of which include, but are not limited to, major depressive disorder (MDD), therapy resistant depression (TRD), anxiety disorders, and substance use disorders (e.g., alcohol use disorder, opioid use disorder, amphetamine use disorder, nicotine use disorder, smoking, and cocaine use disorder).

The compounds of the present disclosure (e.g., a compound of Formula (I), or a pharmaceutically acceptable salt, polymorph, stereoisomer, or solvate thereof), may be administered at sub-psychoactive (yet still potentially serotonergic concentrations) concentrations to achieve durable therapeutic benefits, with decreased toxicity, and may thus be suitable for microdosing. Sub-psychedelic dosing, by mouth or otherwise, may in some embodiments range from about 0.00001 mg/kg, about 0.00005 mg/kg, about 0.0001 mg/kg, about 0.0005 mg/kg, about 0.001 mg/kg, about 0.005 mg/kg, about 0.006 mg/kg, about 0.008 mg/kg, about 0.009 mg/kg, about 0.01 mg/kg, and less than about 0.083 mg/kg, about 0.08 mg/kg, about 0.075 mg/kg, about 0.07 mg/kg, about 0.06 mg/kg, about 0.05 mg/kg, about 0.04 mg/kg, about 0.03 mg/kg, about 0.02 mg/kg of the compound of Formula (I) (on an active basis). Typically, sub-psychedelic doses are administered orally up to every day, for a treatment course (e.g., 1 month). However, there is no limitation on the number of doses at sub-psychedelic doses-dosing can be less frequent or more frequent as deemed appropriate. Courses can be repeated as necessary, with or without a drug holiday.

Sub-psychedelic dosing can also be carried out, for example, by transdermal delivery, subcutaneous administration, etc., via modified, controlled, slow, or extended release dosage forms, including, but not limited to, depot dosage forms, implants, patches, and pumps, which can be optionally remotely controlled. Here, doses would achieve similar blood levels as low oral dosing, but would nevertheless be sub-psychedelic.

Sub-psychedelic doses can be used, e.g., for the chronic treatment a variety of diseases or disorders disclosed herein, examples of which include, but are not limited to, depression (e.g., MDD), inflammation, pain and neuroinflammation. In such settings where chronic administration is performed over extended periods of time, the stabilized forms of the compounds provided in the present disclosure become increasingly valuable.

The compounds of the present disclosure (e.g., a compound of Formula (I), or a pharmaceutically acceptable salt, polymorph, stereoisomer, or solvate thereof), may be used for a maintenance regimen. As used herein, a “maintenance regimen” generally refers to the administration of the compounds of the present disclosure (e.g., a compound of Formula (I), or a pharmaceutically acceptable salt, polymorph, stereoisomer, or solvate thereof) following achievement of a target dose, e.g., following completion of an up-titration regimen, and/or following a positive clinical response, e.g., improvement of the patient's condition, either to the same drug or to a different drug. In some embodiments, the patient is administered a first drug for a therapeutic regimen and a second drug for a maintenance regimen, wherein the first and second drugs are different. For example, the patient may be administered a therapeutic regimen of a first drug which is not a compound of the present disclosure (e.g., the first drug is a serotonergic psychedelic such as LSD, psilocybin, MDMA, dimethyltryptamine, etc., or a non-psychedelic drug), followed by a compound of the present disclosure (as the second drug) in a maintenance regimen. In another example, a different compound of the present disclosure is used for the therapeutic regimen (first drug) than is used for the maintenance regimen (second drug). In some embodiments, the patient is administered the same compound of the present disclosure for both a therapeutic regimen and a maintenance regimen. In any case, the maintenance dose of the compounds of the present disclosure may be used to ‘maintain’ the therapeutic response and/or to prevent occurrences of relapse. When the same compound of the present disclosure is used for both the original therapeutic regimen and for the maintenance regimen, the maintenance dose of the compound may be at or below the therapeutic dose. In some embodiments, the maintenance dose is a psychedelic dose. In some embodiments, the maintenance dose is a sub-psychedelic dose. Generally, dosing is carried out daily or intermittently for the maintenance regimen, however, maintenance regimens can also be carried out continuously, for example, over several days, weeks, months, or years. Moreover, the maintenance dose may be given to a patient over a long period of time, even chronically.

The subjects treated herein may have a disease or disorder associated with a serotonin 5-HT₂ receptor.

In some embodiments, the disease or disorder is a neuropsychiatric disease or disorder or an inflammatory disease or disorder. In some embodiments, the neuropsychiatric disease or disorder is not schizophrenia or cognitive deficits in schizophrenia.

In some embodiments, the disease or disorder is a central nervous system (CNS) disorder, including, but not limited to, major depressive disorder (MDD), treatment-resistant depression (TRD), post-traumatic stress disorder (PTSD), bipolar and related disorders (including, but not limited to, bipolar I disorder, bipolar II disorder, cyclothymic disorder), obsessive-compulsive disorder (OCD), generalized anxiety disorder (GAD), social anxiety disorder, substance use disorders (including, but not limited to, alcohol use disorder, opioid use disorder, amphetamine use disorder, nicotine use disorder, smoking, and cocaine use disorder), eating disorders (including, but not limited to anorexia nervosa, bulimia nervosa, binge-eating disorder, etc.), Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, suicidal ideation, suicidal behavior, major depressive disorder with suicidal ideation or suicidal behavior, melancholic depression, atypical depression, dysthymia, non-suicidal self-injury disorder (NSSID), chronic fatigue syndrome, Lyme's disease, gambling disorder, paraphilic disorders (including, but not limited to, pedophilic disorder, exhibitionistic disorder, voyeuristic disorder, fetishistic disorder, sexual masochism or sadism disorder, and transvestic disorder, etc.), sexual dysfunction (e.g., low libido, hypoactive sexual desire disorder (HSDD), etc.), peripheral neuropathy, and obesity.

In some embodiments, the methods provided herein are used to treat a subject with a depressive disorder. As used herein, the terms “depressive disorder” or “depression” refers to a group of disorders characterized by low mood that can affect a person's thoughts, behavior, feelings, and sense of well-being lasting for a period of time. In some embodiments, the depressive disorder disrupts the physical and psychological functions of a person. In some embodiments, the depressive disorder causes a physical symptom such as weight loss, aches or pains, headaches, cramps, or digestive problems. In some embodiments, the depressive disorder causes a psychological symptom such as persistent sadness, anxiety, feelings of hopelessness and irritability, feelings of guilt, worthlessness, or helplessness, loss of interest or pleasure in hobbies and activities, difficulty concentrating, remembering, or making decisions. In some embodiments, the depressive disorder is major depressive disorder (MDD), atypical depression, bipolar disorder, catatonic depression, depressive disorder due to a medical condition, postpartum depression, premenstrual dysphoric disorder, seasonal affective disorder, or treatment-resistant depression (TRD).

In some embodiments, the disease or disorder is major depressive disorder (MDD). As used herein, the term “major depressive disorder” refers to a condition characterized by a time period of low mood that is present across most situations. Major depressive disorder is often accompanied by low self-esteem, loss of interest in normally enjoyable activities, low energy, and pain without a clear cause. In some instances, major depressive order is characterized by symptoms of depression lasting at least two weeks. In some instances, an individual experiences periods of depression separated by years. In some instances, an individual experiences symptoms of depression that are nearly always present. -Major depressive disorder can negatively affect a person's personal, work, or school life, as well as sleeping, eating habits, and general health. Approximately 2-7% of adults with major depressive disorder commit suicide, and up to 60% of people who commit suicide had major depressive disorder or another related mood disorder. Dysthymia is a subtype of major depressive disorder consisting of the same cognitive and physical problems as major depressive disorder with less severe but longer-lasting symptoms. Exemplary symptoms of a major depressive disorder include, but are not limited to, feelings of sadness, tearfulness, emptiness or hopelessness, angry outbursts, irritability or frustration, even over small matters, loss of interest or pleasure in most or all normal activities, sleep disturbances, including insomnia or sleeping too much, tiredness and lack of energy, reduced appetite, weight loss or gain, anxiety, agitation or restlessness, slowed thinking, speaking, or body movements, feelings of worthlessness or guilt, fixating on past failures or self-blame, trouble thinking, concentrating, making decisions, and remembering things, frequent thoughts of death, suicidal thoughts, suicide attempts, or suicide, and unexplained physical problems, such as back pain or headaches.

As used herein, the term “atypical depression” refers to a condition wherein an individual shows signs of mood reactivity (i.e., mood brightens in response to actual or potential positive events), significant weight gain, increase in appetite, hypersomnia, heavy, leaden feelings in arms or legs, and/or long-standing pattern of interpersonal rejection sensitivity that results in significant social or occupational impairment. Exemplary symptoms of atypical depression include, but are not limited to, daily sadness or depressed mood, loss of enjoyment in things that were once pleasurable, major changes in weight (gain or loss) or appetite, insomnia or excessive sleep almost every day, a state of physical restlessness or being rundown that is noticeable by others, daily fatigue or loss of energy, feelings of hopelessness, worthlessness, or excessive guilt almost every day, problems with concentration or making decisions almost every day, recurring thoughts of death or suicide, suicide plan, or suicide attempt.

As used herein, the term “bipolar disorder” refers to a condition that causes an individual to experience unusual shifts in mood, energy, activity levels, and the ability to carry out day-to day tasks. Individuals with bipolar disorder experience periods of unusually intense emotion, changes in sleep patterns and activity levels, and unusual behaviors. These distinct periods are called “mood episodes.” Mood episodes are drastically different from the moods and behaviors that are typical for the person. Exemplary symptoms of mania, excessive behavior, include, but are not limited to, abnormally upbeat, jumpy, or wired behavior, increased activity, energy, or agitation, exaggerated sense of well-being and self-confidence, decreased need for sleep, unusual talkativeness, racing thoughts, distractibility, and poor decision-making—for example, going on buying sprees, taking sexual risks, or making foolish investments. Exemplary symptoms of depressive episodes or low mood, include, but are not limited to, depressed mood, such as feelings of sadness, emptiness, hopelessness, or tearfulness; marked loss of interest or feeling no pleasure in all-or almost all-activities, significant weight loss, weight gain, or decrease or increase in appetite, insomnia or hypersomnia (excessive sleeping or excessive sleepiness), restlessness or slowed behavior, fatigue or loss of energy, feelings of worthlessness or excessive or inappropriate guilt, decreased ability to think or concentrate, or indecisiveness, and thinking about, planning or attempting suicide. Bipolar disorder includes bipolar I disorder, bipolar II disorder, and cyclothymic disorder. Bipolar I disorder is defined by manic episodes that last at least 7 days or by severe manic symptoms that require hospitalization. A subject with bipolar I disorder may also experience depressive episodes typically lasting at least 2 weeks. Episodes of depression with mixed features, i.e., depressive and manic symptoms at the same time, are also possible. Bipolar II disorder is characterized by a pattern of depressive and hypomanic episodes, but not severe manic episodes typical of bipolar I disorder. Cyclothymic disorder (also referred to as cyclothymia) is characterized by periods of hypomanic symptoms (elevated mood and euphoria) and depressive symptoms lasting over a period of at least 2 years. The mood fluctuations are not sufficient in number, severity, or duration to meet the full criteria for a hypomanic or depressive episode.

As used herein, the term “catatonic depression” refers to a condition causing an individual to remain speechless and motionless for an extended period. Exemplary symptoms of catatonic depression include, but are not limited to, feelings of sadness, which can occur daily, a loss of interest in most activities, sudden weight gain or loss, a change in appetite, trouble falling asleep, trouble getting out of bed, feelings of restlessness, irritability, feelings of worthlessness, feelings of guilt, fatigue, difficulty concentrating, difficulty thinking, difficulty making decisions, thoughts of suicide or death, and/or a suicide attempt.

As used herein, the term “depressive disorder due to a medical condition” refers to a condition wherein an individual experiences depressive symptoms caused by another illness. Examples of medical conditions known to cause a depressive disorder include, but are not limited to, HIV/AIDS, diabetes, arthritis, strokes, brain disorders such as Parkinson's disease, Huntington's disease, multiple sclerosis, and Alzheimer's disease, metabolic conditions (e.g. vitamin B12 deficiency), autoimmune conditions (e.g., lupus and rheumatoid arthritis), viral or other infections (hepatitis, mononucleosis, herpes), back pain, and cancer (e.g., pancreatic cancer).

As used herein, the term “postpartum depression” refers to a condition as the result of childbirth and hormonal changes, psychological adjustment to parenthood, and/or fatigue. Postpartum depression is often associated with women, but men can also suffer from postpartum depression as well. Exemplary symptoms of postpartum depression include, but are not limited to, feelings of sadness, hopeless, emptiness, or overwhelmed; crying more often than usual or for no apparent reason; worrying or feeling overly anxious; feeling moody, irritable, or restless; oversleeping, or being unable to sleep even when the baby is asleep; having trouble concentrating, remembering details, and making decisions; experiencing anger or rage; losing interest in activities that are usually enjoyable; suffering from physical aches and pains, including frequent headaches, stomach problems, and muscle pain; eating too little or too much; withdrawing from or avoiding friends and family; having trouble bonding or forming an emotional attachment with the baby; persistently doubting his or ability to care for the baby; and thinking about harming themselves or the baby.

As used herein, the term “premenstrual dysphoric disorder” refers to a condition wherein an individual expresses mood lability, irritability, dysphoria, and anxiety symptoms that occur repeatedly during the premenstrual phase of the cycle and remit around the onset of menses or shortly thereafter. Exemplary symptoms of premenstrual dysphoric disorder includes, but are not limited to, lability (e.g., mood swings), irritability or anger, depressed mood, anxiety and tension, decreased interest in usual activities, difficulty in concentration, lethargy and lack of energy, change in appetite (e.g., overeating or specific food cravings), hypersomnia or insomnia, feeling overwhelmed or out of control, physical symptoms (e.g., breast tenderness or swelling, joint or muscle pain, a sensation of‘bloating’ and weight gain), self-deprecating thoughts, feelings of being keyed up or on edge, decreased interest in usual activities (e.g., work, school, friends, hobbies), subjective difficulty in concentration, and easy fatigability.

As used herein, the term “seasonal affective disorder” refers to a condition wherein an individual experiences mood changes based on the time of the year. In some instances, an individual experiences low mood, low energy, or other depressive symptoms during the fall and/or winter season. In some instances, an individual experiences low mood, low energy, or other depressive symptoms during the spring and/or summer season. Exemplary symptoms of seasonal affective disorder include, but are not limited to, feeling depressed most of the day or nearly every day, losing interest in activities once found enjoyable, having low energy, having problems with sleeping, experiencing changes in appetite or weight, feeling sluggish or agitated, having difficulty concentrating, feeling hopeless, worthless, or guilty, and having frequent thoughts of death or suicide.

In some embodiments, a depressive disorder comprises a medical diagnosis based on the criteria and classification from Diagnostic and Statistical Manual of Mental Disorders, 5th Ed. In some embodiments, a depressive disorder comprises a medical diagnosis based on an independent medical evaluation.

In some embodiments, the methods described herein are provided to a subject with depression that is resistant to treatment. In some embodiments, the subject has been diagnosed with treatment-resistant depression (TRD). The term “treatment-resistant depression” refers to a kind of depression that does not respond or is resistant to at least one or more treatment attempts of adequate dose and duration. In some embodiments, the subject with treatment-resistant depression has failed to respond to 1 treatment attempt, 2 treatment attempts, 3 treatment attempts, 4 treatment attempts, 5 treatment attempts, or more. In some embodiments, the subject with treatment-resistant depression has been diagnosed with major depressive disorder and has failed to respond to 3 or more treatment attempts. In some embodiments, the subject with treatment resistant depression has been diagnosed with bipolar disorder and has failed to respond to 1 treatment attempt.

In some embodiments, the methods provided herein reduce at least one sign or symptom of a depressive disorder. In some embodiments, the methods provided herein reduce at least one sign or symptom of a depressive disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the disease or disorder is an anxiety disorder. As used herein, the term “anxiety disorder” refers to a state of apprehension, uncertainty, and/or fear resulting from the anticipation of an event and/or situation. Anxiety disorders cause physiological and psychological signs or symptoms. Non-limiting examples of physiological symptoms include muscle tension, heart palpitations, sweating, dizziness, shortness of breath, tachycardia, tremor, fatigue, worry, irritability, and disturbed sleep. Non-limiting examples of psychological symptoms include fear of dying, fear of embarrassment or humiliation, fear of an event occurring, etc. Anxiety disorders also impair a subject's cognition, information processing, stress levels, and immune response. In some embodiments, the methods disclosed herein treat chronic anxiety disorders. As used herein, a “chronic” anxiety disorder is recurring. Examples of anxiety disorders include, but are not limited to, generalized anxiety disorder (GAD), social anxiety disorder, panic disorder, panic attack, a phobia-related disorder (e.g., phobias related to flying, heights, specific animals such as spiders/dogs/snakes, receiving injections, blood, etc., agoraphobia), separation anxiety disorder, selective mutism, anxiety due to a medical condition, post-traumatic stress disorder (PTSD), obsessive-compulsive disorder (OCD), substance-induced anxiety disorder, etc.

In some embodiments, the subject in need thereof develops an anxiety disorder after experiencing the effects of a disease. The effects of a disease include diagnosis of an individual with said disease, diagnosis of an individual's loved ones with said disease, social isolation due to said disease, quarantine from said disease, or social distancing as a result of said disease. In some embodiments, an individual is quarantined to prevent the spread of the disease. In some embodiments, the disease is COVID-19, SARS, or MERS. In some embodiments, a subject develops an anxiety disorder after job loss, loss of housing, or fear of not finding employment.

In some embodiments, the disease or disorder is generalized anxiety disorder (GAD). Generalized anxiety disorder is characterized by excessive anxiety and worry, fatigue, restlessness, increased muscle aches or soreness, impaired concentration, irritability, and/or difficulty sleeping. In some embodiments, a subject with generalized anxiety disorder does not have associated panic attacks. In some embodiments, after treating the symptom is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the disease or disorder is social anxiety disorder. As used herein, “social anxiety disorder” is a marked fear or anxiety about one or more social situations in which the individual is exposed to possible scrutiny by others. Non-limiting examples of situations which induce social anxiety include social interactions (e.g., having a conversation, meeting unfamiliar people), being observed (e.g., eating or drinking), and performing in front of others (e.g., giving a speech). In some embodiments, the social anxiety disorder is restricted to speaking or performing in public. In some embodiments, treating according to the methods of the disclosure reduces or ameliorates a symptom of social anxiety disorder. In some embodiments, after treating the symptom is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the disease or disorder is a compulsive disorder, such as obsessive-compulsive disorder (OCD), body-focused repetitive behavior, hoarding disorder, gambling disorder, compulsive buying, compulsive internet use, compulsive video gaming, compulsive sexual behavior, compulsive eating, compulsive exercise, body dysmorphic disorder, hoarding disorder, dermatillomania, trichotillomania, excoriation, substance-induced obsessive compulsive and related disorder, or an obsessive-compulsive disorder due to another medical condition, etc., or a combination thereof. In some embodiments, the disease or disorder is obsessive-compulsive disorder (OCD).

In some embodiments, at least one sign or symptom of an anxiety disorder is improved following the administration of a compound as disclosed herein. In some embodiments, a sign or symptom of an anxiety disorder is measured according to a diary assessment, an assessment by a clinician or caregiver, or a clinical scale. In some embodiments, treatment causes a demonstrated improvement in one or more of the following: State-Trait Anxiety Inventory (STAI), Beck Anxiety Inventory (BAI), Hospital Anxiety and Depression Scale (HADS), Generalized Anxiety Disorder questionnaire-IV (GADQ-IV), Hamilton Anxiety Rating Scale (HARS), Leibowitz Social Anxiety Scale (LSAS), Overall Anxiety Severity and Impairment Scale (OASIS), Hospital Anxiety and Depression Scale (HADS), Patient Health Questionnaire 4 (PHQ-4), Social Phobia Inventory (SPIN), Brief Trauma Questionnaire (BTQ), Combat Exposure Scale (CES), Mississippi Scale for Combat-Related PTSD (M-PTSD), Posttraumatic Maladaptive Beliefs Scale (PMBS), Perceived Threat Scale (DRRI-2 Section: G), PTSD Symptom Scale-Interview for DSM-5 (PSS-1-5), Structured Interview for PTSD (SI-PTSD), Davidson Trauma Scale (DTS), Impact of Event Scale-Revised (IES-R), Posttraumatic Diagnostic Scale (PDS-5), Potential Stressful Events Interview (PSEI), Stressful Life Events Screening Questionnaire (SLESQ), Spielberger's Trait and Anxiety, Generalized Anxiety Dis-order 7-Item Scale, The Psychiatric Institute Trichotillomania Scale (PITS), The MGH Hairpulling Scale (MGH-HPS), The NIMH Trichotillomania Severity Scale (NIMH-TSS), The NIMH Trichotillomania Impairment Scale (NIMH-TIS), The Clinical Global Impression (CGI), the Brief Social Phobia Scale (BSPS), The Panic Attack Questionnaire (PAQ), Panic Disorder Severity Scale, Florida Obsessive-Compulsive Inventory (FOCI), The Leyton Obsessional Inventory Survey Form, The Vancouver Obsessional Compulsive Inventory (VOCI), The Schedule of Compulsions, Obsessions, and Pathological Impulses (SCOPI), Padua Inventory-Revised (PI-R), Quality of Life (QoL), The Clinical Global Improvement (CGI) scale, The Yale-Brown Obsessive-Compulsive Scale (Y-BOCS), The Yale-Brown Obsessive-Compulsive Scale Second Edition (Y-BOCS-II), The Dimensional Yale-Brown Obsessive-Compulsive Scale (DY-BOCS), The National Institute of Mental Health-Global Obsessive-Compulsive Scale (NIMH-GOCS), The Yale-Brown Obsessive-Compulsive Scale Self-Report (Y-BOCS-SR), The Obsessive-Compulsive Inventory-Re-vised (OCI-R), and the Dimensional Obsessive-Compulsive Scale (DOCS), or a combination thereof. In some embodiments, treating according to the methods of the disclosure results in an improvement in an anxiety disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art.

In some embodiments, the disease or disorder is a headache disorder. As used herein, the term “headache disorder” refers to a disorder characterized by recurrent headaches. Headache disorders include migraine, tension-type headache, cluster headache, and chronic daily headache syndrome.

In some embodiments, a method of treating cluster headaches in a subject in need thereof is disclosed herein. In some embodiments, at least one sign or symptom of cluster headache is improved following treatment. In some embodiments, the sign or symptom of cluster headache is measured according to a diary assessment, a physical or psychological assessment by clinician, an imaging test, or a neurological examination. Cluster headache is a primary headache disorder and belongs to the trigeminal autonomic cephalalgias. The definition of cluster headaches is a unilateral headache with at least one autonomic symptom ipsilateral to the headache. Attacks are characterized by severe unilateral pain predominantly in the first division of the trigeminal nerve—the fifth cranial nerve whose primary function is to provide sensory and motor innervation to the face. Attacks are also associated with prominent unilateral cranial autonomic symptoms and subjects often experience agitation and restlessness during attacks. In some embodiments, a subject with cluster headaches also experiences nausea and/or vomiting. In some embodiments, a subject with cluster headaches experiences unilateral pain, excessive tearing, facial flushing, a droopy eyelid, a constricted pupil, eye redness, swelling under or around one or both eyes, sensitivity to light, nausea, agitation, and restlessness.

In some embodiments, a method of treating migraines in a subject in need thereof is disclosed herein. A migraine is a moderate to severe headache that affects one half or both sides of the head, is pulsating in nature, and last from 2 to 72 hours. Symptoms of migraine include headache, nausea, sensitivity to light, sensitivity to sound, sensitivity to smell, dizziness, difficulty speaking, vertigo, vomiting, seizure, distorted vision, fatigue, or loss of appetite. Some subjects also experience a prodromal phase, occurring hours or days before the headache, and/or a postdromal phase following headache resolution. Prodromal and postdromal symptoms include hyperactivity, hypoactivity, depression, cravings for particular foods, repetitive yawning, fatigue and neck stiffness and/or pain. In some embodiments, the migraine is a migraine without aura, a migraine with aura, a chronic migraine, an abdominal migraine, a basilar migraine, a menstrual migraine, an ophthalmoplegic migraine, an ocular migraine, an ophthalmic migraine, or a hemiplegic migraine. In some embodiments, the migraine is a migraine without aura. A migraine without aura involves a migraine headache that is not accompanied by a headache. In some embodiments, the migraine is a migraine with Aura. A migraine with aura is primarily characterized by the transient focal neurological symptoms that usually precede or sometimes accompany the headache. Less commonly, an aura can occur without a headache, or with a non-migraine headache. In some embodiments, the migraine is a hemiplegic migraine. A hemiplegic migraine is a migraine with aura and accompanying motor weakness. In some embodiments, the hemiplegic migraine is a familial hemiplegic migraine or a sporadic hemiplegic migraine. In some embodiments, the migraine is a basilar migraine. A subject with a basilar migraine has a migraine headache and an aura accompanied by difficulty speaking, world spinning, ringing in ears, or a number of other brainstem-related symptoms, not including motor weakness. In some embodiments, the migraine is a menstrual migraine. A menstrual migraine occurs just before and during menstruation. In some embodiments, the subject has an abdominal migraine. Abdominal migraines are often experienced by children. Abdominal migraines are not headaches, but instead stomach aches. In some embodiments, a subject with abdominal migraines develops migraine headaches. In some embodiments, the subject has an ophthalmic migraine also called an “ocular migraine.” Subjects with ocular migraines experience vision or blindness in one eye for a short time with or after a migraine headache. In some embodiments, a subject has an ophthalmoplegic migraine. Ophthalmoplegic migraines are recurrent attacks of migraine headaches associated with paresis of one or more ocular cranial nerves. In some embodiments, the subject in need of treatment experiences chronic migraines. As defined herein, a subject with chronic migraines has more than fifteen headache days per month. In some embodiments, the subject in need of treatment experiences episodic migraines. As defined herein, a subject with episodic migraines has less than fifteen headache days per month.

In some embodiments, a method of treating chronic daily headache syndrome (CDHS) in a subject in need thereof is disclosed herein. A subject with CDHS has a headache for more than four hours on more than 15 days per month. Some subjects experience these headaches for a period of six months or longer. CHDS affects 4% of the general population. Chronic migraine, chronic tension-type headaches, new daily persistent headache, and medication overuse headaches account for the vast majority of chronic daily headaches.

In some embodiments, after treating according to the methods of the disclosure, the frequency of headaches and/or related symptoms decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, after treating according to the methods of the disclosure, the length of a headache attack decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, at least one sign or symptom of headache disorder is improved following administration of a compound disclosed herein. In some embodiments, a sign or symptom of a headache disorder is measured according to a diary assessment, an assessment by a clinician or caregiver, or a clinical scale. In some embodiments, treatment of the present disclosure causes a demonstrated improvement in one or more of the following: the Visual Analog Scale, Numeric Rating Scale, the Short Form Health Survey, Profile of Mood States, the Pittsburgh Sleep Quality Index, the Major Depression Inventory, the Perceived Stress Scale, the 5-Level EuroQoL-SD, the Headache Impact Test; the ID-migraine; the 3-item screener; the Minnesota Multiphasic Personality Inventory; the Hospital Anxiety and Depression Scale (HADS), the 50 Beck Depression Inventory (BDI; both the original BD151 and the second edition, BDI-1152), the 9-item Patient Health Questionnaire (PHQ-9), the Migraine Disability Assessment Questionnaire (MI-DAS), the Migraine-Specific Quality of Life Questionnaire version 2.1 (MSQ v2.1), the European Quality of Life-S Dimensions (EQ-SD), the Short-form 36 (SF-36), or a combination thereof. In some embodiments, treating according to the methods of the disclosure results in an improvement in a headache disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art. In some embodiments, the sign or symptom of the headache disorder is measured according to a diary assessment, a physical or psychological assessment by clinician, an imaging test, an electroencephalogram, a blood test, a neurological examination, or combination thereof. In some embodiments, the blood test evaluates blood chemistry and/or vitamins.

In some embodiments, the disease or disorder is a substance use disorder. Substance addictions which can be treated using the methods herein include addictions to addictive substances/agents such as recreational drugs and addictive medications. Examples of addictive substances/agents include, but are not limited to, alcohol, e.g., ethyl alcohol, gamma hydroxybutyrate (GHB), caffeine, nicotine, cannabis (marijuana) and cannabis derivatives, opiates and other morphine-like opioid agonists such as heroin, phencyclidine and phencyclidine-like compounds, sedative hypnotics such as benzodiazepines, methaqualone, mecloqualone, etaqualone and barbiturates and psychostimulants such as cocaine, amphetamines and amphetamine-related drugs such as dextroamphetamine and methylamphetamine. Examples of addictive medications include, e.g., benzodiazepines, barbiturates, and pain medications including alfentanil, allylprodine, alphaprodine, anileridine benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaheptyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, OXYCONTIN®, oxymorphone, papaveretum, pentazocine, phenadoxone, phenomorphan, phenazocine, phenoperidine, piminodine, piritramide, propheptazine, promedol, properidine, propiram, propoxyphene sufentanil, tramadol, and tilidine. In some embodiments, the disease or disorder is alcohol use disorder. In some embodiments, the disease or disorder is nicotine use (e.g., smoking) disorder, and the therapy is used for e.g., smoking cessation.

In some embodiments, the disclosure provides for the management of sexual dysfunction, which may include, but is not limited to, sexual desire disorders, for example, decreased libido; sexual arousal disorders, for example, those causing lack of desire, lack of arousal, pain during intercourse, and orgasm disorders such as anorgasmia; and erectile dysfunction; particularly sexual dysfunction disorders stemming from psychological factors.

In some embodiments, the disease or disorder is an eating disorder. As used herein, the term “eating disorder” refers to any of a range of psychological disorders characterized by abnormal or disturbed eating habits. Non-limiting examples of eating disorders include pica, anorexia nervosa, bulimia nervosa, rumination disorder, avoidant/restrictive food intake disorder, binge-eating disorder, other specified feeding or eating disorder, unspecified feeding or eating disorder, or combinations thereof. In some embodiments, the eating disorder is pica, anorexia nervosa, bulimia nervosa, rumination disorder, avoidant/restrictive food intake disorder, binge-eating disorder, or combinations thereof. In some embodiments, the methods disclosed herein treat chronic eating disorders. As used herein, a “chronic” eating disorder is recurring. In some embodiments, at least one sign or symptom of an eating disorder is improved following administration of a compound disclosed herein. In some embodiments, a sign or symptom of an eating disorder is measured according to a diary assessment, an assessment by a clinician or caregiver, or a clinical scale. Non-limiting examples of clinical scales, diary assessments, and assessments by a clinician or caregiver include: the Mini International Neuropsychiatric Interview (MINI), the McLean Screening Instrument for Borderline Personality Disorder (MSI-BPD), the Eating Disorder Examination (EDE), the Eating Disorder Questionnaire (EDE-Q), the Eating Disorder Examination Questionnaire Short Form (EDE-QS), the Physical Appearance State and Trait Anxiety Scale-State and Trait version (PASTAS), Spielberger State-Trait Anxiety Inventory (STAI), Eating Disorder Readiness Ruler (ED-RR), Visual Analogue Rating Scales (VAS), the Montgomery-Asberg Depression Rating Scale (MADRS), Yale-Brown Cornell Eating Disorder Scale (YBC-EDS), Yale-Brown Cornell Eating Disorder Scale Self Report (YBC-EDS-SRQ), the Body Image State Scale (BISS), Clinical impairment assessment (CIA) questionnaire, the Eating Disorder Inventory (EDI) (e.g. version 3: EDI-3), the Five Dimension Altered States of Consciousness Questionnaire (5D-ASC), the Columbia-Suicide Severity Rating Scale (C-SSRS), the Life Changes Inventory (LCI), and combinations thereof. In some embodiments, treating according to the methods of the disclosure results in an improvement in an eating disorder compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art.

In some embodiments, the disease or disorder is a disease or disorder characterized by, or otherwise associated with, neuroinflammation. Compounds and compositions of the present disclosure may provide cognitive benefits to subject's suffering from neurological and neurodegenerative diseases such as Alzheimer's disease and other dementia subtypes, Parkinson's disease, and others where neuroinflammation is a hallmark of disease pathophysiology and progression. For example, emerging psychedelic research/clinical evidence indicates that psychedelics may be useful as disease-modifying treatments in subjects suffering from neurodegenerative diseases such as Alzheimer's disease and other forms of dementia. See Vann Jones, S. A. and O'Kelly, A. “Psychedelics as a Treatment for Alzheimer's Disease Dementia” Front. Synaptic Neurosci., 21, August 2020; Kozlowska, U., Nichols, C., Wiatr, K., and Figiel, M. (2021), “From psychiatry to neurology: Psychedelics as prospective therapeutics for neurodegenerative disorders” Journal of Neurochemistry, 00, 1-20; Garcia-Romeu, A., Darcy, S., Jackson, H., White, T., Rosenberg, P. (2021), “Psychedelics as Novel Therapeutics in Alzheimer's Disease: Rationale and Potential Mechanisms” In: Current Topics in Behavioral Neurosciences. Springer, Berlin, Heidelberg. For example, psychedelics are thought to stimulate neurogenesis, provoke neuroplastic changes, and to reduce neuroinflammation. Thus, in some embodiments, the compounds of the present disclosure (e.g., a compound of Formula (I)) are used for the treatment of neurological and neurodegenerative disorders such as Alzheimer's disease, dementia subtypes, and Parkinson's disease, where neuroinflammation is associated with disease pathogenesis. In some embodiments, the compounds of the present disclosure are used for the treatment of Alzheimer's disease. In some embodiments, the compounds of the present disclosure are used for the treatment of dementia. In some embodiments, the compounds of the present disclosure are used for the treatment of Parkinson's disease. As described above, such treatment may stimulate neurogenesis, provoke neuroplastic changes, and/or provide neuroinflammatory benefits (e.g., reduced neuroinflammation compared to prior to the beginning of treatment), and as a result, may slow or prevent disease progression, slow or reverse brain atrophy, and reduce symptoms associated therewith (e.g., memory loss in the case of Alzheimer's and related dementia disorders). While not limited thereto, pharmaceutical compositions adapted for oral and/or extended-release dosing are appropriate for such treatment methods, with sub-psychedelic dosing being preferred. In some embodiments, treating according to the methods of the disclosure results in an improvement in cognition in subject's suffering from a neurological or neurodegenerative disease compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of a diary assessments, assessments by a clinical or caregiver, or clinical scales, described herein or known in the art.

Further, many of the behavioral issues associated with chronic and/or life-threatening illnesses, including neurodegenerative disorders such as Alzheimer's disease, may benefit from treatment with the compounds disclosed herein. Indeed, depression, anxiety, or stress can be common among patients who have chronic and/or life-threatening illnesses such as Alzheimer's disease, autoimmune diseases (e.g., systemic lupus erythematosus, rheumatoid arthritis, and psoriasis), cancer, coronary heart disease, diabetes, epilepsy, HIV/AIDS, hypothyroidism, multiple sclerosis, Parkinson's disease, and stroke. For example, depression is common in Alzheimer's disease as a consequence of the disease; as well as being a risk factor for the disease itself. Symptoms of depression, anxiety, or stress can occur after diagnosis with the disease or illness. Patients that have depression, anxiety, or stress concurrent with another medical disease or illness can have more severe symptoms of both illnesses and symptoms of depression, anxiety, or stress can continue even as a patient's physical health improves. Compounds described herein can be used to treat depression, anxiety, and/or stress associated with a chronic or life-threatening disease or illness.

Accordingly, in some embodiments, the methods herein are used to treat symptoms, e.g., depression, anxiety, and/or stress, associated with a chronic and/or life-threatening disease or disorder, including neurological and neurodegenerative diseases. In some embodiments, the methods provided herein reduce at least one sign or symptom of a neurological and/or neurodegenerative disease. In some embodiments, the methods provided herein reduce at least one sign or symptom of a neurological and/or neurodegenerative disease (e.g., depression, anxiety, and/or stress) by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment, e.g., according to any one of the diary assessments, assessments by a clinical or caregiver, or clinical scales, described herein or known in the art.

In some embodiments, the disease or disorder is Alzheimer's disease. In some embodiments, the methods herein are used for the treatment of depression, anxiety, and/or stress associated with Alzheimer's disease. In some embodiments, the disease or disorder is Parkinson's disease. In some embodiments, the methods herein are used for the treatment of depression, anxiety, and/or stress associated with Parkinson's disease. In some embodiments, the disease or disorder is cancer related depression and anxiety. As discussed above, oral and/or extended-release dosing is appropriate for such applications, particularly when blood concentrations of active ingredient (e.g., a compound of Formula (I)) are kept below the psychedelic threshold.

In some embodiments, the disease or disorder is a neurological and developmental disorder such as autism spectrum disorder, including Asperger's syndrome. For example, Asperger's syndrome is a subtype of autism spectrum disorder that is treatable with anxiety drugs. Subjects with autism spectrum disorder may present with various signs and symptoms, including, but not limited to, a preference for non-social stimuli, aberrant non-verbal social behaviors, decreased attention to social stimuli, irritability, anxiety (e.g., generalized anxiety and social anxiety in particular), and depression. In some embodiments, the autism spectrum disorder comprises a medical diagnosis based on the criteria and classification from Diagnostic and Statistical Manual of Mental Disorders, 5th Ed (DSM-5). Current evidence supports the use of psychedelics for ameliorating behavior atypicalities of autism spectrum disorder, including reduced social behavior, anxiety, and depression (see Markopoulos A, Inserra A, De Gregorio D, Gobbi G. Evaluating the Potential Use of Serotonergic Psychedelics in Autism Spectrum Disorder. Front Pharmacol. 2022; 12:749068). The signs and symptoms of autism spectrum disorder may be treated with the methods herein.

In some embodiments, the disease or disorder is a genetic condition that causes learning disabilities and cognitive impairment. An example of such a genetic condition is fragile X syndrome, caused by changes in the gene Fragile X Messenger Ribonucleoprotein 1 (FMR1), which can cause mild to moderate intellectual disabilities in most males and about one-third of affected females. Fragile X syndrome and autism spectrum disorder are closely associated because the FMR1 gene is a leading genetic cause of autism spectrum disorder (see Markopoulos A, Inserra A, De Gregorio D, Gobbi G. Evaluating the Potential Use of Serotonergic Psychedelics in Autism Spectrum Disorder. Front Pharmacol. 2022; 12:749068). Subjects with fragile X syndrome may display anxiety, hyperactive behavior (e.g., fidgeting and impulsive actions), attention deficit disorder, mood and aggression abnormalities, poor recognition memory, and/or features of autism spectrum disorder, and these signs and symptoms may be treated with the methods herein. Clinical trials with psychedelics for the treatment of fragile X syndrome and autism spectrum disorder are currently ongoing (ClinicalTrials.gov, number NCT04869930).

In some embodiments, the disease or disorder is mental distress, e.g., mental distress in frontline healthcare workers.

In some embodiments, the disclosure provides for the management of different kinds of pain, including but not limited to cancer pain, e.g., refractory cancer pain; neuropathic pain; postoperative pain; opioid-induced hyperalgesia and opioid-related tolerance; neurologic pain; postoperative/post-surgical pain; complex regional pain syndrome (CRPS); shock; limb amputation; severe chemical or thermal burn injury; sprains, ligament tears, fractures, wounds and other tissue injuries; dental surgery, procedures and maladies; labor and delivery; during physical therapy; radiation poisoning; acquired immunodeficiency syndrome (AIDS); epidural (or peridural) fibrosis; orthopedic pain; back pain; failed back surgery and failed laminectomy; sciatica; painful sickle cell crisis; arthritis; autoimmune disease; intractable bladder pain; pain associated with certain viruses, e.g., shingles pain or herpes pain; acute nausea, e.g., pain that may be causing the nausea or the abdominal pain that frequently accompanies sever nausea; migraine, e.g., with aura; and other conditions including depression (e.g., acute depression or chronic depression), depression along with pain, alcohol dependence, acute agitation, refractory asthma, acute asthma (e.g., unrelated pain conditions can induce asthma), epilepsy, acute brain injury and stroke, Alzheimer's disease and other disorders. The pain may be persistent or chronic pain that lasts for weeks to years, in some cases even though the injury or illness that caused the pain has healed or gone away, and in some cases despite previous medication and/or treatment. In addition, the disclosure includes the treatment/management of any combination of these types of pain or conditions.

In some embodiments, the pain treated/managed is acute breakthrough pain or pain related to wind-up that can occur in a chronic pain condition. In some embodiments, the pain treated/managed is cancer pain, e.g., refractory cancer pain. In some embodiments, the pain treated/managed is post-surgical pain. In some embodiments, the pain treated/managed is orthopedic pain. In some embodiments, the pain treated/managed is back pain. In some embodiments, the pain treated/managed is neuropathic pain. In some embodiments, the pain treated/managed is dental pain. In some embodiments, the condition treated/managed is depression. In some embodiments, the pain treated/managed is chronic pain in opioid-tolerant patients.

In some embodiments, the disease or disorder includes conditions of the autonomic nervous system (ANS).

In some embodiments, the disease or disorder includes pulmonary disorders including asthma and chronic obstructive pulmonary disorder (COPD).

In some embodiments, the disease or disorder includes cardiovascular disorders including atherosclerosis.

The administering physician can provide a method of treatment that is prophylactic or therapeutic by adjusting the amount and timing of any of the compounds/salt forms described herein on the basis of observations of one or more symptoms of the disorder or condition being treated. In some embodiments, the subject is a mammal. In some embodiments, the mammal is a human.

In some embodiments, the compounds/compositions of the disclosure may be used as a standalone therapy. In some embodiments, the compounds/compositions of the disclosure may be used as an adjuvant/combination therapy. In some embodiments, the subject with a disorder is administered the compound/composition of the present disclosure and at least one additional therapy and/or therapeutic. In some embodiments, administration of an additional therapy and/or therapeutic is prior to administration of the compound/composition of the present disclosure. In some embodiments, administration of an additional therapy and/or therapeutic is after administration of the compound/composition of the present disclosure. In some embodiments, administration of an additional therapy and/or therapeutic is concurrent with administration of the compound/composition of the present disclosure. In some embodiments, the additional therapy is an antidepressant, an anticonvulsant, lisdexamfetamine dimesylate, an antipsychotic, an anxiolytic, an anti-inflammatory drug, a benzodiazepine, an analgesic drug, a cardiovascular drug, an opioid antagonist, or combinations thereof.

In some embodiments, the additional therapy is a benzodiazepine. In some embodiments, the benzodiazepine is diazepam or alprazolam.

In some embodiments, the additional therapy is a N-methyl-D-aspartate (NMDA) receptor antagonist. In some embodiments, the NMDA receptor antagonist is ketamine. In some embodiments, the NMDA receptor antagonist is nitrous oxide.

In some embodiments, the additional therapy is an antidepressant. In some embodiments, an antidepressant indirectly affects a neurotransmitter receptor, e.g., via interactions affecting the reactivity of other molecules at a neurotransmitter receptor. In some embodiments, an antidepressant is an agonist. In some embodiments, an antidepressant is an antagonist. In some embodiments, an antidepressant acts (either directly or indirectly) at more than one type of neurotransmitter receptor. In some embodiments, an antidepressant is chosen from buproprion, citalopram, duloxetine, escitalopram, fluoxetine, fluvoxamine, milnacipran, mirtazapine, paroxetine, reboxetine, sertraline, and venlafaxine.

In some embodiments, the antidepressant is a tricyclic antidepressant (“TCA”), selective serotonin reuptake inhibitor (“SSRI”), serototfin and noradrenaline reuptake inhibitor (“SNRI”), dopamine reuptake inhibitor (“DRI”), noradrenaline reuptake Monoamine oxidase inhibitor (“MAOI”), including inhibitor (“NRU”), dopamine, serotonin and noradrenaline reuptake inhibitor (“DSNRI”), a reversible inhibitor of monoamine oxidase type A (RIMA), or combination thereof. In some embodiments, the antidepressant is a TCA. In some embodiments, the TCA is imipramine or clomipramine. In some embodiments, the antidepressant is an SRI. In some embodiments, the SSRI is escitalopram, paroxetine, sertraline, fluvoxamine, fluoxetine, or combinations thereof. In some embodiments, the SNRI is venlafaxine. In some embodiments, the additional therapy is pregabalin.

In some embodiments, the additional therapeutic is an anticonvulsant. In some embodiments, the anticonvulsant is gabapentin, carbamazepine, ethosuximide, lamotrigin, felbamate, topiramate, zonisamide, tiagabine, oxcarbazepine, levetiracetam, divalproex sodium, phenytoin, fosphenytoin. In some embodiments, the anticonvulsant is topiramate.

In some embodiments, the additional therapeutic is an antipsychotic. In some embodiments, the antipsychotic is a phenothiazine, butryophenone, thioxanthene, clozapine, risperidone, olanzapine, or sertindole, quetiapine, aripiprazole, zotepine, perospirone, a neurokinin-3 antagonist, such as osanetant and talnetant, rimonabant, or a combination thereof.

In some embodiments, the additional therapeutic is an anti-inflammatory drug. In some embodiments, the anti-inflammatory drug is a nonsteroidal anti-inflammatory drugs (NSAIDS), steroid, acetaminophen (COX-3 inhibitors), 5-lipoxygenase inhibitor, leukotriene receptor antagonist, leukotriene A4 hydrolase inhibitor, angiotensin converting enzyme antagonist, beta blocker, antihistaminic, histamine 2 receptor antagonist, phosphodiesterase-4 antagonist, cytokine antagonist, CD44 antagonist, antineoplastic. agent, 3-hydroxy-3-methylglutaryl coenzyme A inhibitor (statins), estrogen, androgen, antiplatelet agent, antidepressant, Helicobacter pylori inhibitors, proton pump inhibitor, thiazoliditledione, dual-action compounds, or combination thereof.

In some embodiments, the additional therapeutic is an anti-anxiolytic. In some embodiments, an anxiolytic is chosen from alprazolam, an alpha blocker, an antihistamine, a barbiturate, a beta blocker, bromazepam, a carbamate, chlordiazepoxide, clonazepam, clorazepate, diazepam, flurazepam, lorazepam, an opioid, oxazepam, temazepam, or triazolam.

In some embodiments, the additional therapy is an opioid antagonist. Non-limiting examples of opioid antagonists include naloxone, naltrexone, nalmefene, nalorphine, nalrphine dinicotinate, levallrphan, samidorphan, nalodeine, alvimopan, methylnaltrexone, naloxegol, 6-naltrexol, axelopran, bevenopran, methylsamidorphan, naldemedine, buprenorphine, decozine, butorphanol, levorphanol, nalbuphine, pentazocine, and phenazocine.

In some embodiments, the additional therapy is a cardiovascular drug. Non-limiting examples of cardiovascular drugs include digoxin or (3β,5β,12β)-3-[(O-2,6-dideoxy-β-D-ribo-hexopyranosyl-(1→4)-O-2,6-dideoxy-β-D-ribo-hexopyranosyl-(1→4)-2,6-dideoxy-β-D-ribohexopyranosyl)oxy]-12,14-dihydroxy-card-20(22)-enolide, lisinopril, captopril, ramipril, trandolapril, benazepril, cilazapril, enalapril, moexipril, perindopril, quinapril, fludrocortisone, enalaprilate, quinapril, perindopril, apixaban, dabigatran, edoxaban, heparin, rivaroxaban, warfarin, aspirin, clopidogrel, dipyridamole, prasugrel, ticagrelor, azilsartan, candesartan, eprosartan, irbesartan, losartan, olmesartan, telmisartan, valsartanscaubitril, acebutolol, atenolol, betaxolol, bisoprolol, metoprolol, nadolol, propranolol, sotalol, amlodipine, diltiazem, felodipine, nifedipine, nimodipine, nisolidipine, verapamil, statins, nicotinic acids, diuretics, vasodilators, and combinations thereof.

In some embodiments, the subject is administered at least one therapy. Non-limiting examples of therapies include transcranial magnetic stimulation, cognitive behavioral therapy, interpersonal psychotherapy, dialectical behavior therapy, mindfulness techniques, or acceptance, commitment therapy, or combinations thereof.

Also disclosed herein is a method for decreasing time of therapeutic onset relative to a psilocybin-based drug comprising administering a therapeutically effective amount of a compound as disclosed herein (e.g., the compound of Formula (I), or a pharmaceutically acceptable salt, polymorph, stereoisomer, or solvate thereof) to a patient in need thereof.

Also disclosed herein is a method of reducing psychedelic side effects relative to a psilocybin-based drug comprising administering a therapeutically effective amount of a compound as disclosed herein (e.g., the compound of Formula (I), or a pharmaceutically acceptable salt, polymorph, stereoisomer, or solvate thereof) to a patient in need thereof.

The terms “hallucinogenic side effects” and “psychedelic side effects” are used in the present disclosure interchangeably to refer to unwanted and/or unintended secondary effects caused by the administration of a medicament to an individual resulting in subjective experiences being qualitatively different from those of ordinary consciousness. These experiences can include derealization, depersonalization, hallucinations and/or sensory distortions in the visual, auditory, olfactory, tactile, proprioceptive and/or iriteroceptive spheres and/or any other perceptual modifications, and/or any other substantial subjective changes in cognition, memory, emotion and consciousness.

In some embodiments, the administration of the compound of Formula (I), or a pharmaceutically acceptable salt, polymorph, stereoisomer, or solvate thereof causes no hallucinogenic and/or psychedelic side effects and/or less hallucinogenic and/or psychedelic side effects relative to a psilocybin-based drug. In some embodiments, the administration of the compound of Formula (I), or a pharmaceutically acceptable salt, polymorph, stereoisomer, or solvate thereof, alleviates, reduces, removes, and/or eliminates the hallucinogenic and/or psychedelic side effects caused by a psilocybin-based drug.

Also disclosed herein is a method of reducing dose related side-effects, e.g., nausea, relative to treatment with a psilocybin-based drug, comprising administering a therapeutically effective amount of a compound as disclosed herein (e.g., the compound of Formula (I), or a pharmaceutically acceptable salt, polymorph, stereoisomer, or solvate thereof) to a subject in need thereof. The compound of Formula (I), or a pharmaceutically acceptable salt, polymorph, stereoisomer, or solvate thereof has better brain penetration (i.e., a higher brain:plasma ratio) than that obtained from administration of psilocybin. As a result, the effective dosing for the compounds of the present disclosure can be lowered, thereby reducing dose related side effects such as nausea.

Also disclosed herein is a method of decreasing duration of therapeutic effect relative to a psilocybin-based drug comprising administering a therapeutically effective amount of a compound as disclosed herein (e.g., the compound of Formula (I), or a pharmaceutically acceptable salt, polymorph, stereoisomer, or solvate thereof) to a patient in need thereof.

Generally, a duration of therapeutic effect for a psilocybin-based drug is about 6-8 hours. In some embodiments, the duration of therapeutic effect of the compound of Formula (I) is less than the duration of therapeutic effect for a psilocybin-based drug. In some embodiments, the duration of therapeutic effect of the compound of Formula (I) is 7 hours, 6 hours, 5 hours, 4 hours, 3 hours, 2 hours, 1 hour or less, or 50 minutes, 40 minutes, 30 minutes, 20 minutes, 10 minutes, 5 minutes or less. In some embodiments, the duration of therapeutic effect of the compound of Formula (I) is less than the duration of therapeutic effect of a psilocybin-based drug by 7 hours, 6 hours, 5 hours, 4 hours, 3 hours, 2 hours, 1 hour or less, or 50 minutes, 40 minutes, 30 minutes, 20 minutes, 10 minutes, 5 minutes or less.

### EXAMPLES

### I. Analytical Methods

### Differential Scanning Calorimetry (DSC)

DSC data were collected on a Mettler DSC 3+ equipped with a 34 position auto-sampler. The instrument was calibrated for energy and temperature using certified indium. Typically 0.5-3 mg of each sample, in a pin-holed aluminum pan, was heated at 10° C.·min⁻¹ from 30° C. to 300° C. A nitrogen purge at 50 mL·min⁻¹ was maintained over the sample. STARe v15.00 was used for instrument control and data processing.

### X-Ray Powder Diffraction (XRPD)

X-Ray Powder Diffraction patterns were collected on a Bruker AXS D2 diffractometer using CuKα radiation (30 kV, 10 mA), 0-0 geometry, using a LynxEye detector from 5-42° 2θ.

The software used for data collection was DIFFRAC.SUITE and the data were analysed and presented using DIFFRAC EVA v 5.

The details of the data collection are:
Angular range: 5 to 42° 2θ
Step size: 0.024° 2θ
Collection time: 0.1 seconds per step.

Samples were run under ambient conditions and prepared as flat plate specimens using powder as received without grinding. Approximately 1-2 mg of the sample was lightly pressed on a silicon wafer to obtain a flat surface.

### Gravimetric Vapor Sorption (GVS)/Dynamic Vapor Sorption (DVS)

Sorption isotherms were obtained using a SMS DVS Intrinsic moisture sorption analyzer, controlled by SMS Analysis Suite software. The sample temperature was maintained at 25° C. throughout. The humidity was controlled by mixing streams of dry and wet nitrogen, with a total flow rate of 200 mL·min⁻¹. Relative humidity (RH) was measured by a calibrated Rotronic probe (dynamic range of 1.0-100% RH), located near the sample. Weight change (mass relaxation) of the sample as a function of % RH was constantly monitored by the microbalance (accuracy 0.005 mg).

5-20 mg of sample was placed in a pre-tared stainless steel mesh basket under ambient conditions. The sample was loaded and unloaded at 40% RH and 25° C. (typical room conditions). A moisture sorption isotherm was performed as outlined below. The standard isotherm was performed at 25° C. using 10% RH intervals over a 0-90% RH range. The sample was recovered after completion of the isotherm and in some cases re-analyzed by XRPD. Parameters used during GVS/DVS acquisition are presented in Table 4.

TABLE 4

| Parameter | Value |
|---|---|
| RH Profile - Cycle 1 | 40-90, 90-0, 0-40 |
| RH Profile - Cycle 2 | 40-90, 90-0, 0-40 |
| Intervals (% RH) | 10 |
| Flow rate (mL · min⁻¹) | 200 |
| Temperature (° C.) | 25 |
| Stability (° C. · min⁻¹) | 0.2 |
| Sorption Time (hours) | 6 hour time out |

### Nuclear Magnetic Resonance (NMR)

Solution phase ¹H NMR Spectra were obtained using a Bruker AVIIIHD NMR spectrometer, fitted with a 5 mm PABBO probe operating at 400.1326 MHz. Samples were prepared in d₆-DMSO, unless otherwise stated and referenced using a TMS internal standard.

### Thermogravimetric Analysis (TGA)

TGA data were collected on a Mettler TGA 2 equipped with a 34 position auto-sampler. The instrument was temperature calibrated using certified isotherm and nickel. Typically 5-30 mg of each sample was loaded into a pin-holed aluminum pan and heated at 10° C.·min⁻¹ from 30° C. to 400° C. A nitrogen purge at 50 mL·min⁻¹ was maintained over the sample. STARe v15.00 was used for instrument control and data processing.

### Ultra Performance Liquid Chromatography (UPLC)

Purity analysis was performed on a Waters Acquity system equipped with a diode array detector and MicroMass ZQ mass spectrometer using MassLynx software. The UPLC method parameters used for chemical purity analysis are presented in Table 5.

TABLE 5

| Sample Preparation: | 0.2-0.5 mg · mL⁻¹ in DMSO | | |
|---|---|---|---|
| Column: | BEH C18 1.7 μm 100 × 2.1 mm | | |
| Column Temperature (° C.): | 40 | | |
| Injection (μL): | 2 | | |
| Detection: | UV Diode array 200-500 nm | | |
| Wavelength, Bandwidth (nm): | | | |
| Phase A: | 0.1% formic acid in water | | |
| Phase B: | 0.1% formic acid in acetonitrile | | |
| Flow Rate (mL · min⁻¹) | 0.4 | | |
| | Time (min) | % Phase A | % Phase B |
| Timetable: | 0 | 97 | 3 |
| | 0.4 | 97 | 3 |
| | 6.5 | 55 | 45 |
| | 7.0 | 55 | 45 |
| | 7.5 | 95 | 5 |
| | 8.0 | 95 | 5 |

### High Resolution Mass Spectrometry (HRMS) and MS/MS

Samples were dissolved in acetonitrile:water (50:50) at 2 mg/mL and examined by LC-MS under the following conditions listed in Tables 6-8.

TABLE 6

| Instrumenta- | Waters Xevo G2 QTOF with Acquity LC system | | | | |
|---|---|---|---|---|---|
| tion: | | | | | |
| Injection | 1 | | | | |
| Volume (μL): | | | | | |
| Column: | Acquity UPLC HSS T3 1.8 μm 50 × 2.1 mm | | | | |
| Mobile Phase: | A) HPLC Grade Water + 0.1% Formic Acid | | | | |
| | B) Acetonitrile + 0.1% Formic Acid | | | | |
| | Time | Flow | | | |
| | (min) | (mL/min) | % A | % B | Curve |
| Mobile Phase | Initial | 0.600 | 98.0 | 2.0 | |
| Gradient: | 1.00 | 0.600 | 98.0 | 2.0 | 6 |
| | 8.00 | 0.600 | 2.0 | 98.0 | 6 |
| | 9.00 | 0.600 | 2.0 | 98.0 | 6 |
| | 9.10 | 0.600 | 98.0 | 2.0 | 6 |
| | 10.00 | 0.600 | 98.0 | 2.0 | 6 |

TABLE 7

| Tune Method | Ionisation Polarity | ESI+ | |
|---|---|---|---|
| | Analyser Mode | Resolution mode | |
| | Capillary | 0.7 | kV |
| | Sampling Cone | 35 | V |
| | Extraction Cone | 4 | V |
| | Source temperature | 100° | C. |
| | Desolvation temperature | 500° | C. |
| | Cone Gas Flow | 50 | L · hr⁻¹ |
| | Desolvation Gas Flow | 900 | L · hr⁻¹ |
| Lockspray | Analyte | Leucine Enkephalin | |
| | Capillary | 3 | kV |

| | |
|---|---|
| Collision Energy | OFF |

TABLE 8
Scanning Conditions

| | |
|---|---|
| Start Mass (Da): | 50 |
| End Mass (Da): | 1200 |
| Scan Time (s): | 0.1 |
| Set Mass (Da): | [M + H]⁺ |
| Collision Energy (V): Ramp: | 10-30 |

### II. Compounds and Salt Forms

### Example 1 (Free Base)

### 3-(2-(bis(methyl-d₃)amino)ethyl-1,1,2,2-d₄)-1H-indol-4-ol (I-3; psilocin-d₁₀; PI-d₁₀)

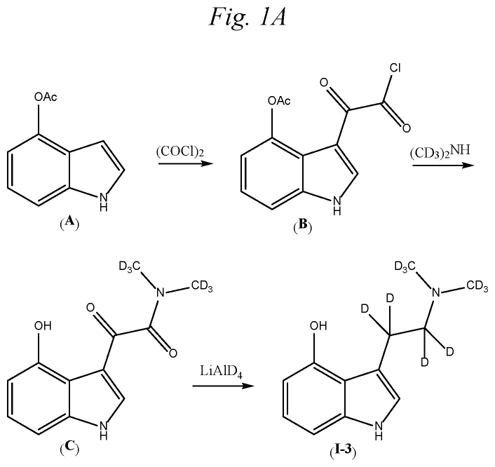

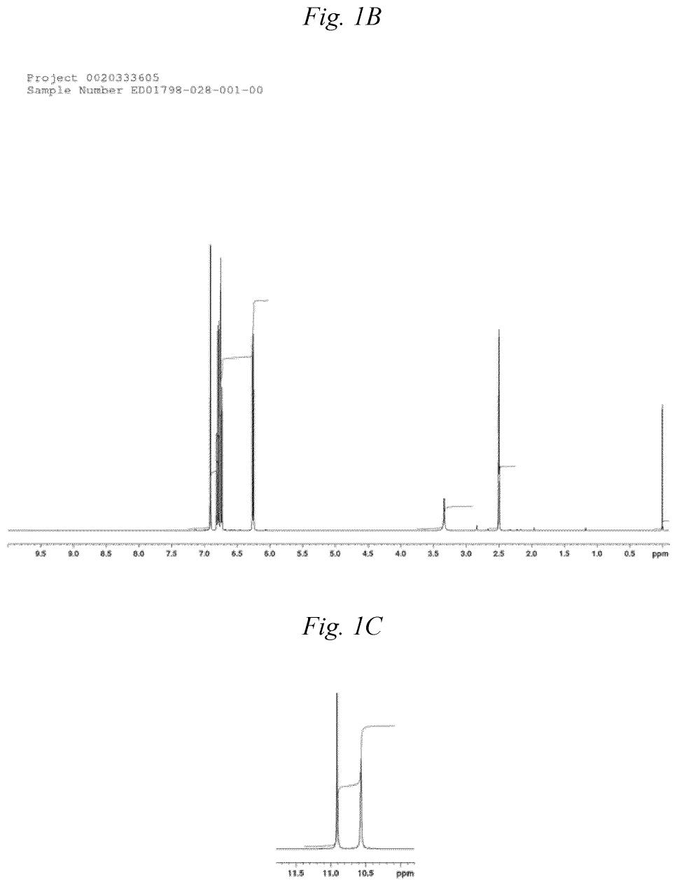

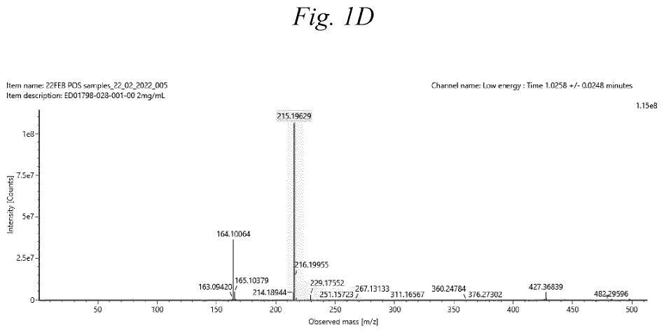

Compound 3-(2-(bis(methyl-d₃)amino)ethyl-1,1,2,2-d₄)-1H-indol-4-ol (I-3; PI-d₁₀) was synthesized according to FIG. 1A. 4-acetoxyindole was acylated using oxalyl chloride producing intermediate B as a yellow solid. Treatment of intermediate B with dimethyl-d-amine (Cambridge Isotopes Labs, Tewksbury, MA) resulted in amidation and de-acetylation to form intermediate C, which was then reduced by LiAlD₄ to form compound I-3 (free base).

The structure of the final product with deuterium enrichment over 90% was confirmed by ¹H NMR (FIGS. 1B-1C) and HRMS (FIG. 1D). The UPLC purity was 99.4%. The tentative structure of molecular ion observed in HRMS is presented in Table 9.

TABLE 9

| Com- | Ion | Proposed | Calculated | Tentative | Error |
|---|---|---|---|---|---|
| pound | Observed | Formula | Mass | Structure | (mDa) |
| Psilocin- | 215.19629 | C₁₂H₇D₁₀N₂O⁺ | 215.1963 | [M + H]⁺ | −0.01 |
| d₁₀ | | | | | |

X-ray powder diffraction (XRPD) pattern of I-3 indicates the material is crystalline, with diffraction peaks of pattern 1 (FIG. 2A). FIGS. 2B and 2C show the zoomed in and annotated XRPD patterns of I-3. Table 10 shows the XRPD peak listing for I-3 (pattern 1).

TABLE 10

| Name | Caption (display) | Angle | d Value | Net Intensity | Rel. Intensity |
|---|---|---|---|---|---|
| Peak #1 | 7.582° | 7.582295 | 11.65008 | 861.3094 | 0.007792 |
| Peak #2 | 8.395° | 8.394759 | 10.52429 | 110533.8 | 1.000000 |
| Peak #3 | 9.647° | 9.646576 | 9.161201 | 269.5037 | 0.002438 |
| Peak #4 | 10.444° | 10.44383 | 8.46358 | 84.23746 | 0.000762 |
| Peak #5 | 11.319° | 11.3193 | 7.81087 | 32.57419 | 0.000295 |
| Peak #6 | 12.614° | 12.61394 | 7.011955 | 432.8757 | 0.003916 |
| Peak #7 | 13.372° | 13.3719 | 6.616152 | 84.5412 | 0.000765 |
| Peak #8 | 14.222° | 14.22235 | 6.222384 | 21.97205 | 0.000199 |
| Peak #9 | 15.157° | 15.15702 | 5.840713 | 744.3998 | 0.006735 |
| Peak #10 | 16.524° | 16.52404 | 5.360464 | 654.2328 | 0.005919 |
| Peak #11 | 16.787° | 16.78656 | 5.277219 | 74073.52 | 0.670143 |
| Peak #12 | 17.693° | 17.69319 | 5.008796 | 284.0384 | 0.002570 |
| Peak #13 | 19.468° | 19.46828 | 4.555921 | 423.9552 | 0.003836 |
| Peak #14 | 19.699° | 19.69928 | 4.503015 | 1017.259 | 0.009203 |
| Peak #15 | 20.901° | 20.90102 | 4.246741 | 389.4514 | 0.003523 |
| Peak #16 | 21.132° | 21.13205 | 4.200831 | 679.8422 | 0.006151 |
| Peak #17 | 21.859° | 21.85903 | 4.062737 | 85.95354 | 0.000778 |
| Peak #18 | 22.547° | 22.54694 | 3.940314 | 67.01072 | 0.000606 |
| Peak #19 | 23.699° | 23.6988 | 3.751339 | 942.5151 | 0.008527 |
| Peak #20 | 24.630° | 24.62965 | 3.611631 | 30.55703 | 0.000276 |
| Peak #21 | 25.034° | 25.03401 | 3.554202 | 51.79423 | 0.000469 |
| Peak #22 | 25.264° | 25.2645 | 3.522297 | 638.7632 | 0.005779 |
| Peak #23 | 26.867° | 26.86674 | 3.315764 | 360.7947 | 0.003264 |
| Peak #24 | 27.399° | 27.39855 | 3.252601 | 28.91455 | 0.000262 |
| Peak #25 | 27.929° | 27.92929 | 3.191985 | 54.06995 | 0.000489 |
| Peak #26 | 28.219° | 28.21871 | 3.159902 | 20.70195 | 0.000187 |
| Peak #27 | 28.871° | 28.87078 | 3.089999 | 43.83303 | 0.000397 |
| Peak #28 | 29.430° | 29.42954 | 3.03259 | 73.03846 | 0.000661 |
| Peak #29 | 30.120° | 30.11959 | 2.964665 | 34.19807 | 0.000309 |
| Peak #30 | 30.675° | 30.67536 | 2.912207 | 207.2589 | 0.001875 |
| Peak #31 | 31.373° | 31.37339 | 2.848986 | 91.50857 | 0.000828 |
| Peak #32 | 32.365° | 32.36467 | 2.763953 | 54.7266 | 0.000495 |
| Peak #33 | 33.880° | 33.88012 | 2.643708 | 132.8692 | 0.001202 |
| Peak #34 | 34.418° | 34.41825 | 2.603594 | 19.10025 | 0.000173 |
| Peak #35 | 34.792° | 34.7921 | 2.576469 | 20.48885 | 0.000185 |
| Peak #36 | 35.884° | 35.88435 | 2.500512 | 136.4738 | 0.001235 |
| Peak #37 | 36.254° | 36.25361 | 2.475885 | 28.93899 | 0.000262 |
| Peak #38 | 37.156° | 37.15607 | 2.417796 | 31.10832 | 0.000281 |
| Peak #39 | 38.200° | 38.20009 | 2.354083 | 34.23606 | 0.000310 |
| Peak #40 | 38.417° | 38.41659 | 2.341313 | 52.08007 | 0.000471 |

### 3-(2-(dimethylamino)ethyl)-1H-indol-4-ol (I-7/psilocin/psilocin-d₀/PI-d₀)

Compound I-7 (PI-d₀ free base) used in the below examples was characterized by X-ray powder diffraction (XRPD) as having an XRPD pattern of pattern 1 (see FIG. 3C). Table 11 shows the XRPD peak listing for I-7 (pattern 1).

TABLE 11

| Caption (display) | Angle | d Value | Net Intensity | Rel. Intensity |
|---|---|---|---|---|
| 7.563° | 7.563431 | 11.67909 | 44.11061 | 0.00672 |
| 8.375° | 8.374504 | 10.5497 | 6563.628 | 1 |
| 12.626° | 12.62642 | 7.005054 | 208.3727 | 0.031747 |
| 13.383° | 13.38263 | 6.610871 | 185.4812 | 0.028259 |
| 15.211° | 15.21114 | 5.820052 | 265.2301 | 0.040409 |
| 16.753° | 16.75303 | 5.287707 | 3690.068 | 0.562199 |
| 17.671° | 17.67111 | 5.015004 | 304.343 | 0.046368 |
| 19.668° | 19.66817 | 4.510068 | 2602.091 | 0.396441 |
| 21.112° | 21.11224 | 4.204728 | 864.1647 | 0.13166 |
| 21.863° | 21.86267 | 4.062068 | 148.0073 | 0.02255 |
| 22.201° | 22.20094 | 4.000934 | 80.56883 | 0.012275 |
| 22.560° | 22.56038 | 3.937997 | 103.738 | 0.015805 |
| 23.711° | 23.71067 | 3.749489 | 1043.58 | 0.158994 |
| 24.592° | 24.59193 | 3.617084 | 48.51187 | 0.007391 |
| 25.415° | 25.41542 | 3.501722 | 60.87042 | 0.009274 |
| 26.820° | 26.81979 | 3.321463 | 226.2585 | 0.034472 |
| 27.357° | 27.35734 | 3.257406 | 63.70338 | 0.009706 |
| 27.921° | 27.92109 | 3.192905 | 111.1571 | 0.016935 |
| 28.228° | 28.22797 | 3.158886 | 18.08015 | 0.002755 |
| 29.253° | 29.25296 | 3.050493 | 70.03584 | 0.01067 |
| 30.653° | 30.65323 | 2.914259 | 174.6236 | 0.026605 |
| 31.364° | 31.3638 | 2.849835 | 103.4405 | 0.01576 |
| 32.401° | 32.40136 | 2.760907 | 58.739 | 0.008949 |
| 33.797° | 33.79747 | 2.649983 | 17.68047 | 0.002694 |
| 34.445° | 34.44516 | 2.601622 | 29.53493 | 0.0045 |
| 39.867° | 39.86696 | 2.259415 | 40.48755 | 0.006168 |

### Example 2

### Synthesis of benzenesulfonate salt of 3-(2-(dimethylamino)ethyl)-1H-indol-4-ol (I-7a)(benzenesulfonate salt of I-7/psilocin/psilocin-d₀/PI-d₀)

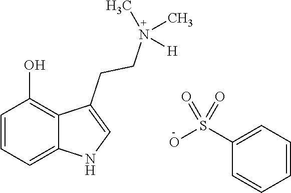

Compound I-7 (PI-d₀, free base) (10 mM) was dissolved in acetonitrile and treated with a solution of benzenesulfonic acid (10 mM) in THF while being stirred at room temperature. Samples were shaken overnight at room temperature, then refrigerated for 11 days. Sample still remained as a solution and so TBME anti-solvent was added and was refrigerated overnight to produce I-7a (mono-benzenesulfonate salt of PI-d₀).

As shown in FIG. 3A, the X-ray powder diffraction (XRPD) pattern of I-7a indicates one crystalline form was produced (pattern 1) having high crystallinity. FIG. 3B shows the zoomed in and annotated XRPD patterns of I-7a. FIG. 3C shows the XRPD pattern of I-7 (PI-d₀, free base) (pattern 1), and FIG. 3D shows a comparison between the XRPD patterns of I-7a (benzenesulfonate salt) and I-7 (PI-d₀, free base) (pattern 1). Table 12 shows the XRPD peak listing for I-7a (pattern 1).

TABLE 12

| Name | Caption (display) | Angle | d Value | Net Intensity | Rel. Intensity |
|---|---|---|---|---|---|
| Peak #1 | 7.002° | 7.00186 | 12.61448 | 24.92258 | 0.001100 |
| Peak #2 | 7.733° | 7.732948 | 11.42345 | 1731.93 | 0.076457 |
| Peak #3 | 11.768° | 11.76836 | 7.513811 | 77.21395 | 0.003409 |
| Peak #4 | 12.516° | 12.51581 | 7.066712 | 92.23495 | 0.004072 |
| Peak #5 | 12.882° | 12.88182 | 6.866737 | 12.68317 | 0.000560 |
| Peak #6 | 13.546° | 13.54551 | 6.531741 | 57.04026 | 0.002518 |
| Peak #7 | 13.968° | 13.96827 | 6.334989 | 229.514 | 0.010132 |
| Peak #8 | 14.788° | 14.78807 | 5.985592 | 86.10961 | 0.003801 |
| Peak #9 | 15.225° | 15.22511 | 5.814743 | 194.4308 | 0.008583 |
| Peak #10 | 15.474° | 15.47401 | 5.721769 | 22652.34 | 1.000000 |
| Peak #11 | 18.370° | 18.37028 | 4.825681 | 815.8047 | 0.036014 |
| Peak #12 | 19.737° | 19.73692 | 4.494512 | 37.58994 | 0.001659 |
| Peak #13 | 20.703° | 20.7033 | 4.286849 | 116.4809 | 0.005142 |
| Peak #14 | 21.050° | 21.04972 | 4.217076 | 68.59189 | 0.003028 |
| Peak #15 | 21.873° | 21.87293 | 4.060185 | 33.76218 | 0.001490 |
| Peak #16 | 21.982° | 21.98196 | 4.040294 | 270.1723 | 0.011927 |
| Peak #17 | 22.315° | 22.31468 | 3.980798 | 55.64486 | 0.002456 |
| Peak #18 | 22.639° | 22.63877 | 3.924538 | 158.6271 | 0.007003 |
| Peak #19 | 23.282° | 23.28249 | 3.817467 | 2953.931 | 0.130403 |
| Peak #20 | 23.775° | 23.77462 | 3.739549 | 179.0302 | 0.007903 |
| Peak #21 | 24.125° | 24.12521 | 3.685993 | 150.6518 | 0.006651 |
| Peak #22 | 25.193° | 25.19252 | 3.532198 | 48.53237 | 0.002142 |
| Peak #23 | 25.475° | 25.47532 | 3.493623 | 141.9634 | 0.006267 |
| Peak #24 | 25.931° | 25.93118 | 3.433232 | 49.37065 | 0.002179 |
| Peak #25 | 26.813° | 26.81276 | 3.322318 | 61.6441 | 0.002721 |
| Peak #26 | 27.778° | 27.77834 | 3.208987 | 27.04133 | 0.001194 |
| Peak #27 | 28.127° | 28.12704 | 3.169992 | 28.03019 | 0.001237 |
| Peak #28 | 30.866° | 30.86559 | 2.89469 | 52.91717 | 0.002336 |
| Peak #29 | 31.207° | 31.20687 | 2.863808 | 2039.447 | 0.090033 |
| Peak #30 | 32.941° | 32.94133 | 2.716873 | 58.67304 | 0.002590 |
| Peak #31 | 33.222° | 33.22205 | 2.694555 | 36.2762 | 0.001601 |
| Peak #32 | 33.698° | 33.69811 | 2.657569 | 31.60854 | 0.001395 |
| Peak #33 | 36.803° | 36.80291 | 2.440182 | 40.07982 | 0.001769 |
| Peak #34 | 38.668° | 38.66787 | 2.326675 | 22.73825 | 0.001004 |
| Peak #35 | 39.289° | 39.28912 | 2.291304 | 227.1359 | 0.010027 |

The DSC curve of I-7a is shown in FIG. 4 and it is evident that the salt has a high melt onset (159.10° C.) and peak (161.68° C.). Equally, no events were observed prior to the melt endotherm, indicating the absence of multiple physical forms in the sample, and also no conversion of physical forms prior to the melt.

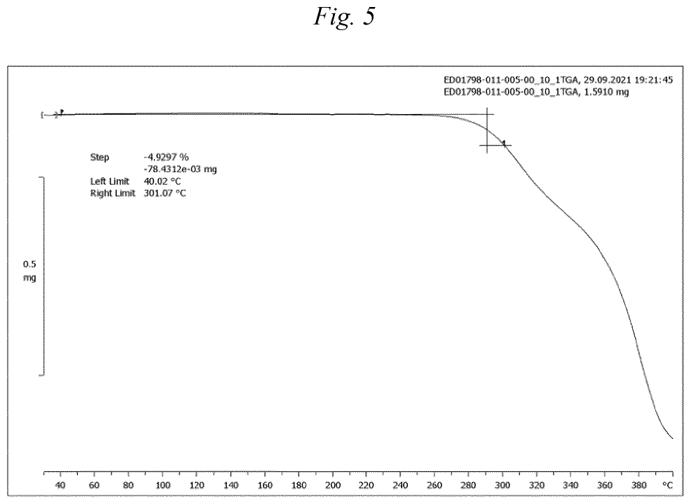

The thermogravimetric analysis (TGA) curve of I-7a is shown in FIG. 5 showed 95% mass remaining at 301° C. at a heating rate of 10° C./min.

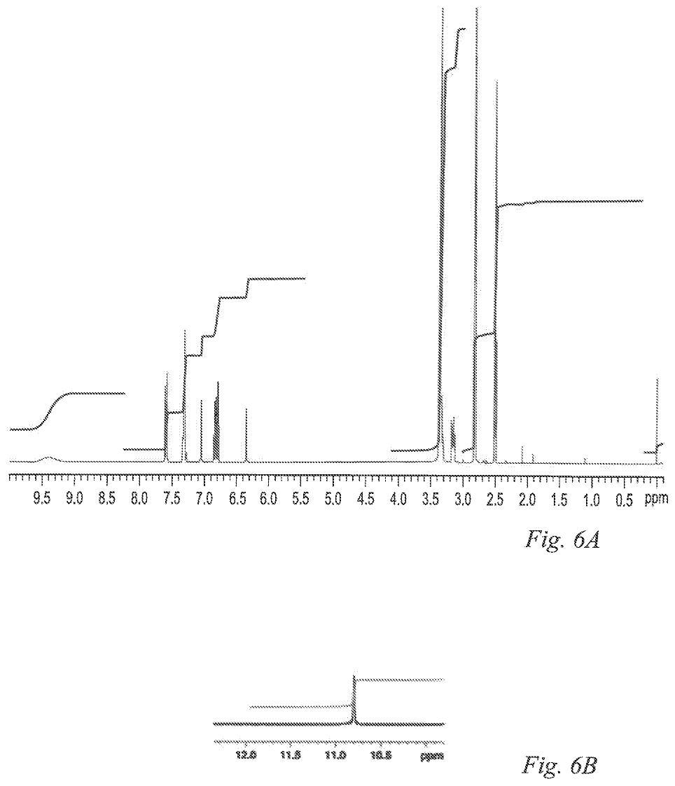

FIGS. 6A and 6B show a ¹H NMR spectrum of I-7a, which indicates protonation and 1 molar equivalent of benzenesulfonate (trace MeCN, 0.02 equivalents).

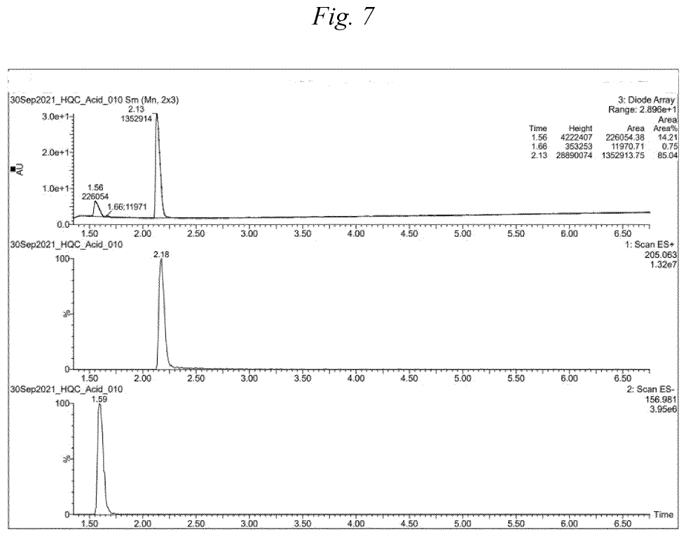

The UPLC chromatogram of I-7a (FIG. 7) indicates a purity including counterion of 99.2%.

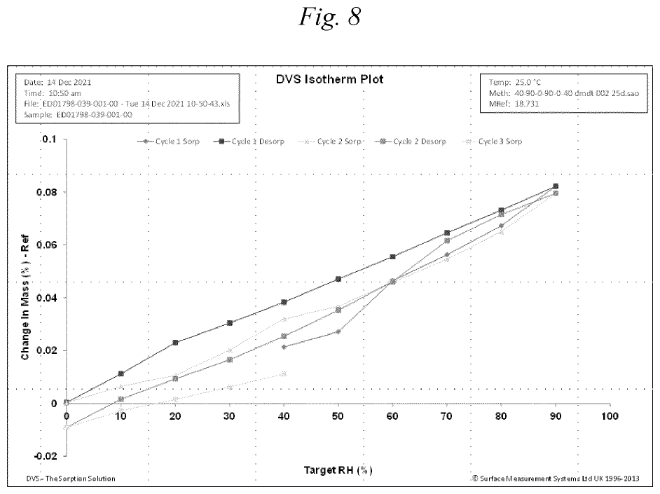

DVS isotherm plot of I-7a is shown in FIG. 8. There is no significant hygroscopicity and the acquisition of water was very low, even on exposure to elevated relative humidities (RH) of >90% RH, with a 0.08% mass increase over 0-90% RH range (second sorption cycle). The change in mass was low over the typical range of ambient relative humidities, and there was no evidence of physical form conversion throughout the cycle. This isotherm plot represents advantageous behavior for pharmaceutical development purposes.

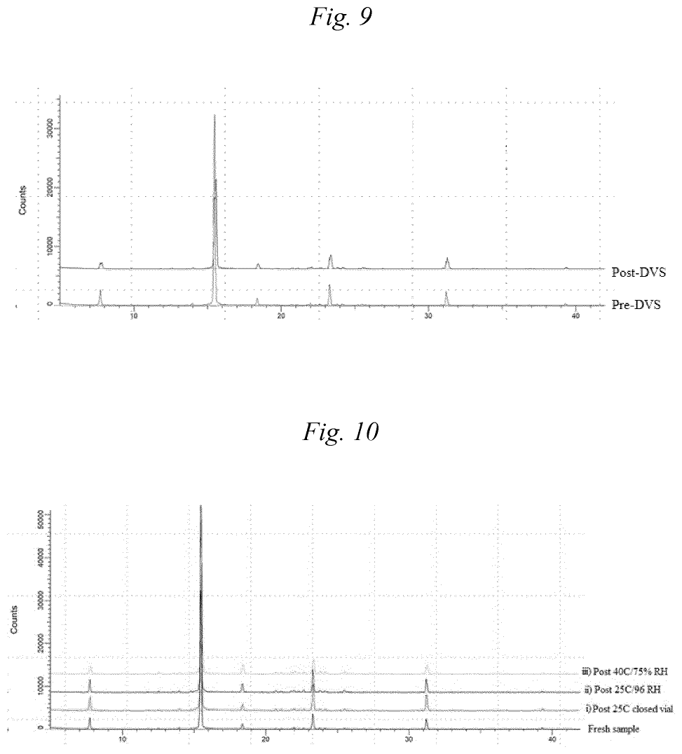

FIG. 9 shows the XRPD pattern of I-7a after being subjected to the DVS conditions above (post-DVS) compared to the XRPD pattern before DVS (Pre-DVS), indicating that no changes to the crystal structure took place. There was also no loss of purity following DVS analysis according to ¹H NMR and UPLC.

The storage stability of I-7a was also assessed by storing the solid samples for 22 days under the following conditions: i) 25° C., closed vial, ii) 25° C./96% RH, and iii) 40° C./75% RH, and comparing to fresh sample by XRPD. The results are presented in FIG. 10, which showed no change in form by XRPD post storage. There was also no loss of purity post storage for any of i) to iii) according to ¹H NMR and UPLC.

The benzenesulfonate salt (I-7a) was also subjected to maturation in 12 different solvents Briefly, 12 portions of I-7a (each ca. 10 mg) were weighed into amber glass vials and treated with the following 12 solvents: TBME, acetone, chloroform, THF, ethyl acetate, ethanol, acetonitrile, heptane, water, toluene, 2-methoxyethanol, and benzyl alcohol. The resulting slurries were subjected to maturation with thermal cycling between room temperature and 50° C. (4 hours at each condition) for 3 days. Solids were then analysed by XRPD. All samples were isolated as solids and retained their initial crystalline form (pattern 1) according to XRPD analysis (FIG. 11).

Seeded. Compound I-7 (PI-d₀, free base) (501.8 mg) was dissolved in acetonitrile (25 mL) and treated with a solution of benzenesulfonic acid (1 equivalent) in THF (2.45 mL). The resulting solution was stirred, treated with seeds of I-7a (pattern 1) and TBME (15 mL), followed by additional seed. A precipitate began to form. The mixture was then refrigerated for 11 days. The precipitate was isolated by filtration under a stream of nitrogen (inverted funnel) and dried in vacuo to give I-7a as a solid (84.8% yield). The isolated solid was crystalline and of the same pattern (pattern 1) as obtained in the non-seeded preparation. ¹H NMR was consistent with 1 eq. of benzenesulfonate counterion. The crystalline I-7a material obtained from the seeded preparation had >99.5% purity (including counterion) by UPLC, and showed the expected M+H⁺ ion (205.1) and for the counterion M−H⁻ (156.9).

### Example 3

### Synthesis of tartrate salt of 3-(2-(dimethylamino)ethyl)-1H-indol-4-ol (I-7b)(Tartrate Salt of I-7/psilocin/psilocin-d₀/PI-d₀)

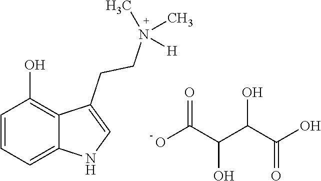

L-tartaric acid salt (1 eq). Compound I-7 (PI-d₀, free base) (10 mM) was dissolved in 1,4-dioxane, acetonitrile, or THF and treated with a solution of L-tartaric acid (10 mM) in THF while being stirred at room temperature. Samples were shaken overnight at room temperature to produce I-7b (as a mono-L-tartrate salt of PI-d₀).

As shown in FIG. 12, the X-ray powder diffraction (XRPD) pattern indicates that two different crystalline polymorphs of I-7b wee formed: a polymorph having pattern 1 (made from acetonitrile or THF), and a polymorph having pattern 2 (made from 1,4-dioxane). The XRPD peak listing of I-7b (pattern 1) is provided in Table 13.

TABLE 13

| Caption (display) | Angle | d Value | Net Intensity | Rel. Intensity |
|---|---|---|---|---|
| 6.798° | 6.798303 | 12.99172 | 81.18942 | 0.038823 |
| 11.360° | 11.36046 | 7.782664 | 38.20749 | 0.01827 |
| 12.764° | 12.76448 | 6.929597 | 187.3042 | 0.089565 |
| 13.535° | 13.53549 | 6.536554 | 359.2127 | 0.171768 |
| 14.837° | 14.83685 | 5.966021 | 518.4961 | 0.247934 |
| 15.973° | 15.97262 | 5.544258 | 221.2755 | 0.105809 |
| 16.351° | 16.35057 | 5.416944 | 88.56068 | 0.042348 |
| 17.367° | 17.36715 | 5.102086 | 2091.27 | 1 |
| 18.937° | 18.93699 | 4.682525 | 859.745 | 0.411111 |
| 20.168° | 20.16842 | 4.399317 | 729.6246 | 0.348891 |
| 20.929° | 20.92882 | 4.241163 | 299.6507 | 0.143287 |
| 21.946° | 21.94606 | 4.046822 | 686.9625 | 0.328491 |
| 22.719° | 22.71909 | 3.910844 | 1124.994 | 0.537948 |
| 23.604° | 23.6036 | 3.766255 | 214.5516 | 0.102594 |
| 23.814° | 23.81428 | 3.733409 | 211.4754 | 0.101123 |
| 24.874° | 24.87359 | 3.57676 | 376.8852 | 0.180218 |
| 25.609° | 25.60921 | 3.475661 | 285.7198 | 0.136625 |
| 26.745° | 26.74474 | 3.330612 | 251.1225 | 0.120081 |
| 27.111° | 27.11058 | 3.286493 | 138.8324 | 0.066387 |
| 27.558° | 27.55802 | 3.23414 | 68.66545 | 0.032834 |
| 28.653° | 28.65312 | 3.112975 | 232.9794 | 0.111406 |
| 29.630° | 29.63035 | 3.012493 | 176.5445 | 0.08442 |
| 31.129° | 31.12856 | 2.870833 | 132.7006 | 0.063455 |
| 31.567° | 31.56692 | 2.831959 | 106.4913 | 0.050922 |
| 32.180° | 32.18027 | 2.779369 | 100.1601 | 0.047894 |
| 33.073° | 33.0727 | 2.706382 | 58.33579 | 0.027895 |
| 34.096° | 34.09647 | 2.627426 | 68.95352 | 0.032972 |
| 34.460° | 34.46035 | 2.60051 | 147.7838 | 0.070667 |
| 36.226° | 36.22648 | 2.477677 | 73.88321 | 0.035329 |
| 37.497° | 37.49681 | 2.396604 | 137.836 | 0.06591 |
| 38.727° | 38.72692 | 2.323263 | 90.82414 | 0.04343 |
| 41.126° | 41.12576 | 2.193118 | 80.7093 | 0.038593 |

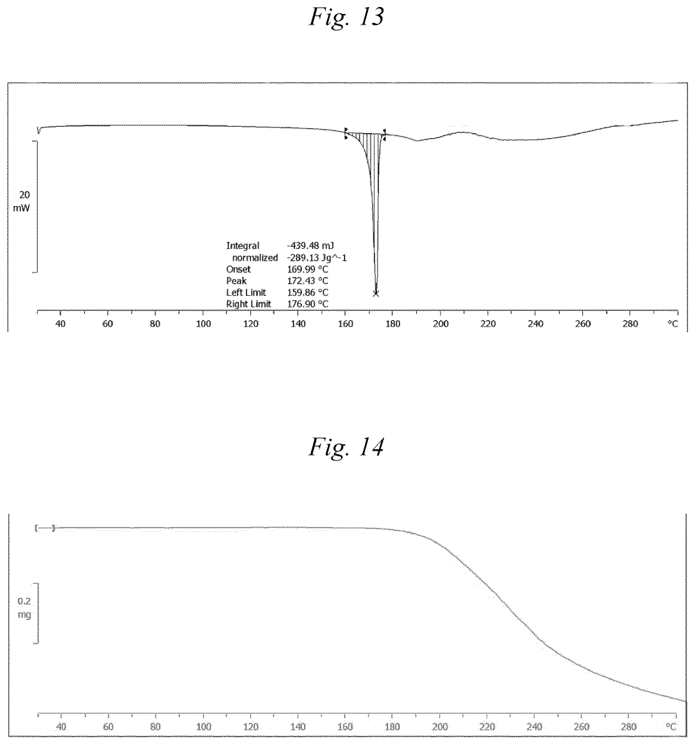

The DSC curve of I-7b (pattern 1) is shown in FIG. 13 and it is evident that the salt has a high melt onset (169.99° C.) and peak (172.43° C.). Equally, no events were observed prior to the melt endotherm, indicating the absence of multiple physical forms in the sample, and also no conversion of physical forms prior to the melt.

The thermogravimetric analysis (TGA) curve of I-7b (pattern 1) as shown in FIG. 14 showed no significant mass lost until about 180° C. at a heating rate of 10° C./min.

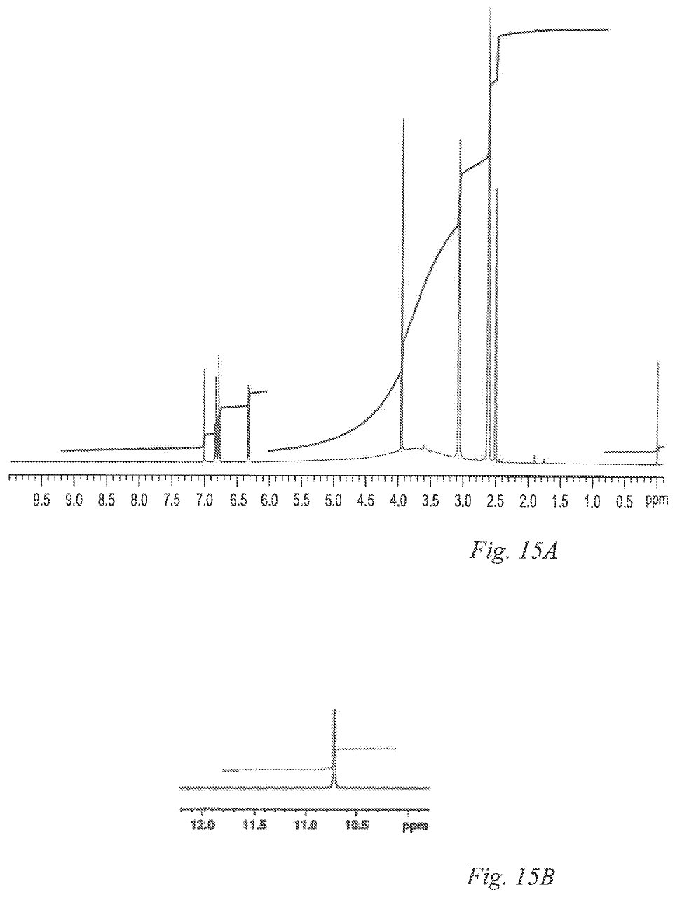

FIGS. 15A and 15B show a ¹H NMR spectrum of I-7b (pattern 1), which indicates protonation and 1 molar equivalent of L-tartrate (trace THF, 0.012 equivalents). The UPLC purity was 98.7%.

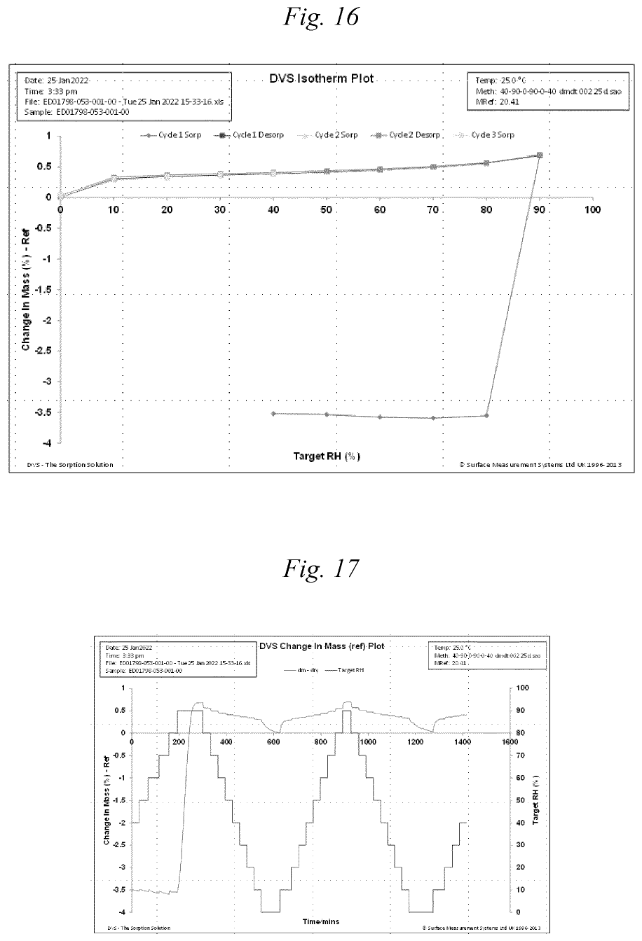

In DVS experiments with polymorph of pattern 1 (FIG. 16), the first sorption cycle showed a large mass change (+4.2%, relative to 0% RH) between 80-90% RH, and a 0.7% mass increase over 0-90% RH range in the second sorption cycle. This can be seen in the DVS change in mass plot of FIG. 17.

The storage stability of I-7b (pattern 1) was also assessed by storing the solid samples for 7 days under the following conditions: i) 25° C., closed vial, ii) 25° C./96% RH, iii) 40° C./75% RH, and comparing to fresh sample and the sample post DVS from above by XRPD. The results are presented in FIG. 18, which showed a change in form (formation of a hydrate; pattern 3) by XRPD post storage under elevated humidity conditions and post DVS. The sample stored at 25° C. in a closed vial showed no change by XRPD. The XRPD peak listing for I-7b (pattern 3) is as follows: 6.479°, 10.486°, 10.862°, 11.913°, 12.222°, 12.972°, 13.161°, 13.467°, 14.230°, 15.372°, 15.736°, 16.053°, 16.457°, 16.613°, 17.009°, 17.695°, 17.913°, 18.486°, 18.795°, 19.479°, 20.101°, 20.416°, 20.818°, 21.352°, 22.106°, 22.320°, 22.629°, 22.964°, 23.698°, 23.950°, 24.175°, 24.439°, 24.818°, 25.079°, 25.880°, 26.528°, 27.297°, 27.752°, 28.124°, 28.349°, 28.631°, 29.075°, 29.819°, 30.202°, 30.562°, 31.025°, 31.207°, 31.650°, 31.953°, 33.721°, 34.362°, 34.651°, 34.994°, 35.512°, 35.982°, 36.450°, 37.476°, 38.287°, 39.699°, 39.980°, 40.951°, and 41.870°.

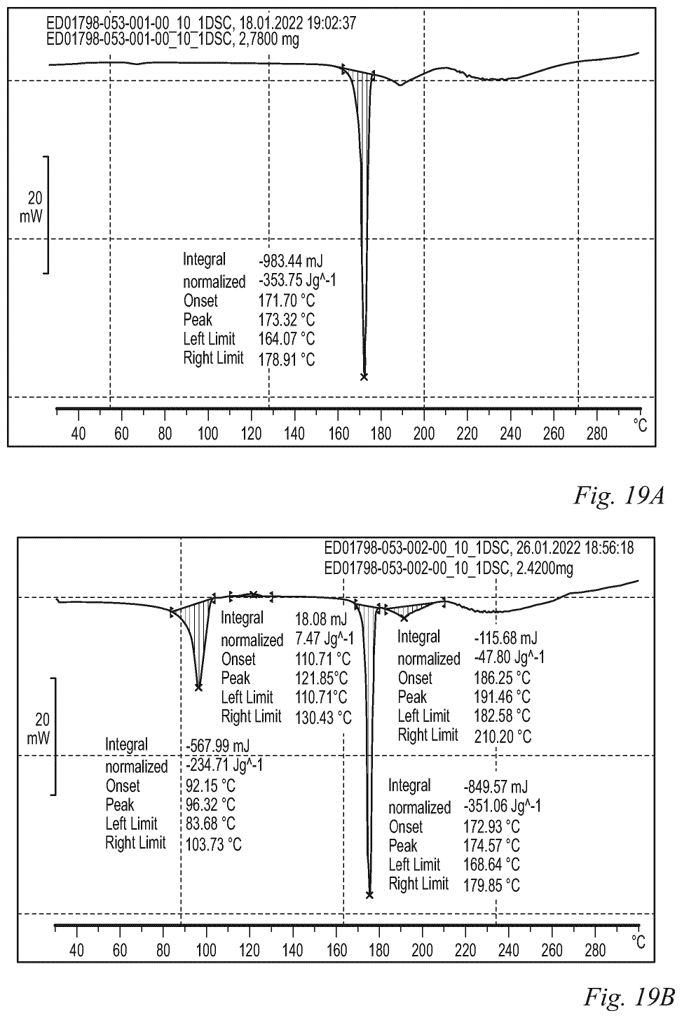

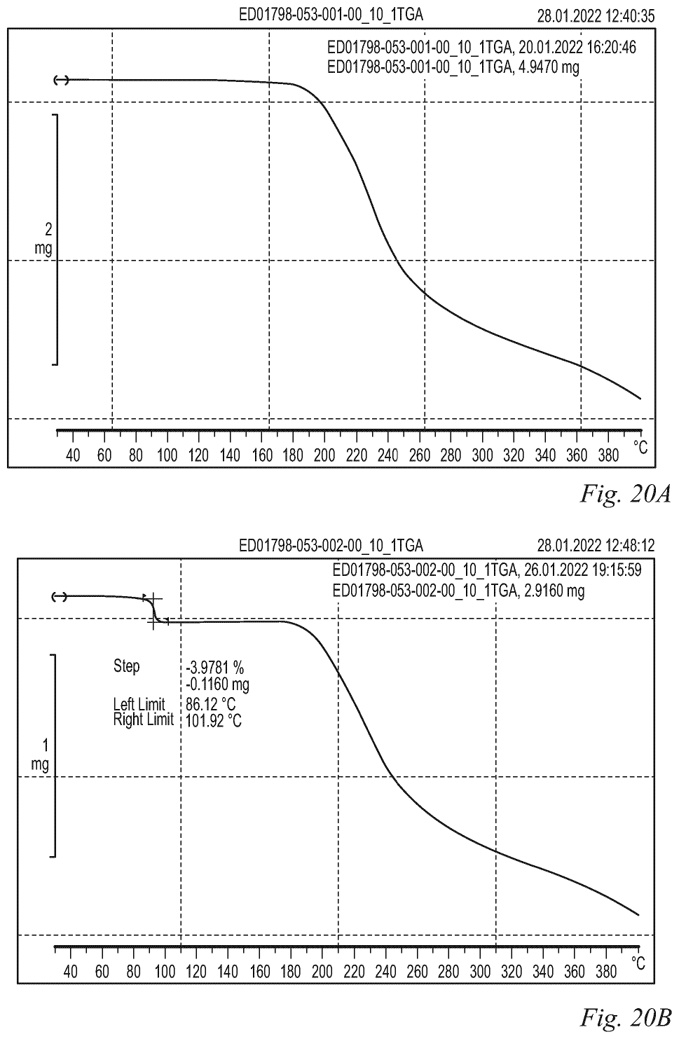

Thermal analysis of I-7b (pattern 3) by DSC showed an endothermic event of onset 92° C., followed by a small shallow exothermic event of onset 111° C. and a major endothermic event of onset 173° C. and a broad endothermic event of onset 186° C. (decomposition)(FIG. 19B). The TGA showed a 4% step mass loss between 86-102° C. corresponding to the initial event in the DSC, followed by decomposition above ca. 175° C. (FIG. 20B).

The changes to I-7b (pattern 1) pre- and post-DVS can be seen in the DSC plots of FIG. 19A and FIG. 19B, respectively, as well as the TGA plots of pre- and post-DVS material in FIG. 20A and FIG. 20B, respectively.

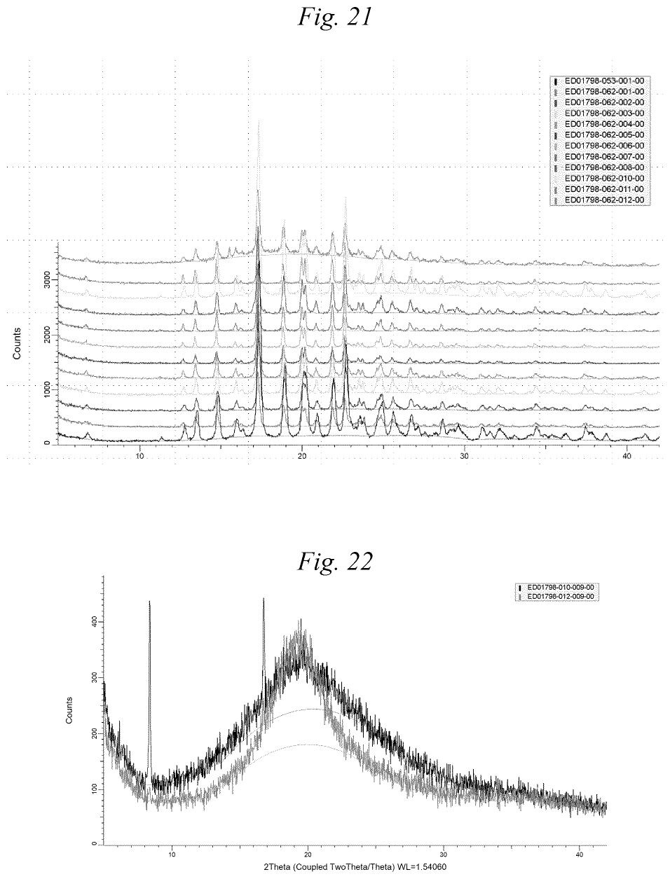

I-7b (pattern 1) was also subjected to maturation in 12 different solvents following the procedure detailed in Example 2. All samples were isolated as solids and retained their initial crystalline form according to XRPD analysis (FIG. 21).

I-7b polymorph having pattern 2 (made from 1,4-dioxane) showed a broad endothermic event by DSC between 33-77° C., a sharp endothermic event of onset 171° C. (peak 172° C.) and a broad shallow endothermic event between 178-208° C. By TGA, this polymorph showed 3.8% mass loss between 43-93° C. followed by decomposition above ca. 167° C. By ¹H NMR, this material showed 1 molar equivalent of L-tartrate (trace 1,4-dioxane, 0.014 equivalents). The UPLC purity was 98.1%.

Seeded. Compound I-7 (PI-d₀, free base) (447.6 mg) was dissolved in THF (11.2 mL), treated with seed of I-7b (pattern 1) and a. solution of L-tartaric acid (1 equivalent) in THF (4.4 mL). Resultant suspension was stirred, treated with further seed and stirred at room temperature for 4 days. The precipitate was isolated by filtration under a nitrogen stream (inverted funnel) and dried in vacuo (ca. 45 minutes) to give I-7b as a white solid (92% yield). The isolated solid was crystalline and of the same XRPD pattern (pattern 1) as obtained from the non-seeded preparation. ¹H NMR was consistent with 1 eq. of tartrate counterion. The crystalline I-7b material obtained from the seeded preparation had >99.5% purity (including counterion) by UPLC.

L-tartaric acid salt (0.5 eq). Compound I-7 (PI-d₀, free base) (10 mM) was dissolved in 1,4-dioxane or THF and treated with a solution of L-tartaric acid (5 mM) in THF while being stirred at room temperature. Samples were shaken overnight at room temperature to produce I-7b (L-tartrate salt of PI-d₀).

As shown in FIG. 22, the X-ray powder diffraction (XRPD) pattern indicates that the obtained salts were largely amorphous, with only some weak diffraction peaks observed in the sample from THF.

### Example 4

### Synthesis of hemi-fumarate salt of 3-(2(dimethylamino)ethyl)-1H-indol-4-ol (I-7c)(Hemi-Fumarate Salt of I-7/psilocin/psilocin-d₀/PI-d₀)

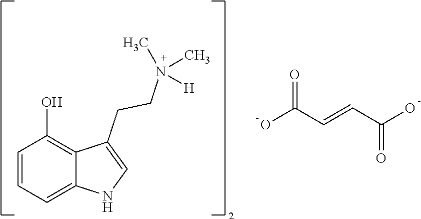

Non-seeded. Compound I-7 (PI-d₀, free base) (10 mM) was dissolved in 1,4-dioxane, acetonitrile, or THF and treated with a solution of fumaric acid (0.25M, 10 mM or 5 mM) in THF while being stirred at room temperature. Samples were shaken overnight at room temperature to produce I-7c (as a hemi-fumarate salt of PI-d₀).

Reactions performed using 0.5 equivalents of fumaric acid formed three different crystalline polymorphs of I-7c: a polymorph having pattern 1 (made from THF), a polymorph having pattern 2 (made from acetonitrile), a polymorph having pattern 3 (made from 1,4-dioxane). X-ray powder diffraction (XRPD) plots for these three polymorphs are shown in FIG. 23.

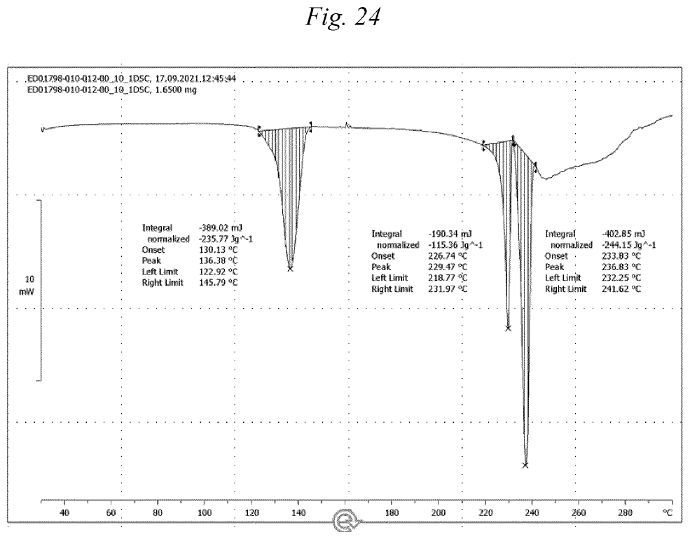

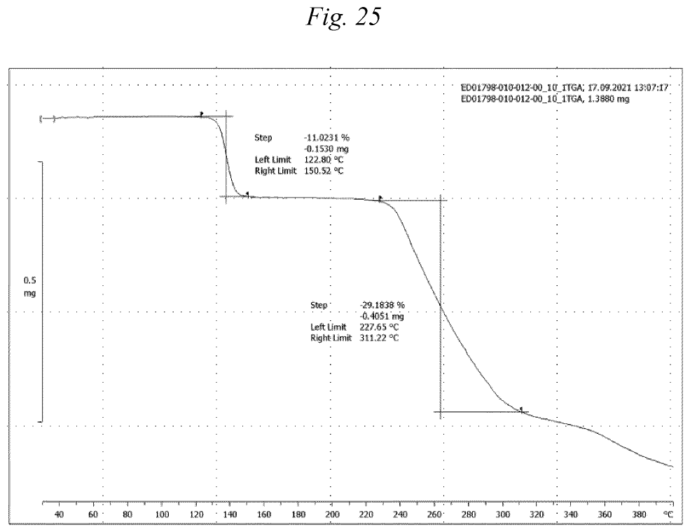

The DSC of I-7c (pattern 1) at 10° C./min (FIG. 24) shows a broad endothermic event of onset at 130° C. (peak 136° C.) and 2 further unresolved endothermic events of onset at 227 and 234° C. TGA of I-7c (pattern 1) at 10° C./min (FIG. 25) shows 11% mass loss between 123-151° C. and 29% mass loss between 228 to 311° C. corresponding to decomposition. ¹H NMR of I-7c (pattern 1) shows ca. 0.5 equivalents of fumarate and 0.4 equivalents of THF. The UPLC purity was 99.3%.

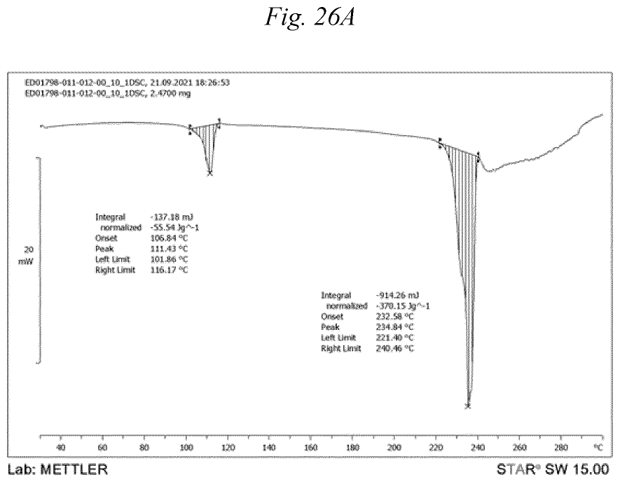

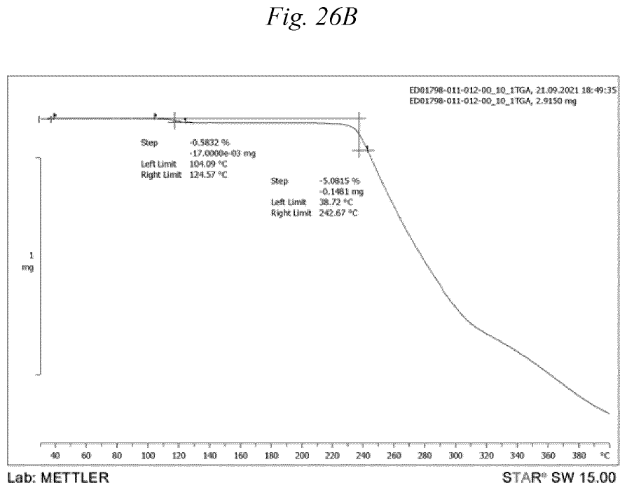

The DSC of I-7c (pattern 2) at 10° C./min (FIG. 26A) shows an endothermic event of onset 107° C. and an endothermic event of onset 233° C. TGA of I-7c (pattern 2) shows 0.6% mass loss between 104-125° C. and decomposition above ea. 220° C. (FIG. 26B). ¹H NMR of I-7c (pattern 2) shows ca. 0.5 equivalents of fumarate and 0.4 equivalents of MeCN. The UPLC purity was 99.5%.

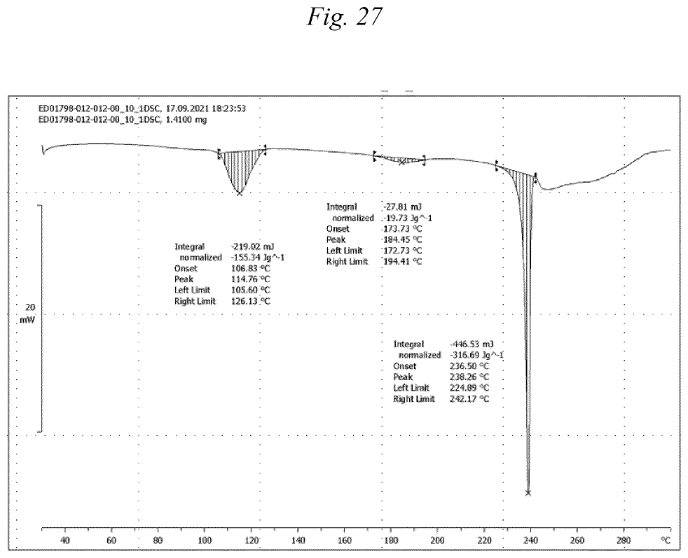

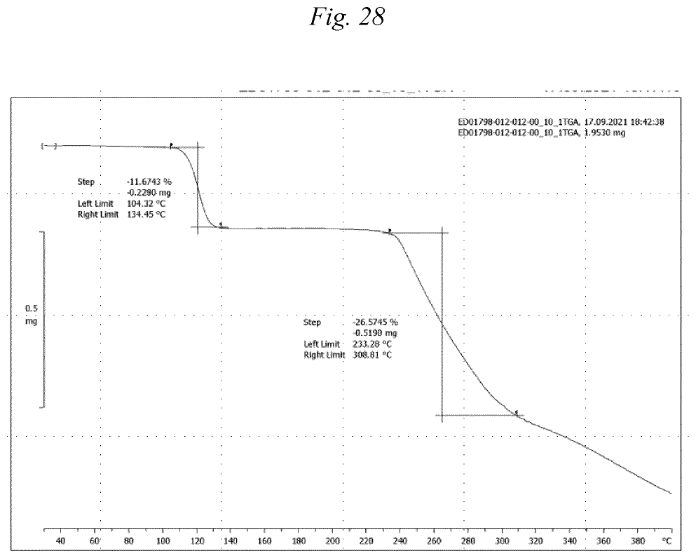

The DSC of I-7c (pattern 3) at 10° C./min (FIG. 27) shows a broad endothermic event of onset at 107° C., a small shallow endothermic event of onset at 174° C. and an endothermic event of onset at 237° C. TGA of I-7c (pattern 3) shows 11.7% mass loss between 104-134° C. and 27% mass loss between 233 to 309° C. (FIG. 28). ¹H NMR of I-7c (pattern 3) shows ca. 0.5 equivalents of fumarate and 0.43 equivalents of 1,4-dioxane. The UPLC purity was 99.5%.

Reactions performed using 1 equivalent of fumaric acid provided the following crystalline polymorphs of I-7c: the polymorph having pattern 1 (made from THF), the polymorph having pattern 3 (made from 1,4-dioxane), and a new polymorph having pattern 4 (made from acetonitrile). Therefore, a summary of polymorph formation of I-7c can be stated as follows: a polymorph having pattern 1 (made from either 0.5 eq or 1 eq fumaric acid and THF), a polymorph having pattern 2 (made from 0.5 eq fumaric acid and acetonitrile), a polymorph having pattern 3 (made from either 0.5 eq or 1 eq fumaric acid in 1,4-dioxane), and a polymorph having pattern 4 (made from 1 eq fumaric acid in acetonitrile). The X-ray powder diffraction (XRPD) pattern of these four crystalline polymorphs of I-7e are shown in FIG. 29.

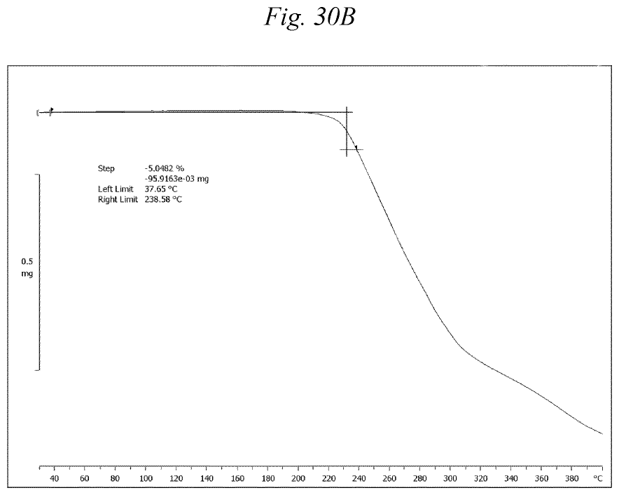

The DSC of I-7c (pattern 4) at 10° C./min shows a small endothermic event at 137° C. and an endothermic event of onset at about 228° C. (FIG. 30A). TGA of I-7c (pattern 4) at 10° C./min shows no significant mass loss until about 220° C. and 95% mass remaining at 239° C. (FIG. 30B). ¹H NMR of I-7c (pattern 4) shows ca. 0.5 equivalents of fumarate. The UPLC purity was 97.9%.

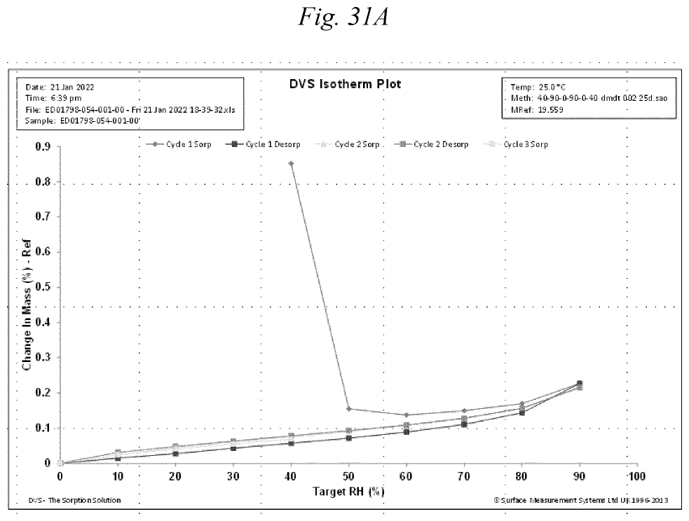

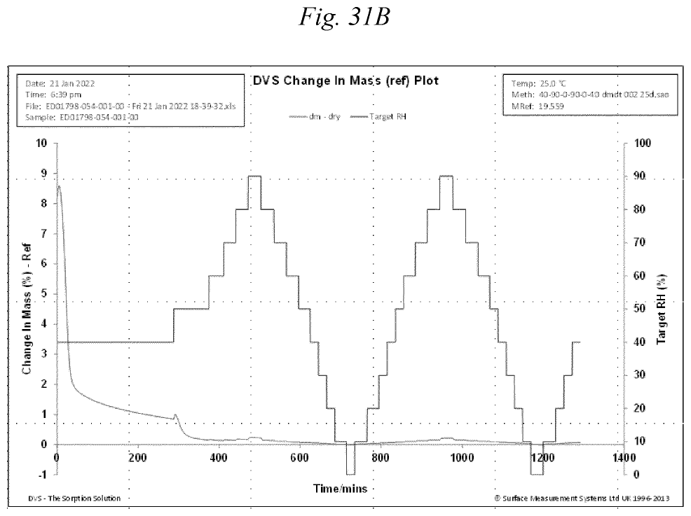

DVS experiments with polymorph of pattern 4 (FIG. 31A) initially showed a decrease in mass of about 0.7% between 40-60% RH, and a 0.2% mass increase over 0-90% RH range (second sorption cycle). This can also be seen in the DVS change in mass plot of FIG. 31B.

### Example 5

### Synthesis of Acetate Salt of 3-(2-(dimethylamino)ethyl)-1H-indol-4-ol (I-7d)(Acetate Salt of I-7/psilocin/psilocin-d₀/PI-d₀)

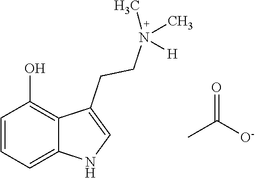

Compound I-7 (PI-d₀, free base) (10 mM) was dissolved in 1,4-dioxane or THF/heptane and treated with a solution of acetic acid (10 mM) in THF while being stirred at room temperature. Samples were shaken overnight at room temperature to produce I-7d (acetate salt of PI-d₀).

As shown in FIG. 32, the X-ray powder diffraction (XRPD) pattern indicates that two different crystalline polymorphs of I-7d were formed: a polymorph having pattern 1 (made from 1,4-dioxane), and a polymorph having pattern 2 (made from THF/heptane).

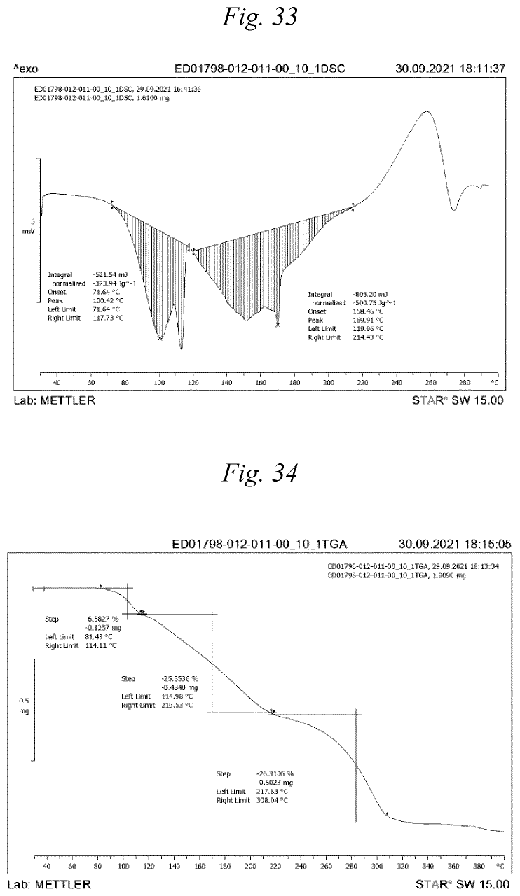

The DSC curve of I-7d (pattern 1) is shown in FIG. 33, which indicates the salt has multiple broad unresolved endotherms, with the earliest melt onset of (71.64° C.). The thermogravimetric analysis (TGA) curve of I-7d (pattern 1) as shown in FIG. 34 showed about 6.5% mass loss between about 81 to 114° C., about 25% mass loss between about 115 to 216° C., and about 26% mass loss between about 217 to 308° C. ¹H NMR of I-7d (pattern 1) shows ca. 1 equivalent of acetate, 0.4 equivalents of 1,4-dioxane and trace THF (0.025 equivalents). The UPLC purity was 99.2%.

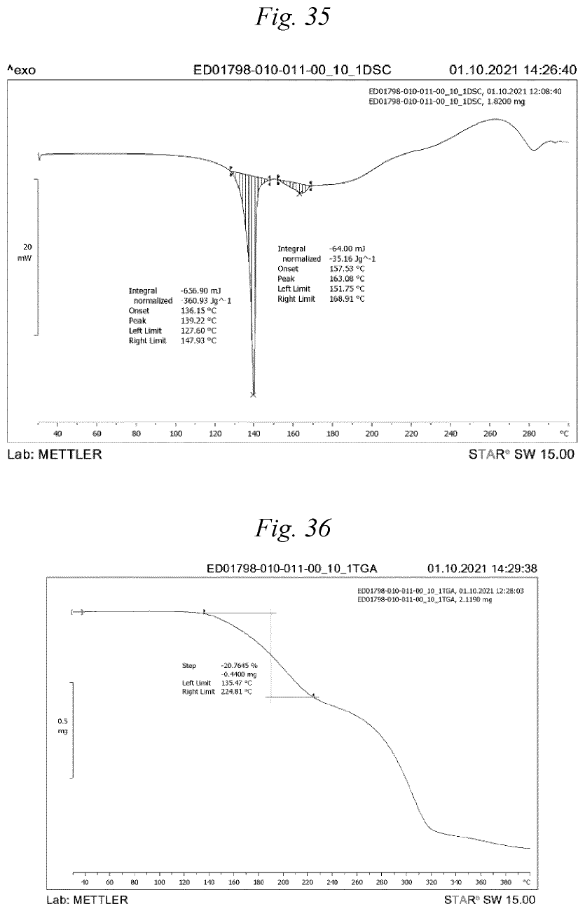

The DSC curve of I-7d (pattern 2) is shown in FIG. 35, which indicates the salt has a main endotherm with a melt onset of 136.15° C., higher than that of the polymorph of pattern 1. The thermogravimetric analysis (TGA) curve of I-7d (pattern 2) as shown in FIG. 36 showed mass loss of about 20.7% between about 135 to 224° C. ¹H NMR of I-7d (pattern 2) shows ca. 1 equivalent of acetate. The UPLC purity was 99.3%.

### Example 6

### Synthesis of Citrate Salt of 3-(2-(dimethylamino)ethyl)-1H-indol-4-ol (I-7e)(Citrate Salt of I-7/psilocin/psilocin-d₀/PI-d₀)

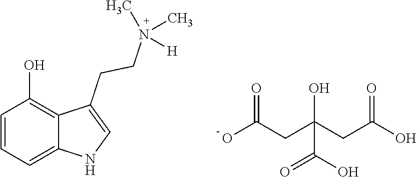

Compound I-7 (PI-d₀, free base) (10 mM) was dissolved in 1,4-dioxane and treated with a solution of citric acid (10 mM) in water while being stirred at room temperature. This gave an oil. Additional water was added until a solution was obtained. Samples were then freeze dried to provide I-7e (citrate salt of PI-d₀) as a white fluffy solid that was hygroscopic and rapidly became sticky. The obtained citrate salt was amorphous as shown by X-ray powder diffraction (XRPD) pattern in FIG. 37A.

The DSC of I-7e at 10° C./min shows large broad unresolved endothermic events between 104-134° C. and 137-212° C. TGA of I-7e at 10° C./min shows 3.3% mass loss between 109-123° C. and 33.5% mass loss between 137-212° C.

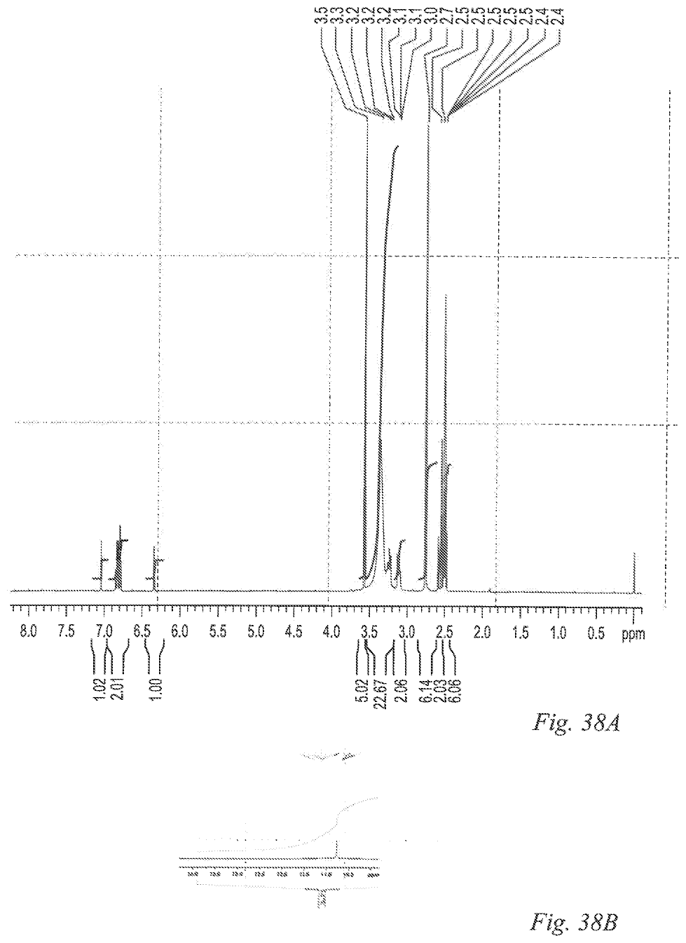

¹H NMR spectrum of I-7e (FIGS. 38A and 38B) shows protonation with citric acid (1 equivalent) along with 1,4-dioxane (0.6 mole equivalent). The UPLC purity was 98.5%.

Amorphous I-7e provided above was portioned and subjected to slurrying conditions in TBME, THF, EtOAc, EtOH, MeCN, IPA, toluene, and heptane with shaking at room temperature for 2 days, followed by refrigeration. The only solids obtained from these experiments were amorphous by XRPD and became sticky on isolation. For example, the material obtained from slurrying with THF was largely amorphous, with only some weak diffraction peaks in the X-ray powder diffraction (XRPD) pattern (FIG. 37B).

### Example 7

### Synthesis of Hemi-Malonate Salt of 3-(2-(dimethylamino)ethyl)-1H-indol-4-ol (I-7f)(Hemi-Malonate Salt of I-7/psilocin/psilocin-d₀/PI-d₀)

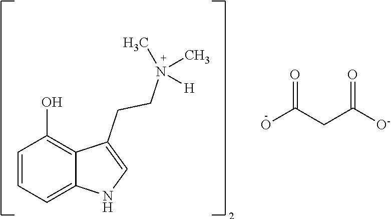

Compound I-7 (PI-d₀, free base) (10 mM) was dissolved in THF, MeCN, or 1,4-dioxane and treated with a 1M solution of malonic acid (5 or 10 mM) in THF while being stirred at room temperature. All samples produced gummy material or sticky amorphous material, except for the sample prepared using 1,4-dioxane solvent, and 0.5 equivalents of malonic acid in THF, which produced solid I-7f (hemi-malonate salt of PI-d₀) following shaking overnight at room temperature.

As shown in FIG. 39, the X-ray powder diffraction (XRPD) pattern indicates the salt prepared from 1,4-dioxane and 0.5 equivalents of malonic acid is crystalline, with only one crystal form observed (pattern 1).

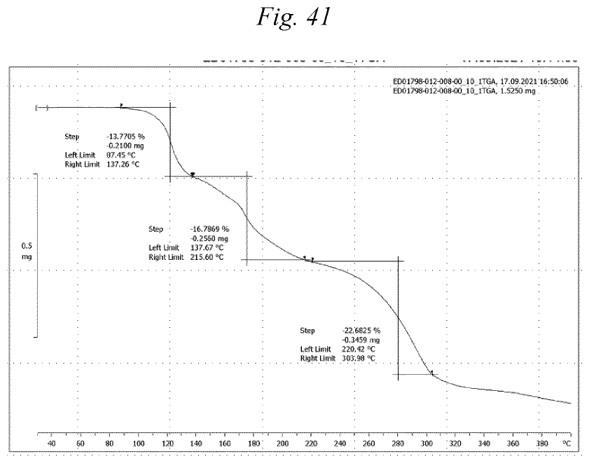

The DSC curve at 10° C./min shows a broad unresolved endothermic event between 98 to 132° C. and an unresolved endothermic event onset of 174° C. with a peak at 175° C. (FIG. 40). The TGA at 10° C./min shows 13.8% mass loss between 87 to 137° C., 16.8% between 138 to 215° C., and 22.7% between 220 to 304° C. (FIG. 41). H NMR of I-7f (pattern 1) shows ca. 0.5 equivalents of malonate and ca. 0.44 equivalents of 1,4-dioxane. The UPLC purity was 99.5%.

### Example 8

### Synthesis of Hemi-Fumarate Salt of 3-(2-(dimethylamino)ethyl)-1H-indol-4-ol (I-7c)(Hemi-Fumarate Salt of I-7/psilocin/psilocin-d₀/PI-d₀)

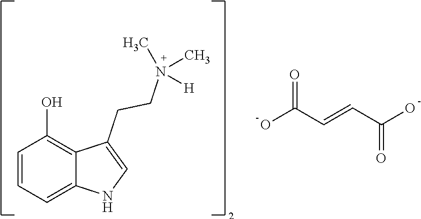

Seeded. Compound I-7 (PI-d₀, free base) (457.0 mg) was dissolved in acetonitrile (22.5 mL) and treated with seeds of I-7c (pattern 4) and p solution of fumaric acid (1 equivalent) in THF (8.9 mL). The resulting suspension was stirred, treated with additional seeds of I-7c (pattern 4) and stirred at room temperature for 4 days. The precipitate was isolated by filtration under a stream of nitrogen (inverted funnel) and dried in vacuo (ca. 40 minutes) to give an off-white solid in 97.5% yield. The isolated solid was moderately crystalline with a different XRPD pattern (pattern 5) from that obtained from non-seeded experiments of Example 4. The XRPD peak listing for I-7c (pattern 5) is provided in Table 14 (and shown in FIG. 42).

TABLE 14

| Caption (display) | Angle | d Value | Net Intensity | Rel. Intensity |
|---|---|---|---|---|
| 8.483° | 8.483156 | 10.41482 | 202.1152 | 0.424545 |
| 8.733° | 8.733033 | 10.11738 | 44.55472 | 0.093588 |
| 11.080° | 11.08012 | 7.978936 | 307.0166 | 0.644891 |
| 11.351° | 11.35111 | 7.789053 | 333.765 | 0.701077 |
| 11.622° | 11.62241 | 7.607838 | 179.431 | 0.376896 |
| 12.615° | 12.61488 | 7.011438 | 55.97579 | 0.117578 |
| 13.258° | 13.25757 | 6.672949 | 73.79655 | 0.15501 |
| 14.977° | 14.97686 | 5.910563 | 476.075 | 1 |
| 15.557° | 15.55708 | 5.691403 | 143.6231 | 0.301682 |
| 16.089° | 16.0892 | 5.504347 | 217.2447 | 0.456324 |
| 16.319° | 16.31855 | 5.4275 | 276.0824 | 0.579914 |
| 16.606° | 16.60595 | 5.334209 | 136.2941 | 0.286287 |
| 17.013° | 17.01313 | 5.207448 | 40.47203 | 0.085012 |
| 18.928° | 18.92837 | 4.684638 | 213.0289 | 0.447469 |
| 18.884° | 18.88391 | 4.695568 | 260.5841 | 0.547359 |
| 19.429° | 19.42858 | 4.565141 | 179.7858 | 0.377642 |
| 19.734° | 19.73422 | 4.495121 | 92.05923 | 0.193371 |
| 20.643° | 20.64288 | 4.299259 | 119.3917 | 0.250783 |
| 21.484° | 21.4845 | 4.132707 | 65.13663 | 0.13682 |
| 22.067° | 22.0669 | 4.024933 | 166.4906 | 0.349715 |
| 23.433° | 23.43334 | 3.793233 | 203.0481 | 0.426504 |
| 24.466° | 24.46598 | 3.635419 | 62.75409 | 0.131816 |
| 24.885° | 24.88459 | 3.575203 | 316.7789 | 0.665397 |
| 26.740° | 26.73984 | 3.331212 | 29.87601 | 0.062755 |
| 27.900° | 27.89971 | 3.195303 | 75.67041 | 0.158946 |
| 28.557° | 28.55742 | 3.123189 | 112.5171 | 0.236343 |
| 29.523° | 29.52253 | 3.02325 | 32.91097 | 0.06913 |
| 32.888° | 32.88833 | 2.721131 | 55.0898 | 0.115717 |
| 34.183° | 34.18291 | 2.62098 | 91.18144 | 0.191528 |
| 36.808° | 36.8076 | 2.439882 | 17.00791 | 0.035725 |

The DSC curve of I-7e (pattern 5) at 10° C./min shows a broad endothermic event below ca. 40° C. and an endothermic event of onset 232° C. The TGA at 10° C./min shows 6% mass loss between 70-111° C. and degradation above ca. 230° C. ¹H NMR of I-7c (pattern 5) shows ca. 0.5 equivalents of fumarate, ca. 0.2 equivalents of MeCN, and trace THF. The UPLC purity was 99.5% with the expected [M+H]⁺ ion (205.0).

A DVS experiment was carried out on I-7c (pattern 5) which showed a large initial mass loss at the beginning of the experiment and then a much smaller change in mass for the remainder of the experiment—with a 0.2% mass increase observed over the second sorption cycle over 0-90% RH range (relative to 0% RH). The sample was analyzed post-DVS. ¹H NMR analysis showed no significant solvent remaining and XRPD analysis showed a change in form to another new pattern (pattern 6). The XRPD peak listing for I-7c (pattern 6) is as follows: 9.746°, 11.354°, 12.338°, 13.762°, 16.111°, 16.644°, 19.929°, 20.180°, 21.576°, 22.758°, 23.348°, 23.938°, 24.724°, 25.226°, 26.203°, 27.910°, 29.056°, 29.499°, 32.753°, 35.567°, 37.279°, 37.347°, and 39.481°.

Thermal analysis of I-7c (pattern 6) by TGA showed no significant mass loss until decomposition above ca. 220° C. and the DSC showed two very close endothermic events of onset 221° C. and 231° C.

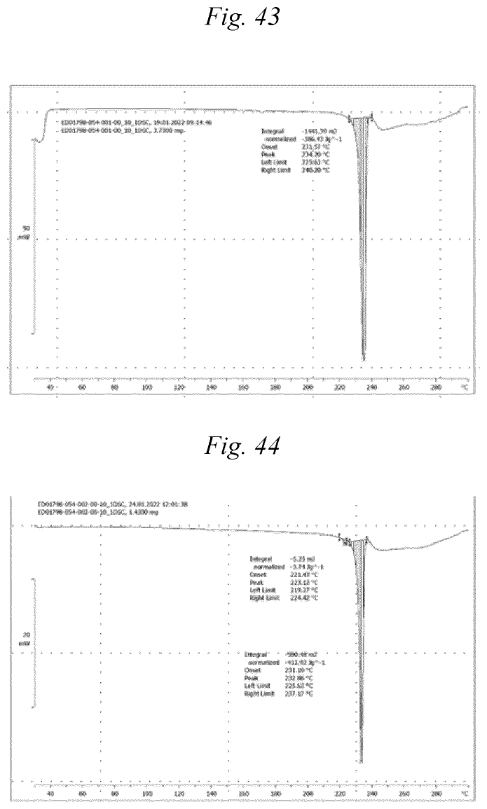

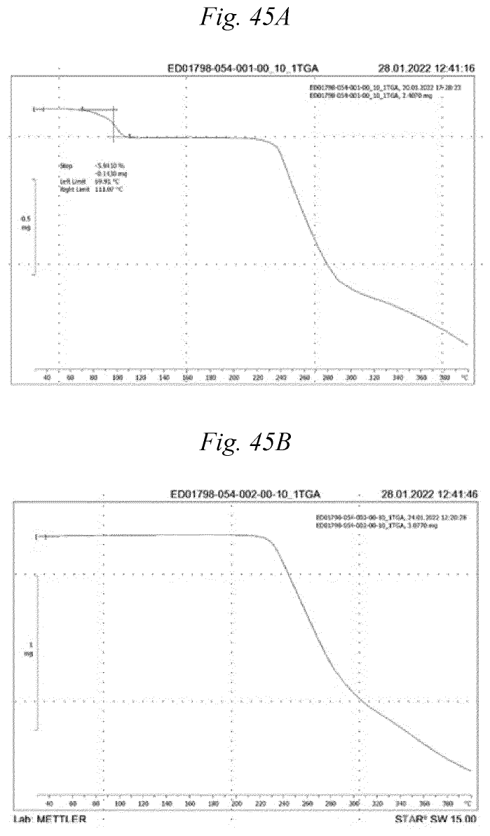

The changes to I-7c (pattern 5) pre- and post-DVS can be seen in the XRPD pattern of FIG. 42, in the DSC plots of pre- and post-DVS material in FIG. 43 and FIG. 44, respectively, as well as the TGA plots of pre- and post-DVS material in FIG. 45A and FIG. 45B, respectively. The TGA plot of pre-DVS material (FIG. 45A) shoed a step mass loss of 5.9%.

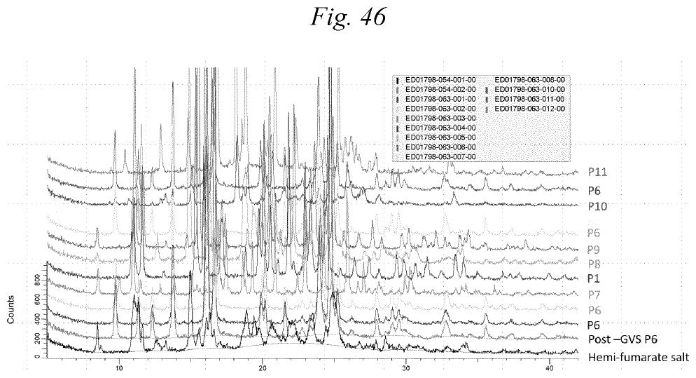

I-7c (pattern 5) was also subjected to maturation in 12 different solvents following the procedure detailed in Example 2. All isolated solids showed complex polymorphism with multiple crystalline patterns obtained (polymorphs of patterns (P) 1, 6, 7, 8, 9, 10, and 11) according to XRPD analysis (FIG. 46).

### Example 9

### Synthesis of Hemi-Succinate Salt of 3-(2-(dimethylamino)ethyl)-1H-indol-4-ol (I-7h)(Hemi-Succinate Salt of I-7/psilocin/psilocin-d₀/PI-d₀)

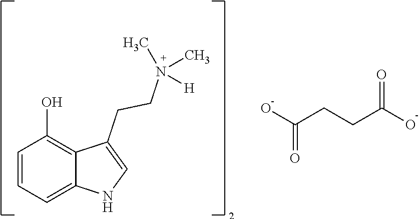

Compound I-7 (PI-4 free base) (10 mM) was dissolved in 1,4-dioxane or THF and treated with a solution of succinic acid (5 mM or 10 mM) in THF while being stirred at room temperature. Samples were refrigerated to produce I-7h (succinate salt of PI-d₀).

The X-ray powder diffraction (XRPD) pattern indicates that a single crystalline form (pattern 1) was formed from either 0.5 or 1 equivalent of succinic acid, in either 1,4-dioxane or THF (FIG. 47).

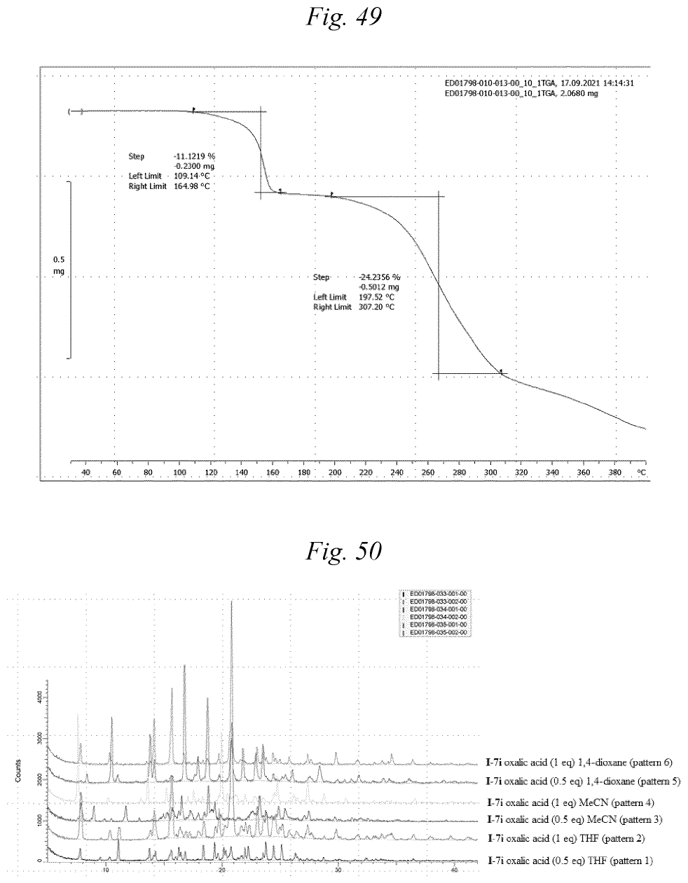

The DSC at 10° C./min shows several small events between 98 to 153° C. and an endothermic event of onset at about 185° C. (FIG. 48). TGA at 10° C./min shows about 11.1% mass loss between 109 to 165° C. and about 24% mass loss between 198 to 307° C. (FIG. 49).

¹H NMR of I-7h (pattern 1) shows ca. 0.5 equivalents of succinate with 0.44-0.46 equivalents of solvate (THF or 1,4-dioxane). The UPLC purity was 99.3-99.4%.

### Example 10

### Synthesis of Oxalate Salt of 3-(2-(dimethylamino)ethyl)-1H-indol-4-ol (I-7i)(Oxalate Salt of I-7/psilocin/psilocin-d₀/PI-d₀)

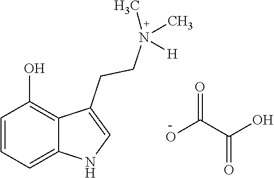

Compound I-7 (PI-d₀, free base) (10 mM) was dissolved in 1,4-dioxane, acetonitrile, or THF and treated with a solution of oxalic acid (10 mM or 5 mM) in THF while being stirred at room temperature. Samples were refrigerated to produce 1-71 (oxalate salt of PI-d₀).

As shown in FIG. 50, the X-ray powder diffraction (XRPD) pattern indicates that six different crystalline polymorphs of I-7i were formed: a polymorph having pattern 1 (made from 0.5 eq oxalic acid and THF), a polymorph having pattern 2 (made from 1 eq oxalic acid and THF), a polymorph having pattern 3 (made from 0.5 eq oxalic acid and acetonitrile), a polymorph having pattern 4 (made from 1 eq oxalic acid and acetonitrile), a polymorph having pattern 5 (made from 0.5 eq oxalic acid and 1,4-dioxane), and a polymorph having pattern 6 (made from 1 eq oxalic acid and 1,4-dioxane).

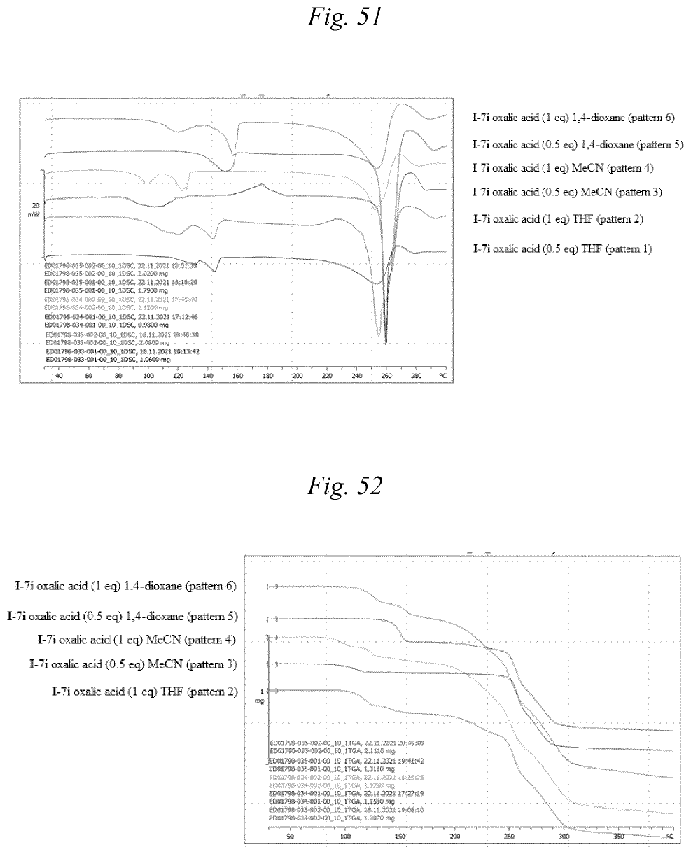

The DSC at 10° C./min shows that all solid crystalline material isolated from the various experiments were solvates, with several events showing in DSC (FIG. 51). Further, evidence that no anhydrous crystalline forms were isolated, the TGA shows several elements of mass loss (FIG. 52). ¹H NMR also indicated the formation of solvates: pattern 1-0.7 equivalents of THF; pattern 2-0.5 equivalents of THF; pattern 3-0.31 equivalents of MeCN; pattern 4-0.8 equivalents of MeCN; pattern 5-0.5 equivalents of 1,4-dioxane; pattern 6-0.45 equivalents of 1,4-dioxane. All polymorphs showed peaks associated with protonation, however it was not possible to quantify the equivalents of oxalate.

### Example 11

### Synthesis of Benzoate Salt of 3-(2-(dimethylamino)ethyl)-1H-indol-4-ol (I-7j)(Benzoate Salt of I-7/psilocin/psilocin-d₀/PI-d₀)

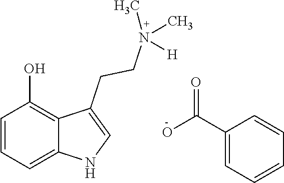

Compound I-7 (PI-d₀, free base) (10 mM) was dissolved in 1,4-dioxane, acetonitrile, or THF and treated with a solution of benzoic acid (10 mM) in THF while being stirred at room temperature. Samples were refrigerated to produce I-7j (benzoate salt of PI-d₀).

As shown in FIGS. 53A, the X-ray powder diffraction (XRPD) pattern indicates the salt is crystalline, with only one crystal form observed regardless of which solvent was employed (pattern 1). FIG. 53B shows the annotated XRPD, and Table 15 shows the XRPD peak listing for I-7j (pattern 1).

TABLE 15

| Caption | | | Gross | Rel. |
|---|---|---|---|---|
| (display) | Angle | d Value | Intensity | Intensity |
| 9.492° | 9.492038 | 9.310002 | 816.1553 | 0.234207 |
| 11.011° | 11.01083 | 8.028995 | 225.1465 | 0.046314 |
| 12.391° | 12.3913 | 7.137437 | 755.566 | 0.210192 |
| 13.440° | 13.4398 | 6.58288 | 292.8689 | 0.061474 |
| 14.609° | 14.60938 | 6.058398 | 1433.992 | 0.417304 |
| 15.432° | 15.43177 | 5.737337 | 182.47 | 0.016946 |
| 16.394° | 16.39421 | 5.402621 | 3297.966 | 1 |
| 18.259° | 18.25882 | 4.854886 | 2252.669 | 0.662731 |
| 18.967° | 18.96731 | 4.675109 | 3023.974 | 0.904632 |
| 19.356° | 19.35627 | 4.582032 | 977.581 | 0.255289 |
| 19.827° | 19.82713 | 4.474266 | 1207.459 | 0.328425 |
| 20.843° | 20.84305 | 4.25842 | 194.7453 | 0.010555 |
| 21.476° | 21.47557 | 4.134406 | 738.8857 | 0.187048 |
| 22.062° | 22.0625 | 4.025727 | 184.4619 | 0.012183 |
| 22.805° | 22.80468 | 3.89636 | 799.9279 | 0.203211 |
| 23.862° | 23.86174 | 3.726092 | 491.5757 | 0.103741 |
| 24.963° | 24.96308 | 3.56414 | 799.8894 | 0.201169 |
| 25.734° | 25.73424 | 3.459058 | 471.1698 | 0.096934 |
| 26.170° | 26.17029 | 3.402404 | 1669.852 | 0.47813 |
| 26.992° | 26.99244 | 3.300608 | 196.1326 | 0.015295 |
| 27.738° | 27.73771 | 3.213596 | 498.7095 | 0.115793 |
| 28.593° | 28.5934 | 3.119341 | 554.2463 | 0.136655 |
| 30.073° | 30.07312 | 2.969141 | 177.3094 | 0.020681 |
| 30.746° | 30.74603 | 2.905674 | 207.6318 | 0.030292 |
| 31.041° | 31.0409 | 2.878741 | 191.1328 | 0.02535 |
| 31.799° | 31.79946 | 2.811779 | 197.1789 | 0.02867 |
| 32.794° | 32.79412 | 2.728732 | 184.0209 | 0.026954 |
| 33.551° | 33.55059 | 2.668916 | 152.5839 | 0.019835 |
| 34.480° | 34.47956 | 2.599105 | 182.2929 | 0.030126 |
| 35.430° | 35.43034 | 2.531506 | 184.8386 | 0.031197 |
| 37.685° | 37.68499 | 2.385068 | 162.8452 | 0.024766 |
| 38.643° | 38.64258 | 2.328139 | 157.4924 | 0.022742 |

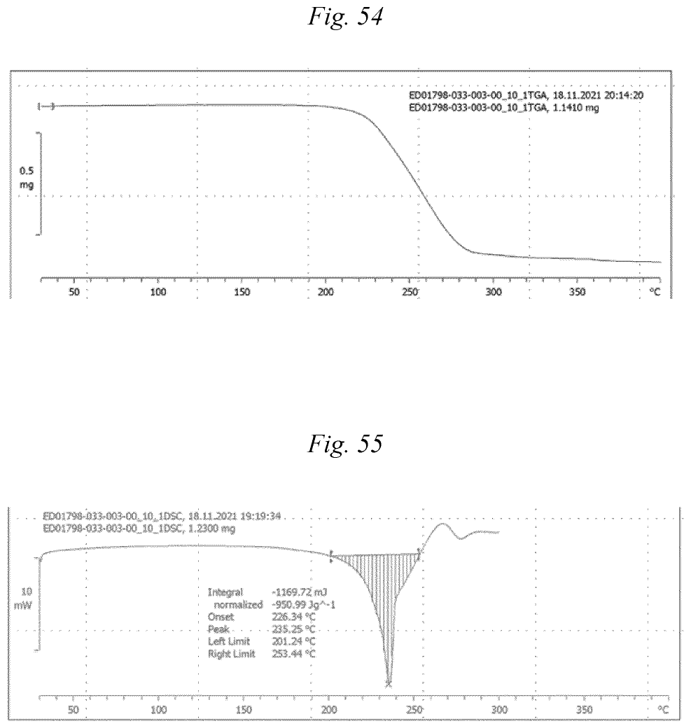

The sample formed from THF was subjected to TGA analysis (FIG. 54), which indicated the crystalline form was stable until about 200° C. This mass loss in TGA associated with melt and decomposition in DSC (FIG. 55), which showed a high melt onset (226.3° C.) and peak (235.2° C.). Equally, no events were observed prior to the melt endotherm, indicating the absence of multiple physical forms in the sample, and also no conversion of physical forms prior to the melt. ¹H NMR confirmed 1 equivalent of benzoic acid (trace THF, ca. 0.07 equivalents). The UPLC purity was >99.5% total including counterion.

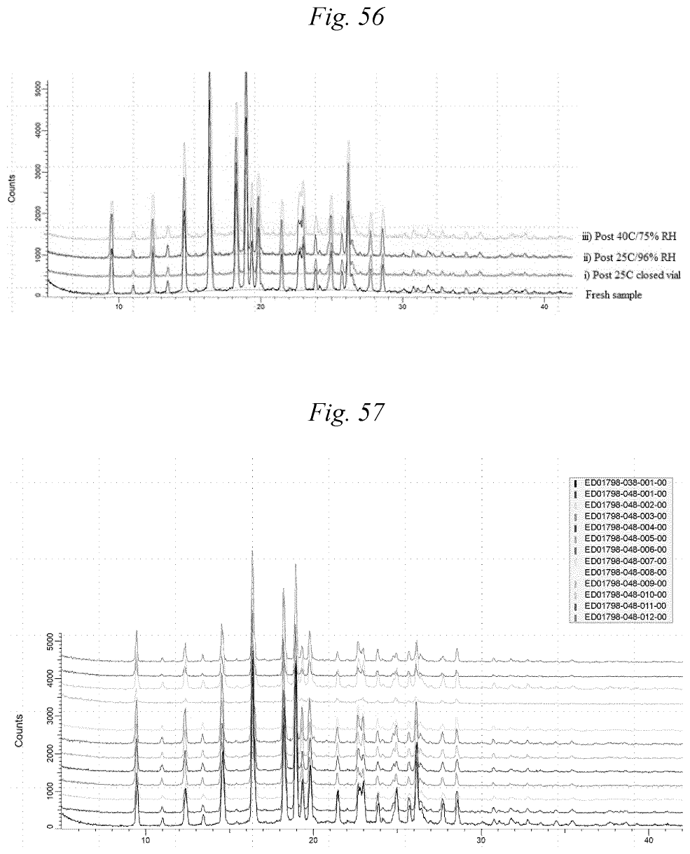

The storage stability of I-7j (sample from THF) was also assessed by storing the solid samples for 22 days under the following conditions: i) 25° C., closed vial, ii) 25° C./96% RH, and iii) 40° C./75% RH, and comparing to fresh sample by XRPD. The results are presented in FIG. 56, which showed no change in form by XRPD post storage. Further, no loss of purity was observed by 1H NMR and UPLC.

I-7j (sample from THF) was also subjected to maturation in 12 different solvents following the procedure detailed in Example 2. All samples were isolated as solids and retained their initial crystalline form according to XRPD analysis (FIG. 57).

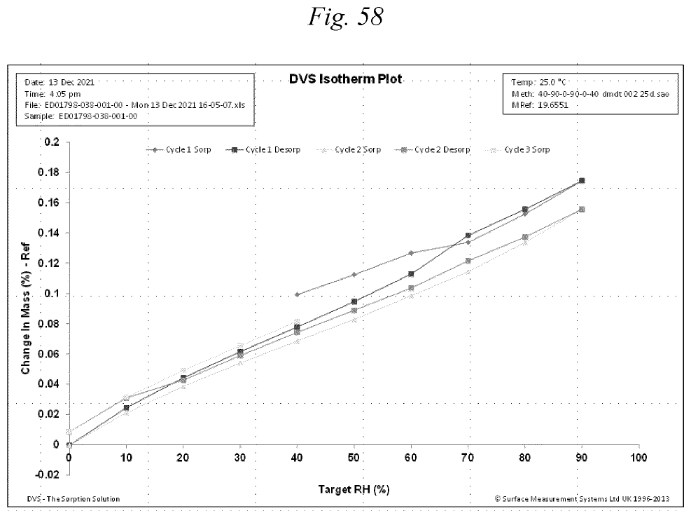

DVS isotherm plot of I-7j (sample from THF) is shown in FIG. 58, which shows a 0.16% mass increase over 0-90% RH range (second sorption cycle).

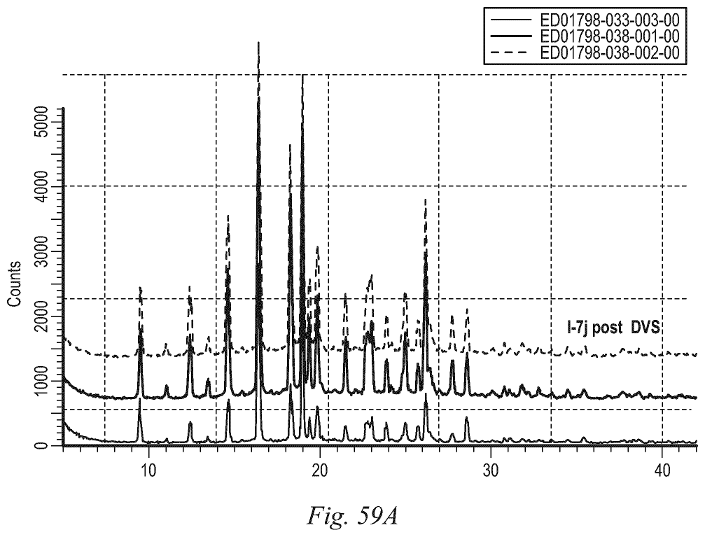

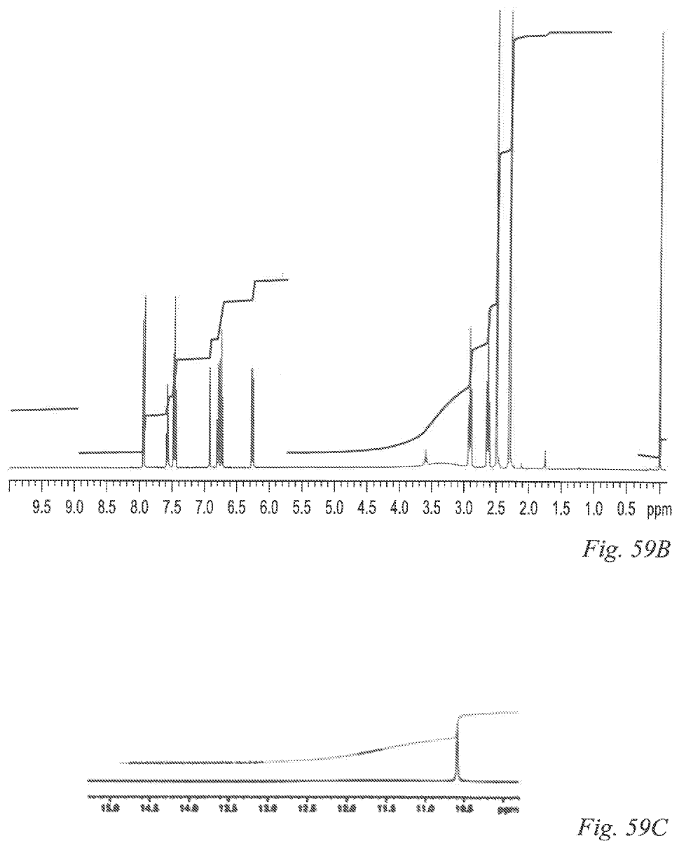

FIG. 59A shows the XRPD pattern of I-7j after being subjected to the DVS conditions above (post-DVS) compared to the XRPD pattern before DVS from material obtained from THF and acetonitrile (see FIG. 53A), indicating that no changes to the crystal structure took place. There was also no loss of purity following DVS analysis according to ¹H NMR (FIGS. 59B-59C) and UPLC. ¹H NMR was consistent with 1 eq of benzoic acid (0.97 eq).

This data represents advantageous behavior for pharmaceutical development purposes.

Seeded. Compound I-7 (PI-d₀, free base) (498.9 mg) was dissolved in THF (12.5 mL), treated with seeds of 1-7j (pattern 1), a solution of benzoic acid (1 equivalent) in THF was added. The resulting suspension was stirred, additional seeds were added, and stirred at room temperature for 11 days. The precipitate was isolated by filtration under a stream of nitrogen (inverted funnel) and dried in vacuo to give I-7j as a white solid (84.6% yield). The isolated solid was crystalline and of the same pattern (pattern 1) as obtained in the non-seeded preparation. ¹H NMR was consistent with 1 eq. of benzoate counterion. The crystalline I-7j material obtained from the seeded preparation had >99.5% purity (including counterion) by UPLC. It showed a broad endothermic event of onset 227° C. in the DSC and no significant mass loss until degradation above ca. 200° C. in the TGA. On exposure to changes in relative humidity in the GVS experiment the material showed a very small increase in mass (0.16%) over the 0-90% relative humidity range (second sorption cycle). It is not hygroscopic and showed no change in form by XRPD after the GVS experiment. Further, no change in form was observed by XRPD on storage under stress conditions after 22 days and 9 weeks. The sample stored at 40° C./75% RH showed very slight discoloration after 9 weeks. Further, the same pattern 1 by XRPD was observed after exposure to solvent maturation following the procedure described in Example 2.

### Example 12

### Synthesis of Salicylate Salt of 3-(2-(dimethylamino)ethyl)-1H-indol-4-ol (I-7k)(Salicylate Salt of I-7/psilocin/psilocin-d₀/PI-d₀)

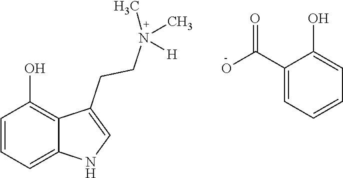

Compound I-7 (PI-d₀, free base) (10. mM) was dissolved in 1,4-dioxane/heptane, acetonitrile/TBME, or THF/heptane and treated with a solution of salicylic acid (10 mM) in THF while being stirred at room temperature. Samples were refrigerated to produce I-7k (salicylate salt of PI-d₀).

As shown in FIG. 60, the X-ray powder diffraction (XRPD) pattern indicates that three different crystalline polymorphs of I-7k were formed: a polymorph having pattern 1 (made from acetonitrile/TBME), a polymorph having pattern 2 (made from THF/heptane), and a polymorph having pattern 3 (made from 1,4-dioxane/heptane).

The DSC of I-7k (pattern 1) at 10° C./min shows endothermic events of onset 145° C. and 183° C. and a broad endothermic event above 200° C. (decomposition). TGA of I-7k (pattern 1) shows no significant mass loss until decomposition above ca. 200° C. ¹H NMR of I-7k (pattern 1) shows ca. 1 equivalent of salicylate. The UPLC purity was >99.5% total including counterion.

The DSC of I-7k (pattern 2) at 10° C./min shows endothermic events of onset 131° C. and 180° C. TGA of I-7k (pattern 2) shows no significant mass loss until decomposition above ca. 210° C. ¹H NMR of I-7k (pattern 2) shows ca. 0.9 equivalents of salicylate (trace THF, 0.025 equivalents). The UPLC purity was >99.5% total including counterion.

The DSC of I-7k (pattern 3) at 10° C./min shows endothermic events of onset 132° C. and 180° C. and a broad endothermic event above ca. 200° C. (decomposition). TGA of I-7k (pattern 3) shows no significant mass loss until decomposition above ca. 200° C. ¹H NMR of I-7k (pattern 3) shows ca. 1 equivalent of salicylate (trace 1,4-dioxane, 0.04 equivalents). The UPLC purity was >99.5% total including counterion.

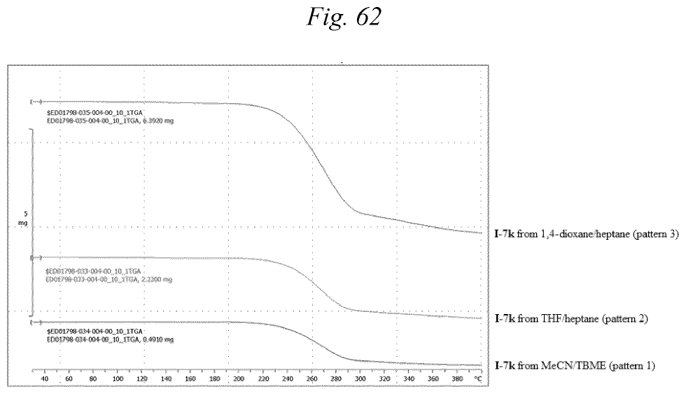

The DSC and TGA plots of these crystalline polymorphs are presented in FIGS. 61 and 62, respectively.

### Example 13

### Synthesis of Benzenesulfonate Salt of 3-(2-(bis(methyl-d₃)amino)ethyl-1,1,2,2-d₄)-1H-indol-4-ol (I-3a)(Benzenesulfonate Salt of I-3/psilocin-d₁₀/PI-d₁₀)

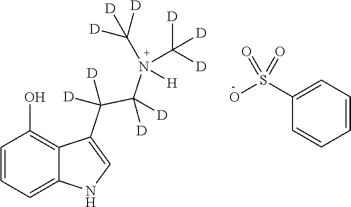

Compound I-3 (PI-d₁₀, free base) (10.49 mg) was dissolved in acetonitrile (500 μL) and treated with a solution of 1M benzenesulfonic acid in THF (1 equivalent). The resultant solution was treated with TBME (300 μL) and refrigerated. After three days, the solution remained as a clear solution. Seeds of I-7a crystalline pattern 1 (see Example 2) were then added. The mixture was further refrigerated overnight and a small quantity of solid had formed. After a further 6 days refrigeration more solid had formed. This was isolated by removal of the supernatant and allowing the residual solid to dry at room temperature. Crystals of I-3a (benzenesulfonate salt of PI-d₁₀) were thus obtained.

The X-ray powder diffraction (XRPD) pattern indicates that a single crystalline form of I-3a was formed (pattern 1) (FIG. 63A). Zoomed in and annotated versions of the XRPD pattern are presented in FIGS. 63B-63C. FIG. 63D shows that the single crystalline form of I-3a has the same pattern (pattern 1) as the I-7a seeds. FIGS. 63E-63F show the single crystal X-ray structure of I-3a (pattern 1). Table 16 shows the XRPD peak listing for I-3a (pattern 1).

TABLE 16

| | Caption | | | Net | Rel. |
|---|---|---|---|---|---|
| Name | (display) | Angle | d Value | Intensity | Intensity |
| Peak #1 | 7.023° | 7.023418 | 12.57581 | 77.75247 | 0.000758426 |
| Peak #2 | 7.767° | 7.767084 | 11.37332 | 7314.893 | 0.07135213 |
| Peak #3 | 11.822° | 11.8224 | 7.479583 | 32.03899 | 0.00031252 |
| Peak #4 | 12.550° | 12.54997 | 7.047554 | 78.68196 | 0.000767493 |
| Peak #5 | 12.860° | 12.85957 | 6.878568 | 21.29807 | 0.000207749 |
| Peak #6 | 13.994° | 13.99379 | 6.323494 | 1152.819 | 0.01124502 |
| Peak #7 | 15.521° | 15.52119 | 5.704481 | 102518.2 | 1 |
| Peak #8 | 18.436° | 18.43564 | 4.808719 | 365.3079 | 0.003563346 |
| Peak #9 | 19.503° | 19.50339 | 4.547799 | 38.2632 | 0.000373233 |
| Peak #10 | 20.760° | 20.7598 | 4.275309 | 47.20708 | 0.000460475 |
| Peak #11 | 21.070° | 21.06976 | 4.21311 | 278.4323 | 0.00271593 |
| Peak #12 | 22.007° | 22.00676 | 4.035797 | 176.9576 | 0.001726109 |
| Peak #13 | 22.745° | 22.74485 | 3.906473 | 76.58357 | 0.000747024 |
| Peak #14 | 23.340° | 23.34011 | 3.808173 | 16264.17 | 0.1586466 |
| Peak #15 | 24.187° | 24.18713 | 3.676697 | 57.4707 | 0.00056059 |
| Peak #16 | 25.532° | 25.5316 | 3.486051 | 48.40523 | 0.000472162 |
| Peak #17 | 26.880° | 26.87974 | 3.314191 | 24.26286 | 0.000236669 |
| Peak #18 | 27.856° | 27.85616 | 3.200199 | 42.07394 | 0.000410405 |
| Peak #19 | 28.163° | 28.16311 | 3.166014 | 143.1989 | 0.001396815 |
| Peak #20 | 31.267° | 31.26673 | 2.858461 | 12789.69 | 0.1247553 |
| Peak #21 | 33.024° | 33.02377 | 2.710279 | 28.16152 | 0.000274698 |
| Peak #22 | 35.030° | 35.02954 | 2.559547 | 19.00768 | 0.000185408 |
| Peak #23 | 36.835° | 36.83464 | 2.438153 | 42.1271 | 0.000410923 |
| Peak #24 | 39.312° | 39.31247 | 2.289996 | 2013.023 | 0.01963576 |
| Peak #25 | 40.545° | 40.54509 | 2.223177 | 83.841 | 0.000817816 |
| Peak #26 | 40.988° | 40.98812 | 2.200164 | 15.99768 | 0.000156047 |

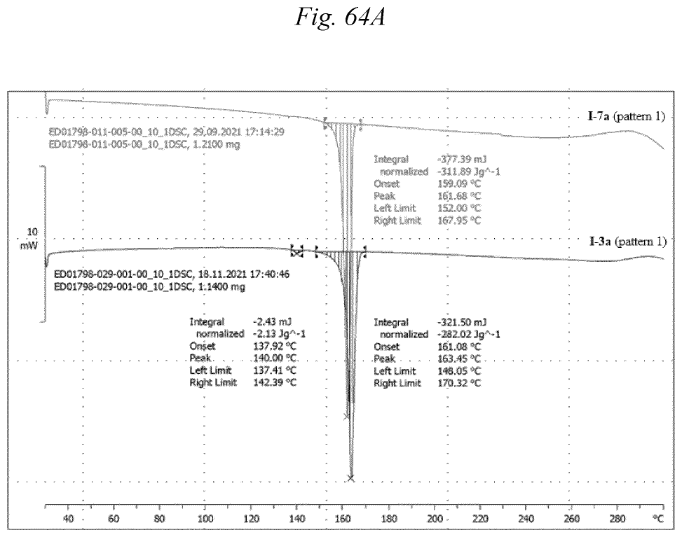

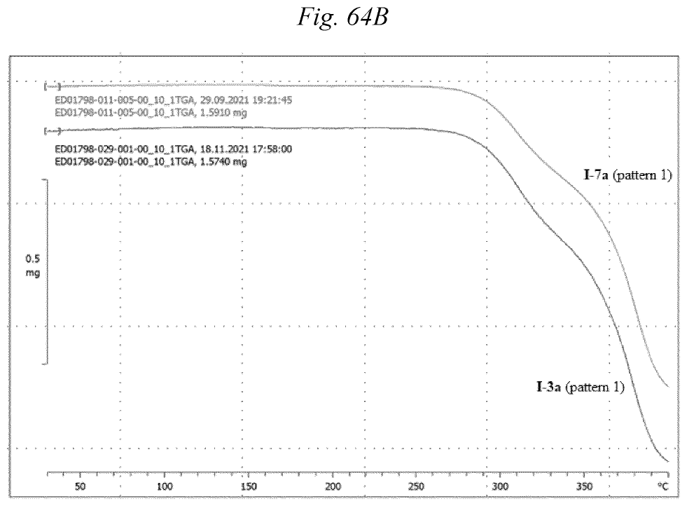

The DSC curve of I-3a is shown in FIG. 64A, compared to that of I-7a and it is evident that the salt has a high melt onset (161.08° C.) and peak (163.45° C.). One small event was observed prior to the melt endotherm with an onset of 137.92° C. The thermogravimetric analysis (TGA) curve of I-3a at 10° C./min is shown in FIG. 64B, which is nearly identical to the TGA curve of I-7a.

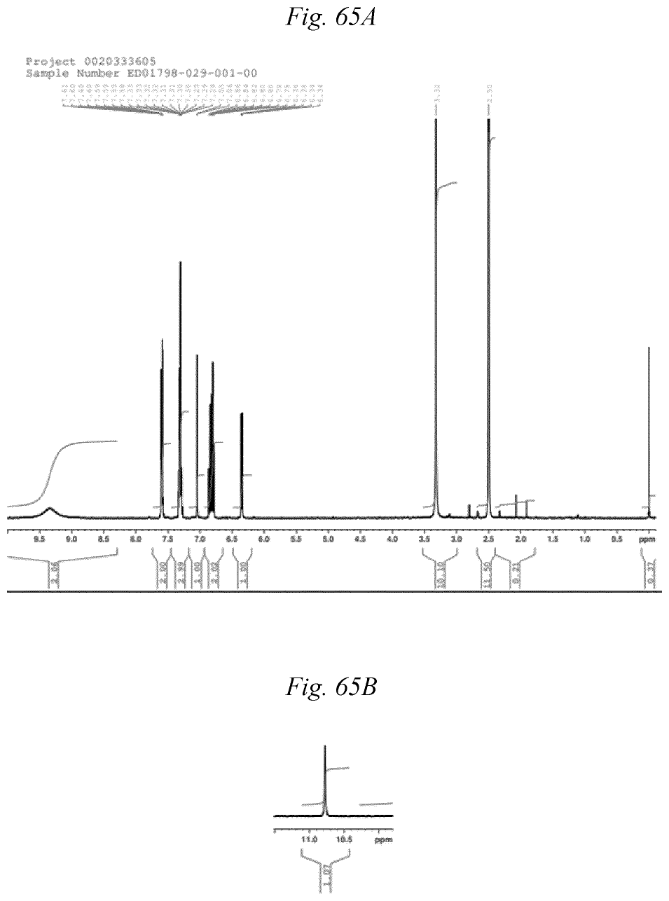

The identity of I-3a was also confirmed by ¹H NMR (FIGS. 65A-65B), which showed ca. 1 equivalent of benzenesulfonate (trace MeCN, 0.016 eq.). The UPLC purity was >99.5% total including counter ion.

### Example 14

### Synthesis of Tartrate Salt of 3-(2-(bis(methyl-d₃)amino)ethyl-1,1,2,2-d₄)-1H-indol-4-ol (I-3b)(Tartrate Salt of I-3/psilocin-d₁₀/PI-d₁₀)

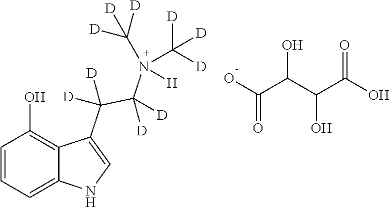

Non-seeded. Compound I-3 (PI-d₁₀, free base) (10.52 mg) was dissolved in THF (250 μL) and treated with a 0.5M solution of L-tartaric acid in THF (1 equivalent) at room temperature. A precipitate formed immediately. The mixture was shaken overnight at room temperature to produce I-3b (as an L-tartrate salt of PI-d₁₀).

As shown in FIG. 66, the X-ray powder diffraction (XRPD) pattern indicates the material I-3b produced from the non-seeded experiment was poorly crystalline (referred to as pattern 1)—the material was poorly crystalline compared to crystalline polymorphs of I-7b of pattern 1 (from THF) and pattern 2 (from 1,4-dioxane) (see Example 3).

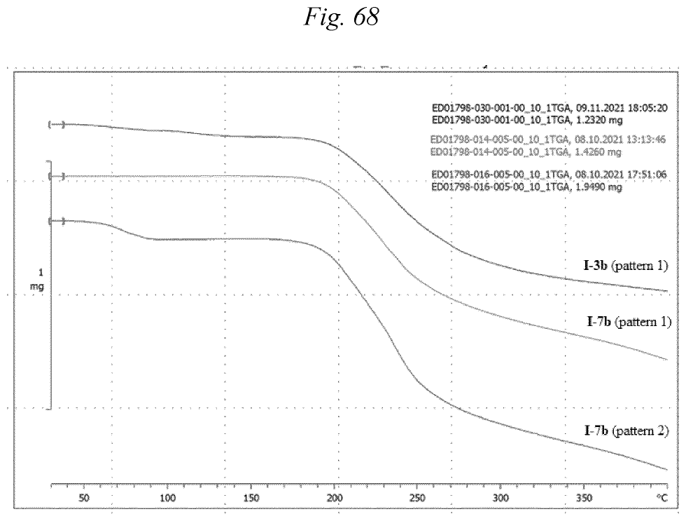

The DSC curve of I-3b (pattern 1) at 10° C./min is shown in FIG. 67, compared to that of the polymorph patterns 1 and 2 of I-7b, and it is again evident that I-3b (pattern 1) was poorly crystalline. The thermogravimetric analysis (TGA) curve of I-3b (pattern 1) at 10° C./min is shown in FIG. 68.

Seeded. Compound I-3 (PI-d₁₀, free base) (10.45 mg) was dissolved in THE (250 μL). Seeds of I-7b crystalline form of pattern 1 (see Example 3) were added followed by a 0.5M solution of L-tartaric acid in THE (1 equivalent) at room temperature. A precipitate formed immediately. A further portion of seeds were added, and the mixture was shaken at room temperature for 3 days. The solid was isolated by filtration to give crystals of I-3b.

The X-ray powder diffraction (XRPD) pattern (FIGS. 69A-69B) indicates that the seeded experiment produced a single crystalline form of I-3b (pattern 2), which was substantially the same as the seeds of crystalline polymorph of I-7b of pattern 1, and having a higher crystallinity compared to the crystalline polymorph of I-3b of pattern 1 obtained from the non-seeded experiments. Table 17 shows the XRPD peak listing for I-3b (pattern 2).

TABLE 17

| Name | Caption (display) | Angle | d Value | Net Intensity | Rel. Intensity |
|---|---|---|---|---|---|
| Peak #1 | 6.732° | 6.7315 | 13.1205 | 21.63181 | 0.01863163 |
| Peak #2 | 12.708° | 12.70753 | 6.960525 | 91.25528 | 0.0785988 |
| Peak #3 | 13.470° | 13.46988 | 6.568243 | 179.8262 | 0.1548855 |
| Peak #4 | 14.774° | 14.77353 | 5.991449 | 265.2903 | 0.2284964 |
| Peak #5 | 15.921° | 15.92107 | 5.562096 | 119.7453 | 0.1031374 |
| Peak #6 | 16.268° | 16.26759 | 5.444388 | 51.88243 | 0.04468669 |
| Peak #7 | 17.295° | 17.2945 | 5.123354 | 1161.026 | 1 |
| Peak #8 | 18.869° | 18.86872 | 4.699313 | 465.4803 | 0.4009213 |
| Peak #9 | 20.079° | 20.0787 | 4.418771 | 216.5187 | 0.186489 |
| Peak #10 | 20.208° | 20.20805 | 4.390777 | 379.5682 | 0.3269247 |
| Peak #11 | 20.877° | 20.87668 | 4.251637 | 136.9393 | 0.1179468 |
| Peak #12 | 21.894° | 21.89416 | 4.056297 | 343.8481 | 0.2961587 |
| Peak #13 | 22.657° | 22.65734 | 3.921363 | 598.8112 | 0.5157602 |
| Peak #14 | 23.491° | 23.49116 | 3.784026 | 184.869 | 0.1592289 |
| Peak #15 | 23.702° | 23.70229 | 3.750796 | 154.3493 | 0.1329421 |
| Peak #16 | 24.636° | 24.63617 | 3.610689 | 137.7375 | 0.1186342 |
| Peak #17 | 24.882° | 24.88205 | 3.575563 | 200.4769 | 0.1726721 |
| Peak #18 | 25.569° | 25.56915 | 3.481015 | 157.8053 | 0.1359188 |
| Peak #19 | 26.685° | 26.68466 | 3.337975 | 173.1976 | 0.1491763 |
| Peak #20 | 27.060° | 27.06003 | 3.292517 | 45.83561 | 0.03947853 |
| Peak #21 | 27.502° | 27.50171 | 3.240634 | 50.41544 | 0.04342317 |
| Peak #22 | 28.179° | 28.17879 | 3.164288 | 30.12094 | 0.02594337 |
| Peak #23 | 28.597° | 28.59672 | 3.118986 | 121.3689 | 0.1045359 |
| Peak #24 | 29.035° | 29.03487 | 3.072908 | 50.59545 | 0.04357821 |
| Peak #25 | 29.257° | 29.25673 | 3.050108 | 65.04179 | 0.05602094 |
| Peak #26 | 29.527° | 29.52666 | 3.022835 | 100.3665 | 0.08644637 |
| Peak #27 | 31.017° | 31.01673 | 2.880928 | 79.85507 | 0.06877972 |
| Peak #28 | 31.527° | 31.52703 | 2.835451 | 45.04162 | 0.03879466 |
| Peak #29 | 32.059° | 32.05877 | 2.789625 | 78.14873 | 0.06731004 |
| Peak #30 | 32.307° | 32.30659 | 2.768789 | 30.37478 | 0.02616201 |
| Peak #31 | 33.012° | 33.01181 | 2.711234 | 24.27336 | 0.02090681 |
| Peak #32 | 34.024° | 34.02446 | 2.632821 | 19.82419 | 0.01707471 |
| Peak #33 | 34.388° | 34.38762 | 2.605843 | 77.70146 | 0.0669248 |
| Peak #34 | 34.905° | 34.90529 | 2.568373 | 19.86779 | 0.01711226 |
| Peak #35 | 35.361° | 35.36144 | 2.53628 | 24.57932 | 0.02117033 |
| Peak #36 | 36.183° | 36.18279 | 2.480568 | 44.07069 | 0.03795839 |
| Peak #37 | 37.372° | 37.37207 | 2.404317 | 74.44651 | 0.06412129 |
| Peak #38 | 37.764° | 37.76379 | 2.380272 | 43.85532 | 0.03777289 |
| Peak #39 | 38.657° | 38.65681 | 2.327315 | 62.80877 | 0.05409762 |
| Peak #40 | 41.049° | 41.04871 | 2.197056 | 32.98244 | 0.028408 |

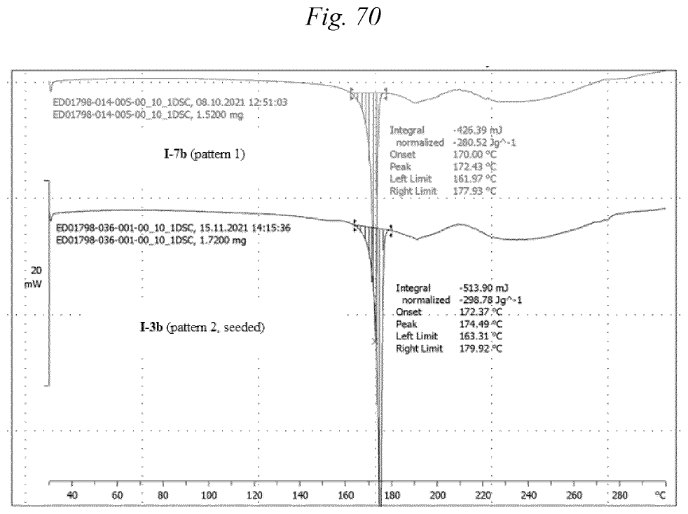

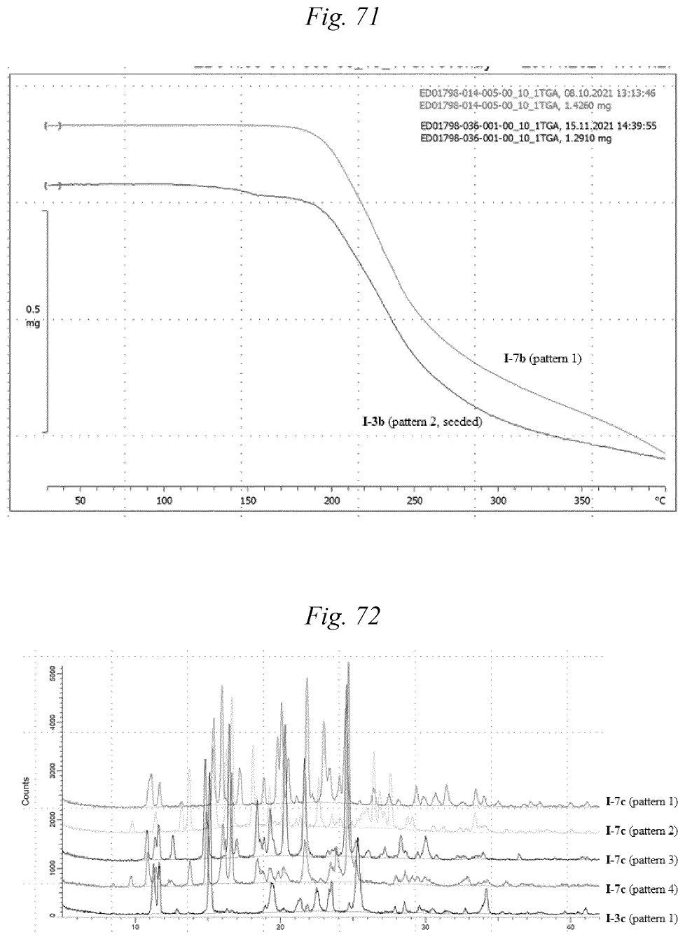

The single crystal X-ray structure of I-3b (pattern 2) is shown in FIGS. 69C-69D. The DSC curve of I-3b (pattern 2) at 10° C./min is shown in FIG. 70, which shows a melt onset of 172.37° C. (peak 174.49° C.), similar to that of the polymorph pattern 1 of I-7b. The thermogravimetric analysis (TGA) curve of I-3b (pattern 2) at 10° C./min is shown in FIG. 71, which is also similar to that of 1-7b (pattern 1).

The identity of I-3b (pattern 2) was also confirmed by ¹H NMR, which showed ca. 1 equivalent of tartrate and 0.1 equivalents of THF. The UPLC purity was 99.5%.

### Example 15

### Synthesis of Hemi-Fumarate Salt of 3-(2-(bis(methyl-d₃)amino)ethyl-1,1,2,2-d₄)-1H-indol-4-ol (I-3c)(Hemi-Fumarate Salt of I-3/psilocin-d₁₀/PI-d₁₀)

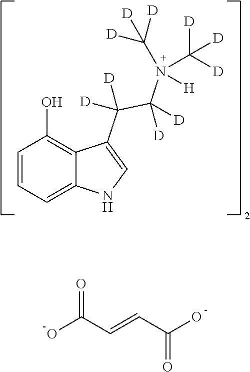

Non-seeded. Compound I-3 (PI-d₁₀, free base) (10.51 mg) was dissolved in acetonitrile (500 μL) and treated with a 0.25M solution of fumaric acid in THF (1 equivalent). After addition of acid, a precipitate formed. The mixture was shaken at room temperature overnight, and then the supernatant was removed by pipette and the solid was dried under a gentle stream of nitrogen to produce I-3c (hemi-fumarate salt of PI-d₁₀).

After characterization by XRPD, crystalline material of I-3c isolated above was designated as crystalline form of pattern 1. The XRPD pattern is presented in FIG. 72 comparing I-3c (pattern 1) to the crystalline polymorphs of I-7c of patterns 1 through 4 (see Example 4).

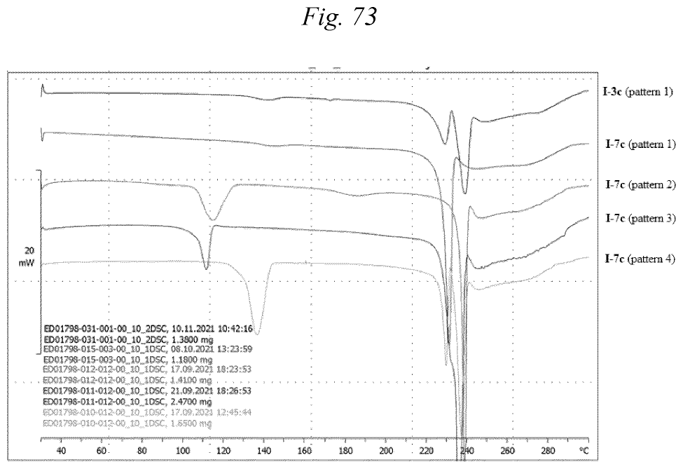

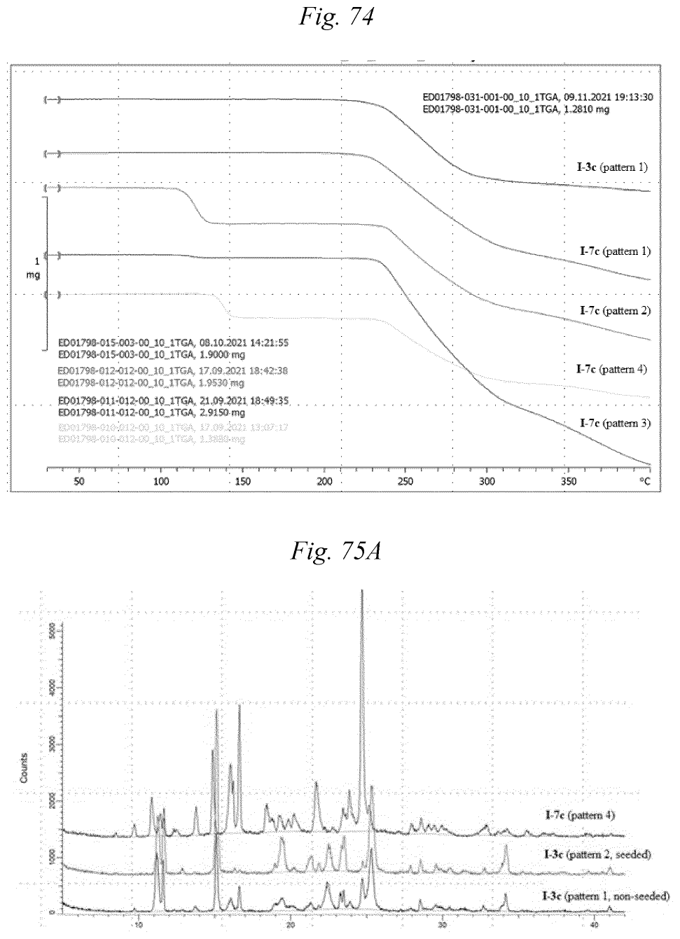

The DSC curve of I-3c (pattern 1) at 10° C./min is shown in FIG. 73, compared to that of the polymorph patterns 1 through 4 of I-7c, which indicates that I-3c (pattern 1) is likely to be a mixture of polymorphs. The thermogravimetric analysis (TGA) curve of I-3c (pattern 1) at 10° C./min is shown in FIG. 74.

Seeded. Compound I-3 (PI-d₁₀, free base) (10.47 mg) was dissolved in acetonitrile (500 μL) and a few seeds of I-7c crystalline polymorph pattern 4 (see Example 4) were added, followed by treatment with a 0.25M solution of fumaric acid in THE (1 equivalent). A precipitate formed immediately. A further portion of seeds were added and then the mixture was shaken at room temperature for 3 days. Then the supernatant was removed by pipette and the solid was dried under a gentle stream of nitrogen to produce I-3c as a white solid.

The X-ray powder diffraction (XRPD) pattern (FIG. 75A) indicates that the seeded experiment produced a single crystalline form of I-3c (pattern 2) which was different from the crystalline polymorph of I-3c of pattern 1 obtained from the non-seeded experiments. The X-ray powder diffraction (XRPD) pattern of I-3c (pattern 2) is shown in FIG. 75B and the peak listing is provided as follows: 9.713°, 11.209°, 11.605°, 12.338°, 12.852°, 13.718°, 15.117°, 16.066°, 16.627°, 19.026°, 19.427°, 20.108°, 21.068°, 21.335°, 21.837°, 22.429°, 23.262°, 23.478°, 23.900°, 24.720°, 25.318°, 27.912°, 28.532°, 29.565°, 30.457°, 32.698°, 34.155°, 37.910°, 39.566°, and 40.999°.

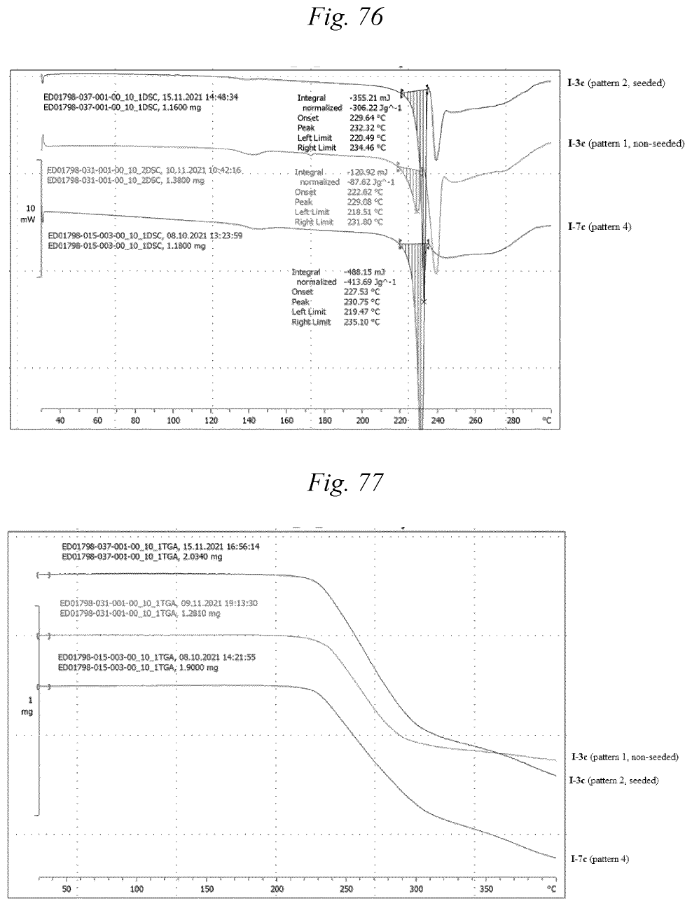

The DSC curve of I-3c (pattern 2) at 10° C./min is shown in FIG. 76, which shows a first melt onset of 229.64° C. (peak 232.32° C.), but the analysis was complicated by polymorphism issues. The thermogravimetric analysis (TGA) curve of I-3c (pattern 2) at 10° C./min is shown in FIG. 77, which is similar to that of I-7c (pattern 1) obtained from the non-seeded experiment.

The identity of I-3c (pattern 2) was also confirmed by ¹H NMR, which showed ca. 0.5 equivalents of fumarate and trace (0.009 equivalents) of MeCN. The UPLC purity was 99.4%.

### Example 16

### Synthesis of Benzoate Salt of 3-(2-(bis(methyl-d₃)amino)ethyl-1,1,2,2-d₄)-1H-indol-4-ol (I-3j)(Benzoate Salt of I-3/psilocin-d₁₀/PI-d₁₀)

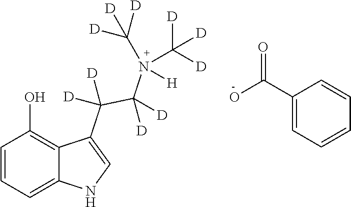

Compound I-3 (PI-d₀, free base) (499 mg) was dissolved in THE (11.9 mL), treated with seeds of I-7j (pattern 1)(see Example 11), then a solution of benzoic acid (284.3 mg, 1 equivalent) in THF (2.35 mL) was added. The resulting suspension was stirred, additional seeds were added, and stirred at room temperature for 4 days. The precipitate was isolated by filtration under a stream of nitrogen (inverted funnel) and dried in vacuo to give I-3j as a white solid (82% yield).

As shown in FIGS. 78A-78B, the X-ray powder diffraction (XRPD) pattern indicates the I-3j salt is crystalline, with only one crystal form observed (pattern 1), which was the same as the pattern of the I-7j seed (FIG. 78C). Table 18 shows the XRPD peak listing for I-3j (pattern 1).

TABLE 18

| Caption | | | Net | Rel. |
|---|---|---|---|---|
| (display) | Angle | d Value | Intensity | Intensity |
| 9.486° | 9.486166 | 9.315752 | 670.1039 | 0.22989 |
| 11.006° | 11.00646 | 8.032168 | 128.1829 | 0.043975 |
| 12.379° | 12.37882 | 7.144608 | 663.2478 | 0.227538 |
| 13.428° | 13.42827 | 6.588506 | 150.0904 | 0.051491 |
| 14.608° | 14.60809 | 6.058931 | 1259.188 | 0.431984 |
| 15.446° | 15.44628 | 5.73198 | 71.64919 | 0.02458 |
| 16.389° | 16.38903 | 5.404319 | 2914.894 | 1 |
| 18.247° | 18.2471 | 4.857978 | 1790.053 | 0.614106 |
| 18.977° | 18.97682 | 4.672788 | 2356.012 | 0.808267 |
| 19.346° | 19.34612 | 4.584413 | 686.9104 | 0.235655 |
| 19.831° | 19.83096 | 4.47341 | 856.2676 | 0.293756 |
| 20.868° | 20.8683 | 4.253326 | 51.75928 | 0.017757 |
| 21.447° | 21.4467 | 4.139906 | 473.447 | 0.162423 |
| 22.860° | 22.85971 | 3.887104 | 355.8473 | 0.122079 |
| 23.878° | 23.87811 | 3.723574 | 292.7112 | 0.100419 |
| 24.944° | 24.94418 | 3.566798 | 571.3039 | 0.195995 |
| 25.737° | 25.73693 | 3.458703 | 237.9127 | 0.08162 |
| 26.144° | 26.14398 | 3.405768 | 1355.461 | 0.465012 |
| 26.341° | 26.3413 | 3.380702 | 164.0407 | 0.056277 |
| 26.990° | 26.98996 | 3.300906 | 45.78438 | 0.015707 |
| 27.708° | 27.70765 | 3.217014 | 286.3526 | 0.098238 |
| 28.595° | 28.59503 | 3.119166 | 340.7419 | 0.116897 |
| 30.048° | 30.04825 | 2.971541 | 43.63537 | 0.01497 |
| 30.763° | 30.76306 | 2.904104 | 92.80374 | 0.031838 |
| 31.127° | 31.12661 | 2.871009 | 64.86243 | 0.022252 |
| 31.839° | 31.83931 | 2.80835 | 90.03876 | 0.030889 |
| 32.800° | 32.8004 | 2.728225 | 61.20367 | 0.020997 |
| 34.460° | 34.45993 | 2.600541 | 49.32027 | 0.01692 |
| 35.444° | 35.44447 | 2.530529 | 70.97894 | 0.02435 |
| 37.725° | 37.7249 | 2.382637 | 42.98652 | 0.014747 |
| 38.597° | 38.59706 | 2.33078 | 75.10263 | 0.025765 |

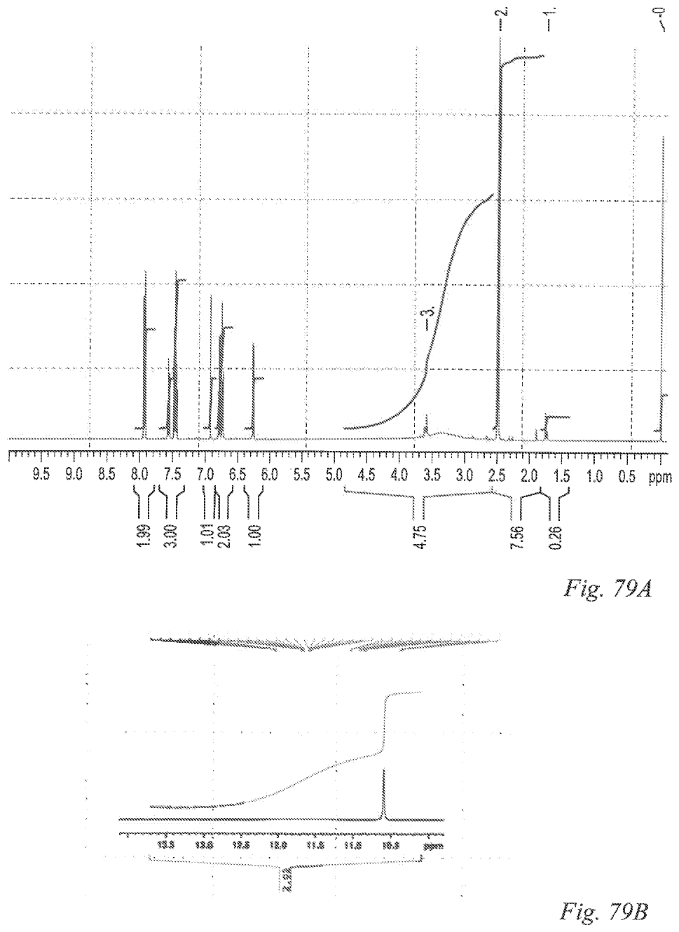

¹H NMR confirmed 1 equivalent of benzoic acid (FIGS. 79A and 79B). UPLC analysis showed a purity of >99.5% (including the counterion) and showed the expected M+H⁺ ion (215.1).

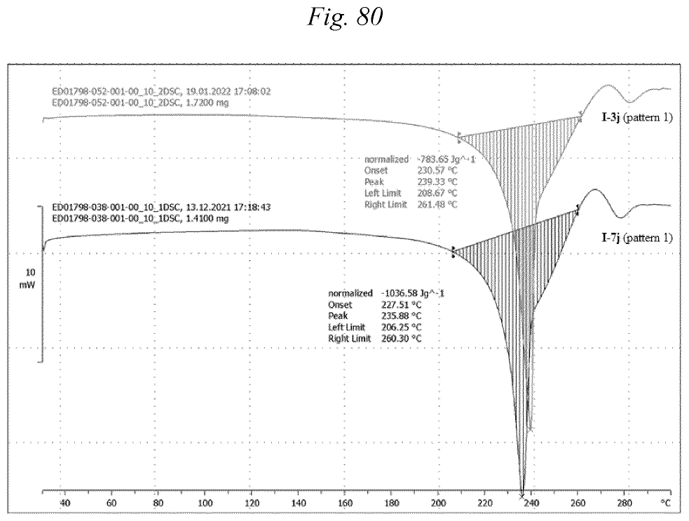

DSC of I-3j showed a high melt onset (230.57° C.) and peak (239.33° C.) (FIG. 80), with no events observed prior to the melt endotherm, indicating the absence of multiple physical forms in the sample, and also no conversion of physical forms prior to the melt, similar to the DSC of I-7j.

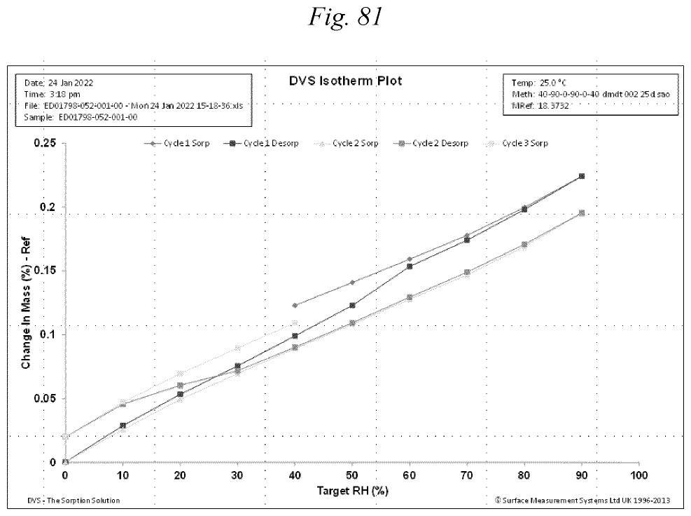

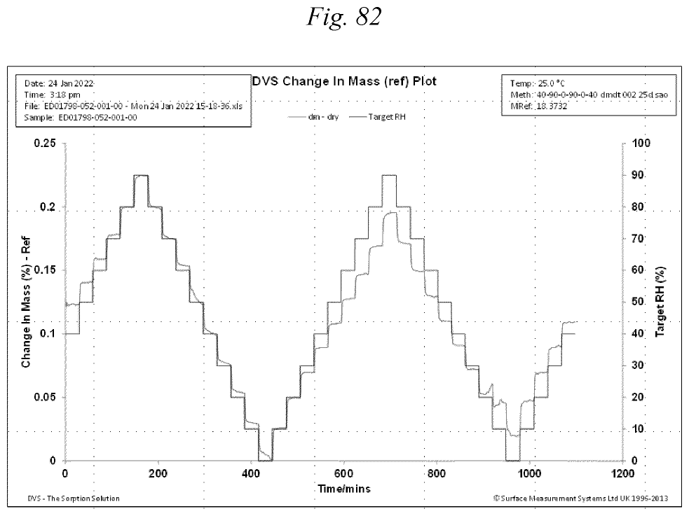

DVS isotherm plot of I-3j is shown in FIG. 81, which shows a 0.2% mass increase over 0-90% RH range (second sorption cycle), and thus the sample had no significant hygroscopicity, similar to I-7j which had a 0.16% mass increase over 0-90% RH range (second sorption cycle). This can be seen in the DVS change in mass plot of FIG. 82.

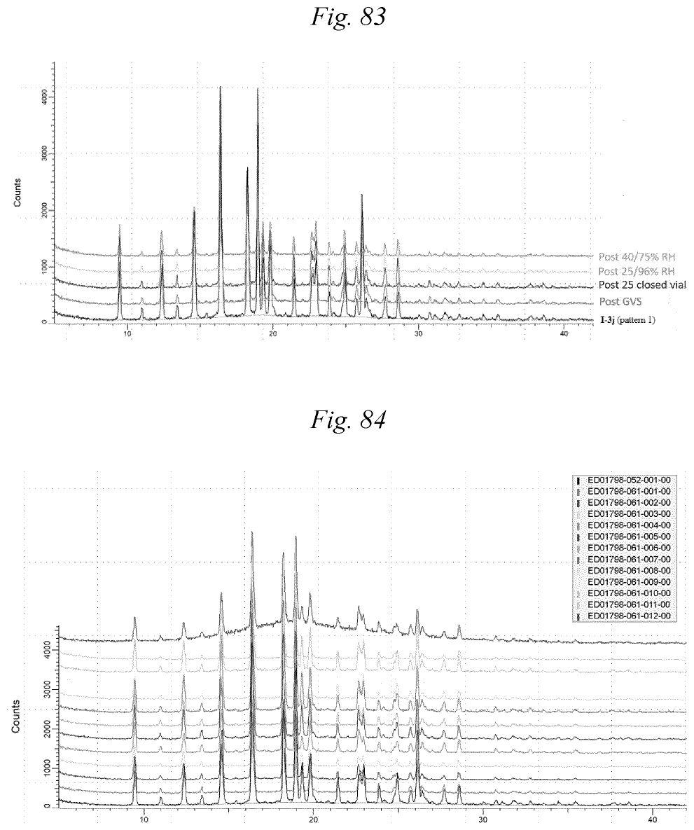

The storage stability of I-3j was also assessed by storing the solid samples for 7 days under the following conditions: i) 25° C., closed vial, ii) 25° C./96% RH, and iii) 40° C./75% RH, and comparing to fresh sample and the sample post DVS from above by XRPD. The results are presented in FIG. 83, which showed no change in form by XRPD post storage or post DVS.

I-3j was also subjected to maturation in 12 different solvents following the procedure detailed in Example 2. All samples were isolated as solids and retained their initial crystalline form (pattern 1) according to XRPD analysis (FIG. 84), which was similar behavior to that observed for I-7j.

Together, this data represents advantageous behavior for pharmaceutical development purposes.

From the single crystals of I-3j grown from THF at room temperature, a suitable crystal was selected and mounted on a glass fiber with Fomblin® oil. This was then placed on a Rigaku Oxford Diffraction SuperNova diffractometer with a dual source (Cu at zero) collection. Using Olex2 (Dolomanov, O. V., Bourhis, L. J., Gildea, R. J, Howard, J. A. K. & Puschmann, H. 2009, J. Appl. Cryst. 42, 339-341), the structure was-solved with the SHELXT (Sheldrick, G. M. 2015, Acta Cryst. A71, 3-8) structure solution program using Intrinsic Phasing and refined with the SHELXL (Sheldrick, G. M. 2015, Acta Cryst. C71, 3-8) refinement package using Least Squares minimization.

The solid-state structure of I-3j was generated. The asymmetric unit contains a protonated 4-hydroxy-N,N-dimethyltryptamine and a benzoate counter ion as shown in FIG. 78D, with four of each in the unit cell. The structure was refined containing both hydrogen and deuterium. There is interesting whole molecule disorder related by a 180-degree rotation about an axis through C5-C8 of the benzene ring. Many of the disordered components overlap but the disorder was modelled with both components sharing the orientation of the benzene ring but with different orientations of the 5 membered ring related by the above mentioned 180 degree rotation. The NH of the indole of one orientation maps onto the OH of the other orientation and vice versa. This disorder was traced right out to the dimethylamine unit. The occupancy of the two components were linked to a free variable which refined to 70:30 and this is depicted in FIG. 78E. The minor component was refined isotropically. Several distance and thermal parameter restraints were used to give the disordered components reasonable bond lengths and thermal parameters. The NHs and OH of the main component were located in a difference map. All NHs and OHs were placed at calculated positions for the refinement. They form short contacts listed in Table 19.

TABLE 19

| D − H | H . . . A | D . . . A | <(DHA) | Bond descriptor |
|---|---|---|---|---|
| 0.88 | 1.94 | 2.765(12) | 156.2 | N2\hat{ }a-H2\hat{ }a . . . O15 $1 |
| 0.84 | 1.86 | 2.657(4) | 157.8 | O7\hat{ }a-H7\hat{ }a . . . O16 $2 |
| 0.88 | 1.91 | 2.735(5) | 154.6 | N2A\hat{ }b-H2A\hat{ }b . . . O16_$2 |
| 1.00 | 1.68 | 2.677(5) | 174.6 | N12\hat{ }a-H12\hat{ }a . . . O16 |
| 1.00 | 1.76 | 2.757(11) | 174.3 | N12A\hat{ }b-H12A\hat{ }b . . . O15 |
| 0.84 | 1.95 | 2.77(3) | 164.2 | O7A\hat{ }b-H7A\hat{ }b . . . O15_$1 |

Symmetry operators used to generate symmetry equivalent atoms in above contact were $1 − X, 0.5 + Y, 0.5 − Z, $2 1 − X, 1 − Y, 1−Z

Crystal Data for C₁₉H₁₂D₁₀N₂O₃ (M=336.23 g/mol): monoclinic, space group P2₁/c (no. 14), a=9.6406(2) Å, b=11.5042(3) Å, c=16.6070(3) Å, β=105.895(2)°, V=1771.42(8) Å3, Z=4, T=200(2) K, μ(CuKα)=0.673 mm−1, Dcalc=1.262 g/cm³, 20733 reflections measured (9.474°≤2θ≤147.084°), 3542 unique (Rint=0.0256, Rsigma=0.0146) which were used in all calculations. The final R₁ was 0.0931 (1>2σ((I)) and wR₂ was 0.2289 (all data).

A complete summary of the geometry coordinates is shown in Tables 20-27. The proposed crystal structure has a very high degree of -certainty and is consistent with expectation in accordance with the determined counter-ion stoichiometry.

TABLE 20
Crystal structure and data refinement

| Empirical formula | C₁₉H₁₂D₁₀N₂O₃ |
|---|---|
| Formula weight | 336.45 |
| Temperature/K | 200(2) |
| Crystal system | monoclinic |
| Space group | P21/c |
| a/Å | 9.6406(2) |
| b/Å | 11.5042(3) |
| c/Å | 16.6070(3) |
| α/c | 90 |
| β/° | 105.895(2) |
| γ/° | 90 |
| Volume/Å3 | 1771.42(8) |
| Z | 4 |
| ρcalcg/cm3 | 1.262 |
| μ/mm−1 | 0.673 |
| F(000): | 696.0 |
| Crystal size/mm3 | 0.2 × 0.1 × 0.08 colourless block |
| Radiation | CuKα (λ = 1.54184) |
| 2Θ range for data collection/° | 9.474 to 147.084 |
| Index ranges | −11 ≤ h ≤ 11, −14 ≤ k ≤ 14, −20 ≤ |
| | l ≤ 19 |
| Reflections collected | 20733 |
| Independent reflections | 3542 [Rint = 0.0256, Rsigma = 0.0146] |
| Data/restraints/parameters | 3542/42/252 |
| Goodness-of-fit on F2 | 1.091 |
| Final R indexes [I >= 2σ (I)] | R1 = 0.0931, wR2 = 0.2265 |
| Final R indexes [all data] | R1 = 0.0967, wR2 = 0.2289 |
| Largest diff. peak/hole/e Å−3 | 0.71/−0.49 |

TABLE 21
Fractional Atomic Coordinates (×10⁴) and Equivalent Isotropic
Displacement Parameters (Å² × 10³)

| Atom | x | y | z | U(eq) |
|---|---|---|---|---|
| O15 | 2746(2) | 1021(2) | 3472.8(13) | 61.4(6) |
| O16 | 4532(3) | 1971(3) | 4306.1(16) | 81.6(9) |
| N2 | −870(17) | 5612(16) | 3089(9) | 61(4) |
| O7 | 3346(4) | 6541(4) | 5052(2) | 66.5(16) |
| C17 | 5015(3) | 923(3) | 3182.1(18) | 53.1(7) |
| C8 | 1183(3) | 6146(2) | 4057.1(16) | 47.0(7) |
| C9A | 1665(6) | 5056(2) | 4430(4) | 63(3) |
| C1A | 3032(8) | 5225(5) | 5015(6) | 88(4) |
| N2A | 3395(5) | 6420(5) | 5003(4) | 107(7) |
| C7 | 2252(3) | 6989(3) | 4411(2) | 60.9(8) |
| C3 | 7(3) | 6525(3) | 3381.6(17) | 51.4(7) |
| C16 | 4034(3) | 1329(3) | 3684.5(18) | 52.5(7) |
| C18 | 4668(4) | −43(3) | 2671(2) | 64.6(9) |
| C4 | −91(4) | 7661(3) | 3093(2) | 65.6(9) |
| C5 | 1004(5) | 8422(3) | 3490(3) | 75.4(11) |
| C22 | 6291(4) | 1498(4) | 3219(3) | 75.1(10) |
| C19 | 5573(6) | −427(4) | 2214(3) | 83.2(12) |
| N12 | 2647(5) | 2173(4) | 5220(2) | 64.8(12) |
| C6 | 2157(5) | 8090(3) | 4129(3) | 72.7(10) |
| C9 | 866(5) | 4966(4) | 4172(2) | 54.4(11) |
| C20 | 6821(6) | 152(4) | 2251(3) | 92.7(15) |
| C11 | 1512(7) | 2982(4) | 4840(3) | 59.3(13) |
| D11A | 1037.97 | 2708.05 | 4265.52 | 71 |
| D11B | 782.26 | 2954.6 | 5159 | 71 |
| C21 | 7177(6) | 1118(4) | 2748(3) | 95.4(15) |
| C1 | −368(6) | 4695(4) | 3593(3) | 58.9(12) |
| C13 | 1978(9) | 999(6) | 5293(5) | 78.4(18) |
| D13A | 1338.46 | 1065.96 | 5658.81 | 118 |
| D13B | 2741.76 | 434.52 | 5530.58 | 118 |
| D13C | 1421.26 | 737.61 | 4736.69 | 118 |

| Atom | x | y | z | U(eq) |
|---|---|---|---|---|
| C10 | 1926(7) | 4168(4) | 4791(3) | 71.8(15) |
| D10A | 2853.42 | 4175.66 | 4642.86 | 86 |
| D10B | 2107.74 | 4512.86 | 5356.82 | 86 |
| C14 | 3512(7) | 2589(5) | 6062(3) | 77.4(17) |
| D14A | 4096.13 | 3259.06 | 5992.82 | 116 |
| D14B | 4146.58 | 1962.38 | 6348.64 | 116 |
| D14C | 2860.13 | 2816.22 | 6394.9 | 116 |
| N12A | 1714(12) | 1809(9) | 4767(7) | 67(3) |
| C10A | 1218(13) | 3891(10) | 4312(8) | 60(3) |
| D10C | 320.32 | 3794.24 | 4484.27 | 72 |
| D10D | 997.79 | 3700.67 | 3708.36 | 72 |
| C14A | 158(14) | 1525(13) | 4485(9) | 77(4) |
| D14D | 37.23 | 704.2 | 4322.84 | 116 |
| D14E | −311.53 | 2012.24 | 4003.48 | 116 |
| D14F | −282.48 | 1670.23 | 4942.03 | 116 |
| C13A | 2620(20) | 1110(20) | 5464(14) | 94(7) |
| D13D | 3618.95 | 1391.71 | 5598.04 | 140 |
| D13E | 2587.35 | 293.87 | 5297.95 | 140 |
| D13F | 2257.49 | 1192.6 | 5957.61 | 140 |
| C11A | 2200(20) | 3155(15) | 4741(13) | 96(4) |
| D11C | 2540.44 | 3439.76 | 5324.35 | 115 |
| D11D | 3038.14 | 3176.97 | 4502.73 | 115 |
| O7A | −1010(30) | 5600(30) | 3127(19) | 55(6) |

TABLE 22
Anisotropic displacement parameters (Å² × 10³)

| Atom | U11 | U22 | U33 | U23 | U13 | U12 |
|---|---|---|---|---|---|---|
| O15 | 48.3(12) | 80.2(16) | 49.9(12) | −9.1(11) | 3.7(9) | −7.2(11) |
| O16 | 62.0(15) | 117(2) | 65.6(15) | −42.9(15) | 17.6(12) | −25.5(15) |
| N2 | 45(4) | 89(6) | 38(3) | 14(2) | −9(3) | 0(3) |
| O7 | 49(2) | 71(2) | 61(2) | −20.4(18) | −15.0(15) | −0.7(16) |
| C17 | 56.8(17) | 60.1(18) | 38.9(14) | 5.2(13) | 7.1(12) | 3.7(14) |
| C8 | 46.8(15) | 52.7(16) | 37.7(13) | −2.8(12) | 5.1(11) | 2.0(12) |
| C7 | 57.5(18) | 68(2) | 55.2(17) | −16.8(16) | 12.6(14) | −3.6(16) |
| C3 | 50.2(16) | 66.7(19) | 36.2(13) | 4.7(13) | 9.9(12) | 5.3(14) |
| C16 | 54.1(17) | 61.2(18) | 37.0(14) | −2.0(13) | 3.4(12) | −2.2(14) |
| C18 | 74(2) | 64(2) | 50.9(17) | −4.2(15) | 8.9(16) | 7.5(17) |
| C4 | 74(2) | 77(2) | 45.8(16) | 13.9(16) | 16.7(15) | 25.5(19) |
| C5 | 114(3) | 49.5(19) | 73(2) | 2.2(17) | 42(2) | 2(2) |
| C22 | 79(3) | 75(2) | 79(2) | −7(2) | 35(2) | −10(2) |
| C19 | 116(4) | 75(3) | 62(2) | −4.3(19) | 29(2) | 18(3) |
| N12 | 85(3) | 65(3) | 39.1(18) | 0.8(17) | 8.0(19) | 29(2) |
| C6 | 86(3) | 62(2) | 75(2) | −14.7(18) | 29(2) | −17.8(19) |
| C9 | 63(3) | 56(3) | 32.9(19) | 2.1(17) | −6.4(18) | 2(2) |
| C20 | 127(4) | 85(3) | 87(3) | 15(2) | 66(3) | 25(3) |
| C11 | 84(3) | 55(3) | 55(2) | 12(2) | 45(2) | 10(2) |
| C21 | 99(3) | 92(3) | 119(4) | −1(3) | 70(3) | −7(3) |
| C1 | 73(3) | 55(3) | 43(2) | 2.7(19) | 7(2) | −13(2) |
| C13 | 97(6) | 76(4) | 68(4) | 11(3) | 33(4) | 2(4) |
| C10 | 91(4) | 57(3) | 48(2) | 2(2) | −14(3) | 11(3) |
| C14 | 92(4) | 91(4) | 36(2) | 1(2) | −4(2) | 34(3) |

TABLE 23
Bond lengths

| Atom | Atom | Length/Å | Atom | Atom | Length/Å |
|---|---|---|---|---|---|
| O15 | C16 | 1.246(4) | C3 | O7A | 1.43(3) |
| O16 | C16 | 1.253(4) | C18 | C19 | 1.377(6) |
| N2 | C3 | 1.352(17) | C4 | C5 | 1.392(6) |
| N2 | C1 | 1.352(17) | C5 | C6 | 1.365(6) |
| O7 | C7 | 1.377(5) | C22 | C21 | 1.378(6) |
| C17 | C16 | 1.497(5) | C19 | C20 | 1.362(7) |
| C17 | C18 | 1.383(5) | N12 | C11 | 1.443(6) |
| C17 | C22 | 1.383(5) | N12 | C13 | 1.516(9) |
| C8 | C9A | 1.4200 | N12 | C14 | 1.496(6) |
| C8 | C7 | 1.4200 | C9 | C1 | 1.345(6) |
| C8 | C3 | 1.427(4) | C9 | C10 | 1.539(6) |
| C8 | C9 | 1.416(5) | C20 | C21 | 1.370(7) |
| C9A | C1A | 1.4200 | C11 | C10 | 1.431(8) |
| C9A | C10A | 1.405(12) | N12A | C14A | 1.481(17) |
| C1A | N2A | 1.4200 | N12A | C13A | 1.48(2) |
| N2A | C7 | 1.4200 | N12A | C11A | 1.62(2) |
| C7 | C6 | 1.344(5) | C10A | C11A | 1.33(2) |

| | | |
|---|---|---|
| C3 | C4 | 1.386(5) |

TABLE 24
Bond angles

| Atom | Atom | Atom | Angle/° | Atom | Atom | Atom | Angle/° |
|---|---|---|---|---|---|---|---|
| C3 | N2 | C1 | 107.5(8) | O15 | C16 | C17 | 119.1(3) |
| C18 | C17 | C16 | 120.6(3) | O16 | C16 | C17 | 119.1(3) |
| C18 | C17 | C22 | 118.3(3) | C19 | C18 | C17 | 120.9(4) |
| C22 | C17 | C16 | 121.0(3) | C3 | C4 | C5 | 117.3(3) |
| C9A | C8 | C3 | 135.0(3) | C6 | C5 | C4 | 122.8(3) |
| C7 | C8 | C9A | 108.0 | C21 | C22 | C17 | 120.3(4) |
| C7 | C8 | C3 | 116.6(3) | C20 | C19 | C18 | 120.2(4) |
| C9 | C8 | C7 | 139.3(3) | C11 | N12 | C13 | 108.7(5) |
| C9 | C8 | C3 | 104.1(2) | C11 | N12 | C14 | 111.4(4) |
| C8 | C9A | C1A | 108.0 | C14 | N12 | C13 | 110.5(5) |
| C10A | C9A | C8 | 136.9(6) | C7 | C6 | C5 | 119.9(4) |
| C10A | C9A | C1A | 114.7(6) | C8 | C9 | C10 | 122.2(4) |
| N2A | C1A | C9A | 108.0 | C1 | C9 | C8 | 107.9(4) |
| C1A | N2A | C7 | 108.0 | C1 | C9 | C10 | 129.4(4) |
| O7 | C7 | C8 | 112.3(3) | C19 | C20 | C21 | 119.8(4) |
| C8 | C7 | N2A | 108.0 | C10 | C11 | N12 | 116.7(5) |
| C6 | C7 | O7 | 125.9(3) | C20 | C21 | C22 | 120.5(4) |
| C6 | C7 | C8 | 121.8(3) | C9 | C1 | N2 | 111.0(8) |
| C6 | C7 | N2A | 129.9(3) | C11 | C10 | C9 | 117.5(5) |
| N2 | C3 | C8 | 109.3(6) | C14A | N12A | C11A | 118.6(11) |
| N2 | C3 | C4 | 129.1(6) | C13A | N12A | C14A | 117.3(12) |
| C8 | C3 | O7A | 109.7(12) | C13A | N12A | C11A | 115.1(14) |
| C4 | C3 | C8 | 121.6(3) | C11A | C10A | C9A | 112.8(13) |
| C4 | C3 | O7A | 128.6(12) | C10A | C11A | N12A | 117.0(15) |

| | | | |
|---|---|---|---|
| O15 | C16 | O16 | 121.8(3) |

TABLE 25
Hydrogen bonds

| D | H | A | d(D—H)/Å | d(H—A)/Å | d(D—A)/Å | D—H—A/° |
|---|---|---|---|---|---|---|
| N2 | H2 | O15¹ | 0.88 | 1.94 | 2.765(12) | 156.2 |
| O7 | H7 | O16² | 0.84 | 1.86 | 2.657(4) | 157.8 |
| N2A | H2A | O16² | 0.88 | 1.91 | 2.735(5) | 154.6 |
| N12 | H12 | O16 | 1.00 | 1.68 | 2.677(5) | 174.6 |
| N12A | H12A | O15 | 1.00 | 1.76 | 2.757(11) | 174.3 |
| O7A | H7A | O15¹ | 0.84 | 1.95 | 2.77(3) | 164.2 |

¹−X, ½ + Y, ½ − Z;
²1 − X, 1 − Y, 1 − Z

TABLE 26
Hydrogen Atom Coordinates (Å × 10⁴) and Isotropic
Displacement Parameters (Å² × 10³)

| Atom | x | y | z | U(eq) |
|---|---|---|---|---|
| H2 | −1628.75 | 5615.1 | 2649.87 | 74 |
| H7 | 3853.1 | 7086.72 | 5313.46 | 100 |
| H1A | 3604.02 | 4641.02 | 5354.36 | 105 |
| H2A | 4188.78 | 6749.65 | 5308.8 | 128 |
| H18 | 3794.52 | −447.67 | 2635.49 | 78 |
| H4 | −875.14 | 7909.18 | 2642.63 | 79 |
| H5 | 947.53 | 9208.16 | 3307.27 | 90 |
| H22 | 6557.96 | 2158.3 | 3571.61 | 90 |
| H19 | 5326.1 | −1097.93 | 1870.56 | 100 |
| H12 | 3301.85 | 2092.76 | 4848.79 | 97 |
| H6 | 2891.82 | 8634.68 | 4374.4 | 87 |
| H20 | 7442.52 | −111.75 | 1933.85 | 111 |
| H21 | 8041.09 | 1528.28 | 2768.33 | 115 |
| H1 | −825.73 | 3956.74 | 3545.65 | 71 |
| H12A | 2037.03 | 1487.8 | 4289 | 140(70) |
| H7A | −1412.87 | 5655.14 | 2612.09 | 82 |

TABLE 27
Atomic occupancy

| Atom | Occupancy | Atom | Occupancy | Atom | Occupancy |
|---|---|---|---|---|---|
| N2 | 0.702(4) | H2 | 0.702(4) | O7 | 0.702(4) |
| H7 | 0.702(4) | C9A | 0.298(4) | C1A | 0.298(4) |
| H1A | 0.298(4) | N2A | 0.298(4) | H2A | 0.298(4) |
| N12 | 0.702(4) | H12 | 0.702(4) | C9 | 0.702(4) |
| C11 | 0.702(4) | D11A | 0.702(4) | D11B | 0.702(4) |
| C1 | 0.702(4) | H1 | 0.702(4) | C13 | 0.702(4) |
| D13A | 0.702(4) | D13B | 0.702(4) | D13C | 0.702(4) |
| C10 | 0.702(4) | D10A | 0.702(4) | D10B | 0.702(4) |
| C14 | 0.702(4) | D14A | 0.702(4) | D14B | 0.702(4) |
| D14C | 0.702(4) | N12A | 0.298(4) | H12A | 0.298(4) |
| C10A | 0.298(4) | D10C | 0.298(4) | D10D | 0.298(4) |
| C14A | 0.298(4) | D14D | 0.298(4) | D14E | 0.298(4) |
| D14F | 0.298(4) | C13A | 0.298(4) | D13D | 0.298(4) |
| D13E | 0.298(4) | D13F | 0.298(4) | C11A | 0.298(4) |
| D11C | 0.298(4) | D11D | 0.298(4) | O7A | 0.298(4) |

| | |
|---|---|
| H7A | 0.298(4) |

### III. Free Base Compound Forms

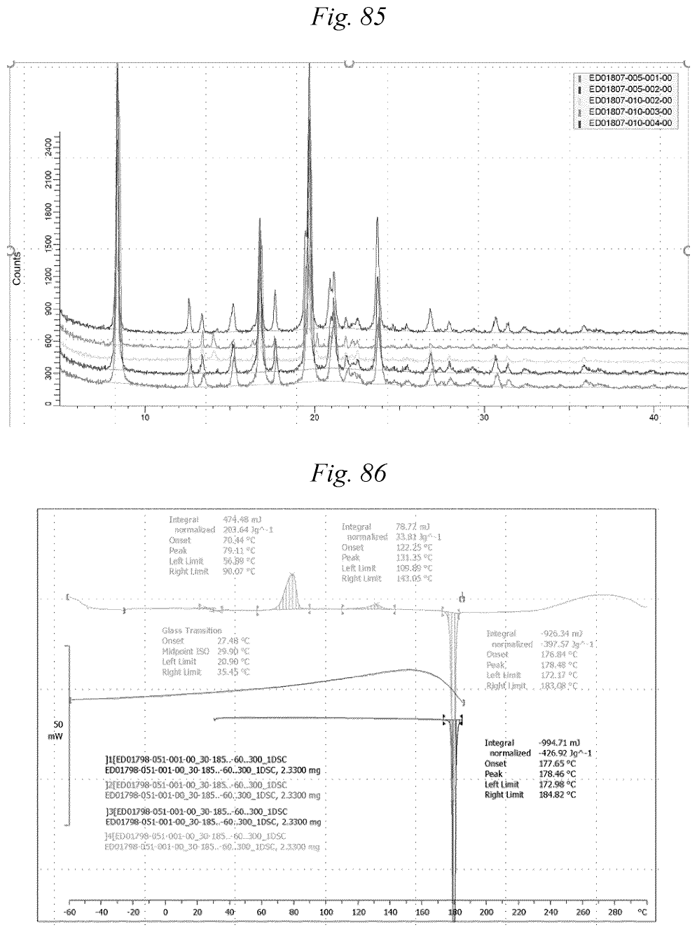

As described in Example 1, I-3 (PI-d₁₀, free base) was only isolated as a crystalline solid having an XRPD diffraction pattern 1 (see FIGS. 2A-2C). Attempts were made to prepare an amorphous form of I-3 (PI-d₁₀, free base) using a variety of techniques using the crystalline form (pattern 1) as input. The following techniques were tried but were not successful in the preparation of amorphous material:
i) Crash cooling/freeze drying. Crash cooling and freeze-drying solutions of I-3 (PI-d₁₀, free base) in 1,4-dioxane, t-BuOH, 1,4-dioxane/water, MeCN/water all gave material that still showed XRPD diffraction peaks of Pattern 1 (FIG. 85).
ii) Fast evaporation. Fast evaporation of a solution of I-3 (PI-d₁₀, free base) in dichloromethane (DCM) gave a crystalline material that still showed diffraction peaks of pattern 1.
iii) Anti-solvent precipitation. The addition of concentrated solutions of I-3 (PI-d₁₀, free base) in either dimethylformamide (DMF) or dimethylsulfoxide (DMSO) to water did not result in precipitation. Instead, solutions were formed which became dark within 4 hours, signifying degradation.

Next, a melt/crash cooling technique was investigated. An initial cyclic DSC experiment was conducted in which a portion of the crystalline input of I-3 (PI-d₁₀, free base) was heated to 185° C. (beyond the melting point with endothermic event onset at 178° C.) and then rapidly cooled to −60° C. The sample was then heated to 300° C. at 10° C./min. As can be seen in the DSC plot (FIG. 86), I-3 (PI-d₁₀, free base) showed a glass transition onset at about 27° C. and 2 exothermic events (onset at 70° C. and 122° C.) prior to the endothermic event onset at 177° C.

A melt/crash cooling experiment (>185° C./30° C.) was then conducted by heating I-3 (PI-d₁₀, free base) in DSC beyond the melting point (to 185° C.) and then rapidly cooled to 30° C. The resulting sample was then analyzed by XRPD, which indicated an amorphous form of I-3 (PI-d₁o, free base) was successfully prepared (FIG. 87). The amorphous material was not stable for prolonged periods, and crystallized overnight to crystal polymorph pattern 2, which was different from the crystalline polymorph pattern 1 used as input in the experiment (FIG. 87).

In summary, attempts to prepare amorphous psilocin-d₁₀ (I-3) by crash cooling/freeze drying or fast evaporation gave only crystalline material. Attempts to prepare amorphous psilocin-d₁₀ (I-3) by anti-solvent precipitation by addition to water did not yield solid material. Amorphous psilocin-d₁₀ (I-3) was successfully prepared by DSC melt/crash cooling (>185° C./30° C.). The amorphous form was unstable upon standing and reverted to a new crystalline form (pattern 2). The amorphous material shows a low glass transition temperature (27° C.).

Preparation of I-3 crystalline pattern 2 by DSC. Eleven portions of I-3 (pattern 1) (each ca. 40 mg) were weighed into 100 μL aluminum DSC pans and the following DSC experiment was performed on each one. (The sample was heated from 30 to 185° C. at 10° C./min, held at 185° C. for 5 minutes, rapidly cooled at −100° C./min to 0° C., heated to 90° C. at 10° C./min and then cooled at 10° C./min to 30° C.) After the experiments the DSC pans were opened and the contents scraped out. A portion of each was analyzed by XRPD to check that Pattern 2 had formed. The eleven samples were then combined to give Psilocin-d₁₀ free base crystals of Pattern 2 as a white solid (356 mg).

FIG. 88 shows the X-ray powder diffraction (XRPD) pattern of I-3 (pattern 2) from the DSC scale-up experiment, with FIG. 89 showing the annotated XRPD version. Table 28 shows the XRPD peak listing for I-3 (pattern 2).

TABLE 28

| Name | Caption (display) | Angle | d Value | Net Intensity | Rel. Intensity |
|---|---|---|---|---|---|
| Peak #1 | 8.124° | 8.123984 | 10.87445 | 1288.204 | 0.5911127 |
| Peak #2 | 8.357° | 8.357478 | 10.57115 | 409.2122 | 0.1877734 |
| Peak #3 | 10.059° | 10.05879 | 8.78668 | 89.95876 | 0.04127898 |
| Peak #4 | 12.630° | 12.63015 | 7.002995 | 40.84302 | 0.01874146 |
| Peak #5 | 13.420° | 13.41959 | 6.592748 | 323.3918 | 0.1483934 |
| Peak #6 | 13.743° | 13.74308 | 6.438281 | 159.5939 | 0.07323217 |
| Peak #7 | 14.053° | 14.05272 | 6.297107 | 739.7607 | 0.3394507 |
| Peak #8 | 15.220° | 15.22 | 5.816685 | 167.2829 | 0.07676034 |
| Peak #9 | 16.272° | 16.27205 | 5.442906 | 523.0821 | 0.2400244 |
| Peak #10 | 16.763° | 16.76291 | 5.284614 | 195.402 | 0.08966326 |
| Peak #11 | 16.954° | 16.95446 | 5.225336 | 235.4412 | 0.1080359 |
| Peak #12 | 17.328° | 17.32756 | 5.113654 | 48.59261 | 0.02229748 |
| Peak #13 | 17.662° | 17.66165 | 5.017669 | 71.53699 | 0.03282586 |
| Peak #14 | 18.062° | 18.06191 | 4.907367 | 97.47566 | 0.04472823 |
| Peak #15 | 18.742° | 18.74222 | 4.730745 | 32.0491 | 0.01470623 |
| Peak #16 | 19.413° | 19.41254 | 4.568877 | 745.5985 | 0.3421295 |
| Peak #17 | 19.658° | 19.65764 | 4.512459 | 521.7926 | 0.2394327 |
| Peak #18 | 20.172° | 20.17238 | 4.398462 | 2179.287 | 1 |
| Peak #19 | 20.836° | 20.8364 | 4.259765 | 152.215 | 0.06984623 |
| Peak #20 | 21.267° | 21.26686 | 4.174506 | 317.3733 | 0.1456317 |
| Peak #21 | 21.833° | 21.83276 | 4.067565 | 271.5293 | 0.1245954 |
| Peak #22 | 22.213° | 22.21281 | 3.998823 | 157.6812 | 0.07235449 |
| Peak #23 | 22.504° | 22.50401 | 3.947733 | 245.7072 | 0.1127466 |
| Peak #24 | 23.334° | 23.3343 | 3.809107 | 73.21411 | 0.03359544 |
| Peak #25 | 23.701° | 23.70123 | 3.750961 | 125.4296 | 0.05755533 |
| Peak #26 | 24.385° | 24.3849 | 3.647322 | 65.75739 | 0.0301738 |
| Peak #27 | 25.431° | 25.43138 | 3.49956 | 94.61557 | 0.04341583 |
| Peak #28 | 25.721° | 25.72122 | 3.46078 | 232.2516 | 0.1065723 |
| Peak #29 | 26.049° | 26.04914 | 3.417951 | 68.57748 | 0.03146784 |
| Peak #30 | 27.291° | 27.29145 | 3.265122 | 29.86893 | 0.01370582 |
| Peak #31 | 28.368° | 28.36801 | 3.14361 | 86.29607 | 0.0395983 |
| Peak #32 | 30.349° | 30.3493 | 2.942747 | 108.3302 | 0.04970898 |
| Peak #33 | 30.656° | 30.65631 | 2.913972 | 42.82265 | 0.01964984 |
| Peak #34 | 31.337° | 31.33691 | 2.85222 | 18.87493 | 0.008661055 |
| Peak #35 | 31.538° | 31.53802 | 2.834489 | 34.52836 | 0.01584388 |
| Peak #36 | 32.091° | 32.09148 | 2.786855 | 70.13274 | 0.0321815 |
| Peak #37 | 35.870° | 35.86994 | 2.501483 | 26.79949 | 0.01229736 |
| Peak #38 | 38.514° | 38.51409 | 2.33561 | 153.147 | 0.07027388 |
| Peak #39 | 41.361° | 41.36089 | 2.181192 | 26.51964 | 0.01216895 |

The structure of I-3 (pattern 2) from the DSC scale-up experiment was confirmed by ¹H NMR. The UPLC purity was determined to be 98.3%, and showed the expected [M+H]⁺ ion at 215. DSC of I-3 (pattern 2) at 10° C./min shows the material is an anhydrous form with an endothermic event of onset 177° C. (peak 178° C.) and a broad shallow exothermic event above ca. 230° C. (decomposition). TGA of I-3 (pattern 2) at 10° C./min shows good thermal stability with no significant mass loss until decomposition above ca. 190° C.

By GVS, I-3 (pattern 2) shows a mass increase of 0.13% over 0-90% RH range (second sorption cycle), indicating the polymorph is not hygroscopic. There were no changes in form by XRPD, or purity by UPLC, following GVS analysis.

### IV. Fatty Acid Salt Forms

Materials. Super Refined® oleic acid commercially available from Croda. Caprylic (octanoic) acid, stearic acid, capric (decanoic) acid, myristic acid, lauric acid, and sodium stearate commercially available from Sigma-Aldrich. Linoleic acid and sodium oleate commercially available from Sigma.

### Example 17

### Synthesis of Laurate Salt of 3-(2-(bis(methyl-d₃)amino)ethyl-1,1,2,2-d₄)-1H-indol-4-ol (I-3m) (Laurate Salt of I-3/psilocin-d₁₀/P-d₁₀)

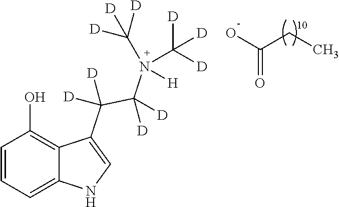

Compound I-3 (PI-d₁₀, free base) (ca. 50 mg) was dissolved in chloroform (ca. 25 mg/mL) in a glass vial. 1 molar equivalent of 1M lauric acid in chloroform was added to the free base solution and the sample was stirred at room temperature overnight. The solvent was then removed using a Biotage® V-10 evaporator. The resulting material was scraped and stored at −20° C. to induce crystallization. Crystals of I-3m were produced, which were then stored at 5° C. prior to analysis.

As shown in FIG. 90, the X-ray powder diffraction (XRPD) pattern indicates the I-3m salt is crystalline, with only one crystal form observed (pattern 1), which was different from the diffraction patterns 1 and 2 of the free base I-3.

### Example 18

### Synthesis of Linoleate Salt of 3-(2-(bis(methyl-d₃)amino)ethyl-1,1,2,2-d₄)-1H-indol-4-ol (I-3n) (Linoleate Salt of I-3/psilocin-d₁₀/PI-d₁₀)

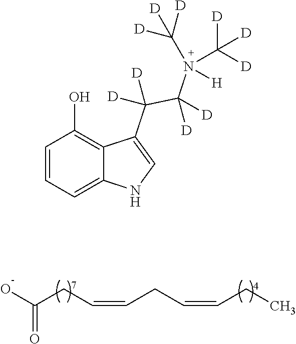

Compound I-3 (PI-d₁₀, free base) (ca. 50 mg) was dissolved in chloroform (ca. 25 mg/mL) in a glass vial. 1 molar equivalent of 1M linoleic acid (commercially available from Aldrich) in chloroform was added to the free base solution and the sample was stirred at room temperature overnight. The solvent was then removed using a Biotage® V-10 evaporator. The resulting material was scraped and stored at −20° C. to induce crystallization. Crystals of I-3n were produced, which were then stored at 5° C. prior to analysis.

As shown in FIG. 91, the X-ray powder diffraction (XRPD) pattern indicates the I-3n salt is crystalline, with only one crystal form observed (pattern 1), which was different from the diffraction patterns 1 and 2 of the free base I-3.

### Example 19

### Synthesis of Myristate Salt of 3-(2-(bis(methyl-d₃)amino)ethyl-1,1,2,2-d₄)-1H-indol-4-ol (I-3o) (Myristate Salt of I-3/psilocin-d₁₀/PI-d₁₀)

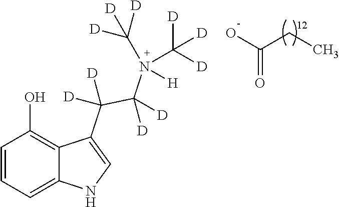

Compound I-3 (PI-d₁₀, free base) (ca. 50 mg) was dissolved in chloroform (ca. 25 mg/mL) in a glass vial. 1 molar equivalent of 1M myristic acid in chloroform was added to the free base solution and the sample was stirred at room temperature overnight. The solvent was then removed using a Biotage® V-10 evaporator. The resulting material was scraped and stored at −20° C. to induce crystallization. Crystals of I-3o were produced, which were then stored at 5° C. prior to analysis.

As shown in FIG. 92, the X-ray powder diffraction (XRPD) pattern indicates the I-3o salt is poorly crystalline, with only one crystal form observed (pattern 1), which was different from the diffraction patterns 1 and 2 of the free base I-3.

### Example 20

### Synthesis of Caprate Salt of 3-(2-(bis(methyl-d₃)amino)ethyl-1,1,2,2-d₄)-1H-indol-4-ol (I-3p) (Caprate Salt of I-3/psilocin-d₁₀/PI-d₁₀)

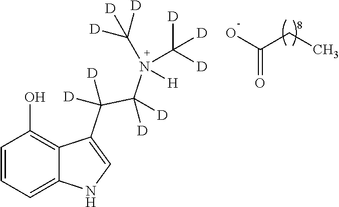

Compound I-3 (PI-d₁₀, free base) (ca. 50 mg) was dissolved in chloroform (ca. 25 mg/mL) in a glass vial. 1 molar equivalent of 1M capric acid in chloroform was added to the free base solution and the sample was stirred at room temperature overnight. The solvent was then removed using a Biotage® V-10 evaporator. The resulting material was scraped and stored at −20° C. to induce crystallization. Crystals of I-3p were produced, which were then stored at 5° C. prior to analysis.

As shown in FIG. 93, the X-ray powder diffraction (XRPD) pattern indicates the I-3p salt is crystalline, with only one crystal form observed (pattern 1), which was different from the diffraction patterns 1 and 2 of the free base I-3.

### Example 21

### Synthesis of stearate salt of 3-(2-(bis(methyl-d₃)amino)ethyl-1,1,2,2-d₄)-1H-indol-4-ol (I-3q) (stearate salt of I-3/psilocin-d₁₀/PI-d₁₀)

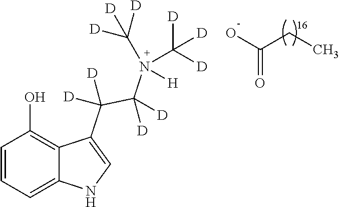

Compound I-3 (PI-d₁₀, free base) (ca. 50 mg) was dissolved in chloroform (ca. 25 mg/mL) in a glass vial. 1 molar equivalent of 0.5 M stearic acid (commercially available stearic acid or stearic acid obtained by desalting sodium stearate with 1M HCl) in chloroform was added to the free base solution and the sample was stirred at room temperature overnight. The solvent was then removed using a Biotage® V-10 evaporator. The resulting material was scraped and stored at −20° C. to induce crystallization. Crystals of I-3q were produced, which were then stored at 5C prior to analysis.

As shown in FIG. 94, the X-ray powder diffraction (XRPD) pattern indicates that two different polymorphs were formed: pattern 1 obtained from commercially available stearic acid, and pattern 2 obtained from desalting sodium stearate. Both polymorphs of I-3q were poorly crystalline and different from the diffraction patterns 1 and 2 of the free base I-3.

### Example 22

### Synthesis of Oleate Salt of 3-(2-(bis(methyl-d₃)amino)ethyl-1,1,2,2-d₄)-1H-indol-4-ol (I-3r) (Oleate Salt of I-3/psilocin-d₁₀/PI-d₁₀)

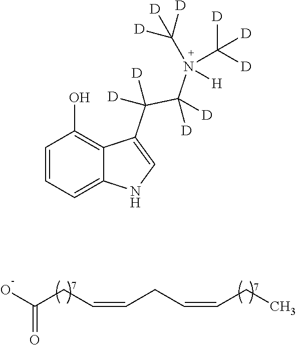

Compound I-3 (PI-d₁₀, free base) (ca. 50 mg) was dissolved in chloroform (ca. 25 mg/mL) in a glass vial. 1 molar equivalent of 1M oleic acid (commercially available Super Refined® oleic acid or oleic acid obtained by desalting sodium oleate with 1M HCl) in chloroform was added to the free base solution and the sample was stirred at room temperature overnight. The solvent was then removed using a Biotage® V-10 evaporator. The resulting material was scraped and stored at −20° C. to induce crystallization. Crystals of I-3r were produced, which were then stored at 5° C. prior to analysis.

As shown in FIG. 95, the X-ray powder diffraction (XRPD) pattern indicates that two different polymorphs were formed: pattern 1 obtained from desalting sodium oleate, and pattern 2 obtained from commercially available oleic acid. Polymorph of pattern 1 of I-3r was poorly crystalline. Polymorph of pattern 2 of I-3r was crystalline. Both polymorphs of I-3r were different from the diffraction patterns 1 and 2 of the free base I-3.

### Example 23

### Synthesis of Caprylate Salt of 3-(2-(bis(methyl-d₃)amino)ethyl-1,1,2,2-d₄)-1H-indol-4-ol (I-3s) (Caprylate Salt of I-3/psilocin-d₁₀/PI-d₁₀)

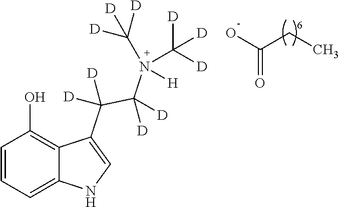

Compound I-3 (PI-d₁₀, free base) (ca. 50 mg) was dissolved in chloroform (ca. 25 mg/mL) in a glass vial. 1 molar equivalent of 1M caprylic acid in chloroform was added to the free base solution and the sample was stirred at room temperature overnight. The solvent was then removed using a Biotage® V-10 evaporator. The resulting material was scraped and stored at −20° C. to induce crystallization. Crystals of I-3s were produced, which were then stored at 5° C. prior to analysis.

As shown in FIG. 96, the X-ray powder diffraction (XRPD) pattern indicates the I-3s salt is crystalline, with only one crystal form observed (pattern 1), which was different from the diffraction patterns 1 and 2 of the free base I-3.

Lipid solubility assessment. Approximately 2-5 mg of test item was added to 0.5 mL of each excipient. The excipients used were corn oil (mixture of unsaturated triglycerides, from Mazola), Crodamol® GTCC (medium chain glyceride, from Croda), and Maisine® CC (mixture of unsaturated mono-, di-, and triglycerides, from Gattefosse). Samples were shaken at room temperature overnight (ca. 18 hours) on an orbital shaker. Where significant solid was present, samples were centrifugated (12500 rpm, 3 min) prior to sampling. A 100 μL aliquot of sample was spiked with 100 μL of internal standard (2 mg/mL benzophenone in 1:1 2-propanol/acetonitrile).

The resulting sample was diluted with diluent (resultant ten-fold dilution). For corn oil the diluent was 3:1 2-propanol/acetonitrile. For Crodamol® GTCC and Maisine® CC, the diluent was 1:1 2-propanol/acetonitrile. The samples were analysed by UPLC. Table 29 presents the lipid solubility data.

TABLE 29

| | |
|---|---|
| Chain length | Salt Concentration (mg/mL) |

| of fatty acid | Test item | Corn oil | GTCC | Maisine CC |
|---|---|---|---|---|
| N/A | I-3 | 0.40 | 1.93 | 1.08 |
| C8 | I-3s | >1.16 | >0.90 | >2.67 |
| C10 | I-3p | 0.40 | 1.33 | 2.17 |
| C12 | I-3m | 0.83 | 2.19 | >1.04 |
| C14 | I-3o | 0.87 | 2.12 | 3.59 |
| C18 | 1-3q | 0.65 | 1.08 | 3.05 |
| | (pattern 2) | | | |
| | 1-3q | 0.64 | 1.17 | 2.48 |
| | (pattern 1) | | | |
| C18, (18:1) | I-3r | 0.71 | 3.03 | >4.02 |
| | (pattern 1) | | | |
| | I-3r | 1.01 | >0.57 | >1.91 |
| | (pattern 2) | | | |
| C18, (18:2) | I-3n | 1.29 | 3.10 | >2.37 |

### V. Psilocin Solution Stability Studies

### Materials and Methods.

Psilocin (I-7/PI/psilocin-d₀/PI-d₀) (free base) (1 mg/ml in acetonitrile:water 1:1, non-schedule) was purchased from Cayman Chemicals. Ascorbic acid (AsA), acetic acid (AcA), tartaric acid (TA), fumaric acid (FA), citric acid (CA), malic acid (MA), benzenesulfonic acid (BSA), stearic acid (SA), sodium citrate dihydrate, monosodium phosphate hydrate, disodium phosphate were purchased from Sigma Aldrich. Aluminum (III) chloride (AlCl₃) and iron (III) chloride (FeCl₃) were purchased from Sigma Aldrich. Antioxidants, i.e., ethylenediaminetetraacetic acid (EDTA), ascorbic acid (AsA), L-cysteine (Cys), sodium metabisulfite (NamBiSO₃) and propyl gallate (PG) were purchased from Sigma Aldrich. Hydroxypropyl-β-cyclodextrin (CAVASOL® W7 HP, CAVITRON® W7 HP7) and methyl-β-cyclodextrin (CAVASOL® W7 M) were provided by DuPont.

The following chromatographic conditions were used:

Column

| | |
|---|---|
| Stationary Phase | ZorbaxSB 18 3.5 mm |
| Material/Dimensions | Stainless steel, 4.6 × 150 mm |
| Mobile Phase A | Water:TFA (100:0.1 v/v) |
| Mobile Phase B | ACN:TFA (100:0.1 v/v) |

| | | | |
|---|---|---|---|
| Gradient Time | Time (Min) | % A | % B |

| | | |
|---|---|---|
| 0.0 | 95 | 5 |
| 1.0 | 95 | 5 |
| 21 | 60 | 40 |
| 27 | 5 | 95 |
| 31 | 5 | 95 |
| 34 | 95 | 5 |
| 36 | 95 | 5 |

| Flow rate | 0.8 | mL/min |
|---|---|---|
| Column Temp | 30° | C. |
| Injection Vol | 10 | μL |
| Needle wash | Diluent | |
| Detection wavelength | 269 | nm |
| Run Time | 36 | min |

Diluent. The diluent used in the below studies was prepared as follows: 100 mL of acetonitrile (ACN) was mixed with 800 mL of water and 100 mL of 1.0 M citric acid and mixed well to prepare 1 L of diluent.

### Stability Studies in Acid Solutions (PI Salt Solutions)

The stability of psilocin in 0.1 M solutions of various acids was tested. Furthermore, psilocin stability was explored in the presence of metal ions (Fe³⁺ and Al³⁺), both with and without the presence of the acids.

Test solutions: To prepare the test solutions, 0.1 M solutions of AsA, AcA, TA, FA, CA, MA, BSA, and SA were prepared in DI water. 10 μM solutions of AlCl₃ and FeCl₃ were prepared in the above solutions. Typically, the psilocin solution was mixed with the 0.1 M acid solution, with or without metal ions, and incubated at 40° C. for designated timepoints (0, 2, 4, 6, 8, 18, 24H). The samples were diluted (1:1) with the diluent before submitting to chromatography to determine the % psilocin remaining.

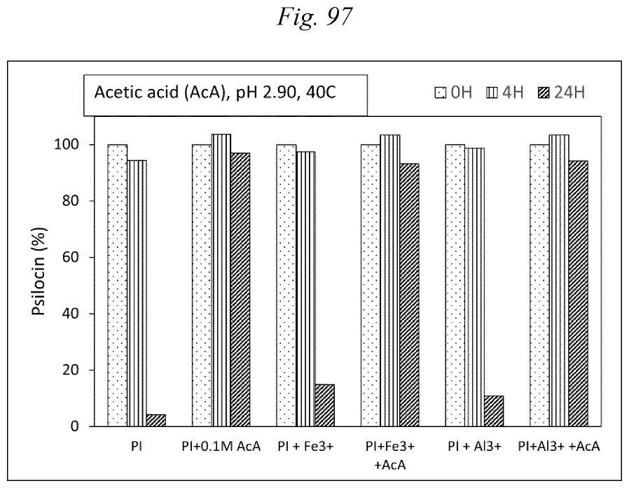

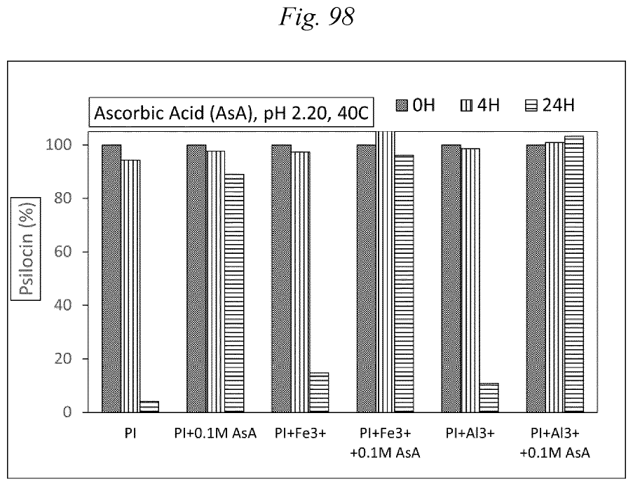

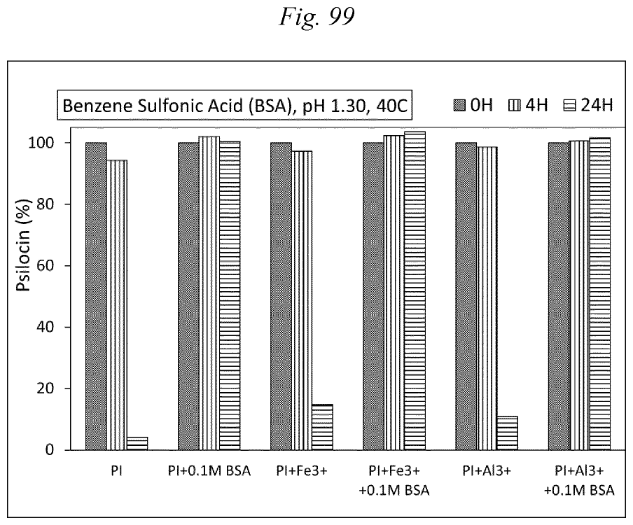

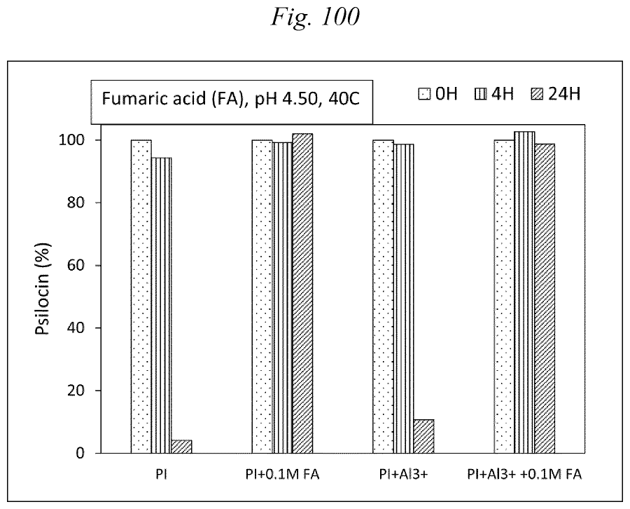

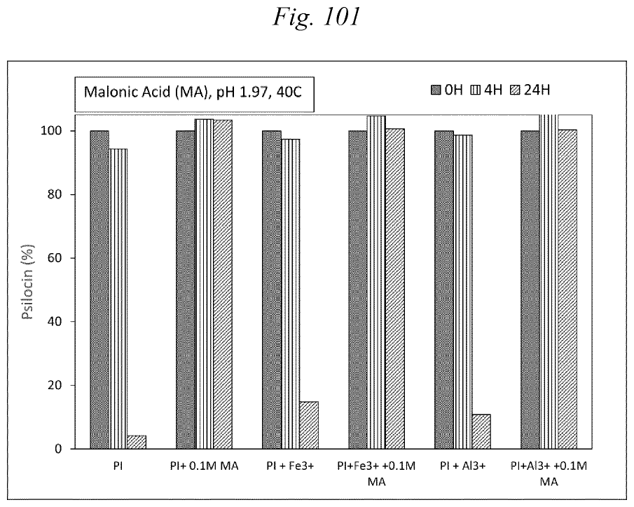

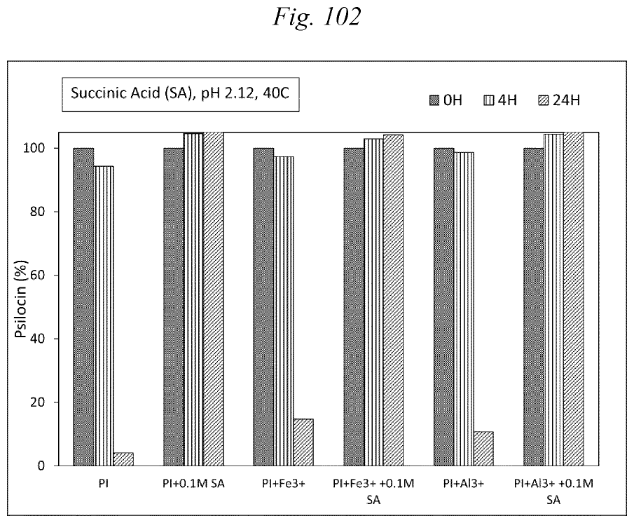

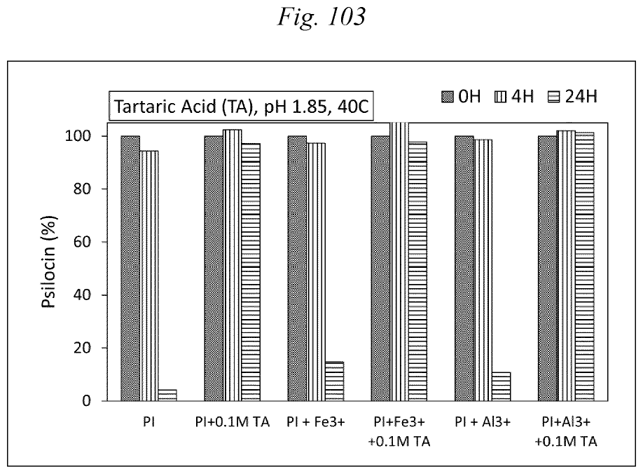

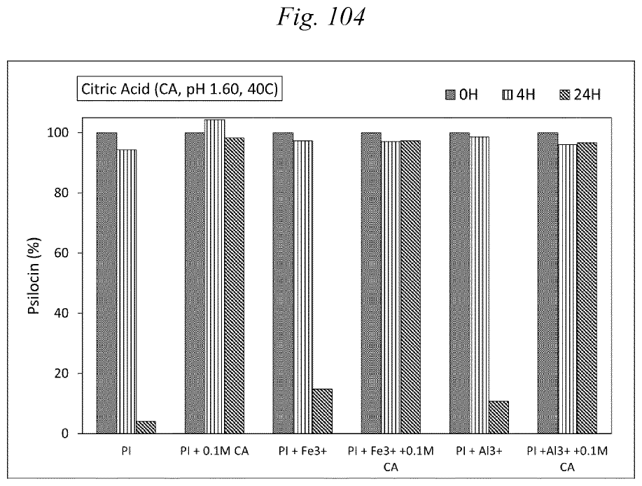

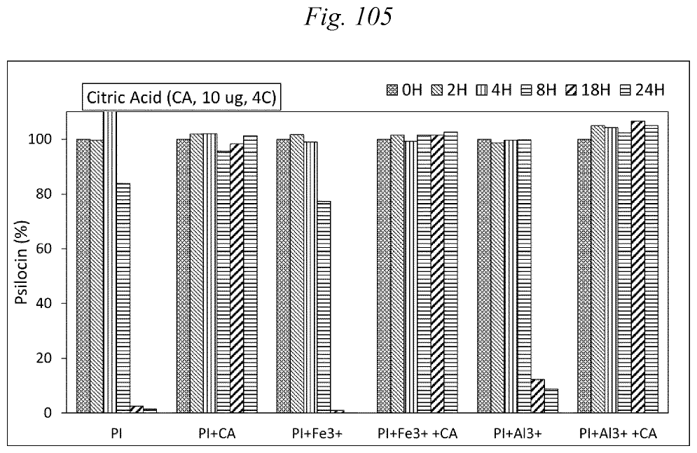

Results: As can be seen from the results presented in FIGS. 97-104, the solution stability of psilocin (free base) without acid was poor, with nearly all psilocin being degraded within 24 hours. The presence of Fe³⁺ and Al³⁺ metal ions in the solutions without acid also resulted in significant psilocin degradation. To the contrary, all tested acid solutions of psilocin (solution-phase salts of psilocin) provided excellent stability for up to 24, the highest time point tested, at 40° C. The stabilizing effect of the various acids was seen in both the acid solutions and those doped with the metal salts.

### Stability Studies in Citric Acid Solution

The stability of psilocin in the presence of citric acid was tested at 4° C., 23° C., and 40° C. Furthermore, psilocin stability was explored in the presence of metal ions (Fe³⁺ and Al³⁺), both with and without the presence of citric acid.

Stock solutions: A 0.2 mg/mI stock solution of citric acid was prepared in DI water with a pH of 3.2. 20 μM stock solutions of AlCl₃ and FeCl₃ were freshly prepared in the above prepared 0.2 mg/ml citric acid solution.

Test solutions: To prepare the test solutions, psilocin solution (1 mg/ml) was mixed with (i) 0.2 mg/ml citric acid solution (1:1 v/v), (ii) 20 μM FeCl₃, and (iii) 20 μM AlCl₃ in 1:1 v/v ratio.

The samples were incubated at 4° C., 23° C., and 40° C. for designated timepoints (0, 2, 4, 6, 8, 18, 24H). The samples were diluted (1:1) with the diluent before submitting to chromatography to determine the % psilocin remaining.

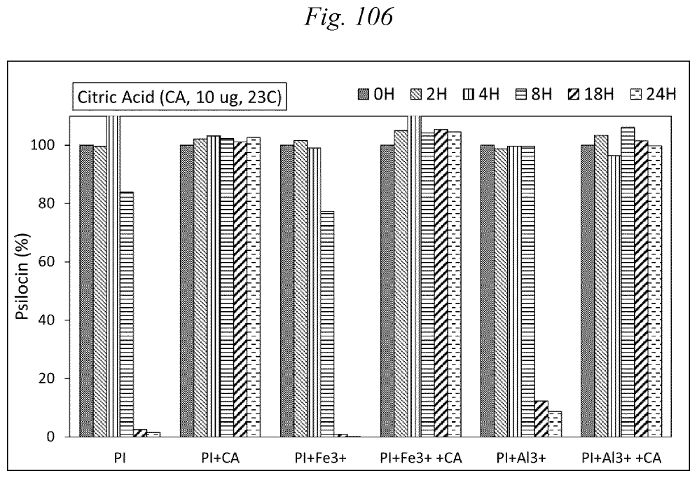

Results: As can be seen from the results presented in FIGS. 105-107, the solution stability of psilocin (free base) in solutions without citric acid was poor, with nearly all psilocin being degraded by the 18 hour time point at all temperatures tested. The presence of Fe³⁺ and Al³⁺ metal ions in these solutions without citric acid also resulted in significant psilocin degradation.

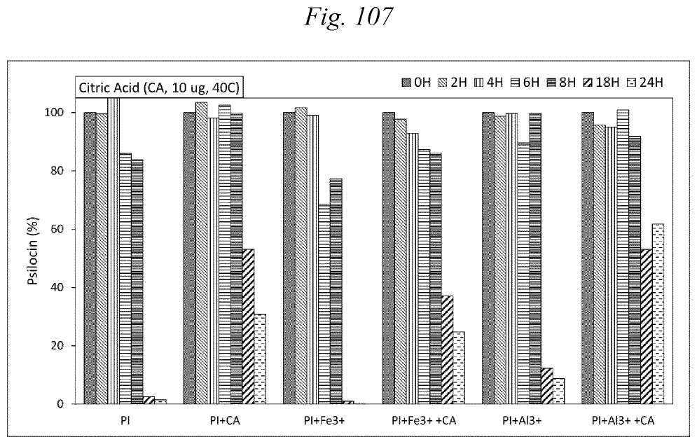

However, even the addition of small quantities of citric acid (10 μg), forming solution-phase citrate salts of psilocin with a pH of 3.2, greatly enhanced the stability of psilocin at 4° C. and room temperature (23° C.). Psilocin stabilization at 40° C. with small quantities of citric acid was not efficient at the 24 hour time point (30%, FIG. 107), but provided increased protection compared to solutions without citric acid. A similar behavior was observed in the presence of trace metals.

### Stability Studies in Sodium Citrate Buffer

The stability of psilocin in the presence of 0.1 M sodium citrate buffer was tested at 4° C., 23° C., and 40° C. Furthermore, psilocin stability was explored in the presence of metal ions (Fe³⁺ and Al³⁺), both with and without the presence of sodium citrate buffer.

Preparation of 0.1 M sodium citrate buffer (pH 6.01): 2.427 g of sodium citrate dihydrate and 0.336 g of citric acid were dissolved in 80 ml of DI water. The pH was adjusted to 6.0 using a 1 M NaOH solution. The final volume was adjusted to 100 ml.

Test solutions: 20 μM stock solutions of AlCl₃ and FeCl₃ were freshly prepared in above prepared 0.1 M sodium citrate buffer. Psilocin (1 mg/ml) was mixed with (i) 0.1 M sodium citrate buffer (1:1 v/v), (ii) 10 μM FeCl₃, and (iii) 10 μM AlCl₃ in 1:1 v/v ratio. The samples were incubated at 4° C., room temperature (RT, 23° C.), and 40° C. for designated timepoints (0, 2, 4, 6, 8, 18, 24H). The samples were diluted (1:1) with the diluent before submitting to chromatography to determine the % psilocin remaining.

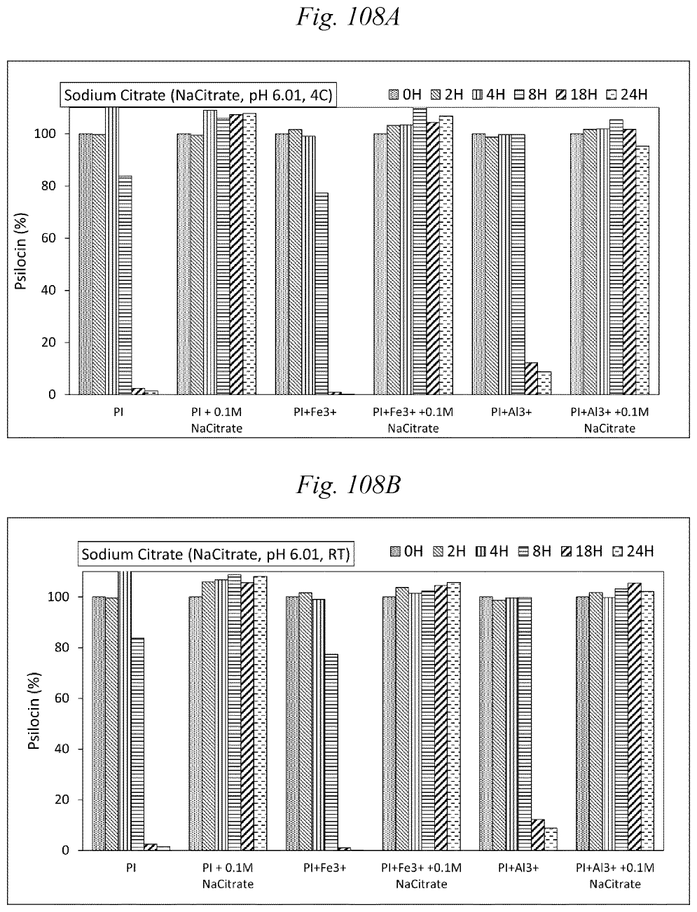

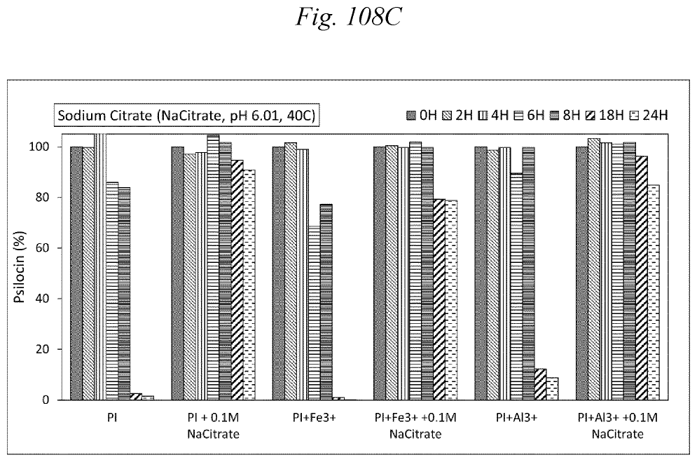

Results: As seen from FIGS. 108A-108C, the solution stability of psilocin (free base) without sodium citrate buffer was poor, with nearly all psilocin being degraded by the 18 hour time point at all temperatures tested. The presence of Fe³⁺ and Al³⁺ metal ions in these non-citrate buffered solutions also resulted in significant psilocin degradation. Conversely, 0.1 M sodium citrate buffer greatly stabilized psilocin at 4° C. and room temperature. Furthermore, no detrimental effects on psilocin were observed due to metal salts (Fe³⁺ and Al³⁺) in the citrate buffer. A minimal degradation (10-15%) of psilocin was observed at the higher temperature (40° C.) condition.

### Stability Comparison Between Sodium Citrate and Phosphate Buffer

The effect of buffer type and pH on the stability of psilocin was assessed.

Preparation of 0.1 M sodium citrate buffer (pH 6.01): 2.427 g of sodium citrate dihydrate and 0.336 g of citric acid were dissolved in 80 ml of DI water. The pH was adjusted to 6.0 using a 1 M NaOH solution. The final volume was adjusted to 100 ml.

Preparation of 0.1 M phosphate buffer (pH 6.0 and pH 7.5): Monosodium phosphate (0.339 g, 0.002 moles) and disodium phosphate (2.021 g, 0.014 moles) were dissolved in 80 ml water and the pH was adjusted as necessary using sodium hydroxide or phosphoric acid. The volume was adjusted to 100 ml using water.

Test solutions: Psilocin (1 mg/ml) was mixed with (i) 0.1 M sodium citrate buffer (pH 6.01), (ii) 0.1 M phosphate buffer (pH 6.0), and (iii) 0.1 M phosphate buffer (pH 7.5) in a 1:1 v/v ratio. The samples were incubated at 40° C. for designated timepoints (0, 2, 4, 6, 8, 18, 24H) and diluted (1:1) with the diluent before submitting to chromatography to determine the % psilocin remaining.

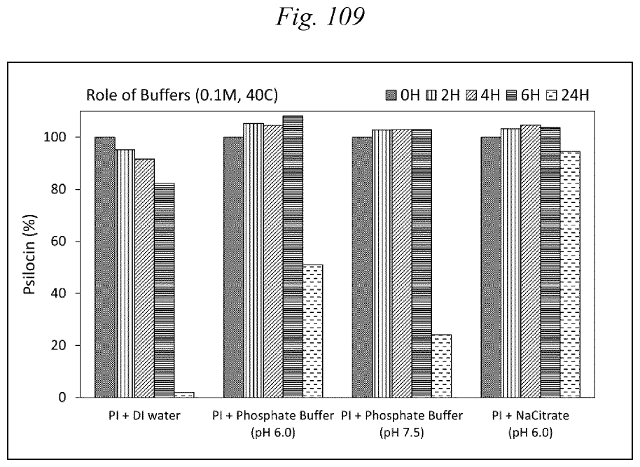

Results: The role of the different buffer solutions and pH on psilocin stability can be seen in FIG. 109. Overall, both phosphate and citrate buffers (pH 6.01 and pH 7.5) provided better stability to psilocin than water at 40° C. after 24 hours. Phosphate buffer at pH 6.0 provided better stability than at pH 7.5. Comparing the two different buffers with the same pH value, citrate buffer (95% efficiency) outperformed phosphate buffer (50%) in stabilizing psilocin at 40° C.

Long Term Stability Studies with Citric Acid and Sodium Citrate Buffer

The long-term stability (up to 25 days) of psilocin in a citric acid solution (0.1 M, pH 1.60) and a sodium citrate buffer (0.1 M, pH 6.01) at 4C and 23° C. was assessed.

Preparation of 0.1 M sodium citrate buffer (pH 6.01): 2.427 g of sodium citrate dihydrate and 0.336 g of citric acid were dissolved in 80 ml of DI water. The pH was adjusted to 6.0 using a 1 M NaOH solution. The final volume was adjusted to 100 ml.

Preparation of 0.1 M citric acid solution (pH 1.60): 0.1 M solution of citric acid was prepared in DI water without any pH adjustment.

Test solutions: Psilocin (1 mg/ml), was mixed with (i) sodium citrate buffer (0.1 M, pH 6.01) and (ii) citric acid solution (0.1 M, pH 1.60) in 1:1 v/v. The samples were stored at 4° C. and 23° C., and aliquots were sampled at designated timepoints (days 0, 7, 17, and 25). The samples were diluted (1:1) with the diluent before submitting to chromatography to determine the % psilocin remaining.

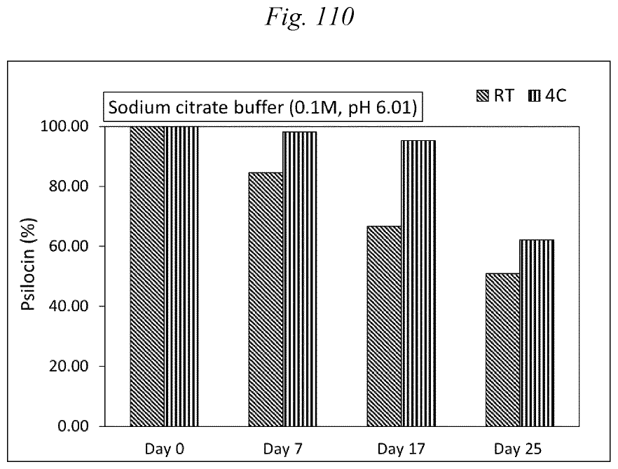

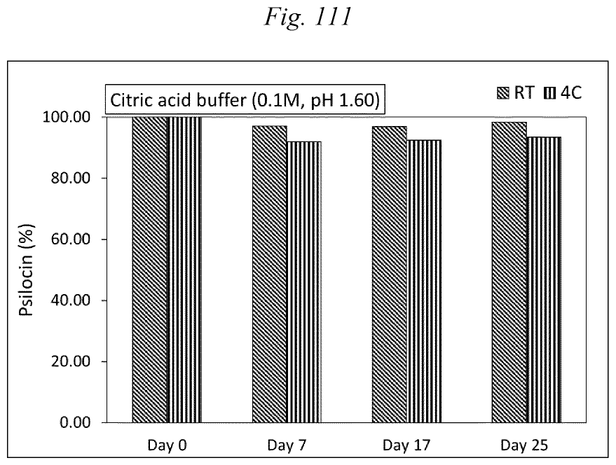

Results: The role of the different buffer solutions on psilocin stability can be seen in FIGS. 110-111. Overall, lower pH (citric acid buffer, pH 1.60) provides the highest level of stabilization to psilocin for both room temperature (23° C.) and 4° C., with only minimal degradation occurring across all 25 day time points at either temperature. Psilocin remained stable for up to 17 days in sodium citrate buffer (pH 6.01) in cold storage (4° C.), but considerable degradation occurred in between the day 17 and 25 day time points. At room temperature, degradation of psilocin began within a week in the sodium citrate buffer conditions.

Stability Studies with Antioxidants

The role of antioxidants in providing stability against metal induced degradation (e.g., oxidation) of psilocin was examined.

Test solutions: 40 μM stock solutions of EDTA, AsA, Cys, NamBiSO₃, and PG were prepared in DI water. 20 μM stock solutions of AlCl₃ and FeCl₃ were freshly prepared in DI water.

Typically, antioxidant was premixed with metal salt in a 1:1 v/v before introducing psilocin. The solution mix was incubated at 40° C. for the designated timepoints (0, 2, 4, 6, 8, 18, 24H). The samples were diluted (1:1) with the diluent before submitting to chromatography to determine the % psilocin remaining.

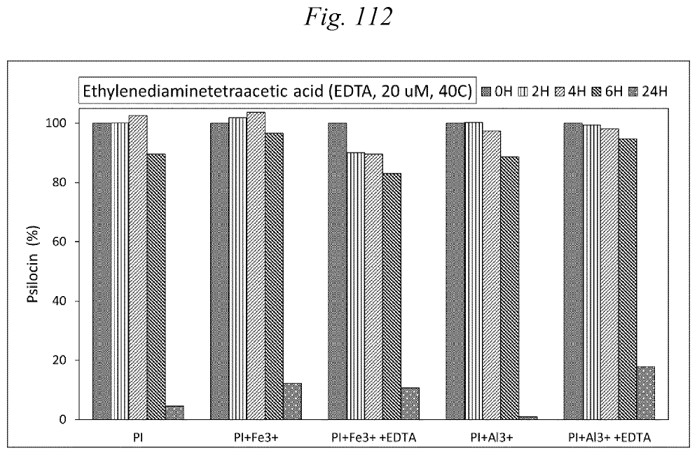

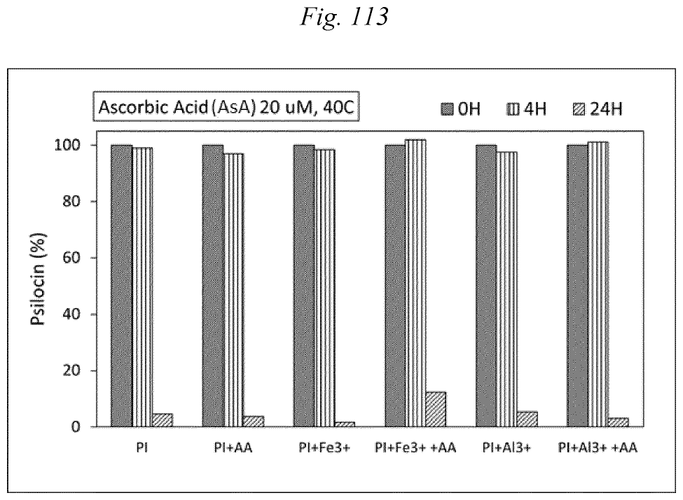

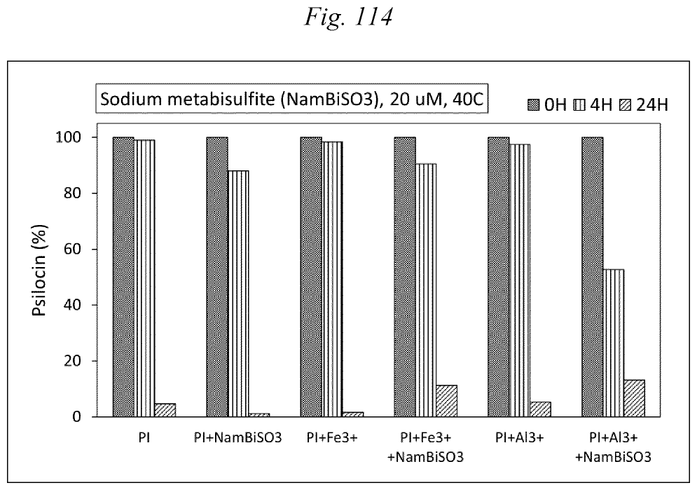

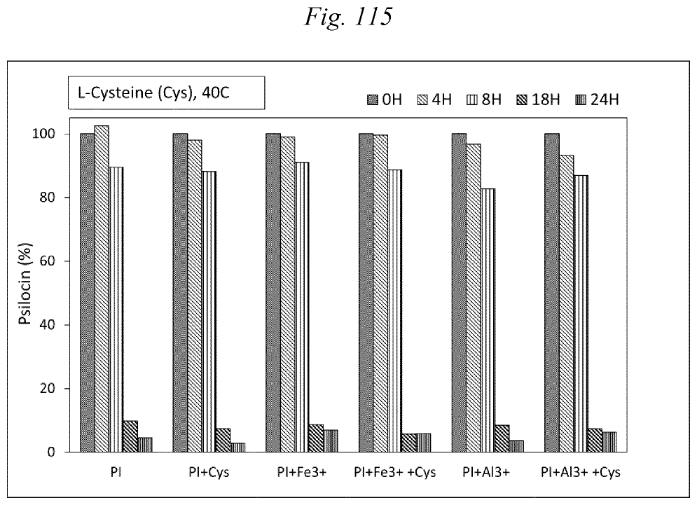

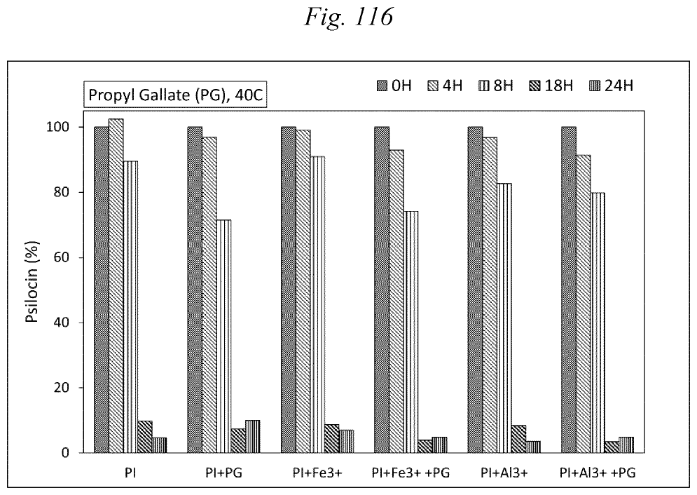

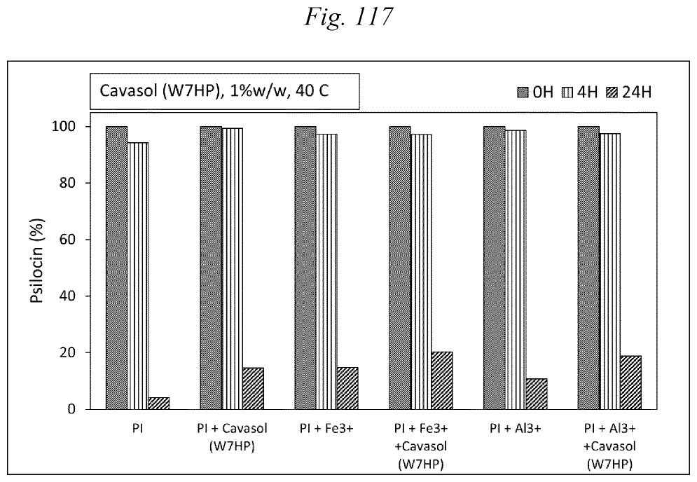

Results: As is evident from FIGS. 112-116, none of the antioxidants tested were able to provide stability to psilocin base against metal salts under the tested accelerated degradation conditions.

Stability Studies with Cyclodextrin Complexes

The role of cyclodextrin complexes in providing stability against metal induced degradation (e.g., oxidation) of psilocin was examined at elevated temperature (40° C.).

Test solutions: Cyclodextrin and metals salt (AlCl₃ and FeCl₃) solutions were prepared in DI water. Typically, psilocin solution (1 mg/ml) was mixed with a 2% (w/w) cyclodextrin in 1:1 (v/v) and incubated at 40° C. for designated timepoints (0, 4, and 24H). Metal salts (10 μM) were also coincubated with the psilocin/cyclodextrin mixture under similar conditions mentioned above. The test samples were diluted (1:1) with the diluent before submitting to chromatography to determine the % psilocin remaining.

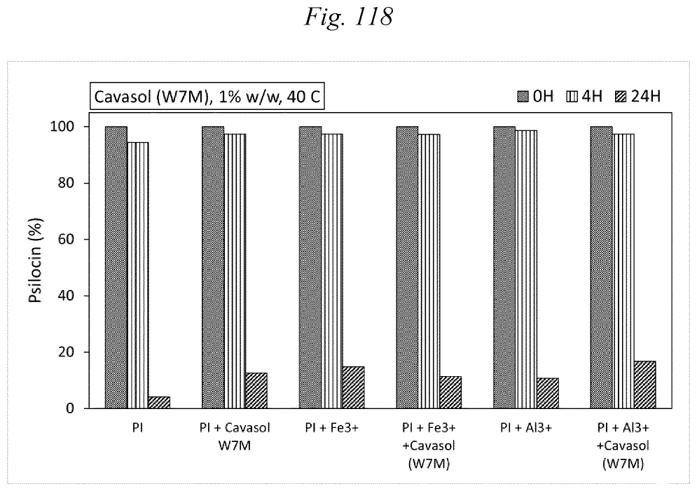

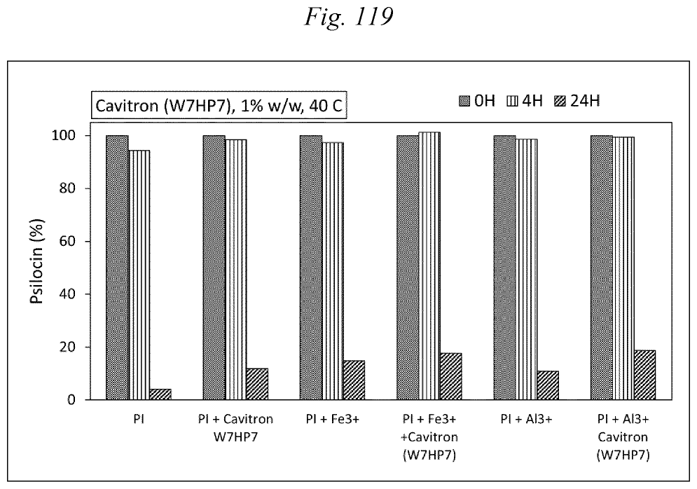

Results: As is evident from FIGS. 117-119, cyclodextrins did not impart stability to psilocin against high temperature (40° C.) or metal induced degradation.

### VI. Simulated Gastric Fluid and Water Solubility Studies

The solubility of free bases, I-3 (PI-d₁₀) (pattern 1) and I-7 (PI-d₀) (pattern 1), and salts, I-3j (pattern 1), I-7a (pattern 1), I-7b (pattern 1), I-7c (pattern 5), I-7j (pattern 1) were determined in FaSSGF (Fasted State Simulated Gastric Fluid) and in water.

Preparation of solutions: Solubility tests in FaSSGF were carried out at 37° C. and in water at room temperature for 2 hour and 6 hour timepoints. 50 mg solid was added to 0.5 mL media and incubated on an orbital shaker for 6 hours. (For free base in water 20 mg samples were used). Slurry/solution was sampled at 2 and 6 hours and aliquots were filtered through 0.45 micron PTFE filters. The filtrate was diluted 500 fold with the appropriate (same) media and injected on to UPLC. Table 30 provides the details of the FaSSGF media.

TABLE 30

| | | Component concentration, mM | | | |
|---|---|---|---|---|---|
| Media | pH | Taurocholate | Phospholipids | Sodium | Chloride |
| FaSSGF | 1.6 | 0.08 | 0.02 | 34 | 59 |

Chromatographic conditions. Aqueous solubility was determined by suspending sufficient compound in water or media to give a maximum final concentration of ≥100 mg·mL⁻¹ of the salt form of the compound. (For free base in water ≥40 mg·mL⁻¹). Solubility was calculated in QuanLynx using the peak areas determined by integration of the peak found at the same retention time as the principal peak in the standard injection. Results were calculated based on a standard calibration curve of appropriate free base. Values quoted are for the free base component of each salt and are average of two (n=2) determinations. The method parameters used are presented in Table 31.

TABLE 31

| Instrumentation | Waters Acquity I class with Quattro-Micro mass | | |
|---|---|---|---|
| | spectrometer and PDAdetector | | |
| Column | BEH C18 1.7 μm 2.1 × 100 mm | | |
| Mobile Phase A | 0.1% aqueous formic acid | | |
| Mobile Phase B | 0.1% formic acid in acetonitrile | | |
| Flow Rate | 0.4 | | |
| (mL · min⁻¹) | | | |
| Gradient Program | Time (mins) | % A | % B |
| | 0.0 | 95 | 5 |
| | 0.1 | 95 | 5 |
| | 1.5 | 5 | 95 |
| | 1.9 | 5 | 95 |
| | 2 | 95 | 5 |
| | 2.75 | 95 | 5 |

| | |
|---|---|
| Injection Volume | 1 |
| (μL) | |
| Detectors | UV, diode array 200-500 nm |
| | MS, mass 100-800 in ES⁺ |
| Data Analysis | Peak area percentage (APCT) with an integration |
| | threshold of 0.2% (relative) |

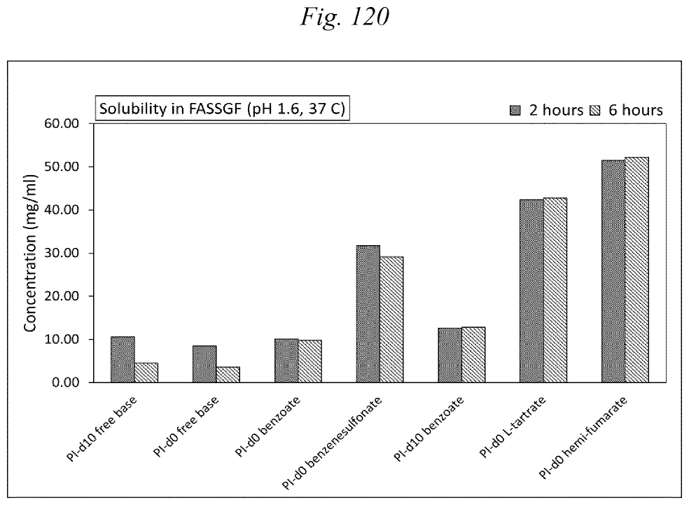

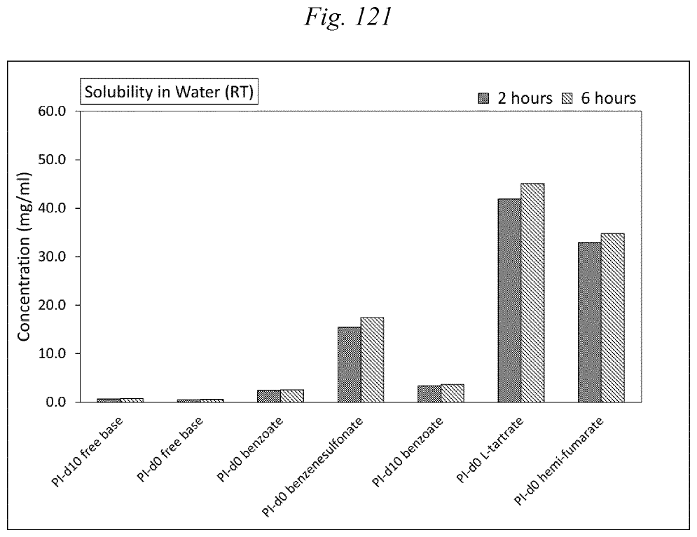

Results: The results are presented in Table 32 and also graphically in FIGS. 120-121. It was observed that salts were generally more soluble than free base and that the solubility in FaSSGF was generally higher than in water. The solubility in FaSSGF (2h) was in the order: I-7<I-7j<I-3<I-3j<I-7a<I-7b=I-7c. Solubility for the free base in FaSSGF decreased at 6h compared to at 2h probably due to decomposition (discoloration observed over time). The following solubility trend was observed in water: I-7<I-3<I-7j<I-3j<I-7a<I-7c<I-7b.

TABLE 32

| | FaSSGF | FaSSGF | Water | Water |
|---|---|---|---|---|
| Solubility (mg/mL) | 2 hours | 6 hours | 2 hours | 6 hours |
| I-3 (PI-d₁₀ free base)(pattern 1) | 10.63 | 4.50 | 0.71 | 0.74 |
| I-7 (PI-d₀ free base)(pattern1) | 8.54 | 3.60 | 0.53 | 0.56 |
| I-7j (pattern 1) | 10.10 | 9.79 | 2.43 | 2.58 |
| I-7a (pattern 1) | 31.71 | 29.11 | 15.46 | 17.49 |
| I-3j (pattern 1) | 12.65 | 12.79 | 3.38 | 3.70 |
| I-7b (pattern 1) | 42.41* | 42.81* | 41.95 | 45.08 |
| I-7c (pattern 5) | 51.51* | 52.25* | 32.94 | 34.78 |

*samples became visually clear with no solid remaining in FaSSGF, so solubility is equal to or greater than the recorded value

### VII. Compositions/Formulations

### Immediate Release (IR) Dosage Form

Immediate release (IR) tablets were formulated with psilocin benzoate (I-7j) (crystalline pattern 1) at a dose of 5.0 mg free base (equivalent to 6.3712 mg of benzoate salt form), with a tablet weight of 80 mg.

Table 33 is a list of materials used for the IR tablet formulations. Tables 34 and 35 provides two different formulations prepared with varying excipients.

TABLE 33

| | | Manufac- | Function- |
|---|---|---|---|
| Material | Type | turer | ality |
| Psilocin benzoate (I-7j) | — | — | API |
| Carboxymethylcellulose, | Ac-Di-Sol ® | Dupont | Disintegrant |
| sodium | SD-711 | | |
| Microcrystalline | Avicel ® | Dupont | Diluent |
| cellulose | PH-102 | | |
| Magnesium stearate | — | Avantor | Lubricant |
| Mannitol | Pearlitol ® | Roquette | Diluent |
| | SD 100 | | |
| Crospovidone | Polyplasdone ® | Ashland | Disintegrant |
| | Ultra | | |
| Sodium stearyl fumarate | PRUV ® | JRS | Lubricant |

TABLE 34
Lot: FS22-001-1A

| Material | % w/w | Per tablet (mg) |
|---|---|---|
| Psilocin benzoate (1-7j) | 7.964 | 6.3712 |
| Microcrystalline cellulose | 88.536 | 70.8288 |
| Carboxymethylcellulose, sodium | 3.000 | 2.4000 |
| Magnesium stearate | 0.500 | 0.4000 |
| Total | 100.000 | 80.000 |

TABLE 35
Lot: FS22-001-1B

| Material | % w/w | Per tablet (mg) |
|---|---|---|
| Psilocin benzoate (I-7j) | 7.964 | 6.3712 |
| Mannitol | 88.036 | 70.4288 |
| Crospovidone | 3.000 | 2.4000 |
| Sodium stearyl fumarate | 1.000 | 0.8000 |
| Total | 100.000 | 80.000 |

Procedure. To prepare Lot: FS22-001-1A, approximately 2 g of psilocin benzoate (I-7j) was delumped by passing it through a 20 mesh sieve and was set aside. Sodium carboxymethylcellulose was delumped by passing through the same 20 mesh sieve and was set aside. The sieve was ‘dry washed’ with all of the dispensed microcrystalline cellulose and set aside. Approximately half of the microcrystalline cellulose was charged in a Turbula blender bottle and was blended for 1 minute to coat the surfaces of the bottle. In order, 1.00 g of the psilocin benzoate (I-7j), sodium carboxymethyl cellulose, and the remainder of the microcrystalline cellulose was charged into the Turbula bottle and blended for 15 minutes. Magnesium stearate was delumped through a 40 mesh sieve and charged into the center of the blend in the Turbula bottle (a hole was dug, the lubricant was added, and the added lubricant was covered over). Blended for 5 minutes. The resulting blend was characterized for bulk and manual tapped density. The 7 mm tooling was inserted into a Carver press and the final blend was compressed at 2 kN, 5 kN, and 8 kN compression levels, which is approximately 500, 1,000, and 1,500 lbs on the Carver press, by weighing out individual aliquots of blend and compressing at the tablet weight of 80 mg.

To prepare Lot: FS22-001-1B, crospovidone was delumped by passing through a 20 mesh sieve and was set aside. The sieve was ‘dry washed’ with all of the dispensed mannitol and set aside. Approximately half of the mannitol was charged in a Turbula blender bottle and was blended for 1 minute to coat the surfaces of the bottle. Sodium stearyl fumarate was delumped through a 40 mesh sieve and set aside. In order, 1.00 g of the previously delumped psilocin benzoate (I-7j) (from protocol above), crospovidone, sodium stearyl fumarate, and the remainder of the mannitol was charged into the Turbula bottle and blended for 15 minutes. The resulting blend was characterized for bulk and manual tapped density. The 7 mm tooling was inserted into a Carver press and the final blend was compressed at 2 kN, 5 kN, and 8 kN compression levels, which is approximately 500, 1,000, and 1,500 lbs on the Carver press, by weighing out individual aliquots of blend and compressing at the tablet weight of 80 mg.

### Orally Disintegrating Tablet (ODT) Dosage Form

Orally disintegrating tablets (ODT) were formulated with psilocin (I-7) as API and either L-tartaric acid or citric acid in Zydis® (Catalent) ODT format, along with a placebo. Six active batches were made using stock mixes at pH 4.5 sub-batched and adjusted to different pH points. Three dose strengths were treated as dose proportional, with wet fill weights of 250 mg, 500 mg, and 1,000 mg for 5 mg, 10 mg, and 20 mg dose strengths, respectively.

Table 36 provides formulation details for stock pre-mix batches 1 and 2 (Z5193/133/1 & 2) and placebo batch (Z5193/133/3). Table 37 provides formulation details for sub-batches Z5193/133/1a-c & 2a-c.

TABLE 36

| | Batch number | | |
|---|---|---|---|
| | Z5193/133/1 | Z5193/133/2 | Z5193/133/1 |
| | (stock | (stock | (Placebo |
| | pre-mix 1) | pre-mix 1) | batch) |
| Material | % w/w | % w/w | % w/w |
| Purified water | 79.00 | 79.00 | 90.49 |
| Gelatin EP/USP/JP | 5.00 | 5.00 | 5.00 |
| (fish HMW) | | | |
| Mannitol EP/USP | 4.00 | 4.00 | 4.00 |
| Psilocin (1-7) | 2.00 | 1.99 | — |
| Citric acid anhydrous | 1.28 | — | 0.51 |
| EP/USP | | | |
| Tartaric acid | — | 1.57 | — |
| Total | 91.28 | 91.56 | 100.00 |

TABLE 37

| | Batch number | | | | | |
|---|---|---|---|---|---|---|
| | 1a | 1b | 1c | 2a | 2b | 2c |
| Material | % w/w | % w/w | % w/w | % w/w | % w/w | % w/w |
| Citric acid anhydrous EP/USP | 1.22 | — | — | — | — | — |
| Tartaric acid | — | — | — | 1.11 | — | — |
| 5% w/w NaOH solution | — | — | 5.99 | — | 3.69 | 6.84 |
| Purified water | 7.50 | 8.72 | 2.73 | 7.33 | 4.75 | 1.60 |
| Total | 100 | 100 | 100 | 100 | 100 | 100 |

To prepare, all excipients were dispensed. Gelatin and mannitol were added to the purified water and the solution was heated to 60° C. and held for 10 mini, while being stirred. The solution was cooled to 12° C. and psilocin was added for preparing active batches. The stock mixes were aliquoted into sub-batches. As indicated, the pH of each sub-batch was adjusted as necessary using a pH modifier (sodium hydroxide). Final water was then added.

Mix pH was measured according to solution hold (SH) times: at the end of mixing (SH0) and again at 24 and 48 hours, SH24 and SH48, with the pH of all acidic batches remaining stable, as shown in Table 38. This stability demonstrates suitability for a commercial manufacturing process, where stability up to a 48-hour solution hold time would typically be required. For the alkaline batches, a trend towards more acidic pH over time was observed, which is typical of alkaline Zydis® formulations. Results for placebo are not given as the properties of the placebo formulation are equivalent to batch 1b.

TABLE 38

| Batch | | Batch pH | Batch pH | Batch pH |
|---|---|---|---|---|
| ID | pH Modifier | at SH0 | at SH24 | at SH48 |
| 1a | Citric acid | 3.55 | 3.55 | 3.61 |
| 1b | Citric acid | 4.53 | 4.50 | 4.55 |
| 1c | Citric acid/NaOH | 7.64 | 7.56 | 7.42 |
| 2a | Tartaric acid | 3.02 | 3.13 | 3.10 |
| 2b | Tartaric acid/NaOH | 4.42 | 4.33 | 4.37 |
| 2c | Tartaric acid/NaOH | 8.11 | 7.94 | 7.75 |

Significant color changes were observed in the formulations over time; this was especially significant in formulations under alkaline conditions. For both acid modifiers, the most acidic condition was consistent in color over 48 hours, but at pH 4.5, both citric acid and tartaric acid formulations saw some darkening; this was more pronounced for the tartaric acid formulation, which was similarly darkened to the alkaline condition by 48 hours. None of the mixes demonstrated precipitation of API over time Dosing and Freezing: The resulting products were dosed into blister pockets with a wet dose weight of 250 mg or 500 mg (proportional dosing). The product was frozen at −90° C. for 4 minutes. The frozen product was placed in a freezer (0° C.) for storage for ≥12 hours.

Freeze Drying: the frozen product was dried in a freeze dryer at a shelf temperature of 0° C. for 12 hours. The dried product was stored in dry storage cabinets at either ambient, fridge (2-8° C.) or freezer (<−20° C.) temperatures.

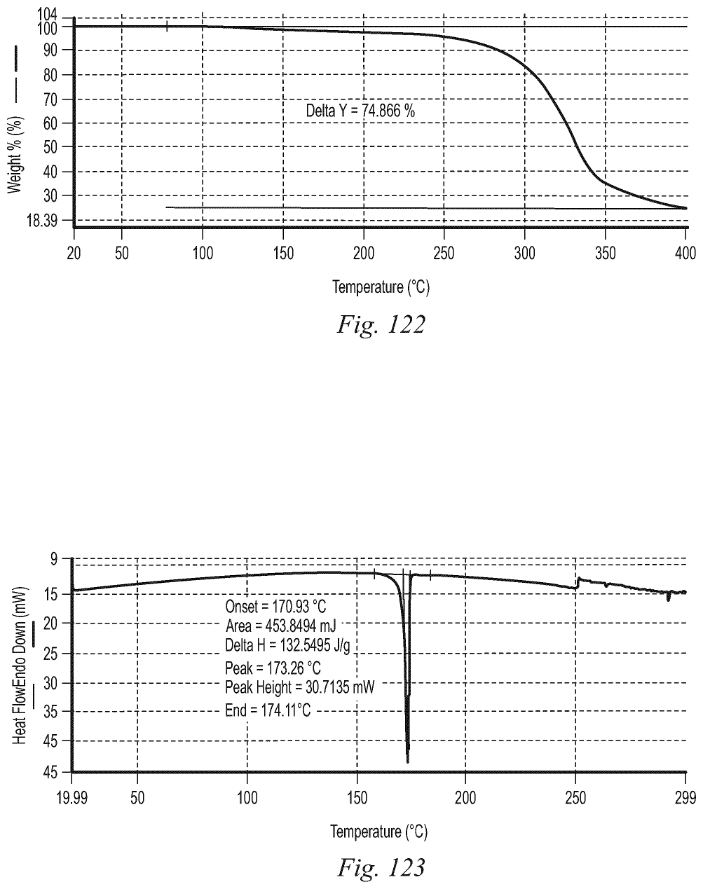

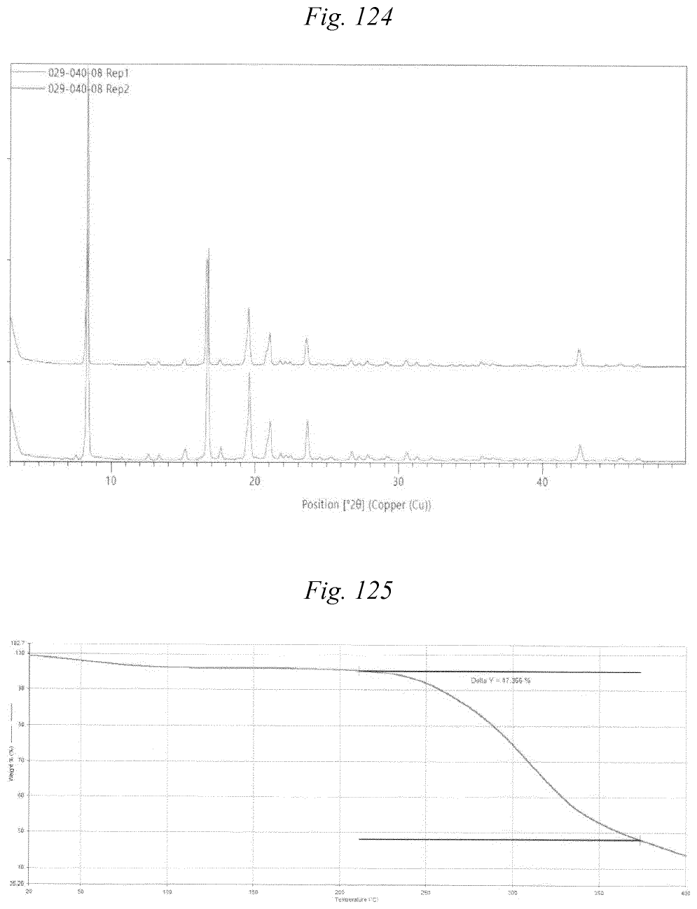

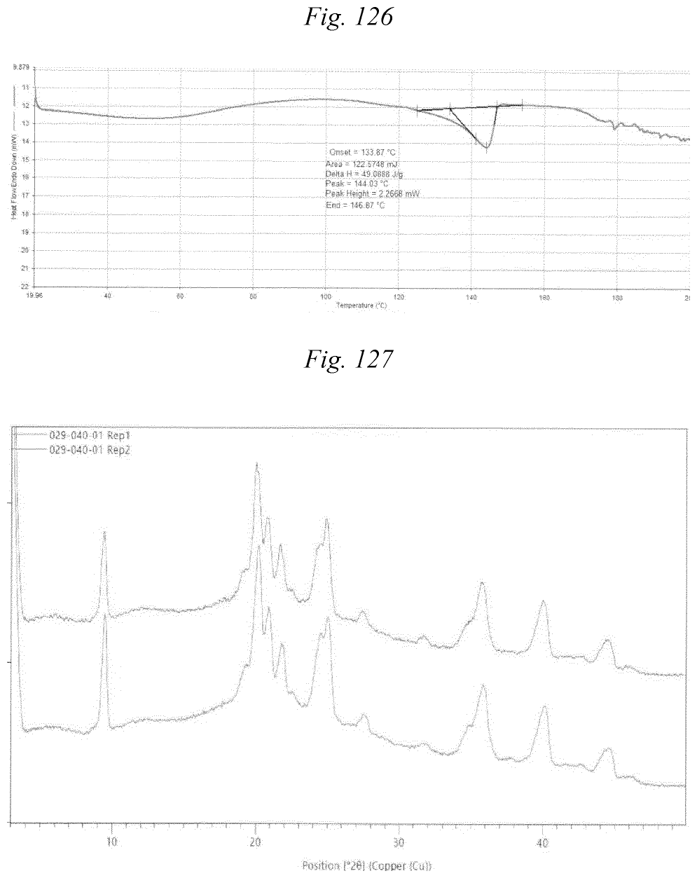

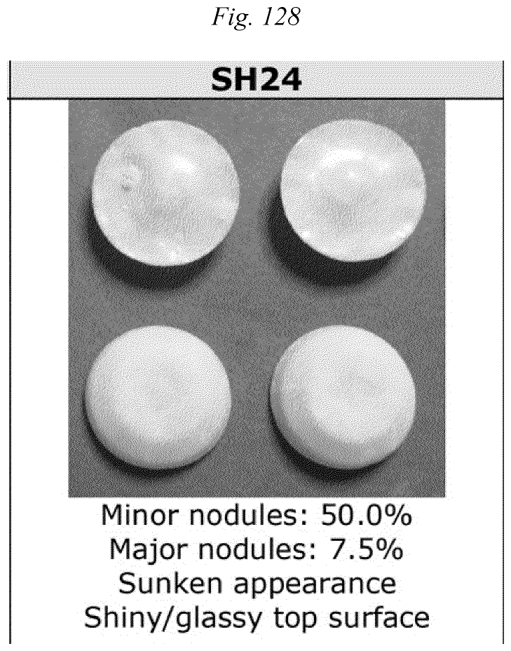

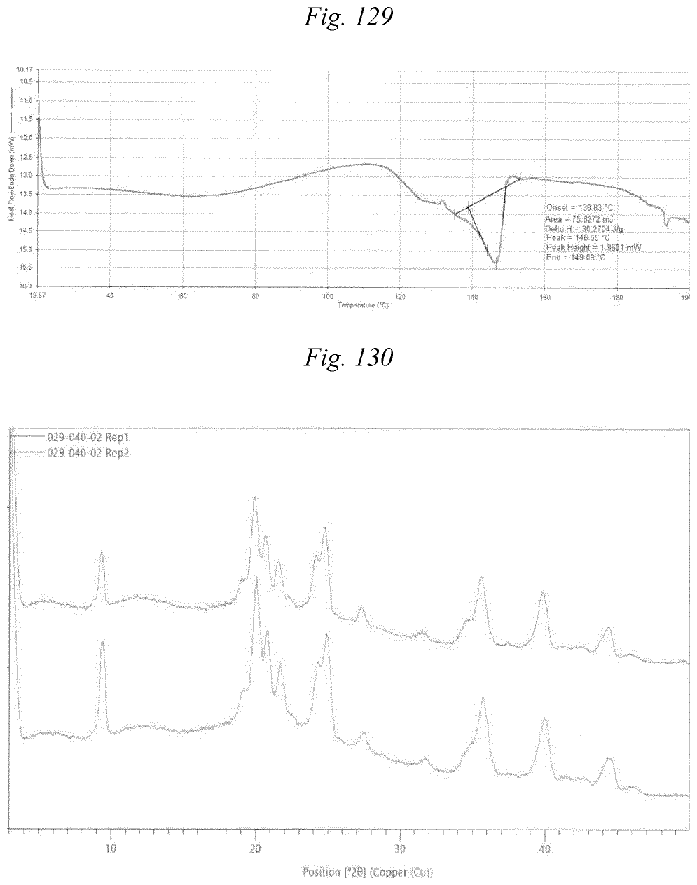

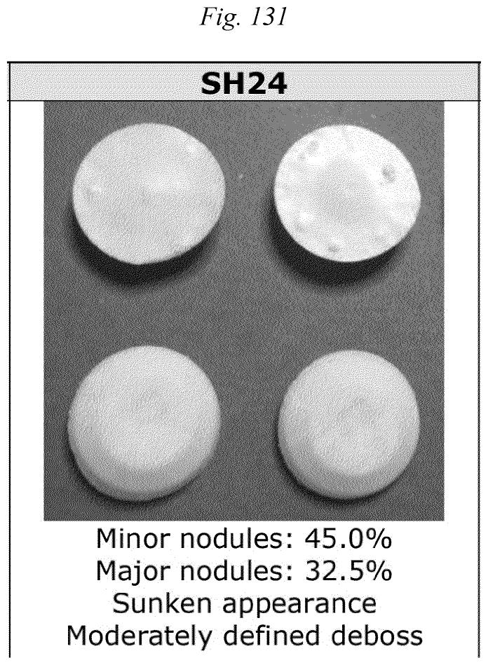

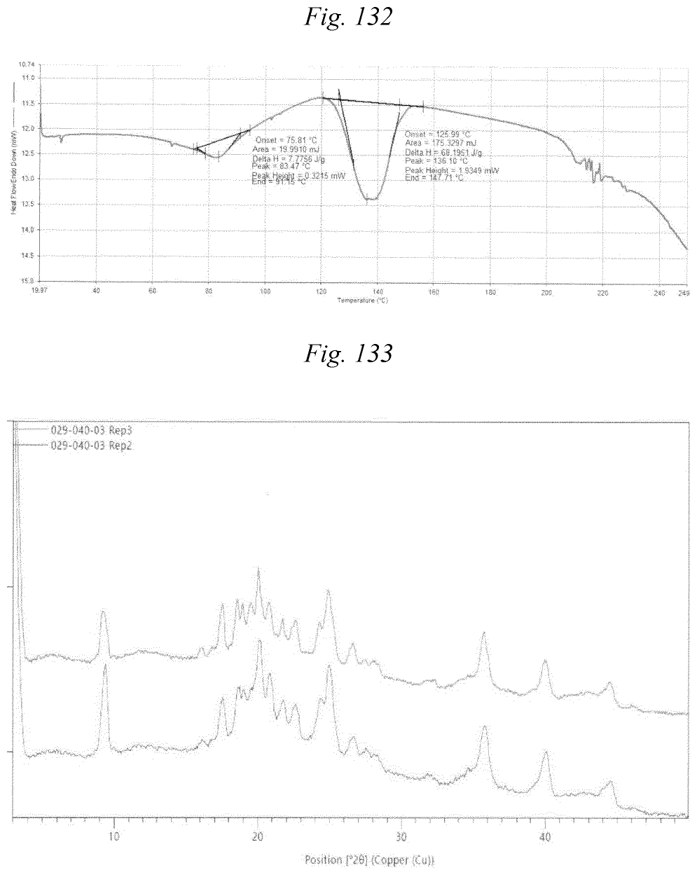

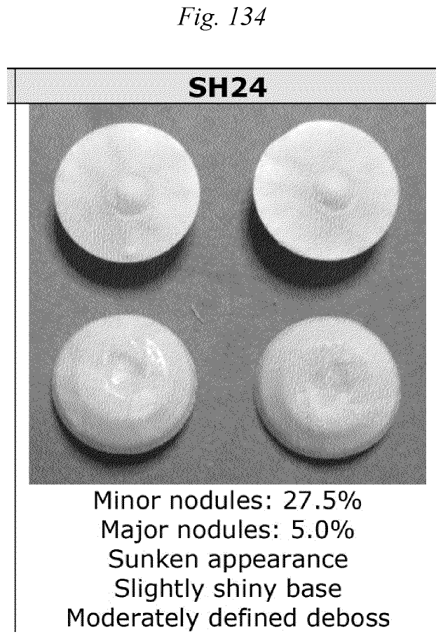

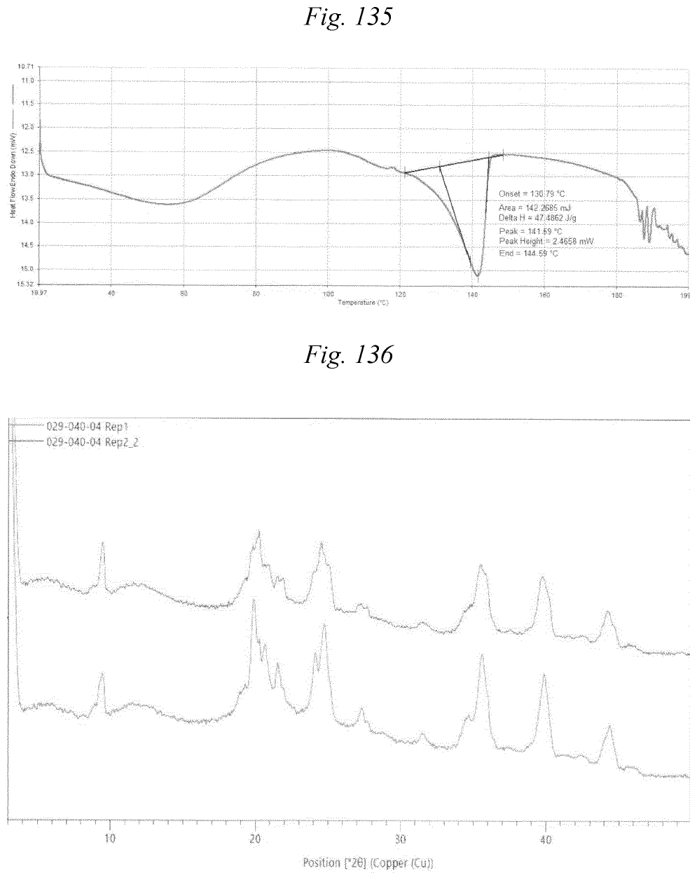

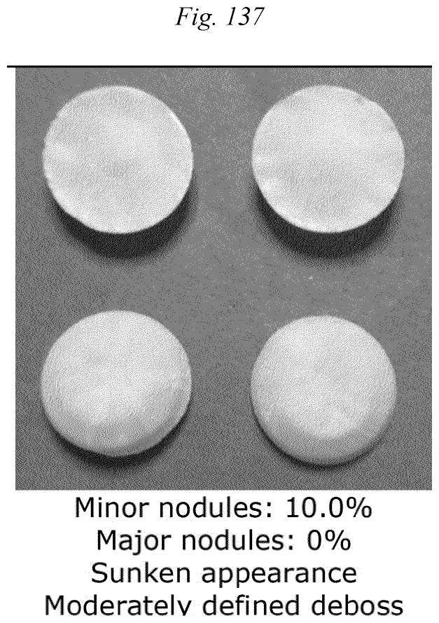

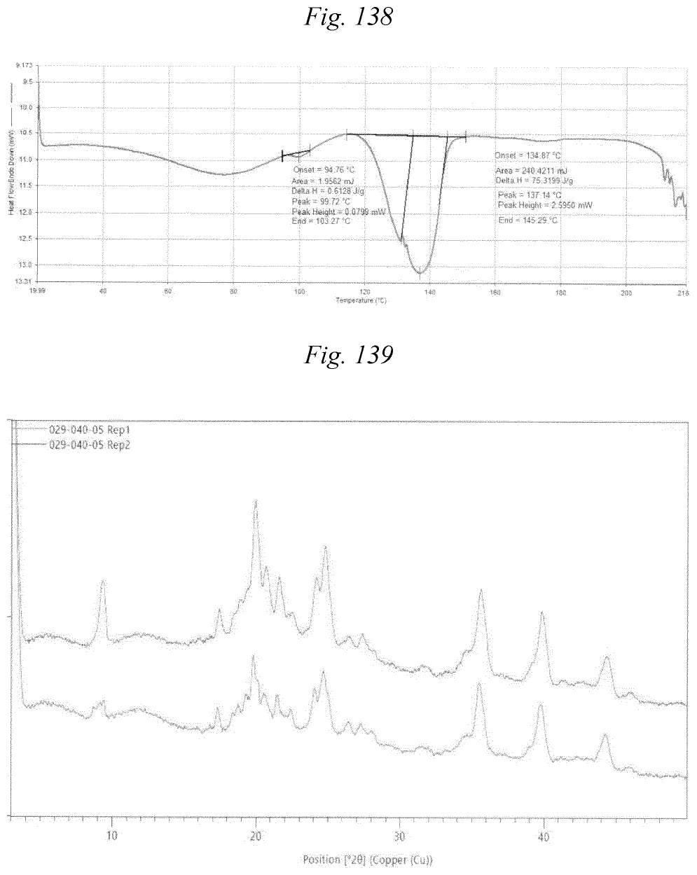

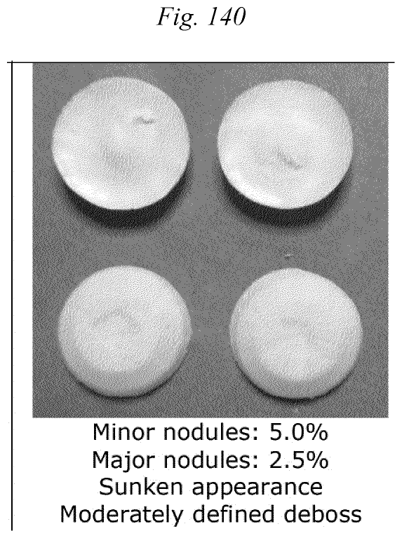

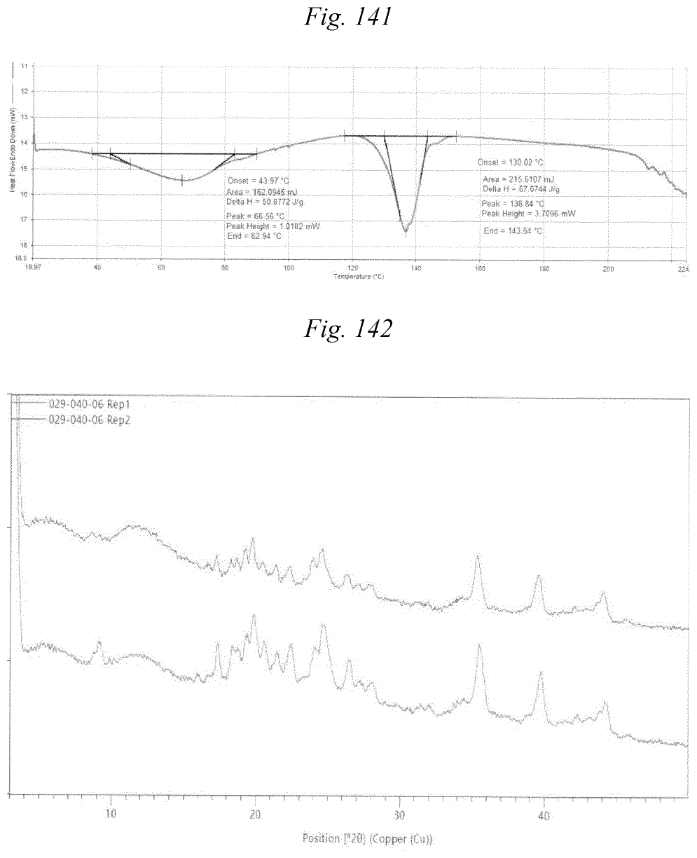

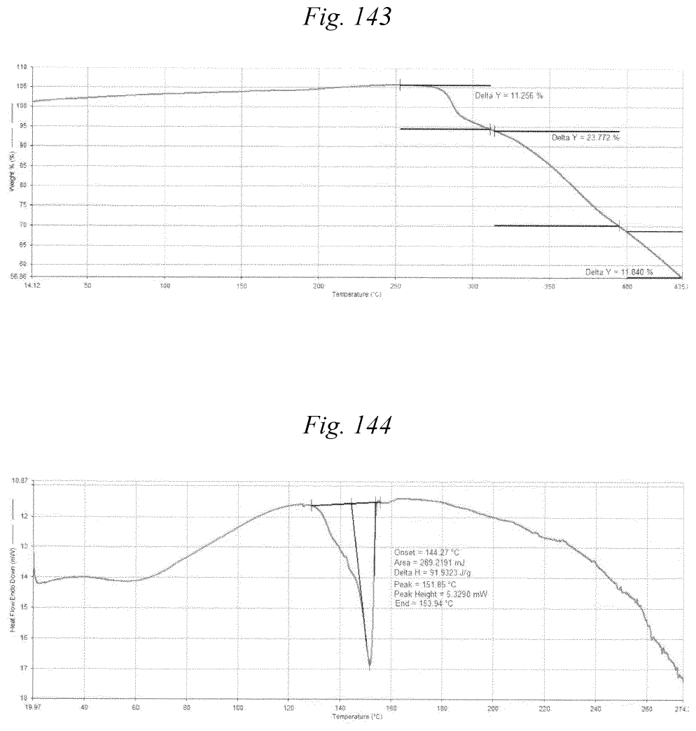

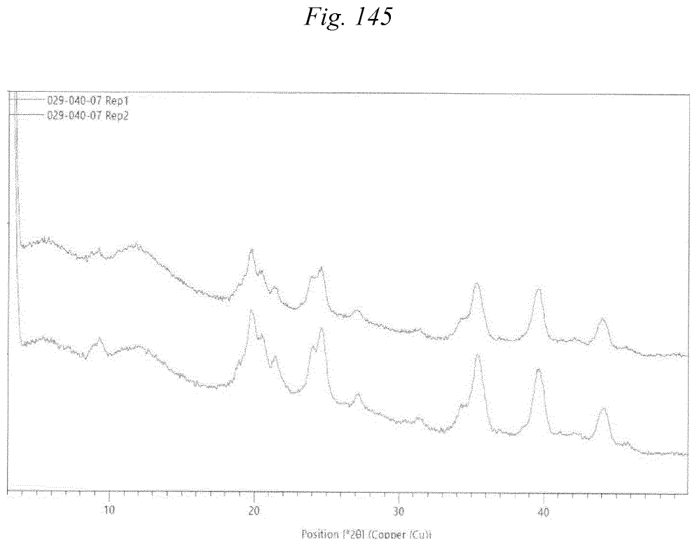

Finished product analysis. FIGS. 122-124 show the TGA, DSC, and XRPD of I-7 (pattern 1) (API) used in the ODT formulations, respectively. FIGS. 125-128 show the TGA, DSC, XRPD, and appearance, respectively, of the ODT dosage form formed from batch 1a (SH24) formulated with the citrate salt of psilocin at pH 3.55. FIGS. 129-131 show the DSC, XRPD, and appearance, respectively, of the ODT dosage form formed from batch 1b (SH24) formulated with the citrate salt of psilocin at pH 4.50. FIGS. 132-134 show the DSC, XRPD, and appearance, respectively, of the ODT dosage form formed from batch 1c (SH24) formulated with the citrate salt of psilocin at pH 7.56. FIGS. 135-137 show the DSC, XRPD, and appearance, respectively, of the ODT dosage form formed from batch 2a (SH24) formulated with the tartrate salt of psilocin at pH 3.13. FIGS. 138-140 show the DSC, XRPD, and appearance, respectively, of the ODT dosage form formed from batch 2b (SH24) formulated with the tartrate salt of psilocin at pH 4.33. FIGS. 141-142 show the DSC and XRPD, respectively, of the ODT dosage form formed from batch 2c (SH24) formulated with the tartrate salt of psilocin at pH 7.94. FIGS. 143-145 show the TGA, DSC and XRPD, respectively, of the placebo ODT dosage form.

ODT unit dispersion testing was performed, whereby the units were placed bottom surface facing down in a beaker filled with purified water at 20° C. f 5° C. The length of time for the unit to disperse was timed using a calibrated stopwatch. This process is carried out for five units in total. The mean dispersion times for various ODT units is presented in Table 39.

TABLE 39

| | Mean dispersion time | | |
|---|---|---|---|
| | (n = 5) (seconds) | | |
| Batch | SH0 | SH24 | SH48 |
| 1a 5 mg | <5 | <8 | <20 |
| 1a 10 mg | <6 | <11 | <16 |
| 1b 5 mg | <18 | <17 | <47 |
| 1a 10 mg | <12 | <16 | <34 |
| 1c 5 mg | <20 | <15 | <43 |
| 1c 10 mg | <32 | <23 | <30 |
| 2a 5 mg | <8 | <12 | <31 |
| 2a 10 mg | <12 | <15 | <35 |
| 2b 5 mg | <30 | <34 | <35 |
| 2b 10 mg | <47 | <48 | <15 |
| 2c 5 mg | <2 | — | <3 |
| 2c 10 mg | <4 | <41 | <9 |

### Sublingual Tablet Dosage Form

Sublingual tablets are formulated with PI-d₁₀ free base (I-3) (pattern 1) and directly compressible vehicles according to Table 40. Alternatively, PI-d₁₀ free base (I-3) (pattern 2) or a pharmaceutically acceptable salt of I-3 may be used as API. Citrocoat® N is a granular powder made from citric acid as core material with a layer of monosodium citrate (1.5-3.5%) as a shell (available from Jungbunzlauer). Povidone K-30, USP is polyvinylpyrrolidone (PVP) with a K-value range from 27.0-32.4, available from Spectrum™ Chemical Mfg. Corp.

TABLE 40

| Material | Functionality | % w/w |
|---|---|---|
| PI-d₁₀ free base (I-3) | API | 5.0 |
| Citrocoat ® N | Coated organic acid agent | 20.0 |
| Lactose | Binder/filler | 10.0 |
| Mannitol | Diluent | 57.0 |
| Povidone K-30, USP | Binder/filler | 7.0 |
| Magnesium stearate | Lubricant | 1.0 |
| Total | — | 100.0 |

Procedure. Sublingual tablets are prepared by direct compression. All ingredients are passed through a #80 mesh separately. The ingredients are then weighed and mixed in geometrical order and compressed into tablets of 100 mg by direct compression method using 6-mm bi-concave punches on a hand-held single table compression machine.

### Effervescent Tablet Dosage Form

### (i) Direct Compression

Two different effervescent tablet formulations formulated with PI-d₁₀ free base (I-3) (pattern 1) and directly compressible vehicles are prepared according to Tables 41 and 42.

Alternatively, PI-d₁₀ free base (I-3) (pattern 2) or a pharmaceutically acceptable salt of I-3 may be used as API. Citrocoat® EP is an effervescent couple in the form of agglomerated granules made by bringing together Citrocoat® N (coated organic acid agent; citric acid core coated with a layer of monosodium citrate, 1.5-3.5%, as a shell) and sodium bicarbonate (source of carbon dioxide) using gum arabic as binder (available from Jungbunzlauer). Ludipress® LCE is a mixture of lactose monohydrate (96.5%) and Kollidon® 30 (3.5%) in free-flowing granule form (available from BASF). Kollidon® 30 is an amorphous, water-soluble polyvinylpyrrolidone with a weight average molecular weight of 44,000-54,000 g/mol and a compendial K-value range of 28-32 (available from BASF). Mannogem® XL is compendial grade direct compression spray dried mannitol (available from SPI Pharma).

TABLE 41

| Material | Functionality | % w/w |
|---|---|---|
| PI-d₁₀ free base (I-3) | API | 5.0 |
| Citrocoat ® EP | Effervescent couple | 30.0 |
| Ludipress ® LCE | Binder/filler | 60.0 |
| PEG 4,000 | Lubricant | 5.0 |
| Total | — | 100.0 |

TABLE 42

| Material | Functionality | % w/w |
|---|---|---|
| PI-d₁₀ free base (I-3) | API | 5.0 |
| Citrocoat ® EP | Effervescent couple | 40.0 |
| Mannogem ® XL | Diluent | 43.0 |
| Orange flavor | Flavoring agent | 1.2 |
| Sucralose | Sweetening/Flavoring agent | 0.8 |
| PEG 8,000 | Lubricant | 10.0 |
| Total | — | 100.0 |

Procedure. Effervescent tablets formulated according to Table 41 or Table 42 are prepared by direct compression. All raw materials except for lubricant are filled into a bin blender and rotated for 5 minutes at 6-10 rpm to form a preblend. Lubricant will be introduced in a later step to avoid over lubrication.

The preblend is passed over an oscillating sieve mill with a screen size of 0.8-1.0 mm to separate non-product related impurities from the raw materials. The preblending step along with the sieving step helps remove lumps and significantly improves the blend uniformity. Sieving is followed by rotating the material in a bin blender for 15-20 minutes at 6-10 rpm. Freshly screened lubricant is added and is blended for 1-2 minutes at 6-10 rpm. Tablets are then punched out using a hand-held single press or rotary press.

### (ii) Wet Granulation

An effervescent tablet formulation formulated with PI-d₁₀ free base (I-3) (pattern 1) is prepared according to Table 43 using a wet granulation method. Alternatively, PI-d₁₀ free base (I-3) (pattern 2) or a pharmaceutically acceptable salt of I-3 may be used as API. Citrocoat® EP is an effervescent couple in the form of agglomerated granules made by bringing together Citrocoat® N (coated organic acid agent; citric acid core coated with a layer of monosodium citrate, 1.5-3.5%, as a shell) and sodium bicarbonate (source of carbon dioxide) using gum arabic as binder (available from Jungbunzlauer). Kollidon® 30 is an amorphous, water-soluble polyvinylpyrrolidone with a weight average molecular weight of 44,000-54,000 g/mol and a compendial K-value range of 28-32 (available from BASF).

TABLE 43

| Material | Functionality | % w/w (wet) | % w/w (dry) |
|---|---|---|---|
| PI-d₁₀ free base (I-3) | API | 5.0 | 5.1 |
| Citrocoat ® EP | Effervescent couple | 40.0 | 40.5 |
| Glucose | Binder/filler | 43.0 | 43.5 |
| Isopropyl alcohol | Granulating solvent | 1.2 | — |
| Kollidon ® 30 | Binder/granulator | 0.8 | 0.8 |
| PEG 6,000 | Lubricant | 10.0 | 10.1 |
| Total | — | 100.0 | 100.0 |

Procedure. Effervescent tablets formulated according to Table 43 are prepared by wet granulation. Fine powdered particles of API, effervescent couple, and binder/filler (in this example, glucose) are mixed. To this mixture is added a solution of binder/granulator (in this example, PVP) in granulating solvent, whereby the fine powdered particles are agglomerated into larger robust wet granules. The wet granules are allowed to dry in hot air oven to remove the granulating solvent, followed by screening to obtain uniform sizes. The lubricant is then mixed with the dried granules before compressing into tablets using either a single punch or a multi-station tablet press fitted with the appropriate punches and dies.

### VIII. In Vivo Studies

Psilocin-d₀/Psilocin-d₁₀ and Psilocybin PK Comparative Study in Rats

The pharmacokinetics and bioavailability of psilocybin, psilocin (PI-d₀), and psilocin-d₁₀ in the rat was investigated following oral gavage and intravenous (bolus) administrations.

The study was designed as shown below in Table 44 to determine if psilocin/psilocin-d₁₀ provides a clinical therapeutic PK profile of fast onset and short duration of action in rats.

TABLE 44

| | | | | Formulated | Volume |
|---|---|---|---|---|---|
| | | | Dose | concentration | dose |
| Group | Treatment | Route | (mg/kg) | (mg/mL) | (mL/kg) |
| 1 | Psilocin + | Intravenous | 1 + 1 | 1 | 1 |
| | psilocin-d10 | (bolus) | | | |
| 2 | Psilocin + | Oral gavage | 5 + 5 | 1 | 5 |
| | psilocin-d10 | | | | |
| 3 | Psilocybin | Intravenous | 2.8 | 2.8 | 1 |
| | | (bolus) | | | |
| 4 | Psilocybin | Oral gavage | 14 | 1.4 | 10 |

Formulations. The vehicle was 0.1 M citrate buffer pH 6 for Groups 1 and 2 and water for injection for Groups 3 and 4.

To make the citrate (0.1M) buffer pH 6 vehicle for Groups 1 and 2, citric acid mono-anhydrous and tri-sodium citrate dihydrate were weighed out and dissolved in water for injection (sterile) to 90% final volume. The pH was checked and adjusted to 6.00±0.1 using NaOH or HCl as require and was then made to final volume and magnetically stirred until visually homogenous. The final pH of the vehicle was adjusted to 6.00±0.1 if required and filtered using 0.22 μm PVDF filter. The required amount of test item was weighed and, using aseptic techniques for the IV preparation, the test item was transferred to a suitable container. The weighing container was rinsed using no more than 15% of final volume of vehicle. It was then made up to 90% of the final volume with the vehicle using sonication and magnetic stirring. The pH was checked and adjusted to 6.00±0.1 using citric acid, then made up to final volume with vehicle. It was stirred for a minimum of 20 minutes using a magnetic stirrer, and whilst under magnetic stirring, the final pH and specific gravity (SG) was checked and recorded. The formulation was then transferred quantitatively to final dispensing container (Amber glass). Prepared on the day of administration and stored refrigerated pending transfer to the animal unit. Formulations were brought to room temperature prior to use.

To make the water vehicle for Groups 3 and 4, the required amount of test item was weighed and, using aseptic techniques for the IV preparation, the test item was transferred to a suitable container. The required volume of water for injection was added and place on a magnetic stirrer. The IV formulation was filtered using 0.22 μm PVDF filter. Formulations were prepared on the day administration and stored refrigerated pending transfer to the animal unit. Formulations were brought to room temperature prior to use.

Animals. Hsd:Sprague Dawley rats from Envigo RMS Limited; 38 males (including 2 spare animals). Spare animals were removed from the study room after treatment commenced.

Rats were given a Teklad 2014C diet, non-restricted. All rats were 7-10 weeks of age at the start of treatment and weight 281 to 319 g.

Administration. Groups I and 3 were given an intravenous (bolus) injection (once) in the lateral tail vein, with a new sterile disposable needle per animal. Animals received constant doses in mg/kg, with a volume dose of 1 mL/kg body weight, calculated from the most recent recorded scheduled body weight. Groups 2 and 4 were dosed by oral gavage (once), using a suitably graduated syringe and a flexible cannula inserted via the mouth. Animals received constant doses in mg/kg, with a volume dose of 5 mL/kg body weight for Group 2 and 10 mL/kg body weight for Group 4, calculated from the most recently recorded scheduled body weight.

Pharmacokinetics. Venous blood samples were taken from animals at the following times in relation to dosing: 0, 5, 15, 30 min, 1, 2, 4 h. Brain samples were taken from animals euthanized at the following time intervals in relation to dosing: 15, 30 min, 1, 2, 4 h (IV); 4 h (Oral). The jugular vein was used as the blood sample site. Terminal bleeds were taken via the sublingual vein to provide larger blood volumes. K₂EDTA was used as anticoagulant. Blood was collected onto wet ice (K₂EDTA tubes). Samples were allowed to stand on wet ice for a minimum of 5 minutes to fully cool, and then harvested to plasma using a refrigerated centrifuge. Centrifugation was performed at 2000 g for 10 minutes at 4° C. Cellular fractions were discarded. After end of centrifugation the samples were returned to wet ice ready for separation. Two 50 μL (serial) or 400 μL (terminal) aliquots were taken per sample, measured accurately using a calibrated pipette and transferred to a plasma tube pre-spiked with 50 μL (serial) or 400 μL (terminal) of stabilizer (200 mM ascorbic acid, 1:1 v:v) and inverted several times to mix thoroughly. Mixing was completed within 30 minutes of plasma separation. Noncompartmental analysis using Phoenix® WinNonlin® was applied to the composite plasma and brain tissue concentration data for Groups 1 and 3 and the individual plasma concentration data for Groups 2 and 4.

Results. The mean pharmacokinetic parameters are summarized in Table 45.

TABLE 45

| | | | | Dose | | | | | | |
|---|---|---|---|---|---|---|---|---|---|---|
| | | Dose | Dose | Level | Cₘₐₓ | Tₘₐₓ | AUC₀₋ₜ | t_{1/2} | CL | Vₛₛ |
| Analyte | Matrix | Group | Route | (mg/kg) | (ng/mL) | (h) | (h*ng/mL) | (h) | (mL/h/kg) | (mL/kg) |
| Psilocin | Plasma | 1 | IV | 1 + 1 | 353 | IAD | 165 | 0.788 | 5910 | 5740 |
| | | 2 | PO | 5 + 5 | 35.2 | 1.67 | 62.0 | NA | — | — |
| | | 3 | IV | 2.8 | 1090 | IAD | 461 | 0.771 | — | — |
| | | 4 | PO | 14 | 116 | 1.33 | 370 | NA | — | — |
| | Brain | 1 | IV | 1 + 1 | 1380 | 0.250 | 1670 | 0.971 | — | — |
| | | 3 | IV | 2.8 | 1940 | 0.500 | 3530 | 0.867 | — | — |
| Psilocin- | Plasma | 1 | IV | 1 + 1 | 308 | IAD | 145 | 0.861 | 6680 | 7020 |
| d₁₀ | | 2 | PO | 5 + 5 | 50.1 | 1.67 | 87.2 | NA | — | — |
| | Brain | 1 | IV | 1 + 1 | 973 | 0.250 | 1530 | 1.27 | — | — |
| Psilocybin | Plasma | 3 | IV | 2.8 | 28700 | IAD | 1910 | NR | NR | NR |
| | | 4 | PO | 14 | 2.88 | 0.528 | 6.54 | NA | — | — |
| | Brain | 3 | IV | 2.8 | 15.0 | 0.250 | 39.2 | NR^{a} | — | — |

IAD Immediately after dosing.
NA Not applicable.
NR Not reportable as there was only one measurable concentration.
NR^{a} Not reportable due to an inability to characterize the elimination phase.
PO Per os (oral gavage)
Groups 1 and 2 were dosed psilocin + psilocin-d10 intravenously and by oral gavage, respectively.
Groups 3 and 4 were dosed psilocybin intravenously and by oral gavage, respectively.

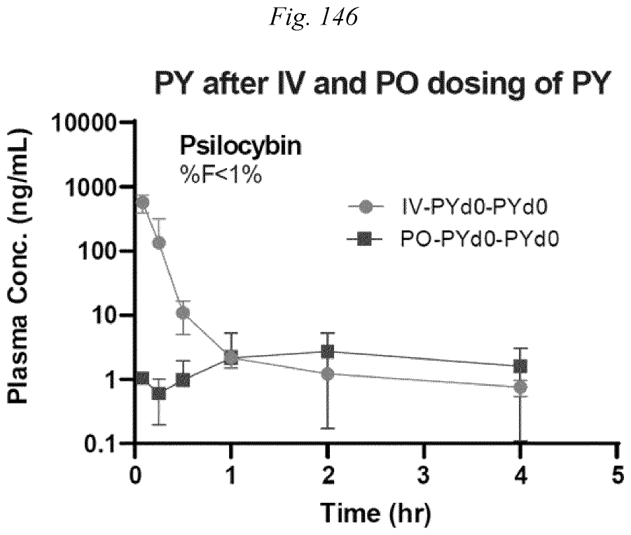

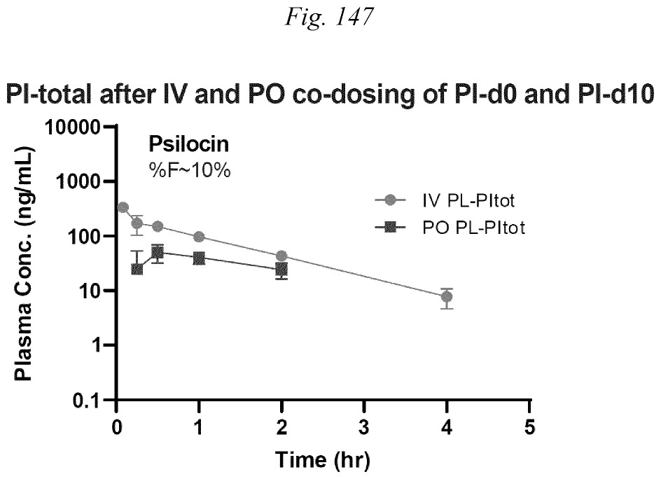

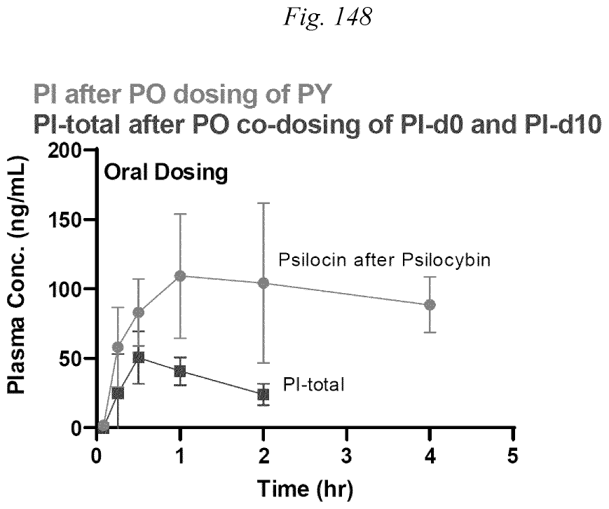

FIGS. 146-147 compare oral bioavailability of psilocin and psilocybin in rats. Psilocybin is not orally bioavailable in rats (<0.1%). By comparison, co-dosing of PI-d₀ and PI-d₁₀ was found to provide oral bioavailabilities of 7.52 and 12.1%, for PI-d₀ and PI-d₁o, respectively. FIG. 148 compares psilocin plasma levels after oral psilocin and oral psilocybin. Oral psilocybin is enzymatically converted to psilocin, thereby adding to variability to psilocin plasma concentrations. The highly variable psilocin plasma concentrations from oral psilocybin can be clearly seen in FIG. 148. On the contrary, oral psilocin, which does not go through an enzymatic step, exhibited less variation in plasma concentrations than oral psilocybin. Oral psilocin also did not display the time-delay effect that burdens psilocybin. These results revealed that oral psilocin exhibits a faster onset, a shorter duration of effect, and less variability in drug exposure than oral psilocybin.

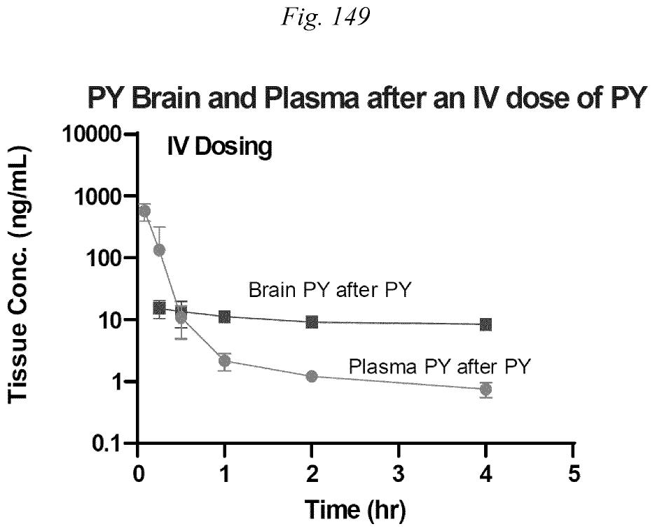

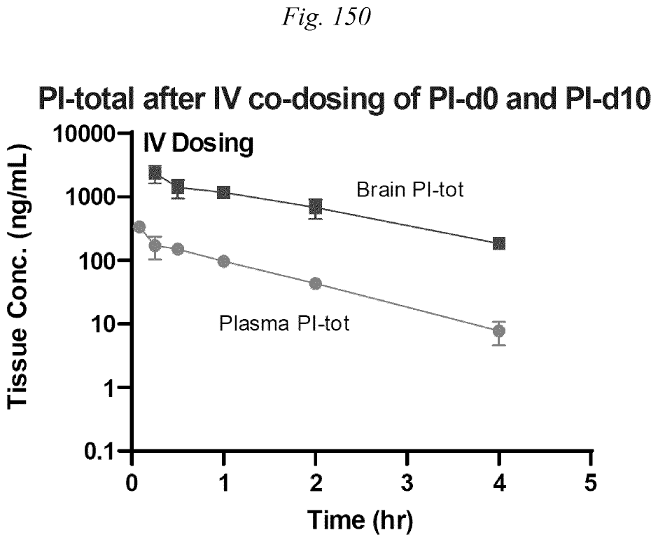

FIGS. 149-150 compares brain and plasma concentration-time profiles after IV dosing. FIG. 149 shows that despite high plasma psilocybin levels, brain concentrations of psilocybin are low after IV dosing. FIG. 150 shows that psilocin (PI-tot) brain concentrations are high compared to plasma concentrations, i.e., psilocin rat brain exposure was much higher than corresponding psilocin plasma levels.

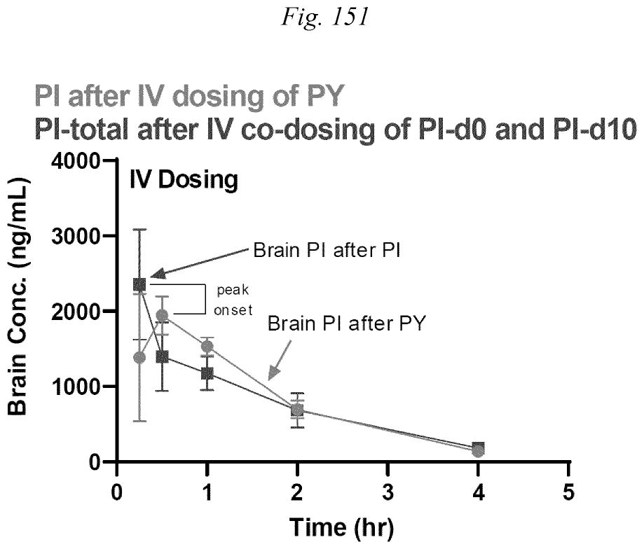

FIG. 151 compares brain levels of PI-tot (PI-d₀+PI-d₀) after IV co-dosing of PI-d₀ and PI-d₁₀, and of brain levels of PI after IV administration of psilocybin (PY). For IV co-dosing of PI-d₀ and PI-d₁₀, brain PI-tot peak concentrations occurred at or before the first sample was taken at 0.25 hr. PI peak levels were at 0.5 hr after PY dosing. It is believed that PI after PY peak levels lag due to metabolism of PY to PI, contributing to slower onset.

The better brain penetration (higher brain:plasma ratio) achieved from administration of psilocin and deuterated psilocin (PI-d₀ and PI-d₁₀) allows for lower effective dosing regimens which would also reduce dose related side-effects, e.g., nausea.

### Psilocin-d₁₀ Pharmacokinetics Study in Dogs Following Oral Gavage and Intravenous (Bolus) Administrations

The pharmacokinetics and bioavailability of psilocin-d₁₀ in dogs were investigated following oral gavage and intravenous (bolus) administrations. The study was designed as shown below in Table 46.

TABLE 46

| | | | | Formulated | Volume | Number/Sex | |
|---|---|---|---|---|---|---|---|
| | | | Dose | concentration | dose | of | Animal |
| Phase* | Treatment | Route | (mg/kg) | (mg/mL)** | (mL/kg) | animals | numbers |
| A | Psilocin- | Intravenous | 0.2 | 0.2 | 1.0 | 3F | 60, 61, |
| | d₁₀ | (bolus) | | | | | 723 |
| B | Psilocin- | Oral gavage | 1.0 | 0.2 | 5.0 | 3F | 60, 61, |
| | d₁₀ | | | | | | 723 |

*7-day stagger between dose administration.
**As supplied, assumes a target volume dose of 5 mL/kg PO, and 1 mL/kg IV

Formulations. The vehicle was 0.1 M citrate buffer pH 6. To make the vehicle, citric acid mono-anhydrous and tri-sodium citrate dehydrate were weighed out and dissolved in water for injection (sterile) to 90% final volume. The pH was checked and adjusted to 6.00±0.1 using NaOH as required, then made to final volume and magnetically stirred until visually homogenous. The final pH of the vehicle was adjusted to 6.00±0.1 when required and filtered using a 0.22 μm PVDF filter. The required amount of test item was weighed out and, using aseptic techniques for the IV preparation, the weighing was transferred to a suitable container and the weighing container rinsed using no more than 15% of final volume of vehicle. This was made up to 90% of the final volume with the vehicle and stirred using magnetic stirring. The pH was checked and no adjustments were required, therefore the formulation was made up to final volume with vehicle. The vehicle was then stirred for a minimum of 20 minutes using a magnetic stirrer and, whilst under magnetic stirring, the final pH and SG were checked and recorded. The formulation was then transferred quantitatively to final dispensing containers (Amber glass). Formulations were prepared on the day of administration and stored refrigerated pending transfer to the animal unit. Formulations were brought to room temperature prior to use.

Animals. Purebred beagle dogs from Marshall BioResources; 3 non-naïve females. Dogs were given a Teklad 2025C Dog Maintenance Diet, 400-500 grams daily. All dogs were 16-20 months of age at the start of treatment and at a weight 8.1 to 9.5 kg.

Administration. Phase A animals were given an intravenous (bolus) injection (once), one hour before feeding in the left or right cephalic vein, with a new sterile disposable needle per animal. Animals were treated at constant dose in mg/kg, with a volume dose of 1 mL/kg body weight, calculated from the most recent recorded scheduled body weight. Phase B animals were dosed by oral gavage (once), one hour before feeding, using a suitably graduated syringe and a rubber catheter inserted via the mouth and down the esophagus. Animals were treated at constant dose in mg/kg, with a volume dose of 5 mL/kg body weight, calculated from the most recently recorded scheduled body weight.

Pharmacokinetics. Psilocin is extremely prone to phenolic oxidation in plasma samples and degradation is extremely rapid in plasma. Therefore, ascorbic acid addition to plasma was required to prevent oxidation and stabilize this analyte. Stabilized incurred plasma samples were then divided into single-use aliquots to avoid repeated freeze-thaw cycles and prolonged bench-top exposure. The analytes have acceptable stability in whole blood on wet ice for the short duration required to process the samples to plasma and stabilize the plasma. To stabilize psilocin-d₁₀ in plasma, a solution of ascorbic acid 200 mM was prepared fresh on the day of use and was added 1:1 (v/v) to control plasma and the harvested plasma from incurred samples. Handling of matrix samples on wet ice was also used. Venous blood samples were obtained from all animals at the following times in relation to dosing: pre-dose (0), 0.25, 0.5, 1, 2, 4, 8 and 24 h. The jugular vein was used as the blood sample site, 1.5 mL blood volume. K₂EDTA was used as anticoagulant. Blood was collected onto wet ice (K₂EDTA tubes). Samples were allowed to stand on wet ice for a minimum of 5 minutes to fully cool, and then harvested to plasma using a refrigerated centrifuge. Centrifugation was performed at 2000 g for 10 minutes at 4° C. within 60 minutes of collection. Cellular fractions were discarded. After end of centrifugation, the samples were returned to wet ice ready for separation. Three 200 μL aliquots were taken per sample, sampled accurately using a calibrated pipette. Plasma tubes used were 0.5 mL, pre spiked with 200 μL of ascorbic acid 200 mM. Ascorbic acid was prepared fresh on the day of use by dissolving 1.76 g of ascorbic acid in 50 mL water, the solution was mixed thoroughly. The solution was stored in amber glass at room temperature and used within 24 hours. Samples were mixed 1:1 v/v with ascorbic acid 200 mM stabilizer solution (200 μL pre-spiked to the plasma tubes). 200 μL of plasma was transferred and measured accurately using a calibrated pipette to a plasma tube pre-spiked with 200 μL of stabilizer and inverted several times to mix thoroughly. Mixing was completed within 30 minutes of plasma separation. The ratio of stabilizer to plasma was 1:1 (v/v); and the addition was checked for accuracy: If the volume of plasma recovered was found to be lower than 200 μL taken, then the amount of stabilizer was adjusted accordingly by removing a volume of stabilizer equal to the difference from the tube using a second calibrated pipette, prior to adding the plasma. Noncompartmental analysis using Phoenix® WinNonlin® was applied to the individual plasma concentration data.

Results. The mean pharmacokinetic parameters for phase A are summarized in Table 47, and the mean pharmacokinetic parameters for phase B are summarized in Table 48.

TABLE 47
Individual and Mean Pharmacokinetic Parameters for Psilocin-d₁₀ in Female
Dog Plasma following a Single Intravenous (Bolus) Administration

| | Dose | | | | DN Cₘₐₓ | | | | DN AUC_{0-inf} | | | |
|---|---|---|---|---|---|---|---|---|---|---|---|---|
| | Level | | C₀ | Cₘₐₓ | (ng/mL)/ | Tₘₐₓ | AUC₀₋ₜ | AUC_{0-inf} | (h*ng/mL)/ | t_{1/2} | CL | Vₛₛ |
| Phase | (mg/kg) | Animal | (ng/mL) | (ng/mL) | (mg/kg) | (h) | (h*ng/mL) | (h*ng/mL) | (mg/kg) | (h) | (mL/h/kg) | (mL/kg) |
| Phase A | 0.20 | 60F | 69.5 | 45.9 | 230 | 0.250 | 67.0 | 73.3 | 366 | 1.16 | 2730 | 3960 |
| | | 61F | 40.5 | 43.9 | 220 | 0.500 | 80.7 | 95.8 | 479 | 1.55 | 2090 | 4280 |
| | | 723F | 62.0 | 47.7 | 239 | 0.250 | 85.8 | 99.9 | 500 | 1.41 | 2000 | 3790 |
| | | Mean | 57.3 | 45.8 | 229 | 0.333 | 77.8 | 89.6 | 448 | 1.37 | 2270 | 4010 |
| | | SD | 15.1 | 1.90 | 9.50 | 0.144 | 9.73 | 14.3 | 71.7 | 0.195 | 398 | 248 |
| | | CV % | 26.3 | 4.15 | 4.15 | 43.3 | 12.5 | 16.0 | 16.0 | 14.2 | 17.5 | 6.18 |
| | | Median | 62.0 | 45.9 | 230 | 0.250 | 80.7 | 95.8 | 479 | 1.41 | 2090 | 3960 |
| | | Min | 40.5 | 43.9 | 220 | 0.250 | 67.0 | 73.3 | 366 | 1.16 | 2000 | 3790 |
| | | Max | 69.5 | 47.7 | 239 | 0.500 | 85.8 | 99.9 | 500 | 1.55 | 2730 | 4280 |
| | | N | 3 | 3 | 3 | 3 | 3 | 3 | 3 | 3 | 3 | 3 |

TABLE 48
Individual and Mean Pharmacokinetic Parameters for Psilocin-d₁₀ in
Female Dog Plasma following a Single Oral (Gavage) Administration

| | Dose Level | | Cₘₐₓ | DN Cₘₐₓ | Tₘₐₓ | AUC₀₋ₜ | AUC_{0-inf} | DN AUC_{0-inf} | t_{1/2} | F |
|---|---|---|---|---|---|---|---|---|---|---|
| Phase | (mg/kg) | Animal | (ng/mL) | (ng/mL)/(mg/kg) | (h) | (h*ng/mL) | (h*ng/mL) | (h*ng/mL)/(mg/kg) | (h) | (%) |
| Phase B | 1.00 | 60F | 132 | 132 | 0.500 | 359 | 374 | 374 | 1.82 | 102 |
| | | 61F | 179 | 179 | 0.500 | 399 | 414 | 414 | 1.69 | 86.5 |
| | | 723F | 126 | 126 | 0.500 | 404 | 426 | 426 | 1.80 | 85.2 |
| | | Mean | 146 | 146 | 0.500 | 387 | 405 | 405 | 1.77 | 91.3 |
| | | SD | 29.0 | 29.0 | 0.00 | 24.9 | 27.1 | 27.1 | 0.0684 | 9.40 |
| | | CV % | 19.9 | 19.9 | 0.00 | 6.42 | 6.71 | 6.71 | 3.86 | 10.3 |
| | | Median | 132 | 132 | 0.500 | 399 | 414 | 414 | 1.80 | 186.5 |
| | | Min | 126 | 126 | 0.500 | 359 | 374 | 374 | 1.69 | 85.2 |
| | | Max | 179 | 179 | 0.500 | 404 | 426 | 426 | 1.82 | 102 |
| | | N | 3 | 3 | 3 | 3 | 3 | 3 | 3 | 3 |

F Bioavailability.
NA Not applicable.

The exposure of psilocin-d₁₀, as assessed by mean Cₘₐₓ and AUC₀₋ₜ values, was 45.8 ng/mL and 146 ng/mL (Cₘₐₓ) and 77.8 h*ng/mL and 387 h*ng/mL (AUC₀₋ₜ) when dosed intravenously and orally, respectively. The mean oral bioavailability of psilocin-d₁₀ at 1 mg/kg was 91.3%. Clearance (CL) was 2270 mL/h/kg, which is similar to the liver blood flow in a 10 kg dog (1854 mL/h/kg), indicating that psilocin-d₁₀ extraction by the liver is a major route of drug clearance. The volume of distribution at steady state (V_{SS}) was 4010 mL/kg, which exceeded the total body water of a 10 kg dog (604 mL/kg), indicating that psilocin-d₁₀ is highly distributed to the tissues following intravenous administration.

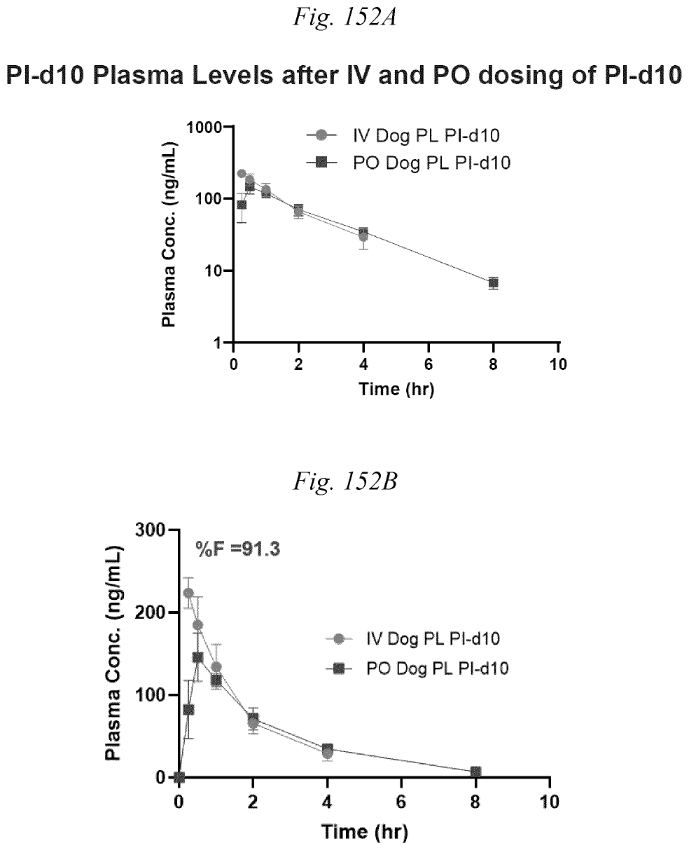

FIG. 152A shows plasma PK profile following IV and oral administration of psilocin-d₁₀. FIG. 152B shows that oral dosing of psilocin-d₁₀ to dogs yielded a fast onset and high bioavailability with elimination similar to an IV dose of psilocin-d₁₀. Peak plasma psilocin-d₁₀ levels occurred at 0.5 hr after oral administration. The last detectable plasma level after oral administration was at 8 hr. The % F of 91.3% indicates that nearly all the given oral dose was distributed to the systemic circulation. Elimination half-lives were similar between IV (1.35 hr) and oral (1.77 hr) doses.

### Pharmacokinetics of Psilocin-do and Psilocybin in the Male Beagle Dog Following Oral Administration by Powder in Capsule (PIC) or Orally Disintegrating Tablet (ODT)

The pharmacokinetic profile of psilocin-d₁₀ and psilocin from psilocybin after oral administration in oral disintegrating tablets (ODTs) or powder in capsule (PIC) dosage forms to male beagle dogs was compared.

Animals. Six, non-naïve, male Beagle dogs aged ca 2-5 years and weighing ca 10-15 kg at dosing were used. These animals were supplied by a recognized supplier of laboratory animals and are currently held as part of a colony (997433). Following study completion, animals were returned to the colony for further use.

Housing. Animals were housed and maintained according to established procedures as detailed in the appropriate Standard Operating Procedures (SOPs). Animals were uniquely identified by tattoo or by microchip. During the pre-trial holding periods, the animals were group housed in caging appropriate to the species. The dogs were housed singly for up to 4 h per day and in this period, had access to their daily ration of diet. The dogs were exercised during the study. Animals were checked regularly throughout the duration of the study. Any clinical signs were closely monitored and recorded. Animals had access to 200-400 g/day of Special Diet Services (SDS) D3 (E) SQC diet throughout the study. Mains quality tap water was available ad libitum.

Test items. Orally disintegrating tablet (ODT) dosage forms were prepared from a stock mix having the following composition; water (86.5% w/w), gelatin (5% w/w, EP/USP/JP (Fish HMW)), mannitol (4% w/w, EP/USP), API (either psilocin-d₁₀ or psilocybin) (2% w/w), citric acid (2.5% w/w, anhydrous EP/USP). Typically, a mixture of gelatin and mannitol was prepared in water and the solution was heated to 60° C. for 10 min. The solution was cooled to 12° C. followed by addition of API. Finally, the pH was adjusted to the desired level. The solution was dosed into blister pockets and subjected to lyophilization by freezing at −90° C. for 4 minutes, placing the frozen product in a freezer (0° C.) for storage for >12 hours, and drying in a freeze dryer at a shelf temperature of 0° C. for 12 hours. Powder in capsule (PIC) dosage forms were prepared using 5 mg of either dry I-3 (psilocin-d₁₀) (pattern 1)(free base) or psilocybin as powder inside a capsule.

### Dose Levels.

Psilocin-d₁₀ as ODT contains 5 mg of active; nominal 0.5 mg/kg active
Psilocin-d₁₀ as PIC contains 5 mg of active; nominal 0.5 mg/kg active
Psilocybin as ODT contains 5 mg of active; nominal 0.5 mg/kg active
Psilocybin as PIC contains 5 mg of active; nominal 0.5 mg/kg active

Experimental design. This is a cross -over study with at least a 7-day washout period between oral administrations. Animals received 5 mg of each test item either via ODT or by PIC. Each animal received a dose level of ca 0.5 mg/kg, but may vary according to the most recent bodyweight of each animal. Bodyweights were recorded for each animal prior to dosing. Oral administration was performed with either an ODT or PIC containing either psilocin-d₁₀ or psilocybin. Capsules were placed at the back of the throat and the animals were encouraged to swallow. Orally disintegrating tablets were placed under the tongue (sublingual). The animal's mouth was held closed for 10 seconds to ensure the tablet was fully dissolved.

Sampling collection. PK samples (ca 1 mL) were collected from the jugular vein by venepuncture into tubes containing K₂EDTA anticoagulant at the following sampling times: Pre-dose, 0.083 (5 min), 0.16 (10 min), 0.25 (15 min), 0.5 (30 min), 1, 2, 4, 8 and 24 hrs post-dose. Immediately following collection, samples were inverted to ensure mixing with anti-coagulant and placed on wet ice. As soon as practically possible, plasma was generated by centrifugation (2500 g, 10 min, 4° C.). All plasma generated was transferred from K₂EDTA tube to aliquot A (per animal/timepoint). Then, 300 μL of plasma and 300 μL (1:1 (v/v)) of 200 mM ascorbic acid were decanted into Aliquot B and stored in a freezer set to maintain a temperature of −65° C., until analysis.

Bioanalysis. Plasma samples were analyzed using an established LC-MS/MS assay (BQL were set at zero prior to Cmax; BQL undefined after Cmax). Plasma samples from the psilocin-d₁₀ ODT and capsule groups were analyzed for psilocin-d₁₀. Plasma samples from the psilocybin ODT and capsule groups were analyzed for psilocybin and psilocin (psilocin-do).

Pharmacokinetic parameters. Noncompartmental pharmacokinetic parameters were determined from the psilocin-d₁₀ and psilocin from psilocybin plasma concentration-time profiles using commercially available software (Phoenix® WinNonlin®).

Results. The data relating to the individual PK parameters are presented in Table 49.

TABLE 49

| | Compound | | | T½ | Tmax | Cmax | AUClast | AUCINF | Vz_F | Cl_F |
|---|---|---|---|---|---|---|---|---|---|---|
| Formulation | Dosed | Analyte | Stats | (hr) | (hr)\hat{ } | (ng/mL) | (hr*ng/mL) | (hr*ng/mL) | (mL/kg) | (mL/kg/hr) |
| ODT | Psilocybin | Psilocin | Mean | 2.06 | 0.25 | 28.9 | 71.8 | 77.0 | 16600 | 5570 |
| | | | SD | 0.475 | 0.25-0.5 | 5.75 | 13.3 | 13.1 | 4420 | 801 |
| PIC | Psilocybin | Psilocin | Mean | 1.8 | 0.5 | 41 | 89 | 93.3 | 12000 | 4580 |
| | | | SD | 0.215 | 0.25-2 | 11.6 | 16.5 | 16.9 | 3400 | 872 |
| ODT | Psilocin- | Psilocin- | Mean | 1.99 | 0.5 | 67.2 | 199 | 213 | 5820 | 2030 |
| | d₁₀ | d₁₀ | SD | 0.21 | 0.25-1 | 12.2 | 41.1 | 44.9 | 1110 | 334 |
| PIC | Psilocin- | Psilocin- | Mean | 1.99 | 1 | 60.0 | 181 | 185 | 6540 | 2270 |
| | d₁₀ | d₁₀ | SD | 0.222 | 0.5-2 | 11.5 | 29.7 | 19.7 | 1070 | 187 |
| PI-d10/PI-PY | | | ODT | 0.966 | | 2.33 | 2.77 | 2.77 | 0.351 | 0.364 |
| | | | PIC | 1.11 | | 1.46 | 2.03 | 1.98 | 0.545 | 0.496 |

*Psilocybin dose of 0.5 mg/kg is equivalent to 0.377 mg/kg of PI-d₁₀; The values in the table have not been corrected to a PI-d₁₀ mg/kg equivalent dose
\hat{ }Tmax values are median (minimum − maximum

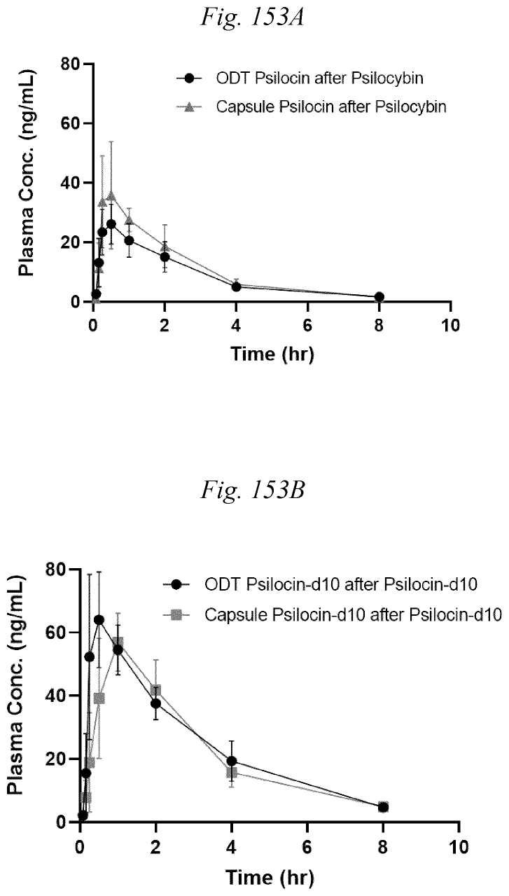

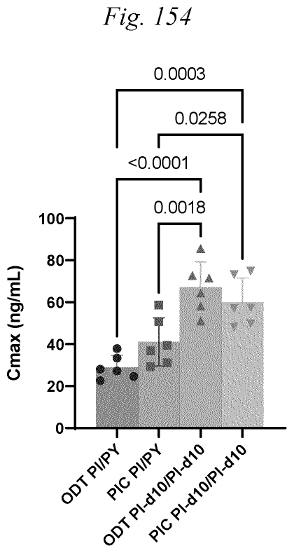

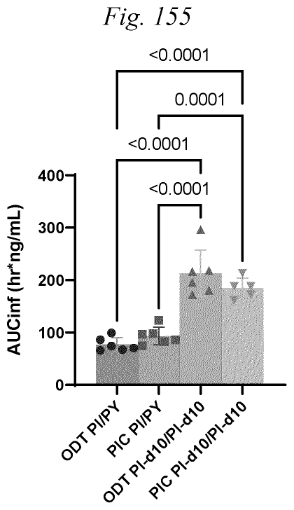

The results are also graphically represented in FIGS. 153A-153B, which show the plasma concentration-time profiles for PI after psilocybin dosing and psilocin-d₁₀ (ODT and PIC dosage forms), respectively, FIG. 154 showing the exposure comparison between psilocybin and psilocin-d₁₀ as assessed by Cmax, and FIG. 155 showing the exposure comparison between psilocybin and psilocin-d₁₀ as assessed by AUCinf.

As can be seen from these graphs, psilocin-d₁₀ and psilocin after psilocybin ODT formulation exposure is not significantly (p>0.5) different than PIC exposure. The ODTs produced a faster onset of action compared to PIC dosage forms as measured by time to maximum plasma concentrations—the time to maximum plasma concentration was twice as fast after ODT compared to PIC (psilocin-d₁₀ median Tmax was 0.5 and 1 hr, ODT and PIC, respectively; psilocin after psilocybin median Tmax was 0.25 and 0.5 hr, ODT and PIC, respectively). However, psilocin-d₁₀ exposure was found to be twice as high as psilocin exposure after administration of psilocybin, independent of formulation.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

Accordingly, the preceding merely illustrates the principles of the methods and compositions. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the following.

1. A pharmaceutical composition, comprising:
a compound of Formula (I), or a pharmaceutically acceptable salt, polymorph, stereoisomer, or solvate thereof, and
a pharmaceutically acceptable vehicle comprising an organic acid agent, 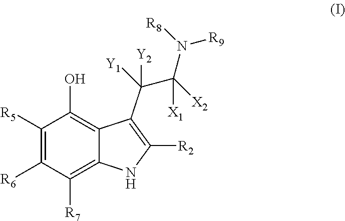
wherein:
R₂, R₅, R₆, and R₇ are independently selected from the group consisting of hydrogen and deuterium,
R₈ and R₉ are independently selected from the group consisting of —CH₃, —CH₂D, —CHD₂, and —CD₃, and
X₁, X₂, Y₁, and Y₂ are independently selected from the group consisting of hydrogen and deuterium.

2-4. (canceled)

5. The pharmaceutical composition of claim 1, wherein R₈ and R₉ are —CD₃.

6. The pharmaceutical composition of claim 1, wherein X₁, X₂, Y₁, and Y₂ are deuterium.

7. The pharmaceutical composition of claim 1, wherein X₁ and X₂are deuterium.

8-9. (canceled)

10. The pharmaceutical composition of claim 1, wherein the compound of Formula (I) is at least one selected from the group consisting of: 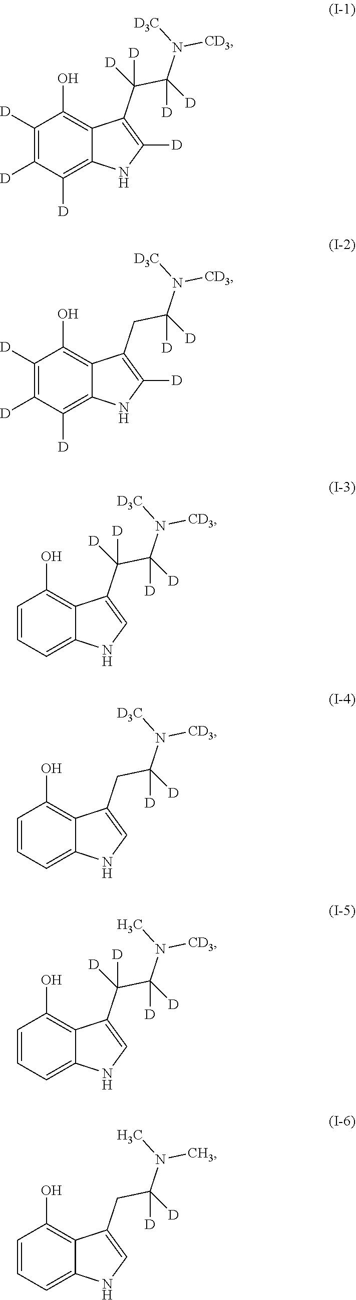 [SMILES: [2H]c1[nH]c2c([2H])c([2H])c([2H])c(O)c2c1C([2H])([2H])C([2H])([2H])N(C([2H])([2H])[2H])C([2H])([2H])[2H] ; [2H]c1[nH]c2c([2H])c([2H])c([2H])c(O)c2c1CC([2H])([2H])N(C([2H])([2H])[2H])C([2H])([2H])[2H] ; [2H]C([2H])([2H])N(C([2H])([2H])[2H])C([2H])([2H])C([2H])([2H])c1c[nH]c2cccc(O)c12 ; [2H]C([2H])([2H])N(C([2H])([2H])[2H])C([2H])([2H])Cc1c[nH]c2cccc(O)c12 ; [2H]C([2H])([2H])N(C)C([2H])([2H])C([2H])([2H])c1c[nH]c2cccc(O)c12 ; [2H]C([2H])(Cc1c[nH]c2cccc(O)c12)N(C)C] 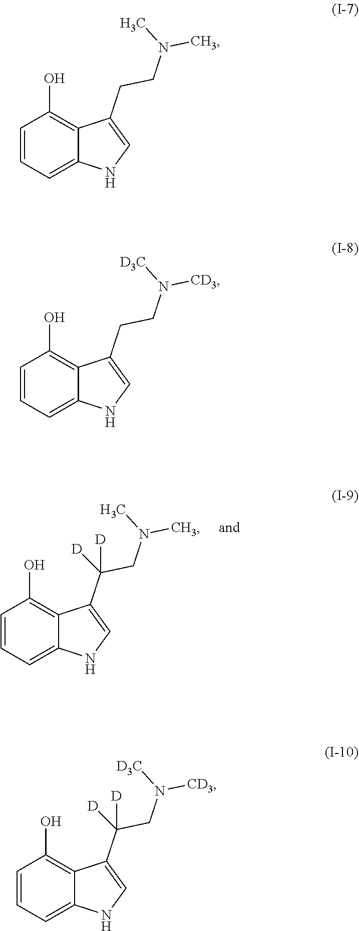 [SMILES: CN(C)CCc1c[nH]c2cccc(O)c12 ; [2H]C([2H])([2H])N(CCc1c[nH]c2cccc(O)c12)C([2H])([2H])[2H] ; [2H]C([2H])(CN(C)C)c1c[nH]c2cccc(O)c12 ; "and" ; [2H]C([2H])(CN(C([2H])([2H])[2H])C([2H])([2H])[2H])c1c[nH]c2cccc(O)c12]
or a pharmaceutically acceptable salt, polymorph, stereoisomer, or solvate thereof.

11. The pharmaceutical composition of claim 1, wherein the compound of Formula (I) is 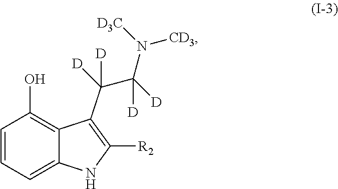
or a pharmaceutically acceptable salt, polymorph, stereoisomer, or solvate thereof.

12-13. (canceled)

14. The pharmaceutical composition of claim 1, wherein the compound of Formula (I) is a crystalline form of 3-(2-(bis(methyl-d₃)amino)ethyl-1,1,2,2-d₄)-1H-indol-4-ol (I-3), as determined by X-ray powder diffraction.

15. The pharmaceutical composition of claim 14, wherein the crystalline form of 3-(2-(bis(methyl-d₃)amino)ethyl-1,1,2,2-d₄)-1H-indol-4-ol (I-3) is:
(i) characterized by an X-ray powder diffraction pattern containing at least three characteristic peaks at diffraction angles (2θ±0.2°) selected from 7.582°, 8.395°, 9.647°, 10.444°, 11.319°, 12.614°, 13.372°, 14.222°, 15.157°, 16.524°, 16.787°, 17.693°, 19.468°, 19.699°, 20.901°, 21.132°, 21.859°, 22.547°, 23.699°, 24.630°, 25.034°, 25.264°, 26.867°, 27.399°, 27.929°, 28.219°, 28.871°, 29.430°, 30.120°, 30.675°, 31.373°, 32.365°, 33.880°, 34.418°, 34.792°, 35.884°, 36.254°, 37.156°, 38.200°, and 38.417°; or
(ii) characterized by an X-ray powder diffraction pattern containing at least three characteristic peaks at diffraction angles (2θ±0.2°) selected from 8.124°, 8.357°, 10.059°, 12.630°, 13.420°, 13.743°, 14.053°, 15.220°, 16.272°, 16.763°, 16.954°, 17.328°, 17.662°, 18.062°, 18.742°, 19.413°, 19.658°, 20.172°, 20.836°, 21.267°, 21.833°, 22.213°, 22.504°, 23.334°, 23.701°, 24.385°, 25.431°, 25.721°, 26.049°, 27.291°, 28.368°, 30.349°, 30.656°, 31.337°, 31.538°, 32.091°, 35.870°, 38.514°, and 41.361°.

16-23. (canceled)

24. The pharmaceutical composition of claim 1, wherein the compound of Formula (I) is present as a pharmaceutically acceptable salt of the compound of Formula (I).

25. The pharmaceutical composition of claim 24, wherein the pharmaceutically acceptable salt of the compound of Formula (I) is crystalline.

26. The pharmaceutical composition of claim 24, wherein the pharmaceutically acceptable salt of the compound of Formula (I) is a benzenesulfonate salt, a tartrate salt, a hemi-fumarate salt, an acetate salt, a citrate salt, a hemi-malonate salt, a fumarate salt, a hemi-succinate salt, an oxalate salt, a benzoate salt, or a salicylate salt of the compound of Formula (I).

27. (canceled)

28. The pharmaceutical composition of claim 24 wherein the pharmaceutically acceptable salt of the compound of Formula (I) is a benzenesulfonate salt of 3-(2-(bis(methyl-d₃)amino)ethyl-1,1,2,2-d₄)-1H-indol-4-ol (I-3a).

29. The pharmaceutical composition of claim 28, wherein the benzenesulfonate salt of 3-(2-(bis(methyl-d₃)amino)ethyl-1,1,2,2-d₄)-1H-indol-4-ol (I-3a) is crystalline and characterized by an X-ray powder diffraction pattern containing at least three characteristic peaks at diffraction angles (2θ±0.2°) selected from 7.023°, 7.767°, 11.822°, 12.550°, 12.860°, 13.994°, 15.521°, 18.436°, 19.503°, 20.760°, 21.070°, 22.007°, 22.745°, 23.340°, 24.187°, 25.532°, 26.880°, 27.856°, 28.163°, 31.267°, 33.024°, 35.030°, 36.835°, 39.312°, 40.545°, and 40.988°.

30-32. (canceled)

33. The pharmaceutical composition of claim 24 wherein the pharmaceutically acceptable salt of the compound of Formula (I) is a tartrate salt of 3-(2-(bis(methyl-d₃)amino)ethyl-1,1,2,2-d₄)-1H-indol-4-ol (I-3b).

34. The pharmaceutical composition of claim 33, wherein the tartrate salt of 3-(2-(bis(methyl-d₃)amino)ethyl-1,1,2,2-d₄)-1H-indol-4-ol (I-3b) is crystalline and characterized by an X-ray powder diffraction pattern containing at least three characteristic peaks at diffraction angles (2θ±0.2°) selected from 6.732°, 12.708°, 13.470°, 14.774°, 15.921°, 16.268°, 17.295°, 18.869°, 20.079°, 20.208°, 20.877°, 21.894°, 22.657°, 23.491°, 23.702°, 24.636°, 24.882°, 25.569°, 26.685°, 27.060°, 27.502°, 28.179°, 28.597°, 29.035°, 29.257°, 29.527°, 31.017°, 31.527°, 32.059°, 32.307°, 33.012°, 34.024°, 34.388°, 34.905°, 35.361°, 36.183°, 37.372°, 37.764°, 38.657°, and 41.049°.

35-38. (canceled)

39. The pharmaceutical composition of claim 24 wherein the pharmaceutically acceptable salt of the compound of Formula (I) is a hemi-fumarate salt of 3-(2-(bis(methyl-d₃)amino)ethyl-1,1,2,2-d₄)-1H-indol-4-ol (I-3c).

40. The pharmaceutical composition of claim 39, wherein the hemi-fumarate salt of 3-(2-(bis(methyl-d₃)amino)ethyl-1,1,2,2-d₄)-1H-indol-4-ol (I-3c) is crystalline and characterized by an X-ray powder diffraction pattern containing at least three characteristic peaks at diffraction angles (2θ±0.2°) selected from 9.713°, 11.209°, 11.605°, 12.338°, 12.852°, 13.718°, 15.117°, 16.066°, 16.627°, 19.026°, 19.427°, 20.108°, 21.068°, 21.335°, 21.837°, 22.429°, 23.262°, 23.478°, 23.900°, 24.720°, 25.318°, 27.912°, 28.532°, 29.565°, 30.457°, 32.698°, 34.155°, 37.910°, 39.566°, and 40.999°.

41-44. (canceled)

45. The pharmaceutical composition of claim 24 wherein the pharmaceutically acceptable salt of the compound of Formula (I) is a citrate salt of 3-(2-(bis(methyl-d₃)amino)ethyl-1,1,2,2-d₄)-1H-indol-4-ol (I-3e), which is amorphous by X-ray powder diffraction.

46-50. (canceled)

51. The pharmaceutical composition of claim 24 wherein the pharmaceutically acceptable salt of the compound of Formula (I) is a benzoate salt of 3-(2-(bis(methyl-d₃)amino)ethyl-1,1,2,2-d₄)-1H-indol-4-ol (I-3j).

52. The pharmaceutical composition of claim 51, wherein the benzoate salt of 3-(2-(bis(methyl-d₃)amino)ethyl-1,1,2,2-d₄)-1H-indol-4-ol (I-3j) is crystalline and characterized by an X-ray powder diffraction pattern containing at least three characteristic peaks at diffraction angles (2θ±0.2°) selected from 9.486°, 11.006°, 12.379°, 13.428°, 14.608°, 15.446°, 16.389°, 18.247°, 18.977°, 19.346°, 19.831°, 20.868°, 21.447°, 22.860°, 23.878°, 24.944°, 25.737°, 26.144°, 26.341°, 26.990°, 27.708°, 28.595°, 30.048°, 30.763°, 31.127°, 31.839°, 32.800°, 34.460°, 35.444°, 37.725°, and 38.597°.

53-56. (canceled)

57. The pharmaceutical composition of claim 1, wherein the organic acid agent is a hydroxy acid and/or an enedioic acid.

58. (canceled)

59. The pharmaceutical composition of claim 1, wherein the organic acid agent is citric acid and/or tartaric acid.

60. The pharmaceutical composition of claim 1, wherein the organic acid agent is citric acid.

61. The pharmaceutical composition of claim 1, wherein the organic acid agent is uncoated.

62-68. (canceled)

69. The pharmaceutical composition of claim 1, wherein the organic acid agent is present in the pharmaceutical composition in an amount of at least 0.5% by weight and up to 6% by weight, based on a total weight of the pharmaceutical composition (on a dry basis).

70. The pharmaceutical composition of claim 1, which is in solid dosage form.

71-77. (canceled)

78. An oral liquid dosage form, prepared by reconstituting the pharmaceutical composition of claim 1 in solid dosage form, in a pharmaceutically acceptable aqueous medium.

79. (canceled)

80. A method of treating a subject with a disease or disorder associated with a serotonin 5-HT₂ receptor, comprising:
administering to the subject a therapeutically effective amount of the pharmaceutical composition of claim 1.

81. The method of claim 80, wherein the disease or disorder is a central nervous system (CNS) disorder.

82. The method of claim 81, wherein the central nervous system (CNS) disorder is at least one selected from the group consisting of major depressive disorder (MDD), treatment-resistant depression (TRD), post-traumatic stress disorder (PTSD), bipolar and related disorders, obsessive-compulsive disorder (OCD), generalized anxiety disorder (GAD), social anxiety disorder, a substance use disorder, an eating disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, suicidal ideation, suicidal behavior, major depressive disorder with suicidal ideation or suicidal behavior, melancholic depression, atypical depression, dysthymia, non-suicidal self-injury disorder (NSSID), chronic fatigue syndrome, Lyme's disease, gambling disorder, a paraphilic disorder, sexual dysfunction, peripheral neuropathy, and obesity.

83. The method of claim 81, wherein the central nervous system (CNS) disorder is major depressive disorder (MDD).

84. The method of claim 81, wherein the central nervous system (CNS) disorder is treatment-resistant depression (TRD).

85-88. (canceled)

89. The method of claim 81, wherein the central nervous system (CNS) disorder is a substance use disorder.

90-91. (canceled)

92. The method of claim 80, wherein the pharmaceutical composition is administered orally to the subject.

93-94. (canceled)

95. The method of claim 80, wherein the pharmaceutical composition is administered to the subject in an amount which provides the compound of Formula (I) at a psychedelic dose of about 0.083 mg/kg to about 5 mg/kg.

96-98. (canceled)
